(19) United States
(12) Patent Application Publication
(10) Pub. No.: US 2026/0274955 A1
(43) Pub. Date: Sep. 17, 2026

(54) ANTIBODIES FOR TARGETING CD28 AND USES THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 19/659,950

(22) Filed: Apr. 27, 2026

Related U.S. Application Data
(63) Continuation of application No. PCT/US24/54219, filed on Nov. 1, 2024.
(60) Provisional application No. 63/595,927, filed on Nov. 3, 2023.

(51) Int. Cl. C07K 16/28; C07K 17/08

(52) U.S. Cl. CPC C07K 16/2818; C07K 17/08; C07K 2317/21; C07K 2317/24; C07K 2317/622

(57) ABSTRACT

Disclosed herein are isolated polypeptide and polypeptide complex compositions that comprise a CD28 binding domain that is linked to a peptide that impairs binding of the CD28 binding domain to CD28.

### CROSS-REFERENCE

The present application is a continuation of International Patent Application No. PCT/US24/54219, filed Nov. 1, 2024, which claims the benefit of U.S. Provisional Application No. 63/595,927 filed on Nov. 3, 2023 which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 25, 2024 is named 52426-761_3011SL.xml and is 1,794,735 bytes in size.

### SUMMARY

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26. In some embodiments, the peptide comprises an amino acid sequence selected from any one of SEQ ID NOs: 1-26. In some embodiments, the peptide consists of an amino acid sequence selected from any one of SEQ ID NOs: 1-26. In some embodiments, the peptide comprises the amino acid sequence of SEQ ID NO: 1. In some embodiments, the peptide consists of the amino acid sequence of SEQ ID NO: 1.

In some embodiments, the peptide comprises an amino acid sequence selected from any one of SEQ ID NOs: 2-19 and 22-26 or the peptide comprises 1, 2, or 3 amino acid substitutions, additions, or deletions relative to an amino acid sequence selected from any one of SEQ ID NOs: 2-19 and 22-26. In some embodiments, the peptide comprises an amino acid sequence selected from any one of SEQ ID NOs: 2-19 and 22-26. In some embodiments, the peptide comprises an amino acid sequence selected from any one of SEQ ID NOs: 2-19. In some embodiments, the peptide consists of an amino acid sequence selected from any one of SEQ ID NOs: 2-19. In some embodiments, the peptide comprises an amino acid sequence selected from any one of SEQ ID NOs: 2-9 and 12-18. In some embodiments, the peptide consists of an amino acid sequence selected from any one of SEQ ID NOs: 2-9 and 12-18. In some embodiments, the peptide comprises an amino acid sequence selected from any one of SEQ ID NOs: 3, 6-7, 9-13, and 16-19. In some embodiments, the peptide comprises an amino acid sequence selected from any one of SEQ ID NOs: 3, 6, 11-12, and 16-17.

In some embodiments, the peptide comprises an amino acid sequence selected from any one of SEQ ID NOs: 22-26. In some embodiments, the peptide consists of an amino acid sequence selected from any one of SEQ ID NOs: 22-26. In some embodiments, the peptide comprises an amino acid sequence selected from any one of SEQ ID NOs: 22-24 and 26. In some embodiments, the peptide comprises an amino acid sequence selected from any one of SEQ ID NOs: 22-23 and 26. In some embodiments, the peptide comprises the amino acid sequence of SEQ ID NO: 23. In some embodiments, the peptide consists of the amino acid sequence of SEQ ID NO: 23.

Disclosed herein, in some embodiments, are isolated polypeptides or polypeptide complexes comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T. In some embodiments, X₁ is selected from A, R, N, D, Q, E, G, H, K, F, P, S, T, W, and Y. In some embodiments, X₁ is selected from R, Q, E, H, I, L, K, F, T, W, Y, and V; X₂ is D; X₃ is P; X₄ is R; X₅ is D; X₆ is L; X₇ is W; X₈ is V; X₉ is H; X₁₀ is F; and X₁₁ is A. In some embodiments, X₁ is selected from R, Q, E, L, K, T, and W; X₂ is D; X₃ is P; X₄ is R; X₅ is D; X₆ is L; X₇ is W; X₈ is V; X₉ is H; X₁₀ is F; and X₁₁ is A. In some embodiments, X₁ is L or I; X₂ is D or Y; X₃ is P; X₄ is R, L, or I; X₅ is H, Y, or D; X₆ is L or R; X₇ is W; X₈ is A, D, H, or N; X₉ is L, E, or D; X₁₀ is F or Y; and X₁₁ is N or H. In some embodiments, X₁ is L or I; X₂ is D or Y; X₃ is P; X₄ is R or I; X₅ is H, Y, or D; X₆ is L or R; X₇ is W; X₈ is A, D, or N; X₉ is L, E, or D; X₁₀ is F or Y; and X₁₁ is N.

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26. In some embodiments, the peptide comprises an amino acid sequence selected from any one of SEQ ID NOs: 2-26. In some embodiments, the peptide consists of an amino acid sequence selected from any one of SEQ ID NOs: 2-26.

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W; Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W; Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is Y; Z₂ is selected from D, E, A, and Q; Z₃ is G; Z₄ is H; Z₅ is selected from L, S, A, T, I, V, and M; Z₆ is selected from P, A, E, Q, S, L, W, G, V, and D; Z₇ is selected from E, Q, I, M, V, A, and L; Z₈ is selected from Y and H; Z₉ is selected from M, I, and L; Z₁₀ is selected from A, Q, S, W, E, L, G, D, T, M, and N; Z₁₁ is K; and Z₁₂ is selected from L and M. In some embodiments, Z₁ is Y; Z₂ is selected from D, E, and A; Z₃ is G; Z₄ is H; Z₅ is selected from L, S, A, and T; Z₆ is selected from P, A, E, Q, S, L, and W; Z₇ is selected from E, Q, I, and M; Z₈ is Y; Z₉ is selected from M, I, and L; Z₁₀ is selected from A, Q, S, W, E, L, G, and D; Z₁₁ is K; and Z₁₂ is L. In some embodiments, Z₁ is Y; Z₂ is selected from D and E; Z₃ is G; Z₄ is H; Z₅ is selected from L, S, and A; Z₆ is selected from P, A, E, Q, S, and L; Z₇ is selected from E, Q, and I; Z₈ is Y; Z₉ is selected from M, I, and L; Z₁₀ is selected from A, Q, S, W, E, L, G, and D; Z₁₁ is K; and Z₁₂ is L.

In some embodiments, the peptide comprises the amino acid sequence of SEQ ID NO: 132. In some embodiments, the peptide consists of the amino acid sequence of SEQ ID NO: 132. In some embodiments, the peptide comprises an amino acid sequence according to any one of SEQ ID NOs: 162-181. In some embodiments, the peptide comprises the amino acid sequence according to SEQ ID NO: 172. In some embodiments, the peptide consists of the amino acid sequence according to SEQ ID NO: 172. In some embodiments, the peptide comprises an amino acid sequence according to any one of SEQ ID NOs: 152 to 161.

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A. In some embodiments, U₁ is selected from T, S, L, M, W, Q, P, R, G, N, I, Y, D, E, V, and H; U₂ is selected from A, S, T, K, E, R, G, N, W, and D; U₃ is selected from A, E, P, D, G, W, Q, V, and F; U₄ is selected from G, V, F, D, E, and R; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, and I; U₈ is selected from P, S, T, A, E, K, Q, N, V, G, H, L, D, Y, I, M, and R; U₉ is selected from W, L, and A; U₁₀ is selected from P, S, R, L, and E; U₁₁ is selected from L, A, T, M, V, W, Y, E, Q, I, F, S, P, K, R, D, H, N, and G; and U₁₂ is selected from L, F, V, M, I, Y, E, and W. In some embodiments, U₁ is selected from T, S, L, M, and W; U₂ is selected from A, S, and T; U₃ is selected from A and E; U₄ is selected from G, V, and F; U₅ is selected from A, F, and V; U₆ is selected from T, S, H, M, A, and N; U₇ is selected from Y, W, and F; U₈ is selected from P, S, T, A, E, K, Q, N, and V; U₉ is selected from W and L; U₁₀ is selected from P and S; U₁₁ is selected from L, A, T, M, V, W, Y, E, Q, I, F, and S; and U₁₂ is selected from L, F, V, and M. In some embodiments, U₁ is selected from T and S; U₂ is selected from A and S; U₃ is A; U₄ is selected from G and V; U₅ is selected from A and F; U₆ is selected from T, S, and H; U₇ is selected from Y and W; U₈ is selected from P, S, T, and A; U₉ is W; U₁₀ is P; U₁₁ is selected from L, A, T, M, V, W, and Y; and U₁₂ is selected from L and F.

In some embodiments, the peptide comprises the amino acid sequence of SEQ ID NO: 137. In some embodiments, the peptide consists of the amino acid sequence of SEQ ID NO: 137. In some embodiments, the peptide comprises an amino acid sequence according to any one of SEQ ID NOs: 144 to 151. In some embodiments, the peptide comprises an amino acid sequence according to any one of SEQ ID NOs: 182-201.

Described herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to any one of SEQ ID NOs: 120-201 and 204-1392.

Described herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

In some embodiments, the CD28 binding domain comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 27, HC-CDR2: SEQ ID NO: 28, and HC-CDR3: SEQ ID NO: 29, and the anti-CD28 antibody comprises an anti-CD28 light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprises amino acid sequences of LC-CDR1: SEQ ID NO: 30, LC-CDR2: (KAS), and LC-CDR3: SEQ ID: 32. In some embodiments, the anti-CD28 heavy chain variable domain comprises an amino acid sequence with at least 80%, 85%, 90%, 95%, 98%, 99%, or 100% sequence identity to SEQ ID NO: 33. In some embodiments, the anti-CD28 light chain variable domain comprises an amino acid sequence with at least 80%, 85%, 90%, 95%, 98%, 99%, or 100% sequence identity to SEQ ID NO: 34.

In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 35. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 67. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 68. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 69. In some embodiments, the anti-CD28 antibody comprises the Fab, wherein the Fab comprises an immunoglobulin light chain with at least 80%, 85%, 90%, 95%, 98%, 99%, or 100% sequence identity to SEQ ID NO: 202, and wherein the Fab comprises an immunoglobulin heavy chain with 80%, 85%, 90%, 95%, 98%, 99%, or 100% sequence identity to SEQ ID NO: 203. In some embodiments, the isolated polypeptide or polypeptide complex is human or humanized.

In some embodiments, the peptide is connected to the CD28 binding domain in a configuration according to A₁-L₁-P₁ wherein A₁ comprises the CD28 binding domain, P₁ comprises the peptide that impairs binding of the CD28 binding domain to CD28, and L₁ comprises a cleavable linker. In some embodiments, P₁ is connected N-terminal to the cleavable linker and A₁ is connected C-terminal to the cleavable linker. In some embodiments, P₁ is connected C-terminal to the cleavable linker and A₁ is connected N-terminal to the cleavable linker.

In some embodiments, P₁ is bound to A₁ through ionic interactions, electrostatic interactions, hydrophobic interactions, Pi-stacking interactions, H-bonding interactions, or a combination thereof. In some embodiments, P₁ is bound to A₁ at or near an antigen binding site. In some embodiments, P₁ becomes unbound from A₁ when L₁ is cleaved by a tumor specific protease thereby exposing A₁ to CD28. In some embodiments, P₁ has less than 75% sequence identity to CD28. In some embodiments, P₁ comprises a de novo amino acid sequence that shares less than 10% sequence identity to CD28. In some embodiments, P₁ comprises a modified amino acid, or a modified non-natural amino acid, or a combination thereof. In some embodiments, P₁ does not comprise albumin or an albumin fragment. In some embodiments, P₁ does not comprise an albumin binding domain.

In some embodiments, L₁ is bound to the N-terminus of A₁. In some embodiments, L₁ is bound to the C-terminus of A₁. In some embodiments, L₁ is a peptide sequence having at least 5 to no more than 50 amino acids. In some embodiments, L₁ is a peptide sequence having at least 10 to no more than 30 amino acids. In some embodiments, L₁ is a peptide sequence having at least 10 amino acids. In some embodiments, L₁ is a peptide sequence having at least 18 amino acids. In some embodiments, L₁ is a peptide sequence having at least 26 amino acids. In some embodiments, L₁ comprises a formula comprising (G₂S)ₙ, wherein n is an integer from 1 to 3 (SEQ ID NO: 1993). In some embodiments, L₁ comprises a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1. In some embodiments, L₁ comprises an amino acid sequence selected from any one of SEQ ID NOs: 36-65. In some embodiments, L₁ comprises the amino acid sequence of SEQ ID NO: 40. In some embodiments, L₁ comprises the amino acid sequence of SEQ ID NO: 65.

In some embodiments, L₁ is a substrate for a tumor specific protease. In some embodiments, P₁ becomes unbound from A₁ when L₁ is cleaved by the tumor specific protease thereby exposing A₁ to CD28. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, or a matrix metalloprotease cleavable amino acid sequence.

In some embodiments, the isolated polypeptide or polypeptide complex comprises an amino acid sequence with at least 80%, 85%, 90%, 95%, 99%, or 100% sequence identity to any one of SEQ ID NOs: 74-78, 80, 82, 84-85, 88, 90, and 1393-1450. In some embodiments, the isolated polypeptide or polypeptide complex comprises the amino acid sequence of any one of SEQ ID NOs: 74-78, 80, 82, 84-85, 88, 90, and 1393-1450. In some embodiments, the isolated polypeptide or polypeptide complex comprises an amino acid sequence with at least 80%, 85%, 90%, 95%, 99%, or 100% sequence identity to any one of SEQ ID NOs: 1445-1450. In some embodiments, the isolated polypeptide or polypeptide complex comprises an amino acid sequence with at least 80%, 90%, 95%, 99%, or 100% identity to any one of SEQ ID NOs: 1393-1450.

In some embodiments, the isolated polypeptide or polypeptide complex further comprises a half-life extending molecule, H₁. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin.

In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgGl, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10G or 10E. In some embodiments, the single domain antibody is SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 92, HC-CDR2: SEQ ID NO: 93, and HC-CDR3: SEQ ID NO: 94. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 96, HC-CDR2: SEQ ID NO: 97, and HC-CDR3: SEQ ID NO: 98. In some embodiments, the single domain antibody comprises an amino acid sequence with at least 80%, 85%, 90%, 95%, 99%, or 100% sequence identity to SEQ ID NO: 95. In some embodiments, the single domain antibody comprises the amino acid sequence of SEQ ID NO: 95. In some embodiments, the single domain antibody comprises an amino acid sequence with at least 80%, 85%, 90%, 95%, 99%, or 100% sequence identity to SEQ ID NO: 118. In some embodiments, the single domain antibody comprises the amino acid sequence of SEQ ID NO: 118. In some embodiments, the single domain antibody comprises an amino acid sequence with at least 80%, 85%, 90%, 95%, 99%, or 100% sequence identity to SEQ ID NO: 1509. In some embodiments, the single domain antibody comprises the amino acid sequence of SEQ ID NO: 1509.

In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

In some embodiments, H₁ comprises a linking moiety (L₂) that connects H₁ to P₁. In some embodiments, L₂ is a peptide sequence having at least 5 to no more than 50 amino acids. In some embodiments, L₂ is a peptide sequence having at least 10 to no more than 30 amino acids. In some embodiments, L₂ is a peptide sequence having at least 10 amino acids. In some embodiments, L₂ is a peptide sequence having at least 18 amino acids. In some embodiments, L₂ has a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1. In some embodiments, L₂ comprises an amino acid sequence according to GGGGSGGGS (SEQ ID NO: 1998).

In some embodiments, the isolated polypeptide or polypeptide complex comprises an amino acid sequence with at least 80%, 85%, 90%, 95%, 99%, or 100% sequence identity to any one of SEQ ID NOs: 100-104, 106, 108, 110-111, 114, 116, and 1451-1502. In some embodiments, the isolated polypeptide or polypeptide complex comprises the amino acid sequence of any one of SEQ ID NOs: 100-104, 106, 108, 110-111, 114, 116, and 1451-1502. In some embodiments, the isolated polypeptide or polypeptide complex comprises an amino acid sequence with at least 80%, 85%, 90%, 95%, 99%, or 100% sequence identity to any one of SEQ ID NOs: 1503-1508. In some embodiments, the isolated polypeptide or polypeptide complex comprises an amino acid sequence according to any one of SEQ ID NOs: 1503-1508.

Disclosed herein, in some embodiments, are pharmaceutical compositions comprising the isolated polypeptide or polypeptide complex of any one of the embodiments disclosed herein, and a pharmaceutically acceptable excipient.

Disclosed herein, in some embodiments, are isolated recombinant nucleic acid molecules encoding an isolated polypeptide or polypeptide complex of any one of the embodiments disclosed herein. Disclosed herein, in some embodiments, are vectors comprising an isolated recombinant nucleic acid molecule encoding an isolated polypeptide or polypeptide complex of any one of the embodiments disclosed herein. Disclosed herein, in some embodiments, are host cells comprising an isolated recombinant nucleic acid molecule encoding an isolated polypeptide or polypeptide complex of any one of the embodiments disclosed herein.

Disclosed herein, in some embodiments, are methods of treating cancer in a subject in need thereof comprising administering to the subject an isolated polypeptide or polypeptide complex of any one of the embodiments disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates binding of Ab-1 to peptides as measured by ELISA.

FIG. 2 illustrates binding of Ab-1 to peptides as measured by ELISA.

FIG. 3 illustrates binding of Ab-1 to peptides as measured by ELISA.

FIG. 4 illustrates biolayer interferometry (BLI) titration data for Ab-1 binding to peptide-1.

FIG. 5 illustrates BLI titration data for Ab-1 binding to peptide-3.

FIG. 6 illustrates BLI titration data for Ab-1 binding to peptide-6.

FIG. 7 illustrates BLI titration data for Ab-1 binding to peptide-7.

FIG. 8 illustrates BLI titration data for Ab-1 binding to peptide-12.

FIG. 9 illustrates BLI titration data for Ab-1 binding to peptide-16.

FIG. 10 illustrates BLI titration data for Ab-1 binding to peptide-23.

FIG. 11 illustrates BLI titration data for Ab-1 binding to peptide-1.

FIG. 12 illustrates BLI titration data for Ab-1 binding to peptide-20.

FIG. 13 illustrates BLI titration data for Ab-1 binding to peptide-21.

FIG. 14 illustrates inhibition of Ab-1 binding to CD28 by peptides of the present disclosure.

FIG. 15 illustrates inhibition of Ab-1 binding to CD28 by peptides of the present disclosure.

FIG. 16 illustrates inhibition of Ab-1 binding to CD28 by peptides of the present disclosure.

FIG. 17 illustrates binding of anti-CD28 Fab to peptides as measured by ELISA.

FIG. 18 illustrates binding of anti-CD28 Fab to peptides as measured by ELISA.

FIG. 19 illustrates binding of anti-CD28 Fab to peptides as measured by ELISA.

FIG. 20 illustrates binding of anti-CD28 Fab to peptides as measured by ELISA.

FIG. 21 illustrates inhibition of anti-CD28 Fab binding to CD28 by peptides of the present disclosure.

FIG. 22 illustrates inhibition of anti-CD28 Fab binding to CD28 by peptides of the present disclosure.

FIG. 23 illustrates inhibition of anti-CD28 Fab binding to CD28 by peptides of the present disclosure.

FIG. 24 illustrates inhibition of anti-CD28 Fab binding to CD28 by peptides of the present disclosure.

FIG. 25 illustrates inhibition of anti-CD28 scFv disulfide to CD28 by peptides of the present disclosure.

FIG. 26 illustrates BLI titration data for anti-CD28 Fab binding to peptide-39.

FIG. 27 illustrates BLI titration data for anti-CD28 Fab binding to peptide-72.

FIG. 28 illustrates BLI titration data for anti-CD28 Fab binding to peptide-73.

FIG. 29 illustrates BLI titration data for anti-CD28 Fab binding to peptide-78.

FIG. 30 illustrates BLI titration data for anti-CD28 Fab binding to peptide-79.

FIG. 31 illustrates BLI titration data for anti-CD28 Fab binding to peptide-84.

FIG. 32 illustrates BLI titration data for anti-CD28 Fab binding to peptide-86.

FIG. 33 illustrates the core sequence motif of peptide-39 sequences generated using WebLogo 3.7.12.

FIG. 34 illustrates the core sequence motif of anti-CD28 Fab peptide-44 sequences generated using WebLogo 3.7.12.

FIG. 35 illustrates a schematic for identifying peptides that can be attached to anti-CD28 multispecific antibodies for selective activation in tumor microenvironments. The schematic illustrates a directed evolution and phage display technology to identify peptides that block antigen recognition by antigen binding domains.

FIG. 36 illustrates anti-CD28 scFv binding to peptides measured by ELISA.

FIG. 37 illustrates anti-CD28 scFv binding to peptides measured by ELISA.

FIGS. 38-39 illustrate peptide inhibition of CD28 scFv binding to CD28 as measured by ELISA.

FIGS. 40-41 illustrate kinetic binding of anti-CD28 scFv to Peptide-116 and Peptide-118 as measured by Octet.

FIGS. 42A-42B illustrate binding of anti-CD28 scFv to Ala scan peptides of Peptide-116.

FIGS. 43A-43B illustrate inhibition of anti-CD28 scFv by Ala scan peptides of Peptide-116.

FIG. 44 illustrates the core sequence motif of optimized anti-CD28 scFv Peptide-116 sequences generated using WebLogo 3.7.4.

FIGS. 45-47 illustrate peptides that inhibit the anti-CD28 scFv from binding the CD28 antigen measured by ELISA.

FIGS. 48A-48U illustrate kinetic binding of anti-CD28 scFv binding to peptides as measured by Octet.

### DETAILED DESCRIPTION

Protein-based therapies such as antibodies and bispecific or multispecific antibodies, such as T cell engagers, have proven effective for a variety diseases and disorders. As with any therapy, there is a need to minimize off-target effects of the protein-based therapy in healthy tissue while maintaining activity of the protein-based therapy in disease tissue. One such strategy is to create an inactive form of the protein-based therapy in which a necessary binding site on the protein-based therapy is blocked with a molecule linked to the protein-based therapy, thereby preventing the protein-based therapy from binding or interacting with its cognate receptor or target antigen when in healthy tissue. For activating the protein-based therapy in the desired disease-state microenvironment, the molecule is cleaved from the protein-based therapy by a protease that is specific to the disease-state microenvironment. The molecule is then released from the protein-based therapy and the protein-based therapy is free to interact with its cognate receptor or target antigen in the diseased tissue.

CD28 is a protein expressed on T cells that provides co-stimulatory signals required for T cell activation and survival. CD28 is considered a key therapeutic target for many diseases including immune diseases and cancer. Disclosed herein are peptides that bind to and impair binding of CD28 binding domains to CD28. In some embodiments, the peptides are attached to the CD28 binding domain via a cleavable linker that is selectively cleaved in tumor microenvironments such that the CD28 binding domain is available for binding to the CD28 target in tumor microenvironments. The peptides as disclosed herein can be applied to a variety of antibody formats that bind to CD28 to reduce the off-target effects of the antibodies in healthy tissue while maintaining activity of the anti-CD28 in diseased tissue.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Phickthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Isolated Polypeptide or Polypeptide Complexes

Disclosed herein are polypeptide or polypeptide complexes comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26. In some embodiments, the peptide comprises an amino acid sequence selected from any one of SEQ ID NOs: 1-26. In some embodiments, the peptide consists of an amino acid sequence selected from any one of SEQ ID NOs: 1-26. In some embodiments, the peptide comprises the amino acid sequence of SEQ ID NO: 1. In some embodiments, the peptide consists of the amino acid sequence of SEQ ID NO: 1. In some embodiments, the peptide comprises an amino acid sequence selected from any one of SEQ ID NOs: 2-19 and 22-26 or the peptide comprises 1, 2, or 3 amino acid substitutions, additions, or deletions relative to an amino acid sequence selected from any one of SEQ ID NOs: 2-19 and 22-26. In some embodiments, the peptide comprises an amino acid sequence selected from any one of SEQ ID NOs: 2-19 and 22-26. In some embodiments, the peptide comprises an amino acid sequence selected from any one of SEQ ID NOs: 2-19. In some embodiments, the peptide consists of an amino acid sequence selected from any one of SEQ ID NOs: 2-19. In some embodiments, the peptide comprises an amino acid sequence selected from any one of SEQ ID NOs: 2-9 and 12-18. In some embodiments, the peptide consists of an amino acid sequence selected from any one of SEQ ID NOs: 2-9 and 12-18. In some embodiments, the peptide comprises an amino acid sequence selected from any one of SEQ ID NOs: 3, 6-7, 9-13, and 16-19. In some embodiments, the peptide comprises an amino acid sequence selected from any one of SEQ ID NOs: 3, 6, 11-12, and 16-17.

TABLE 1
Peptide Mask Sequences (P₁) for CD28 Binding Domain¹

| Construct | Amino Acid Sequence | |
|---|---|---|
| Description | (N to C) | SEQ ID NO: |
| Peptide-1 | MDWCPRDLWVHCFA | 1 |
| Peptide-2 | ADWCPRDLWVHCFA | 2 |
| Peptide-3 | RDWCPRDLWVHCFA | 3 |
| Peptide-4 | NDWCPRDLWVHCFA | 4 |
| Peptide-5 | DDWCPRDLWVHCFA | 5 |
| Peptide-6 | QDWCPRDLWVHCFA | 6 |
| Peptide-7 | EDWCPRDLWVHCFA | 7 |
| Peptide-8 | GDWCPRDLWVHCFA | 8 |
| Peptide-9 | HDWCPRDLWVHCFA | 9 |
| Peptide-10 | IDWCPRDLWVHCFA | 10 |
| Peptide-11 | LDWCPRDLWVHCFA | 11 |
| Peptide-12 | KDWCPRDLWVHCFA | 12 |
| Peptide-13 | FDWCPRDLWVHCFA | 13 |
| Peptide-14 | PDWCPRDLWVHCFA | 14 |
| Peptide-15 | SDWCPRDLWVHCFA | 15 |
| Peptide-16 | TDWCPRDLWVHCFA | 16 |
| Peptide-17 | WDWCPRDLWVHCFA | 17 |
| Peptide-18 | YDWCPRDLWVHCFA | 18 |
| Peptide-19 | VDWCPRDLWVHCFA | 19 |
| Peptide-20 | Met(O)-DWCPRDLWVHCFA | 20 |
| Peptide-21 | Met(O)₂-DWCPRDLWVHCFA | 21 |
| Peptide-22 | LDWCPRHLWALCFN | 22 |
| Peptide-23 | IDWCPRYLWDECYN | 23 |
| Peptide-24 | IDWCPLHLWHECYH | 24 |
| Peptide-25 | VPWCQIISGLECLT | 25 |
| Peptide-26 | IYWCPIDRWNDCYN | 26 |
| Peptide-27 | YWSCFQIPFTCFYF | 120 |
| Peptide-28 | WECLKYTEANACTY | 121 |
| Peptide-29 | FMCLPQNPHPLCFK | 122 |
| Peptide-30 | PDCQTILFKPWCVR | 123 |
| Peptide-31 | ACDTYFAGMINECF | 124 |
| Peptide-32 | GCQYPWAFDAMSCP | 125 |
| Peptide-33 | WCWDYKLWVPTQCP | 126 |
| Peptide-34 | WCWDYREWVPMTCP | 127 |
| Peptide-35 | ECHWSMWAVGHACF | 128 |
| Peptide-36 | YCAGHSPINIGKCL | 129 |
| Peptide-37 | WCWDYKLWVPMTCP | 130 |
| Peptide-38 | DCWWMFESYPGRCI | 131 |
| Peptide-39 | YCAGHAWQYINKCL | 132 |
| Peptide-40 | WCWYYREWVPMTCP | 133 |
| Peptide-41 | YWYCSPSIVRCVLV | 134 |
| Peptide-42 | PDCHTILFKPWCVR | 135 |
| Peptide-43 | WICDKSGSIMLCRA | 136 |
| Peptide-44 | SCAAGASYAWPYCL | 137 |
| Peptide-45 | DCPWLSGFSRPLLC | 138 |
| Peptide-46 | YCAGMSTIQLWLKC | 139 |
| Peptide-47 | YCAGHMPQYEVKCL | 140 |
| Peptide-48 | YCAGHTEEHYVKCM | 141 |
| Peptide-49 | YCMGHSHEHIWKCL | 142 |
| Peptide-50 | YCAGHSHEYLAKCE | 143 |
| Peptide-109 | LICKSGSILILCAQ | 1510 |
| Peptide-110 | SPCGLFQWMEICEF | 1511 |
| Peptide-111 | SFCGLFLDLWICEF | 1512 |
| Peptide-112 | DNCYVIWGFEWQCR | 1513 |
| Peptide-113 | VNCMRVHRTLTWCV | 1514 |
| Peptide-114 | FCTPREWSFLNFVC | 1515 |
| Peptide-115 | CFAYLWIDSWIRVC | 1516 |
| Peptide-116 | MDWCPRERWVDCFF | 1517 |
| Peptide-117 | GQHCATSMWRYCMF | 1518 |
| Peptide-118 | GYCHYWGDMVMWCG | 1519 |
| Peptide-119 | DNCHYIWGFEWQCG | 1520 |
| Peptide-120 | IDCIMVHMVKPWCF | 1521 |
| Peptide-121 | NCQPWYWNMFAFGC | 1522 |
| Peptide-122 | NCQPWYWNMIAFGC | 1523 |
| Peptide-123 | GCFTWSQRTFPFTC | 1524 |
| Peptide-124 | CFYAEYYDQVYSFC | 1525 |
| Peptide-125 | ADWCPRERWVDCFF | 1526 |
| Peptide-126 | MAWCPRERWVDCFF | 1527 |
| Peptide-127 | MDACPRERWVDCFF | 1528 |
| Peptide-128 | MDWCARERWVDCFF | 1529 |
| Peptide-129 | MDWCPAERWVDCFF | 1530 |
| Peptide-130 | MDWCPRARWVDCFF | 1531 |
| Peptide-131 | MDWCPREAWVDCFF | 1532 |
| Peptide-132 | MDWCPRERAVDCFF | 1533 |
| Peptide-133 | MDWCPRERWADCFF | 1534 |
| Peptide-134 | MDWCPRERWVACFF | 1535 |
| Peptide-135 | MDWCPRERWVDCAF | 1536 |
| Peptide-136 | MDWCPRERWVDCFA | 1537 |
| Peptide-137 | MDWCPIDLWNECFF | 1538 |
| Peptide-138 | MDWCPIHLWHVCFN | 1539 |
| Peptide-139 | MDWCPIYLWSECFN | 1540 |
| Peptide-140 | MNWCPKDIWYLCFN | 1541 |
| Peptide-141 | MDWCPLHMWHECFS | 1542 |
| Peptide-142 | MDWCPLYLWNECFN | 1543 |
| Peptide-143 | MDWCPRDLWDLCFA | 1544 |
| Peptide-144 | MDWCPRDLWHECFA | 1545 |
| Peptide-145 | MDWCPRDLWHLCFS | 1546 |
| Peptide-146 | MDWCPRDLWSECFF | 1547 |
| Peptide-147 | MDWCPRDMWDECFA | 1548 |
| Peptide-148 | MDWCPRDMWSECFA | 1549 |
| Peptide-149 | MDWCPRDMWSVCFS | 1550 |
| Peptide-150 | MDWCPRFMWDECFN | 1551 |
| Peptide-151 | MDWCPRHMWNYCFA | 1552 |
| Peptide-152 | MDWCPRSLWHECFA | 1553 |
| Peptide-153 | MDWCPRYLWHVCFA | 1554 |
| Peptide-154 | MHWCPVDLWYLCYN | 1555 |
| Peptide-155 | MDWCPVHLWSVCFA | 1556 |
| Peptide-156 | MDWCPRERWVDCFF | 1557 |

¹Met(O) is methionine sulfoxide; Met(O)2 is methionine sulfone.

TABLE 2
Peptide Mask Sequences for CD28 Binding Domain
(Alanine Scanning Peptides of Peptide-44)

| Construct | Amino Acid Sequence | SEQ ID |
|---|---|---|
| Description | (N to C) | NO: |
| Peptide-51 | ACAAGASYAWPYCL | 144 |
| Peptide-52 | SCAAAASYAWPYCL | 145 |
| Peptide-53 | SCAAGAAYAWPYCL | 146 |
| Peptide-54 | SCAAGASAAWPYCL | 147 |
| Peptide-55 | SCAAGASYAAPYCL | 148 |
| Peptide-56 | SCAAGASYAWAYCL | 149 |
| Peptide-57 | SCAAGASYAWPACL | 150 |
| Peptide-58 | SCAAGASYAWPYCA | 151 |

TABLE 3
Peptide Mask Sequences for CD28 Binding Domain
(Alanine Scanning Peptides of Peptide-39)

| Construct | Amino Acid Sequence | SEQ ID |
|---|---|---|
| Description | (N to C) | NO: |
| Peptide-59 | ACAGHAWQYINKCL | 152 |
| Peptide-60 | YCAAHAWQYINKCL | 153 |
| Peptide-61 | YCAGAAWQYINKCL | 154 |
| Peptide-62 | YCAGHAAQYINKCL | 155 |
| Peptide-63 | YCAGHAWAYINKCL | 156 |
| Peptide-64 | YCAGHAWQAINKCL | 157 |
| Peptide-65 | YCAGHAWQYANKCL | 158 |
| Peptide-66 | YCAGHAWQYIAKCL | 159 |
| Peptide-67 | YCAGHAWQYINACL | 160 |
| Peptide-68 | YCAGHAWQYINKCA | 161 |

TABLE 4
Peptide Mask Sequences for CD28 Binding
Domain (Optimization Library of
Peptide-39)

| Construct | Amino Acid Sequence | SEQ ID |
|---|---|---|
| Description | (N to C) | NO: |
| Peptide-69 | YCEGHSPEYILKCL | 162 |
| Peptide-70 | YCEGHRPEYILKCL | 163 |
| Peptide-71 | YCEGHEGIYFWKCL | 164 |
| Peptide-72 | YCEGHLPEYIEKCL | 165 |
| Peptide-73 | YCEGHAPEYIAKCL | 166 |
| Peptide-74 | YCEGHAPEYIRKCL | 167 |
| Peptide-75 | YCEGHAPEYIWKCL | 168 |
| Peptide-76 | YCEGHSQENIWKCL | 169 |
| Peptide-77 | YCEGHLPEYLGKCL | 170 |
| Peptide-78 | YCEGHLPEYREKCL | 171 |
| Peptide-79 | YCEGHLPEYLAKCL | 172 |
| Peptide-80 | YCEGHSQEYLWKCL | 173 |
| Peptide-81 | YCEGHSSEYIQKCL | 174 |
| Peptide-82 | HCSGHHWLQVWKCF | 175 |
| Peptide-83 | YCWSHSPPFIKKCY | 176 |
| Peptide-84 | YCEGHLPEYRNKCL | 177 |
| Peptide-85 | YCEGHLPEYLEKCV | 178 |
| Peptide-86 | YCEGHLPEYIQKCL | 179 |
| Peptide-87 | YCEGHLPEYIWKCL | 180 |
| Peptide-88 | YCEGHAPEYIGKCL | 181 |

TABLE 5
Peptide Mask Sequences for CD28 Binding Domain
(Optimization Library of Peptide-44)

| Construct | Amino Acid Sequence | SEQ ID |
|---|---|---|
| Description | (N to C) | NO: |
| Peptide-89 | TCSAGATWPWPICL | 182 |
| Peptide-90 | LCAAGASWPWPVCL | 183 |
| Peptide-91 | TCSAGASYPWPLCL | 184 |
| Peptide-92 | TCAAGAAWPWPQCL | 185 |
| Peptide-93 | TCSAGATYTWPKCF | 186 |
| Peptide-94 | TCAAGATWSWPACF | 187 |
| Peptide-95 | TCAAGAAWGWPLCF | 188 |
| Peptide-96 | LCAAGATYPWPLCL | 189 |
| Peptide-97 | TCSAGAAYPWPLCL | 190 |
| Peptide-98 | TCSAGAAYSWPACF | 191 |
| Peptide-99 | LCAAGATYPWPTCL | 192 |
| Peptide-100 | SCSAGAHYPWPQCF | 193 |
| Peptide-101 | TCSAGATYEWPRCL | 194 |
| Peptide-102 | TCSAGAHYPWPICL | 195 |
| Peptide-103 | SCSAGAAYYWPWCL | 196 |
| Peptide-104 | TCSAGATWAWPLCF | 197 |
| Peptide-105 | TCAAGATYLWPTCL | 198 |
| Peptide-106 | TCAAGAHYPWPACL | 199 |
| Peptide-107 | LCAAGFTYPWPLCL | 200 |
| Peptide-108 | LCKPGVPYKWPTCL | 201 |

In some embodiments, the peptide comprises an amino acid sequence selected from any one of SEQ ID NOs: 22-26. In some embodiments, the peptide consists of an amino acid sequence selected from any one of SEQ ID NOs: 22-26. In some embodiments, the peptide comprises an amino acid sequence selected from any one of SEQ ID NOs: 22-24 and 26. In some embodiments, the peptide comprises an amino acid sequence selected from any one of SEQ ID Nos: 22-23 and 26. In some embodiments, the peptide comprises the amino acid sequence of SEQ ID NO: 23. In some embodiments, the peptide consists of the amino acid sequence of SEQ ID NO: 23.

In some embodiments, the peptide comprises the amino acid sequence of SEQ ID NO: 1. In some embodiments, the peptide comprises the amino acid sequence of SEQ ID NO: 2. In some embodiments, the peptide comprises the amino acid sequence of SEQ ID NO: 3. In some embodiments, the peptide comprises the amino acid sequence of SEQ ID NO: 4. In some embodiments, the peptide comprises the amino acid sequence of SEQ ID NO: 5. In some embodiments, the peptide comprises the amino acid sequence of SEQ ID NO: 6. In some embodiments, the peptide comprises the amino acid sequence of SEQ ID NO: 7. In some embodiments, the peptide comprises the amino acid sequence of SEQ ID NO: 8. In some embodiments, the peptide comprises the amino acid sequence of SEQ ID NO: 9. In some embodiments, the peptide comprises the amino acid sequence of SEQ ID NO: 10. In some embodiments, the peptide comprises the amino acid sequence of SEQ ID NO: 11. In some embodiments, the peptide comprises the amino acid sequence of SEQ ID NO: 12. In some embodiments, the peptide comprises the amino acid sequence of SEQ ID NO: 13. In some embodiments, the peptide comprises the amino acid sequence of SEQ ID NO: 14. In some embodiments, the peptide comprises the amino acid sequence of SEQ ID NO: 15. In some embodiments, the peptide comprises the amino acid sequence of SEQ ID NO: 16. In some embodiments, the peptide comprises the amino acid sequence of SEQ ID NO: 17. In some embodiments, the peptide comprises the amino acid sequence of SEQ ID NO: 18. In some embodiments, the peptide comprises the amino acid sequence of SEQ ID NO: 19. In some embodiments, the peptide comprises the amino acid sequence of SEQ ID NO: 20. In some embodiments, the peptide comprises the amino acid sequence of SEQ ID NO: 21. In some embodiments, the peptide comprises the amino acid sequence of SEQ ID NO: 22. In some embodiments, the peptide comprises the amino acid sequence of SEQ ID NO: 23. In some embodiments, the peptide comprises the amino acid sequence of SEQ ID NO: 24. In some embodiments, the peptide comprises the amino acid sequence of SEQ ID NO: 25. In some embodiments, the peptide comprises the amino acid sequence of SEQ ID NO: 26.

Disclosed herein are isolated polypeptide or polypeptide complexes comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T. In some embodiments, X₁ is selected from A, R, N, D, Q, E, G, H, K, F, P, S, T, W, and Y. In some embodiments, X₁ is selected from R, Q, E, H, I, L, K, F, T, W, Y, and V; X₂ is D; X₃ is P; X₄ is R; X₅ is D; X₆ is L; X₇ is W; X₈ is V; X₉ is H; X₁₀ is F; and X₁₁ is A. In some embodiments, X₁ is selected from R, Q, E, L, K, T, and W; X₂ is D; X₃ is P; X₄ is R; X₅ is D; X₆ is L; X₇ is W; X₈ is V; X₉ is H; X₁₀ is F; and X₁₁ is A. In some embodiments, X₁ is L or I; X₂ is D or Y; X₃ is P; X₄ is R, L, or I; X₅ is H, Y, or D; X₆ is L or R; X₇ is W; X₈ is A, D, H, or N; X₉ is L, E, or D; X₁₀ is F or Y; and X₁₁ is N or H. In some embodiments, X₁ is L or I; X₂ is D or Y; X₃ is P; X₄ is R or I; X₅ is H, Y, or D; X₆ is L or R; X₇ is W; X₈ is A, D, or N; X₉ is L, E, or D; X₁₀ is F or Y; and X_{II} is N.

Disclosed herein are isolated polypeptide or polypeptide complexes comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26. In some embodiments, the peptide comprises an amino acid sequence selected from any one of SEQ ID NOs: 2-26. In some embodiments, the peptide consists of an amino acid sequence selected from any one of SEQ ID NOs: 2-26.

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆ -Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W; Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W; Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is Y; Z₂ is selected from D, E, A, and Q; Z₃ is G; Z₄ is H; Z₅ is selected from L, S, A, T, I, V, and M; Z₆ is selected from P, A, E, Q, S, L, W, G, V, and D; Z₇ is selected from E, Q, I, M, V, A, and L; Z₈ is selected from Y and H; Z₉ is selected from M, I, and L; Z₁₀ is selected from A, Q, S, W, E, L, G, D, T, M, and N; Z₁₁ is K; and Z₁₂ is selected from L and M. In some embodiments, Z₁ is Y; Z₂ is selected from D, E, and A; Z₃ is G; Z₄ is H; Z₅ is selected from L, S, A, and T; Z₆ is selected from P, A, E, Q, S, L, and W; Z₇ is selected from E, Q, I, and M; Z₈ is Y; Z₉ is selected from M, I, and L; Z₁₀ is selected from A, Q, S, W, E, L, G, and D; Z₁₁ is K; and Z₁₂ is L. In some embodiments, Z₁ is Y; Z₂ is selected from D and E; Z₃ is G; Z₄ is H; Z₅ is selected from L, S, and A; Z₆ is selected from P, A, E, Q, S, and L; Z₇ is selected from E, Q, and I; Z₈ is Y; Z₉ is selected from M, I, and L; Z₁₀ is selected from A, Q, S, W, E, L, G, and D; Z₁₁ is K; and Z₁₂ is L.

In some embodiments, the peptide comprises the amino acid sequence of SEQ ID NO: 132. In some embodiments, the peptide consists of the amino acid sequence of SEQ ID NO: 132. In some embodiments, the peptide comprises an amino acid sequence according to any one of SEQ ID NOs: 162-181. In some embodiments, the peptide comprises the amino acid sequence according to SEQ ID NO: 172. In some embodiments, the peptide consists of the amino acid sequence according to SEQ ID NO: 172. In some embodiments, the peptide comprises an amino acid sequence according to any one of SEQ ID NOs: 152 to 161.

In some embodiments, the peptide comprises the amino acid sequence of SEQ ID NO: 162. In some embodiments, the peptide comprises the amino acid sequence of SEQ ID NO: 163. In some embodiments, the peptide comprises the amino acid sequence of SEQ ID NO: 164. In some embodiments, the peptide comprises the amino acid sequence of SEQ ID NO: 165. In some embodiments, the peptide comprises the amino acid sequence of SEQ ID NO: 166. In some embodiments, the peptide comprises the amino acid sequence of SEQ ID NO: 167. In some embodiments, the peptide comprises the amino acid sequence of SEQ ID NO: 168. In some embodiments, the peptide comprises the amino acid sequence of SEQ ID NO: 169. In some embodiments, the peptide comprises the amino acid sequence of SEQ ID NO: 170. In some embodiments, the peptide comprises the amino acid sequence of SEQ ID NO: 171. In some embodiments, the peptide comprises the amino acid sequence of SEQ ID NO: 173. In some embodiments, the peptide comprises the amino acid sequence of SEQ ID NO: 174. In some embodiments, the peptide comprises the amino acid sequence of SEQ ID NO: 175. In some embodiments, the peptide comprises the amino acid sequence of SEQ ID NO: 176. In some embodiments, the peptide comprises the amino acid sequence of SEQ ID NO: 177. In some embodiments, the peptide comprises the amino acid sequence of SEQ ID NO: 178. In some embodiments, the peptide comprises the amino acid sequence of SEQ ID NO: 179. In some embodiments, the peptide comprises the amino acid sequence of SEQ ID NO: 180. In some embodiments, the peptide comprises the amino acid sequence of SEQ ID NO: 181.

In some embodiments, the peptide comprises the amino acid sequence of SEQ ID NO: 152. In some embodiments, the peptide comprises the amino acid sequence of SEQ ID NO: 153. In some embodiments, the peptide comprises the amino acid sequence of SEQ ID NO: 154. In some embodiments, the peptide comprises the amino acid sequence of SEQ ID NO: 155. In some embodiments, the peptide comprises the amino acid sequence of SEQ ID NO: 156. In some embodiments, the peptide comprises the amino acid sequence of SEQ ID NO: 157. In some embodiments, the peptide comprises the amino acid sequence of SEQ ID NO: 158. In some embodiments, the peptide comprises the amino acid sequence of SEQ ID NO: 159. In some embodiments, the peptide comprises the amino acid sequence of SEQ ID NO: 160. In some embodiments, the peptide comprises the amino acid sequence of SEQ ID NO: 161.

Disclosed herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A. In some embodiments, U₁ is selected from T, S, L, M, W, Q, P, R, G, N, I, Y, D, E, V, and H; U₂ is selected from A, S, T, K, E, R, G, N, W, and D; U₃ is selected from A, E, P, D, G, W, Q, V, and F; U₄ is selected from G, V, F, D, E, and R; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, and I; U₈ is selected from P, S, T, A, E, K, Q, N, V, G, H, L, D, Y, I, M, and R; U₉ is selected from W, L, and A; U₁₀ is selected from P, S, R, L, and E; U₁₁ is selected from L, A, T, M, V, W, Y, E, Q, I, F, S, P, K, R, D, H, N, and G; and U₁₂ is selected from L, F, V, M, I, Y, E, and W. In some embodiments, U₁ is selected from T, S, L, M, and W; U₂ is selected from A, S, and T; U₃ is selected from A and E; U₄ is selected from G, V, and F; U₅ is selected from A, F, and V; U₆ is selected from T, S, H, M, A, and N; U₇ is selected from Y, W, and F; U₈ is selected from P, S, T, A, E, K, Q, N, and V; U₉ is selected from W and L; U₁₀ is selected from P and S; U₁₁ is selected from L, A, T, M, V, W, Y, E, Q, I, F, and S; and U₁₂ is selected from L, F, V, and M. In some embodiments, U₁ is selected from T and S; U₂ is selected from A and S; U₃ is A; U₄ is selected from G and V; U₅ is selected from A and F; U₆ is selected from T, S, and H; U₇ is selected from Y and W; U₈ is selected from P, S, T, and A; U₉ is W; U₁₀ is P; U₁₁ is selected from L, A, T, M, V, W, and Y; and U₁₂ is selected from L and F.

In some embodiments, the peptide comprises the amino acid sequence of SEQ ID NO: 137. In some embodiments, the peptide consists of the amino acid sequence of SEQ ID NO: 137. In some embodiments, the peptide comprises an amino acid sequence according to any one of SEQ ID NOs: 144 to 151. In some embodiments, the peptide comprises an amino acid sequence according to any one of SEQ ID NOs: 182-201.

In some embodiments, the peptide comprises the amino acid sequence of SEQ ID NO: 144. In some embodiments, the peptide comprises the amino acid sequence of SEQ ID NO: 145. In some embodiments, the peptide comprises the amino acid sequence of SEQ ID NO: 146. In some embodiments, the peptide comprises the amino acid sequence of SEQ ID NO: 147. In some embodiments, the peptide comprises the amino acid sequence of SEQ ID NO: 148. In some embodiments, the peptide comprises the amino acid sequence of SEQ ID NO: 149. In some embodiments, the peptide comprises the amino acid sequence of SEQ ID NO: 150. In some embodiments, the peptide comprises the amino acid sequence of SEQ ID NO: 151.

In some embodiments, the peptide comprises the amino acid sequence of SEQ ID NO: 182. In some embodiments, the peptide comprises the amino acid sequence of SEQ ID NO: 183. In some embodiments, the peptide comprises the amino acid sequence of SEQ ID NO: 184. In some embodiments, the peptide comprises the amino acid sequence of SEQ ID NO: 185. In some embodiments, the peptide comprises the amino acid sequence of SEQ ID NO: 186. In some embodiments, the peptide comprises the amino acid sequence of SEQ ID NO: 187. In some embodiments, the peptide comprises the amino acid sequence of SEQ ID NO: 188. In some embodiments, the peptide comprises the amino acid sequence of SEQ ID NO: 189. In some embodiments, the peptide comprises the amino acid sequence of SEQ ID NO: 190. In some embodiments, the peptide comprises the amino acid sequence of SEQ ID NO: 191. In some embodiments, the peptide comprises the amino acid sequence of SEQ ID NO: 192. In some embodiments, the peptide comprises the amino acid sequence of SEQ ID NO: 193. In some embodiments, the peptide comprises the amino acid sequence of SEQ ID NO: 194. In some embodiments, the peptide comprises the amino acid sequence of SEQ ID NO: 195. In some embodiments, the peptide comprises the amino acid sequence of SEQ ID NO: 196. In some embodiments, the peptide comprises the amino acid sequence of SEQ ID NO: 197. In some embodiments, the peptide comprises the amino acid sequence of SEQ ID NO: 198. In some embodiments, the peptide comprises the amino acid sequence of SEQ ID NO: 199. In some embodiments, the peptide comprises the amino acid sequence of SEQ ID NO: 200. In some embodiments, the peptide comprises the amino acid sequence of SEQ ID NO: 201.

Described herein, in some embodiments, are isolated polypeptides or polypeptide complexes comprising a CD28 binding domain that is linked to a peptide that impairs binding of the CD28 binding domain to CD28, wherein the peptide comprises an amino acid sequence according to any one of SEQ ID NOs: 120-201 and 204-1392.

Described herein, in some embodiments, are isolated polypeptides or polypeptide complexes comprising a CD28 binding domain that is linked to a peptide that impair binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

### CD28 Binding Domain

In some embodiments, the CD28 binding domain comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise the amino acid sequences of HC-CDR1: SEQ ID NO: 27, HC-CDR2: SEQ ID NO: 28, and HC-CDR3: SEQ ID NO: 29, and the anti-CD28 antibody comprises an anti-CD28 light chain variable domain that comprises complementarity determining regions (CDRs): LC-CDR1, LC-CDR2, and LC-CDR3, wherein the LC-CDR1, the LC-CDR2, and the LC-CDR3 comprise the amino acid sequences of LC-CDR1: SEQ ID NO: 30, LC-CDR2: (KAS), and LC-CDR3: SEQ ID: 32.

TABLE 6
Exemplary amino acid sequence of a CD28 Binding Domain (A1).
CDRs determined by IMGT definition.

| Construct | Amino Acid Sequence | SEQ ID |
|---|---|---|
| Description | (N to C) | NO: |
| anti-CD28: HC: CDR1 | GYTFTSYY | 27 |
| anti-CD28: HC: CDR2 | IYPGNVNT | 28 |
| anti-CD28: HC: CDR3 | TRSHYGLDWNFDV | 29 |
| anti-CD28: LC: CDR1 | QNIYVW | 30 |
| anti-CD28: LC: CDR2 | KAS | |
| anti-CD28: LC: CDR3 | QQGQTYPYT | 32 |
| anti-CD28 HC | QVQLVQSGAEVKKPGASVKVSCKASGYTFTSYYIHW | 33 |
| | VRQAPGQGLEWIGSIYPGNVNTNYNEKFKDRATLTV | |
| | DTSISTAYMELSRLRSDDTAVYFCTRSHYGLDWNFD | |
| | VWGQGTTVTVSS | |
| anti-CD28 LC | DIQMTQSPSSLSASVGDRVTITCHASQNIYVWLNWYQ | 34 |
| | QKPGKAPKLLIYKASNLHTGVPSRFSGSGSGTDFTLTIS | |
| | SLQPEDFATYYCQQGQTYPYTFGGGTKVEIK | |
| anti-CD28 scFv wt | QVQLVQSGAEVKKPGASVKVSCKASGYTFTSYYIHW | 35 |
| Vh-linker-Vl | VRQAPGQGLEWIGSIYPGNVNTNYNEKFKDRATLTV | |
| | DTSISTAYMELSRLRSDDTAVYFCTRSHYGLDWNFD | |
| | VWGQGTTVTVSSGGGGSGGGGSGGGGSDIQMTQSPSS | |
| | LSASVGDRVTITCHASQNIYVWLNWYQQKPGKAPKL | |
| | LIYKASNLHTGVPSRFSGSGSGTDFTLTISSLQPEDFAT | |
| | YYCQQGQTYPYTFGGGTKVEIK | |
| anti-CD28 scFv | QVQLVQSGAEVKKPGASVKVSCKASGYTFTSYYIHW | 67 |
| Vh-linker-Vl | VRQAPGQCLEWIGSIYPGNVNTNYNEKFKDRATLTVD | |
| (with disulfide mutation) | TSISTAYMELSRLRSDDTAVYFCTRSHYGLDWNFDV | |
| G44C, G235C | WGQGTTVTVSSGGGGSGGGGSGGGGSDIQMTQSPSSL | |
| | SASVGDRVTITCHASQNIYVWLNWYQQKPGKAPKLLI | |
| | YKASNLHTGVPSRFSGSGSGTDFTLTISSLQPEDFATYY | |
| | CQQGQTYPYTFGCGTKVEIK | |
| anti-CD28 scFv wt | DIQMTQSPSSLSASVGDRVTITCHASQNIYVWLNWYQ | 68 |
| V1-linker-Vh | QKPGKAPKLLIYKASNLHTGVPSRFSGSGSGTDFTLTIS | |
| | SLQPEDFATYYCQQGQTYPYTFGGGTKVEIKGGGGS | |
| | GGGGSGGGGSQVQLVQSGAEVKKPGASVKVSCKASG | |
| | YTFTSYYIHWVRQAPGQGLEWIGSIYPGNVNTNYNE | |
| | KFKDRATLTVDTSISTAYMELSRLRSDDTAVYFCTRS | |
| | HYGLDWNFDVWGQGTTVTVSS | |
| anti-CD28 scFv | DIQMTQSPSSLSASVGDRVTITCHASQNIYVWLNWYQ | 69 |
| Vl-linker-Vh | QKPGKAPKLLIYKASNLHTGVPSRFSGSGSGTDFTLTIS | |
| (with disulfide mutation) | SLQPEDFATYYCQQGQTYPYTFGCGTKVEIKGGGGS | |
| G100C, G166C | GGGGSGGGGSQVQLVQSGAEVKKPGASVKVSCKASG | |
| | YTFTSYYIHWVRQAPGQCLEWIGSIYPGNVNTNYNEK | |
| | FKDRATLTVDTSISTAYMELSRLRSDDTAVYFCTRSH | |
| | YGLDWNFDVWGQGTTVTVSS | |
| Anti-CD28 Fab | DIQMTQSPSSLSASVGDRVTITCHASQNIYVWLNWYQ | 202 |
| LC | QKPGKAPKLLIYKASNLHTGVPSRFSGSGSGTDFTLTIS | |
| | SLQPEDFATYYCQQGQTYPYTFGGGTKVEIKRTVAAP | |
| | SVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| Anti-CD28 Fab | QVQLVQSGAEVKKPGASVKVSCKASGYTFTSYYIHW | 203 |
| HC | VRQAPGQGLEWIGSIYPGNVNTNYNEKFKDRATLTVD | |
| | TSISTAYMELSRLRSDDTAVYFCTRSHYGLDWNFDVW | |
| | GQGTTVTVSSASTKGPSVFPLAPSSKSTSGGTAALGCL | |
| | VKDYFPEPVTVSWNSGALTSGVHTFPAVLQSSGLYSLS | |
| | SVVTVPSSSLGTQTYICNVNHKPSNTKVDKKVEPKSC | |

In some embodiments, the anti-CD28 heavy chain variable domain comprises an amino acid sequence with at least 80%, 85%, 90%, 95%, 98%, 99%, or 100% sequence identity to SEQ ID NO: 33. In some embodiments, the anti-CD28 light chain variable domain comprises an amino acid sequence with at least 80%, 85%, 90%, 95%, 98%, 99%, or 100% sequence identity to SEQ ID NO: 34. In some embodiments, the anti-CD28 heavy chain variable domain comprises the amino acid sequence of SEQ ID NO: 33 and the anti-CD28 light chain variable domain comprises the amino acid sequence of SEQ ID NO: 34.

In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 35. In some embodiments, the anti-CD28 antibody comprises the scFv, and the scFv comprises the amino acid of SEQ ID NO: 35. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 67. In some embodiments, the anti-CD28 antibody comprises the scFv, and the scFv comprises the amino acid sequence of SEQ ID NO: 67. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 68. In some embodiments, the anti-CD28 antibody comprises the scFv, and the scFv comprises the amino acid sequence of SEQ ID NO: 68. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 69. In some embodiments, the anti-CD28 antibody comprises the scFv, and the scFv comprises the amino acid sequence of SEQ ID NO: 69. In some embodiments, the anti-CD28 antibody comprises the Fab, wherein the Fab comprises an immunoglobulin light chain with at least 80%, 85%, 90%, 95%, 98%, 99%, or 100% sequence identity to SEQ ID NO: 202, and wherein the Fab comprises an immunoglobulin heavy chain with 80%, 85%, 90%, 95%, 98%, 99%, or 100% sequence identity to SEQ ID NO: 203. In some embodiments, the isolated polypeptide or polypeptide complex is human or humanized.

A₁-L₁-P₁ Configuration

In some embodiments, the peptide is connected to the CD28 binding domain in a configuration according to A₁-L₁-P₁ wherein A₁ comprises the CD28 binding domain, P₁ comprises the peptide that impairs binding of the CD28 binding domain to CD28, and L₁ comprises a cleavable linker. In some embodiments, P₁ is connected N-terminal to the cleavable linker and A₁ is connected C-terminal to the cleavable linker. In some embodiments, P₁ is connected C-terminal to the cleavable linker and A₁ is connected N-terminal to the cleavable linker. In some embodiments, P₁ is bound to A₁ through ionic interactions, electrostatic interactions, hydrophobic interactions, Pi-stacking interactions, H-bonding interactions, or a combination thereof. In some embodiments, P₁ is bound to A₁ at or near an antigen binding site. In some embodiments, P₁ becomes unbound from A₁ when L₁ is cleaved by a tumor specific protease thereby exposing A₁ to CD28. In some embodiments, P₁ has less than 750% sequence identity to CD28. In some embodiments, P₁ comprises a de novo amino acid sequence that shares less than 100 sequence identity to CD28. In some embodiments, P₁ comprises a modified amino acid, or a modified non-natural amino acid, or a combination thereof. In some embodiments, P₁ does not comprise albumin or an albumin fragment. In some embodiments, P₁ does not comprise an albumin binding domain.

In some embodiments, L₁ is bound to the N-terminus of A₁. In some embodiments, L₁ is bound to the C-terminus of A₁. In some embodiments, L₁ is a peptide sequence having at least 5 to no more than 50 amino acids. In some embodiments, L₁ is a peptide sequence having at least 10 to no more than 30 amino acids. In some embodiments, L₁ is a peptide sequence having at least 10 amino acids. In some embodiments, L₁ is a peptide sequence having at least 18 amino acids. In some embodiments, L₁ is a peptide sequence having at least 26 amino acids. In some embodiments, L₁ comprises a formula comprising (G₂S)ₙ, wherein n is an integer from 1 to 3 (SEQ ID NO: 1993). In some embodiments, L₁ comprises a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1. In some embodiments, L₁ comprises an amino acid sequence selected from any one of SEQ ID NOs: 36-65. In some embodiments, L₁ comprises the amino acid sequence of SEQ ID NO: 40. In some embodiments, L₁ comprises the amino acid sequence of SEQ ID NO: 65.

TABLE 7
Linker (L1 or L2) Amino Acid Sequences

| Construct | Amino Acid Sequence | SEQ ID |
|---|---|---|
| Description | (N to C) | NO: |
| Linker 1 | GGGGSGGGGSGGGGS | 36 |
| Linker 2 | GGGGS | 37 |
| Linker 3 | GGGGSGGGS | 38 |
| Cleavable linker | GGGGSGGGLSGRSDAGSPLGLAGSGGGS | 39 |
| Cleavable linker | GSSGGSGGLSGRSDAGSPLGLAGSGGGS | 40 |
| Linker 4 | GGGGSLSGRSDNHGSSGT | 41 |
| Linker 5 | GGGGSSGGSGGSGLSGRSDNHGSSGT | 42 |
| Linker 6 | ASGRSDNH | 43 |
| Linker 7 | LAGRSDNH | 44 |
| Linker 8 | ISSGLASGRSDNH | 45 |
| Linker 9 | ISSGLLAGRSDNH | 46 |
| Linker 10 | LSGRSDNH | 47 |
| Linker 11 | ISSGLLSGRSDNP | 48 |
| Linker 12 | ISSGLLSGRSDNH | 49 |
| Linker 13 | LSGRSDNHSPLGLAGS | 50 |
| Linker 14 | SPLGLAGSLSGRSDNH | 51 |
| Linker 15 | SPLGLSGRSDNH | 52 |
| Linker 16 | LAGRSDNHSPLGLAGS | 53 |
| Linker 17 | LSGRSDNHVPLSLKMG | 54 |
| Linker 18 | LSGRSDNHVPLSLSMG | 55 |
| Linker 19 | GSSGGSGGSGGSGISSGLLSGRSDNHGSSGT | 56 |
| Linker 20 | GSSGGSGGSGGISSGLLSGRSDNHGGGS | 57 |
| Linker 21 | ASGRSDNH | 58 |
| Linker 22 | LAGRSDNH | 59 |
| Linker 23 | ISSGLASGRSDNH | 60 |
| Linker 24 | LSGRSDAG | 61 |
| Linker 25 | ISSGLLSGRSDAG | 62 |
| Linker 26 | AAGLLAPPGGLSGRSDAG | 63 |
| Linker 27 | SPLGLSGRSDAG | 64 |
| Linker 28 | LSGRSDAGSPLGLAG | 65 |
| Non-cleavable linker | SGGGGSGGASSGAGGS | 66 |
| Non-cleavable linker | GGGGSGGGSGGGGSGGASSGAGGSGGGS | 119 |

In some embodiments, L₁ is a substrate for a tumor specific protease. In some embodiments, P₁ becomes unbound from A₁ when L₁ is cleaved by the tumor specific protease thereby exposing A₁ to CD28. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, or a matrix metalloprotease cleavable amino acid sequence.

In some embodiments, the isolated polypeptide or polypeptide complex comprises an amino acid sequence with at least 80%, 85%, 90%, 95%, 99%, or 100% sequence identity to anyone of SEQ ID NOs: 74-90. In some embodiments, the isolated polypeptide or polypeptide complex comprises an amino acid sequence with at least 80%, 85%, 90%, 95%, 99%, or 100% sequence identity to any one of SEQ ID NOs: 74-78, 80, 82, 84-85, 88, 90, and 1393-1450. In some embodiments, the isolated polypeptide or polypeptide complex comprises the amino acid sequence of any one of SEQ ID NOs: 74-78, 80, 82, 84-85, 88, 90, and 1393-1450. In some embodiments, the isolated polypeptide or polypeptide complex comprises an amino acid sequence with at least 80%, 85%, 90%, 95%, 99%, or 100% sequence identity to any one of SEQ ID NOs: 1445-1450. In some embodiments, the isolated polypeptide or polypeptide complex comprises an amino acid sequence with at least 80%, 85%, 90%, 95%, 99%, or 100% sequence identity to any one of SEQ ID NOs: 1393-1450.

TABLE 8
Masked Polypeptide Complexes (A₁-L₁-P₁)

| | | SEQ |
|---|---|---|
| Construct | Amino Acid Sequence | ID |
| Description | (N to C) | NO: |
| Ab-3 | MDWCPRDLWVHCFAGSSGGSGGLSGRSDAGSPLGLAGSGGGSQVQ | 74 |
| Peptide-1 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| Linker-40; | TRSHYGLDWNFDVWGQGTTVTVSSGGGGSGGGGSGGGGSDIQMTQ | |
| wt CD28 scFv | SPSSLSASVGDRVTITCHASQNIYVWLNWYQQKPGKAPKLLIYKASN | |
| Vh-linker-Vl | LHTGVPSRFSGSGSGTDFTLTISSLQPEDFATYYCQQGQTYPYTFGGG | |
| | TKVEIK | |
| Ab-4 | MDWCPRDLWVHCFAGSSGGSGGLSGRSDAGSPLGLAGSGGGSQVQ | 75 |
| Peptide-1 | LVQSGAEVKKPGASVKVSCKASGYTFTSYYIHWVRQAPGQCLEWIG | |
| mask; | SIYPGNVNTNYNEKFKDRATLTVDTSISTAYMELSRLRSDDTAVYFC | |
| Cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| CD28 scFv | LHTGVPSRFSGSGSGTDFTLTISSLQPEDFATYYCQQGQTYPYTFGCG | |
| Vh-linker-Vl | TKVEIK | |
| G44C, G235C | | |
| Ab-23 | MDWCPRDLWVHCFAGSSGGSGGLSGRSDAGSPLGLAGSGGGSQVQ | 76 |
| Peptide-1 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| Cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-V1 | TKVEIK | |
| Ab-24 | MDWCPRDLWVHCFAGSSGGSGGLSGRSDAGSPLGLAGSGGGSQVQ | 77 |
| Peptide-1 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| Cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-Vl | TKVEIK | |
| Ab-25 | MDWCPRDLWVHCFAGSSGGSGGLSGRSDAGSPLGLAGSGGGSQVQ | 78 |
| Peptide-1 | LVQSGAEVKKPGASVKVSCKASGYTFTSYYIHWVRQAPGQCLEWIG | |
| mask; | SIYPGNVNTNYNEKFKDRATLTVDTSISTAYMELSRLRSDDTAVYFC | |
| Cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| CD28 scFv | LHTGVPSRFSGSGSGTDFTLTISSLQPEDFATYYCQQGQTYPYTFGCG | |
| Vh-linker-Vl | TKVEIK | |
| G44C, G235C | | |
| Ab-26 | MDWCPRDLWVHCFAGGGGSGGGSGGGGSGGASSGAGGSGGGSQV | 79 |
| Peptide-1 | QLVQSGAEVKKPGASVKVSCKASGYTFTSYYIHWVRQAPGQCLEWI | |
| mask; | GSIYPGNVNTNYNEKFKDRATLTVDTSISTAYMELSRLRSDDTAVYF | |
| non-cleavable | CTRSHYGLDWNFDVWGQGTTVTVSSGGGGSGGGGSGGGGSDIQMT | |
| linker | QSPSSLSASVGDRVTITCHASQNIYVWLNWYQQKPGKAPKLLIYKAS | |
| CD28 scFv | NLHTGVPSRFSGSGSGTDFTLTISSLQPEDFATYYCQQGQTYPYTFGC | |
| Vh-linker-Vl | GTKVEIK | |
| G44C, G235C | | |
| Ab-27 | MDWCPRDLWVHCFAGSSGGSGGLSGRSDAGSPLGLAGSGGGSQVQ | 80 |
| Peptide-1 | LVQSGAEVKKPGASVKVSCKASGYTFTSYYIHWVRQAPGQCLEWIG | |
| mask; | SIYPGNVNTNYNEKFKDRATLTVDTSISTAYMELSRLRSDDTAVYFC | |
| cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| CD28 scFv | LHTGVPSRFSGSGSGTDFTLTISSLQPEDFATYYCQQGQTYPYTFGCG | |
| Vh-linker-Vl | TKVEIK | |
| G44C, G235C | | |
| Ab-28 | MDWCPRDLWVHCFAGGGGSGGGSGGGGSGGASSGAGGSGGGSQV | 81 |
| Peptide-1 | QLVQSGAEVKKPGASVKVSCKASGYTFTSYYIHWVRQAPGQCLEWI | |
| mask; | GSIYPGNVNTNYNEKFKDRATLTVDTSISTAYMELSRLRSDDTAVYF | |
| non-cleavable | CTRSHYGLDWNFDVWGQGTTVTVSSGGGGSGGGGSGGGGSDIQMT | |
| linker; | QSPSSLSASVGDRVTITCHASQNIYVWLNWYQQKPGKAPKLLIYKAS | |
| CD28 scFv | NLHTGVPSRFSGSGSGTDFTLTISSLQPEDFATYYCQQGQTYPYTFGC | |
| Vh-linker-Vl | GTKVEIK | |
| G44C, G235C | | |
| Ab-29 | MDWCPRDLWVHCFAGSSGGSGGLSGRSDAGSPLGLAGSGGGSQVQ | 82 |
| Peptide-1 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-Vl | TKVEIK | |
| Ab-30 | MDWCPRDLWVHCFAGGGGSGGGSGGGGSGGASSGAGGSGGGSQV | 83 |
| Peptide-1 | QLVQSGAEVKKPGASVKVSCKASGYTFTSYYIHWVRQAPGQGLEWI | |
| mask; | GSIYPGNVNTNYNEKFKDRATLTVDTSISTAYMELSRLRSDDTAVYF | |
| non-cleavable | CTRSHYGLDWNFDVWGQGTTVTVSSGGGGSGGGGSGGGGSDIQMT | |
| linker; | QSPSSLSASVGDRVTITCHASQNIYVWLNWYQQKPGKAPKLLIYKAS | |
| wt CD28 scFv | NLHTGVPSRFSGSGSGTDFTLTISSLQPEDFATYYCQQGQTYPYTFGG | |
| Vh-linker-Vl | GTKVEIK | |
| Ab-31 | MDWCPRDLWVHCFAGSSGGSGGLSGRSDAGSPLGLAGSGGGSDIQ | 84 |
| Peptide-1 | MTQSPSSLSASVGDRVTITCHASQNIYVWLNWYQQKPGKAPKLLIY | |
| mask; | KASNLHTGVPSRFSGSGSGTDFTLTISSLQPEDFATYYCQQGQTYPYT | |
| cleavable | FGGGTKVEIKGGGGSGGGGSGGGGSQVQLVQSGAEVKKPGASVKV | |
| linker; | SCKASGYTFTSYYIHWVRQAPGQGLEWIGSIYPGNVNTNYNEKFKD | |
| wt CD28 scFv | RATLTVDTSISTAYMELSRLRSDDTAVYFCTRSHYGLDWNFDVWGQ | |
| Vl-linker-Vh | GTTVTVSS | |
| Ab-32 | MDWCPRDLWVHCFAGSSGGSGGLSGRSDAGSPLGLAGSGGGSDIQ | 85 |
| Peptide-1 | MTQSPSSLSASVGDRVTITCHASQNIYVWLNWYQQKPGKAPKLLIY | |
| mask; | KASNLHTGVPSRFSGSGSGTDFTLTISSLQPEDFATYYCQQGQTYPYT | |
| cleavable | FGCGTKVEIKGGGGSGGGGSGGGGSQVQLVQSGAEVKKPGASVKV | |
| linker; | SCKASGYTFTSYYIHWVRQAPGQCLEWIGSIYPGNVNTNYNEKFKD | |
| CD28 scFv | RATLTVDTSISTAYMELSRLRSDDTAVYFCTRSHYGLDWNFDVWGQ | |
| Vl-linker-Vh | GTTVTVSS | |
| G100C, | | |
| G166C | | |
| Ab-33 | MDWCPRDLWVHCFAGGGGSGGGSGGGGSGGASSGAGGSGGGSDI | 86 |
| Peptide-1 | QMTQSPSSLSASVGDRVTITCHASQNIYVWLNWYQQKPGKAPKLLI | |
| mask; | YKASNLHTGVPSRFSGSGSGTDFTLTISSLQPEDFATYYCQQGQTYPY | |
| non-cleavable | TFGGGTKVEIKGGGGSGGGGSGGGGSQVQLVQSGAEVKKPGASVK | |
| linker; | VSCKASGYTFTSYYIHWVRQAPGQGLEWIGSIYPGNVNTNYNEKFK | |
| wt CD28 scFv | DRATLTVDTSISTAYMELSRLRSDDTAVYFCTRSHYGLDWNFDVWG | |
| Vl-linker-Vh | QGTTVTVSS | |
| Ab-34 | MDWCPRDLWVHCFAGGGGSGGGSGGGGSGGASSGAGGSGGGSDI | 87 |
| Peptide-1 | QMTQSPSSLSASVGDRVTITCHASQNIYVWLNWYQQKPGKAPKLLI | |
| mask; | YKASNLHTGVPSRFSGSGSGTDFTLTISSLQPEDFATYYCQQGQTYPY | |
| non-cleavable | TFGCGTKVEIKGGGGSGGGGSGGGGSQVQLVQSGAEVKKPGASVK | |
| linker; | VSCKASGYTFTSYYIHWVRQAPGQCLEWIGSIYPGNVNTNYNEKFK | |
| CD28 scFv | DRATLTVDTSISTAYMELSRLRSDDTAVYFCTRSHYGLDWNFDVWG | |
| Vl-linker-Vh | QGTTVTVSS | |
| G100C, | | |
| G166C | | |
| Ab-35 | IDWCPRYLWDECYNAGSGGSGGLSGRSDAGSPLGLAGSGGGSQVQL | 88 |
| Peptide-23 | VQSGAEVKKPGASVKVSCKASGYTFTSYYIHWVRQAPGQGLEWIGS | |
| mask; | IYPGNVNTNYNEKFKDRATLTVDTSISTAYMELSRLRSDDTAVYFCT | |
| cleavable | RSHYGLDWNFDVWGQGTTVTVSSGGGGSGGGGSGGGGSDIQMTQS | |
| linker; | PSSLSASVGDRVTITCHASQNIYVWLNWYQQKPGKAPKLLIYKASNL | |
| wt CD28 scFv | HTGVPSRFSGSGSGTDFTLTISSLQPEDFATYYCQQGQTYPYTFGGGT | |
| Vh-linker-Vl | KVEIK | |
| Ab-36 | IDWCPRYLWDECYNAGGGSGGGSGGGGSGGASSGAGGSGGGSQVQ | 89 |
| Peptide-23 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| non-cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-Vl | TKVEIK | |
| Ab-37 | IDWCPRYLWDECYNAGSGGSGGLSGRSDAGSPLGLAGSGGGSQVQL | 90 |
| Peptide-23 | VQSGAEVKKPGASVKVSCKASGYTFTSYYIHWVRQAPGQCLEWIGS | |
| mask; | IYPGNVNTNYNEKFKDRATLTVDTSISTAYMELSRLRSDDTAVYFCT | |
| cleavable | RSHYGLDWNFDVWGQGTTVTVSSGGGGSGGGGSGGGGSDIQMTQS | |
| linker; | PSSLSASVGDRVTITCHASQNIYVWLNWYQQKPGKAPKLLIYKASNL | |
| CD28 scFv | HTGVPSRFSGSGSGTDFTLTISSLQPEDFATYYCQQGQTYPYTFGCGT | |
| Vh-linker-Vl | KVEIK | |
| G44C, G235C | | |
| Ab-38 | IDWCPRYLWDECYNAGGGSGGGSGGGGSGGASSGAGGSGGGSQVQ | 91 |
| Peptide-23 | LVQSGAEVKKPGASVKVSCKASGYTFTSYYIHWVRQAPGQCLEWIG | |
| mask; | SIYPGNVNTNYNEKFKDRATLTVDTSISTAYMELSRLRSDDTAVYFC | |
| non-cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| CD28 scFv | LHTGVPSRFSGSGSGTDFTLTISSLQPEDFATYYCQQGQTYPYTFGCG | |
| Vh-linker-Vl | TKVEIK | |
| G44C, G235C | | |
| Ab-39 | MDWCPRDLWVHCFAGSSGGSGGLSGRSDAGSPLGLAGSGGGSQVQ | 1393 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-40 | IDWCPRYLWDECYNAGSGGSGGLSGRSDAGSPLGLAGSGGGSQVQL | 1394 |
| Peptide-23 | VQSGAEVKKPGASVKVSCKASGYTFTSYYIHWVRQAPGQGLEWIGS | |
| mask; CD28 | IYPGNVNTNYNEKFKDRATLTVDTSISTAYMELSRLRSDDTAVYFCT | |
| scFv Vh- | RSHYGLDWNFDVWGQGTTVTVSSGGGGSGGGGSGGGGSDIQMTQS | |
| linker | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-41 | MDWCPRDLWVHCFAGSSGGSGGLSGRSDAGSPLGLAGSGGGSQVQ | 1395 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-42 | IDWCPRYLWDECYNAGSGGSGGLSGRSDAGSPLGLAGSGGGSDIQM | 1396 |
| Peptide-23 | TQSPSSLSASVGDRVTITCHASQNIYVWLNWYQQKPGKAPKLLIYKA | |
| mask; CD28 | SNLHTGVPSRFSGSGSGTDFTLTISSLQPEDFATYYCQQGQTYPYTFG | |
| scFv Vl- | GGTKVEIKGGGGSGGGGSGGGGSQVQLVQSGAEVKKPGASVKVSC | |
| linker-Vh | KASGYTFTSYYIHWVRQAPGQGLEWIGSIYPGNVNTNYNEKFKDRA | |
| | TLTVDTSISTAYMELSRLRSDDTAVYFCTRSHYGLDWNFDVWGQGT | |
| | TVTVSS | |
| Ab-43 | MDWCPRDLWVHCFAGSSGGSGGLSGRSDAGSPLGLAGSGGGSQVQ | 1397 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-44 | MDWCPRDLWVHCFAGSSGGSGGLSGRSDAGSPLGLAGSGGGSQVQ | 1398 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-45 | MDWCPRDLWVHCFAGSSGGSGGLSGRSDAGSPLGLAGSGGGSQVQ | 1399 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-46 | MDWCPRDLWVHCFAGSSGGSGGLSGRSDAGSPLGLAGSGGGSQVQ | 1400 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-47 | MDWCPRDLWVHCFAGSSGGSGGLSGRSDAGSPLGLAGSGGGSDIQ | 1401 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-48 | MDWCPRDLWVHCFAGSSGGSGGLSGRSDAGSPLGLAGSGGGSDIQ | 1402 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-49 | MDWCPRDLWVHCFAGSSGGSGGLSGRSDAGSPLGLAGSGGGSDIQ | 1403 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-50 | MDWCPRDLWVHCFAGSSGGSGGLSGRSDAGSPLGLAGSGGGSDIQ | 1404 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-51 | MDWCPRDLWVHCFAGSSGGSGGLSGRSDAGSPLGLAGSGGGSQVQ | 1405 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-52 | MDWCPRDLWVHCFAGSSGGSGGLSGRSDAGSPLGLAGSGGGSQVQ | 1406 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-53 | MDWCPRDLWVHCFAGSSGGSGGLSGRSDAGSPLGLAGSGGGSQVQ | 1407 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-54 | MDWCPRDLWVHCFAGSSGGSGGLSGRSDAGSPLGLAGSGGGSQVQ | 1408 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-55 | MDWCPRDLWVHCFAGSSGGSGGLSGRSDAGSPLGLAGSGGGSDIQ | 1409 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-56 | MDWCPRDLWVHCFAGSSGGSGGLSGRSDAGSPLGLAGSGGGSDIQ | 1410 |
| Cut from 5805 | MTQSPSSLSASVGDRVTITCHASQNIYVWLNWYQQKPGKAPKLLIY | |
| Peptide-1 | KASNLHTGVPSRFSGSGSGTDFTLTISSLQPEDFATYYCQQGQTYPYT | |
| mask; CD28 | FGGGTKVEIKGGGGSGGGGSGGGGSQVQLVQSGAEVKKPGASVKV | |
| scFv Vl- | SCKASGYTFTSYYIHWVRQAPGQGLEWIGSIYPGNVNTNYNEKFKD | |
| linker-Vh | RATLTVDTSISTAYMELSRLRSDDTAVYFCTRSHYGLDWNFDVWGQ | |
| | GTTVTVSS | |
| Ab-57 | MDWCPRDLWVHCFAGSSGGSGGLSGRSDAGSPLGLAGSGGGSDIQ | 1411 |
| Peptide-1 | MTQSPSSLSASVGDRVTITCHASQNIYVWLNWYQQKPGKAPKLLIY | |
| mask; scFv | KASNLHTGVPSRFSGSGSGTDFTLTISSLQPEDFATYYCQQGQTYPYT | |
| Vl-linker-Vh | FGGGTKVEIKGGGGSGGGGSGGGGSQVQLVQSGAEVKKPGASVKV | |
| | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-58 | MDWCPRDLWVHCFAGSSGGSGGLSGRSDAGSPLGLAGSGGGSDIQ | 1412 |
| Peptide-1 | MTQSPSSLSASVGDRVTITCHASQNIYVWLNWYQQKPGKAPKLLIY | |
| mask; scFv | KASNLHTGVPSRFSGSGSGTDFTLTISSLQPEDFATYYCQQGQTYPYT | |
| Vl-linker-Vh | FGGGTKVEIKGGGGSGGGGSGGGGSQVQLVQSGAEVKKPGASVKV | |
| | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-59 | MDWCPRDLWVHCFAGSSGGSGGLSGRSDAGSPLGLAGSGGGSQVQ | 1413 |
| Peptide-1 | LVQSGAEVKKPGASVKVSCKASGYTFTSYYIHWVRQAPGQGLEWIG | |
| mask; scFv | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-60 | MDWCPRDLWVHCFAGSSGGSGGLSGRSDAGSPLGLAGSGGGSQVQ | 1414 |
| Peptide-1 | LVQSGAEVKKPGASVKVSCKASGYTFTSYYIHWVRQAPGQGLEWIG | |
| mask; scFv | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-61 | MDWCPRDLWVHCFAGSSGGSGGLSGRSDAGSPLGLAGSGGGSQVQ | 1415 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-V1 | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-62 | MDWCPRDLWVHCFAGSSGGSGGLSGRSDAGSPLGLAGSGGGSQVQ | 1416 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-63 | MDWCPRDLWVHCFAGSSGGSGGLSGRSDAGSPLGLAGSGGGSDIQ | 1417 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-64 | MDWCPRDLWVHCFAGSSGGSGGLSGRSDAGSPLGLAGSGGGSDIQ | 1418 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-65 | MDWCPRDLWVHCFAGSSGGSGGLSGRSDAGSPLGLAGSGGGSDIQ | 1419 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-66 | MDWCPRDLWVHCFAGSSGGSGGLSGRSDAGSPLGLAGSGGGSDIQ | 1420 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-67 | MDWCPRDLWVHCFAGSSGGSGGLSGRSDAGSPLGLAGSGGGSQVQ | 1421 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-68 | MDWCPRDLWVHCFAGSSGGSGGLSGRSDAGSPLGLAGSGGGSQVQ | 1422 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-69 | MDWCPRDLWVHCFAGSSGGSGGLSGRSDAGSPLGLAGSGGGSQVQ | 1423 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-70 | MDWCPRDLWVHCFAGSSGGSGGLSGRSDAGSPLGLAGSGGGSQVQ | 1424 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-71 | MDWCPRDLWVHCFAGSSGGSGGLSGRSDAGSPLGLAGSGGGSDIQ | 1425 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-72 | MDWCPRDLWVHCFAGSSGGSGGLSGRSDAGSPLGLAGSGGGSDIQ | 1426 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-73 | MDWCPRDLWVHCFAGSSGGSGGLSGRSDAGSPLGLAGSGGGSDIQ | 1427 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-74 | MDWCPRDLWVHCFAGSSGGSGGLSGRSDAGSPLGLAGSGGGSDIQ | 1428 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-75 | MDWCPRDLWVHCFAGSSGGSGGLSGRSDAGSPLGLAGSGGGSQVQ | 1429 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-V1 | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-76 | MDWCPRDLWVHCFAGSSGGSGGLSGRSDAGSPLGLAGSGGGSQVQ | 1430 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-77 | MDWCPRDLWVHCFAGSSGGSGGLSGRSDAGSPLGLAGSGGGSQVQ | 1431 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-78 | MDWCPRDLWVHCFAGSSGGSGGLSGRSDAGSPLGLAGSGGGSQVQ | 1432 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| Vh-linker-Vl | TRSHYGLDWNFDVWGQGTTVTVSSGGGGSGGGGSGGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-79 | MDWCPRDLWVHCFAGSSGGSGGLSGRSDAGSPLGLAGSGGGSDIQ | 1433 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-80 | MDWCPRDLWVHCFAGSSGGSGGLSGRSDAGSPLGLAGSGGGSDIQ | 1434 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-81 | MDWCPRDLWVHCFAGSSGGSGGLSGRSDAGSPLGLAGSGGGSDIQ | 1435 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-82 | MDWCPRDLWVHCFAGSSGGSGGLSGRSDAGSPLGLAGSGGGSDIQ | 1436 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-83 | MDWCPRDLWVHCFAGSSGGSGGLSGRSDAGSPLGLAGSGGGSQVQ | 1437 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-84 | MDWCPRDLWVHCFAGSSGGSGGLSGRSDAGSPLGLAGSGGGSQVQ | 1438 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh-linkr- | TRSHYGLDWNFDVWGQGTTVTVSSGGGGSGGGGSGGGGSDIQMTQ | |
| V1 | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-85 | MDWCPRDLWVHCFAGSSGGSGGLSGRSDAGSPLGLAGSGGGSQVQ | 1439 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-86 | MDWCPRDLWVHCFAGSSGGSGGLSGRSDAGSPLGLAGSGGGSQVQ | 1440 |
| Peptide-1 | LVQSGAEVKKPGASVKVSCKASGYTFTSYYIHWVRQAPGQGLEWIG | |
| mask; CD28 | SIYPGNVNTNYNEKFKDRATLTVDTSISTAYMELSRLRSDDTAVYFC | |
| scFv Vh- | TRSHYGLDWNFDVWGQGTTVTVSSGGGGSGGGGSGGGGSDIQMTQ | |
| linker-Vl | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIK | |
| Ab-87 | MDWCPRDLWVHCFAGSSGGSGGLSGRSDAGSPLGLAGSGGGSDIQ | 1441 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-88 | MDWCPRDLWVHCFAGSSGGSGGLSGRSDAGSPLGLAGSGGGSDIQ | 1442 |
| Peptide-1 | MTQSPSSLSASVGDRVTITCHASQNIYVWLNWYQQKPGKAPKLLIY | |
| mask' CD28 | KASNLHTGVPSRFSGSGSGTDFTLTISSLQPEDFATYYCQQGQTYPYT | |
| Vl-linker-Vh | FGGGTKVEIKGGGGSGGGGSGGGGSQVQLVQSGAEVKKPGASVKV | |
| | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-89 | MDWCPRDLWVHCFAGSSGGSGGLSGRSDAGSPLGLAGSGGGSDIQ | 1443 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-90 | MDWCPRDLWVHCFAGSSGGSGGLSGRSDAGSPLGLAGSGGGSDIQ | 1444 |
| Peptide-1 | MTQSPSSLSASVGDRVTITCHASQNIYVWLNWYQQKPGKAPKLLIY | |
| mask; CD28 | KASNLHTGVPSRFSGSGSGTDFTLTISSLQPEDFATYYCQQGQTYPYT | |
| scFv Vl- | FGGGTKVEIKGGGGSGGGGSGGGGSQVQLVQSGAEVKKPGASVKV | |
| linker-Vh | SCKASGYTFTSYYIHWVRQAPGQGLEWIGSIYPGNVNTNYNEKFKD | |
| | RATLTVDTSISTAYMELSRLRSDDTAVYFCTRSHYGLDWNFDVWGQ | |
| | GTTVTVSS | |
| Ab-91 | YCEGHLPEYLAKCLGSSGGSGGLSGRSDAGSPLGLAGSGGGSQVQL | 1445 |
| Peptide-79 | VQSGAEVKKPGASVKVSCKASGYTFTSYYIHWVRQAPGQGLEWIGS | |
| mask; CD28 | IYPGNVNTNYNEKFKDRATLTVDTSISTAYMELSRLRSDDTAVYFCT | |
| scFv Vh- | RSHYGLDWNFDVWGQGTTVTVSSGGGGSGGGGSGGGGSDIQMTQS | |
| linker-Vl | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-92 | YCEGHLPEYLAKCLGSSGGSGGLSGRSDAGSPLGLAGSGGGSQVQL | 1446 |
| Peptide-79; | VQSGAEVKKPGASVKVSCKASGYTFTSYYIHWVRQAPGQGLEWIGS | |
| CD28 mask | IYPGNVNTNYNEKFKDRATLTVDTSISTAYMELSRLRSDDTAVYFCT | |
| scFv Vh- | RSHYGLDWNFDVWGQGTTVTVSSGGGGSGGGGSGGGGSDIQMTQS | |
| linker-Vl | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-93 | YCEGHLPEYLAKCLGSSGGSGGLSGRSDAGSPLGLAGSGGGSQVQL | 1447 |
| Peptide-79 | VQSGAEVKKPGASVKVSCKASGYTFTSYYIHWVRQAPGQCLEWIGS | |
| mask; CD28 | IYPGNVNTNYNEKFKDRATLTVDTSISTAYMELSRLRSDDTAVYFCT | |
| disulfide Vh- | RSHYGLDWNFDVWGQGTTVTVSSGGGGSGGGGSGGGGSDIQMTQS | |
| linker-V1 | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGCGT | |
| | KVEIK | |
| Ab-94 | YCEGHLPEYLAKCLGSSGGSGGLSGRSDAGSPLGLAGSGGGSDIQMT | 1448 |
| Peptide-79 | QSPSSLSASVGDRVTITCHASQNIYVWLNWYQQKPGKAPKLLIYKAS | |
| mask; CD28 | NLHTGVPSRFSGSGSGTDFTLTISSLQPEDFATYYCQQGQTYPYTFGG | |
| Vl-linker-Vh | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-95 | YCEGHLPEYLAKCLGSSGGSGGLSGRSDAGSPLGLAGSGGGSDIQMT | 1449 |
| Peptide-79 | QSPSSLSASVGDRVTITCHASQNIYVWLNWYQQKPGKAPKLLIYKAS | |
| mask; CD28 | NLHTGVPSRFSGSGSGTDFTLTISSLQPEDFATYYCQQGQTYPYTFGG | |
| Vl-linker-Vh | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-96 | YCEGHLPEYLAKCLGSSGGSGGLSGRSDAGSPLGLAGSGGGSDIQMT | 1450 |
| Peptide-79 | QSPSSLSASVGDRVTITCHASQNIYVWLNWYQQKPGKAPKLLIYKAS | |
| mask; CD28 | NLHTGVPSRFSGSGSGTDFTLTISSLQPEDFATYYCQQGQTYPYTFGC | |
| scFv disulfide | GTKVEIKGGGGSGGGGSGGGGSQVQLVQSGAEVKKPGASVKVSCK | |
| Vl-linker-Vh | ASGYTFTSYYIHWVRQAPGQCLEWIGSIYPGNVNTNYNEKFKDRAT | |
| | LTVDTSISTAYMELSRLRSDDTAVYFCTRSHYGLDWNFDVWGQGTT | |
| | VTVSS | |

### Half-Life Extending Moiety (H₁)

In some embodiments, the isolated polypeptide or polypeptide complex further comprises a half-life extending molecule, H₁. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin.

In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgGl, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7h-11-15. In some embodiments, the single domain antibody is Alb-1, Alb-8, or Alb-23. In some embodiments, the single domain antibody is 10G or 10E. In some embodiments, the single domain antibody is SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 92, HC-CDR2: SEQ ID NO: 93, and HC-CDR3: SEQ ID NO: 94. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 96, HC-CDR2: SEQ ID NO: 97, and HC-CDR3: SEQ ID NO: 98.

In some embodiments, the single domain antibody comprises an amino acid sequence with at least 80%, 85%, 90%, 95%, 99%, or 100% sequence identity to SEQ ID NO: 95. In some embodiments, the single domain antibody comprises the amino acid sequence of SEQ ID NO: 95. In some embodiments, the single domain antibody comprises an amino acid sequence with at least 80%, 85%, 90%, 95%, 99%, or 100% sequence identity to SEQ ID NO: 118. In some embodiments, the single domain antibody comprises the amino acid sequence of SEQ ID NO: 118. In some embodiments, the single domain antibody comprises an amino acid sequence with at least 80%, 85%, 90%, 95%, 99%, or 100% sequence identity to SEQ ID NO: 1509. In some embodiments, the single domain antibody comprises the amino acid sequence of SEQ ID NO: 1509.

TABLE 9
Half-Life Extending (H₁) Sequences

| Construct Description | Amino Acid Sequence | SEQ ID NO: |
|---|---|---|
| Anti-Albumin: CDR-H1 | GSTFYTAV | 92 |
| Anti-Albumin: CDR-H2 | IRWTALTT | 93 |
| Anti-Albumin: CDR-H3 | AARGTLGLFTTADSYDY | 94 |
| Anti-albumin (HE-1) | EVQLVESGGGLVQPGGSLRLSCAASGSTF | 95 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |
| Anti-Albumin: CDR-H1 | GFTFSKFG | 96 |
| Anti-Albumin: CDR-H2 | ISGSGRDT | 97 |
| Anti-Albumin: CDR-H3 | TIGGSLSV | 98 |
| Anti-albumin (HE-3) | EVQLVESGGGLVQPGNSLRLSCAASGFT | 99 |
| | FSKFGMSWVRQAPGKGLEWVSSISGSGR | |
| | DTLYADSVKGRFTISRDNAKTTLYLQMN | |
| | SLRPEDTAVYYCTIGGSLSVSSQGTLVTV | |
| | SS | |
| Anti-Albumin | EVQLVESGGGLVQPGGSLRLSCAASGSTF | 118 |
| | YTAVMGWVRQAPGKGLEWVAAIRWTA | |
| | LTTSYADSVKGRFTISRDNAKTTLYLQMN | |
| | SLRPEDTAVYYCAARGTLGLFTTADAYD | |
| | YWGQGTLVTVSS | |
| Anti-Albumin (HE-4) | EVQLVESGGGLVQPGNSLRLSCAASGFTF | 1509 |
| | SSFGMSWVRQAPGKGLEWVSSISGSGSD | |
| | TLYADSVKGRFTISRDNAKTTLYLQMNSL | |
| | RPEDTAVYYCTIGGSLSRSSQGTLVTVSS | |

In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

In some embodiments, H₁ comprises a linking moiety (L₂) that connects H₁ to P₁. In some embodiments, L₂ is a peptide sequence having at least 5 to no more than 50 amino acids. In some embodiments, L₂ is a peptide sequence having at least 10 to no more than 30 amino acids. In some embodiments, L₂ is a peptide sequence having at least 10 amino acids. In some embodiments, L₂ is a peptide sequence having at least 18 amino acids. In some embodiments, L₂ has a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1. In some embodiments, L₂ comprises an amino acid sequence according to GGGGSGGGS (SEQ ID NO: 1998).

In some embodiments, the isolated polypeptide or polypeptide complex comprises an amino acid sequence with at least 80%, 85%, 90%, 95%, 99%, or 100% sequence identity to anyone of SEQ ID NOs: 100-104, 106, 108, 110-111, 114, 116, and 1451-1502. In some embodiments, the isolated polypeptide or polypeptide complex comprises the amino acid sequence ofany one of SEQ ID NOs: 100-104, 106, 108, 110-111, 114, 116, and 1451-1502. In some embodiments, the isolated polypeptide or polypeptide complex comprises an amino acid sequence with at least 80%, 85%, 90%, 95%, 99%, or 100% sequence identity to any one of SEQ ID NOs: 1503-1508. In some embodiments, the isolated polypeptide or polypeptide complex comprises an amino acid sequence according to any one of SEQ ID NOs: 1503-1508.

TABLE 10
Masked Polypeptide Complexes with Half-Life Extending Molecules (H₁)

| | | SEQ |
|---|---|---|
| Construct | Amino Acid Sequence | ID |
| Description | (N to C) | NO: |
| Ab-5 | EVQLVESGGGLVQPGGSLRLSCAASGSTFYTAVMGWVRQAPGKGL | 100 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGSSGGSGGLSGRSDAGSPLGLAGSGGGSQVQL | |
| mask; | VQSGAEVKKPGASVKVSCKASGYTFTSYYIHWVRQAPGQGLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGSGGGGSGGGGSDIQMTQS | |
| wt CD28 scFv | PSSLSASVGDRVTITCHASQNIYVWLNWYQQKPGKAPKLLIYKASNL | |
| Vh-linker-Vl | HTGVPSRFSGSGSGTDFTLTISSLQPEDFATYYCQQGQTYPYTFGGGT | |
| | KVEIK | |
| Ab-6 | EVQLVESGGGLVQPGGSLRLSCAASGSTFYTAVMGWVRQAPGKGL | 101 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGSSGGSGGLSGRSDAGSPLGLAGSGGGSQVQL | |
| mask; | VQSGAEVKKPGASVKVSCKASGYTFTSYYIHWVRQAPGQCLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGSGGGGSGGGGSDIQMTQS | |
| disulfide | PSSLSASVGDRVTITCHASQNIYVWLNWYQQKPGKAPKLLIYKASNL | |
| CD28 scFv | HTGVPSRFSGSGSGTDFTLTISSLQPEDFATYYCQQGQTYPYTFGCGT | |
| Vh-linker-Vl | KVEIK | |
| G44C, G235C | | |
| Ab-7 | EVQLVESGGGLVQPGGSLRLSCAASGSTFYTAVMGWVRQAPGKGL | 102 |
| albumin | EWVAAIRWTALTTSYADSVKGRFTISRDNAKTTLYLQMNSLRPEDT | |
| binder: SEQ | AVYYCAARGTLGLFTTADAYDYWGQGTLVTVSSGGGGSGGGSGG | |
| ID NO: 118; | MDWCPRDLWVHCFAGSSGGSGGLSGRSDAGSPLGLAGSGGGSQVQ | |
| Peptide-1 | LVQSGAEVKKPGASVKVSCKASGYTFTSYYIHWVRQAPGQGLEWIG | |
| mask: | SIYPGNVNTNYNEKFKDRATLTVDTSISTAYMELSRLRSDDTAVYFC | |
| cleavable | TRSHYGLDWNFDVWGQGTTVTVSSGGGGSGGGGSGGGGSDIQMTQ | |
| linker | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-Vl | TKVEIK | |
| Ab-8 | EVQLVESGGGLVQPGGSLRLSCAASGSTFYTAVMGWVRQAPGKGL | 103 |
| albumin | EWVAAIRWTALTTSYADSVKGRFTISRDNAKTTLYLQMNSLRPEDT | |
| binder: SEQ | AVYYCAARGTLGLFTTADAYDYWGQGTLVTVSSGGGGSGGGSGG | |
| ID NO: 118; | MDWCPRDLWVHCFAGSSGGSGGLSGRSDAGSPLGLAGSGGGSQVQ | |
| Peptide-1 | LVQSGAEVKKPGASVKVSCKASGYTFTSYYIHWVRQAPGQGLEWIG | |
| mask; | SIYPGNVNTNYNEKFKDRATLTVDTSISTAYMELSRLRSDDTAVYFC | |
| cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| wt CD28 scFv | LHTGVPSRFSGSGSGTDFTLTISSLQPEDFATYYCQQGQTYPYTFGGG | |
| Vh-linker-Vl | TKVEIK | |
| Ab-9 | EVQLVESGGGLVQPGGSLRLSCAASGSTFYTAVMGWVRQAPGKGL | 104 |
| albumin | EWVAAIRWTALTTSYADSVKGRFTISRDNAKTTLYLQMNSLRPEDT | |
| binder: SEQ | AVYYCAARGTLGLFTTADAYDYWGQGTLVTVSSGGGGSGGGSGG | |
| ID NO: 118; | MDWCPRDLWVHCFAGSSGGSGGLSGRSDAGSPLGLAGSGGGSQVQ | |
| Peptide-1 | LVQSGAEVKKPGASVKVSCKASGYTFTSYYIHWVRQAPGQCLEWIG | |
| mask; | SIYPGNVNTNYNEKFKDRATLTVDTSISTAYMELSRLRSDDTAVYFC | |
| cleavable | TRSHYGLDWNFDVWGQGTTVTVSSGGGGSGGGGSGGGGSDIQMTQ | |
| linker; | SPSSLSASVGDRVTITCHASQNIYVWLNWYQQKPGKAPKLLIYKASN | |
| CD28 scFv | LHTGVPSRFSGSGSGTDFTLTISSLQPEDFATYYCQQGQTYPYTFGCG | |
| Vh-linker-Vl | TKVEIK | |
| (disulfide) | | |
| Ab-10 | EVQLVESGGGLVQPGGSLRLSCAASGSTFYTAVMGWVRQAPGKGL | 105 |
| albumin | EWVAAIRWTALTTSYADSVKGRFTISRDNAKTTLYLQMNSLRPEDT | |
| binder: SEQ | AVYYCAARGTLGLFTTADAYDYWGQGTLVTVSSGGGGSGGGSGG | |
| ID NO: 118; | MDWCPRDLWVHCFAGGGGSGGGSGGGGSGGASSGAGGSGGGSQV | |
| Peptide-1 | QLVQSGAEVKKPGASVKVSCKASGYTFTSYYIHWVRQAPGQCLEWI | |
| mask; | GSIYPGNVNTNYNEKFKDRATLTVDTSISTAYMELSRLRSDDTAVYF | |
| non-cleavable | CTRSHYGLDWNFDVWGQGTTVTVSSGGGGSGGGGSGGGGSDIQMT | |
| linker; | QSPSSLSASVGDRVTITCHASQNIYVWLNWYQQKPGKAPKLLIYKAS | |
| CD28 scFv | NLHTGVPSRFSGSGSGTDFTLTISSLQPEDFATYYCQQGQTYPYTFGC | |
| Vh-linker-Vl | GTKVEIK | |
| (disulfide) | | |
| Ab-11 | EVQLVESGGGLVQPGGSLRLSCAASGSTFYTAVMGWVRQAPGKGL | 106 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGSSGGSGGLSGRSDAGSPLGLAGSGGGSQVQL | |
| mask; | VQSGAEVKKPGASVKVSCKASGYTFTSYYIHWVRQAPGQCLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGSGGGGSGGGGSDIQMTQS | |
| CD28 scFv | PSSLSASVGDRVTITCHASQNIYVWLNWYQQKPGKAPKLLIYKASNL | |
| Vh-linker-Vl | HTGVPSRFSGSGSGTDFTLTISSLQPEDFATYYCQQGQTYPYTFGCGT | |
| (disulfide) | KVEIK | |
| Ab-12 | EVQLVESGGGLVQPGGSLRLSCAASGSTFYTAVMGWVRQAPGKGL | 107 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGGGGSGGGSGGGGSGGASSGAGGSGGGSQVQ | |
| mask; | LVQSGAEVKKPGASVKVSCKASGYTFTSYYIHWVRQAPGQCLEWIG | |
| non-cleavable | SIYPGNVNTNYNEKFKDRATLTVDTSISTAYMELSRLRSDDTAVYFC | |
| linker; | TRSHYGLDWNFDVWGQGTTVTVSSGGGGSGGGGSGGGGSDIQMTQ | |
| CD28 scFv | SPSSLSASVGDRVTITCHASQNIYVWLNWYQQKPGKAPKLLIYKASN | |
| Vh-linker-Vl | LHTGVPSRFSGSGSGTDFTLTISSLQPEDFATYYCQQGQTYPYTFGCG | |
| (disulfide) | TKVEIK | |
| Ab-13 | EVQLVESGGGLVQPGGSLRLSCAASGSTFYTAVMGWVRQAPGKGL | 108 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGSSGGSGGLSGRSDAGSPLGLAGSGGGSQVQL | |
| mask; | VQSGAEVKKPGASVKVSCKASGYTFTSYYIHWVRQAPGQGLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGSGGGGSGGGGSDIQMTQS | |
| wt CD28 scFv | PSSLSASVGDRVTITCHASQNIYVWLNWYQQKPGKAPKLLIYKASNL | |
| Vh-linker-Vl | HTGVPSRFSGSGSGTDFTLTISSLQPEDFATYYCQQGQTYPYTFGGGT | |
| | KVEIK | |
| Ab-14 | EVQLVESGGGLVQPGGSLRLSCAASGSTFYTAVMGWVRQAPGKGL | 109 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGGGGSGGGSGGGGSGGASSGAGGSGGGSQVQ | |
| mask; | LVQSGAEVKKPGASVKVSCKASGYTFTSYYIHWVRQAPGQGLEWIG | |
| non-cleavable | SIYPGNVNTNYNEKFKDRATLTVDTSISTAYMELSRLRSDDTAVYFC | |
| linker; | TRSHYGLDWNFDVWGQGTTVTVSSGGGGSGGGGSGGGGSDIQMTQ | |
| wt CD28 scFv | SPSSLSASVGDRVTITCHASQNIYVWLNWYQQKPGKAPKLLIYKASN | |
| Vh-linker-Vl | LHTGVPSRFSGSGSGTDFTLTISSLQPEDFATYYCQQGQTYPYTFGGG | |
| | TKVEIK | |
| Ab-15 | EVQLVESGGGLVQPGGSLRLSCAASGSTFYTAVMGWVRQAPGKGL | 110 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1; | DWCPRDLWVHCFAGSSGGSGGLSGRSDAGSPLGLAGSGGGSDIQMT | |
| cleavable | QSPSSLSASVGDRVTITCHASQNIYVWLNWYQQKPGKAPKLLIYKAS | |
| linker; | NLHTGVPSRFSGSGSGTDFTLTISSLQPEDFATYYCQQGQTYPYTFGG | |
| wt CD28 scFv | GTKVEIKGGGGSGGGGSGGGGSQVQLVQSGAEVKKPGASVKVSCK | |
| Vl-linker-Vh | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-16 | EVQLVESGGGLVQPGGSLRLSCAASGSTFYTAVMGWVRQAPGKGL | 111 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGSSGGSGGLSGRSDAGSPLGLAGSGGGSDIQMT | |
| mask; | QSPSSLSASVGDRVTITCHASQNIYVWLNWYQQKPGKAPKLLIYKAS | |
| cleavable | NLHTGVPSRFSGSGSGTDFTLTISSLQPEDFATYYCQQGQTYPYTFGC | |
| linker; | GTKVEIKGGGGSGGGGSGGGGSQVQLVQSGAEVKKPGASVKVSCK | |
| CD28 scFv | ASGYTFTSYYIHWVRQAPGQCLEWIGSIYPGNVNTNYNEKFKDRAT | |
| Vl-linker-Vh | LTVDTSISTAYMELSRLRSDDTAVYFCTRSHYGLDWNFDVWGQGTT | |
| (disulfide) | VTVSS | |
| Ab-17 | EVQLVESGGGLVQPGGSLRLSCAASGSTFYTAVMGWVRQAPGKGL | 112 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGGGGSGGGSGGGGSGGASSGAGGSGGGSDIQ | |
| mask; | MTQSPSSLSASVGDRVTITCHASQNIYVWLNWYQQKPGKAPKLLIY | |
| non-cleavable | KASNLHTGVPSRFSGSGSGTDFTLTISSLQPEDFATYYCQQGQTYPYT | |
| linker; | FGGGTKVEIKGGGGSGGGGSGGGGSQVQLVQSGAEVKKPGASVKV | |
| wt CD28 scFv | SCKASGYTFTSYYIHWVRQAPGQGLEWIGSIYPGNVNTNYNEKFKD | |
| Vl-linker-Vh | RATLTVDTSISTAYMELSRLRSDDTAVYFCTRSHYGLDWNFDVWGQ | |
| | GTTVTVSS | |
| Ab-18 | EVQLVESGGGLVQPGGSLRLSCAASGSTFYTAVMGWVRQAPGKGL | 113 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGM | |
| Peptide-1 | DWCPRDLWVHCFAGGGGSGGGSGGGGSGGASSGAGGSGGGSDIQ | |
| mask; | MTQSPSSLSASVGDRVTITCHASQNIYVWLNWYQQKPGKAPKLLIY | |
| non-cleavable | KASNLHTGVPSRFSGSGSGTDFTLTISSLQPEDFATYYCQQGQTYPYT | |
| linker; | FGCGTKVEIKGGGGSGGGGSGGGGSQVQLVQSGAEVKKPGASVKV | |
| CD28 scFv | SCKASGYTFTSYYIHWVRQAPGQCLEWIGSIYPGNVNTNYNEKFKD | |
| Vl-linker-Vh | RATLTVDTSISTAYMELSRLRSDDTAVYFCTRSHYGLDWNFDVWGQ | |
| (disulfide) | GTTVTVSS | |
| Ab-19 | EVQLVESGGGLVQPGGSLRLSCAASGSTFYTAVMGWVRQAPGKGL | 114 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGI | |
| Peptide-23 | DWCPRYLWDECYNAGSGGSGGLSGRSDAGSPLGLAGSGGGSQVQL | |
| mask; | VQSGAEVKKPGASVKVSCKASGYTFTSYYIHWVRQAPGQGLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGSGGGGSGGGGSDIQMTQS | |
| wt CD28 scFv | PSSLSASVGDRVTITCHASQNIYVWLNWYQQKPGKAPKLLIYKASNL | |
| Vh-linker-Vl | HTGVPSRFSGSGSGTDFTLTISSLQPEDFATYYCQQGQTYPYTFGGGT | |
| | KVEIK | |
| Ab-20 | EVQLVESGGGLVQPGGSLRLSCAASGSTFYTAVMGWVRQAPGKGL | 115 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGI | |
| Peptide-23 | DWCPRYLWDECYNAGGGSGGGSGGGGSGGASSGAGGSGGGSQVQ | |
| mask; | LVQSGAEVKKPGASVKVSCKASGYTFTSYYIHWVRQAPGQGLEWIG | |
| non-cleavable | SIYPGNVNTNYNEKFKDRATLTVDTSISTAYMELSRLRSDDTAVYFC | |
| linker; | TRSHYGLDWNFDVWGQGTTVTVSSGGGGSGGGGSGGGGSDIQMTQ | |
| wt CD28 scFv | SPSSLSASVGDRVTITCHASQNIYVWLNWYQQKPGKAPKLLIYKASN | |
| Vh-linker-Vl | LHTGVPSRFSGSGSGTDFTLTISSLQPEDFATYYCQQGQTYPYTFGGG | |
| | TKVEIK | |
| Ab-21 | EVQLVESGGGLVQPGGSLRLSCAASGSTFYTAVMGWVRQAPGKGL | 116 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGI | |
| Peptide-23 | DWCPRYLWDECYNAGSGGSGGLSGRSDAGSPLGLAGSGGGSQVQL | |
| mask; | VQSGAEVKKPGASVKVSCKASGYTFTSYYIHWVRQAPGQCLEWIGS | |
| cleavable | IYPGNVNTNYNEKFKDRATLTVDTSISTAYMELSRLRSDDTAVYFCT | |
| linker; | RSHYGLDWNFDVWGQGTTVTVSSGGGGGGGGSGGGGSDIQMTQS | |
| CD28 scFv | PSSLSASVGDRVTITCHASQNIYVWLNWYQQKPGKAPKLLIYKASNL | |
| Vh-linker-Vl | HTGVPSRFSGSGSGTDFTLTISSLQPEDFATYYCQQGQTYPYTFGCGT | |
| (disulfide) | KVEIK | |
| Ab-22 | EVQLVESGGGLVQPGGSLRLSCAASGSTFYTAVMGWVRQAPGKGL | 117 |
| albumin | EWVAAIRWTALTTSYADSVKGRFTISRDGAKTTLYLQMNSLRPEDT | |
| binder: HE-1; | AVYYCAARGTLGLFTTADSYDYWGQGTLVTVSSGGGGSGGGSGGI | |
| Peptide-23 | DWCPRYLWDECYNAGGGSGGGSGGGGSGGASSGAGGSGGGSQVQ | |
| mask; | LVQSGAEVKKPGASVKVSCKASGYTFTSYYIHWVRQAPGQCLEWIG | |
| non-cleavable | SIYPGNVNTNYNEKFKDRATLTVDTSISTAYMELSRLRSDDTAVYFC | |
| linker; | TRSHYGLDWNFDVWGQGTTVTVSSGGGGSGGGGSGGGGSDIQMTQ | |
| CD28 scFv | SPSSLSASVGDRVTITCHASQNIYVWLNWYQQKPGKAPKLLIYKASN | |
| Vh-linker-VI | LHTGVPSRFSGSGSGTDFTLTISSLQPEDFATYYCQQGQTYPYTFGCG | |
| (disulfide) | TKVEIK | |
| Ab-97 | EVQLVESGGGLVQPGGSLRLSCAASGSTFYTAVMGWVRQAPGKGL | 1451 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-98 | EVQLVESGGGLVQPGNSLRLSCAASGFTFSSFGMSWVRQAPGKGLE | 1452 |
| HE-4; | WVSSISGSGSDTLYADSVKGRFTISRDNAKTTLYLQMNSLRPEDTAV | |
| Peptide-23 | YYCTIGGSLSRSSQGTLVTVSSGGGGSGGGSGGIDWCPRYLWDECY | |
| mask; CD28 | NAGSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-99 | EVQLVESGGGLVQPGNSLRLSCAASGFTFSSFGMSWVRQAPGKGLE | 1453 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-100 | EVQLVESGGGLVQPGGSLRLSCAASGSTFYTAVMGWVRQAPGKGL | 1454 |
| HE-1; | EWVAAIRWTALTTSYADSVKGRFTISRDGAKTTLYLQMNSLRPEDT | |
| Peptide-23 | AVYYCAARGTLGLFTTADSYDYWGQGTLVTVSSGGGGSGGGSGGI | |
| mask; CD28 | DWCPRYLWDECYNAGSGGSGGLSGRSDAGSPLGLAGSGGGSDIQM | |
| scFv Vl- | TQSPSSLSASVGDRVTITCHASQNIYVWLNWYQQKPGKAPKLLIYKA | |
| linker-Vh | SNLHTGVPSRFSGSGSGTDFTLTISSLQPEDFATYYCQQGQTYPYTFG | |
| | GGTKVEIKGGGGSGGGGSGGGGSQVQLVQSGAEVKKPGASVKVSC | |
| | KASGYTFTSYYIHWVRQAPGQGLEWIGSIYPGNVNTNYNEKFKDRA | |
| | TLTVDTSISTAYMELSRLRSDDTAVYFCTRSHYGLDWNFDVWGQGT | |
| | TVTVSS | |
| Ab-101 | EVQLVESGGGLVQPGGSLRLSCAASGSTFYTAVMGWVRQAPGKGL | 1455 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-102 | EVQLVESGGGLVQPGGSLRLSCAASGSTFYTAVMGWVRQAPGKGL | 1456 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-103 | EVQLVESGGGLVQPGNSLRLSCAASGFTFSSFGMSWVRQAPGKGLE | 1457 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-104 | EVQLVESGGGLVQPGNSLRLSCAASGFTFSSFGMSWVRQAPGKGLE | 1458 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-105 | EVQLVESGGGLVQPGGSLRLSCAASGSTFYTAVMGWVRQAPGKGL | 1459 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-106 | EVQLVESGGGLVQPGGSLRLSCAASGSTFYTAVMGWVRQAPGKGL | 1460 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-107 | EVQLVESGGGLVQPGNSLRLSCAASGFTFSSFGMSWVRQAPGKGLE | 1461 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-108 | EVQLVESGGGLVQPGNSLRLSCAASGFTFSSFGMSWVRQAPGKGLE | 1462 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-109 | EVQLVESGGGLVQPGGSLRLSCAASGSTFYTAVMGWVRQAPGKGL | 1463 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-110 | EVQLVESGGGLVQPGGSLRLSCAASGSTFYTAVMGWVRQAPGKGL | 1464 |
| HE-1: | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-V1 | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-111 | EVQLVESGGGLVQPGNSLRLSCAASGFTFSSFGMSWVRQAPGKGLE | 1465 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-112 | EVQLVESGGGLVQPGNSLRLSCAASGFTFSSFGMSWVRQAPGKGLE | 1466 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-113 | EVQLVESGGGLVQPGGSLRLSCAASGSTFYTAVMGWVRQAPGKGL | 1467 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-114 | EVQLVESGGGLVQPGGSLRLSCAASGSTFYTAVMGWVRQAPGKGL | 1468 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-115 | EVQLVESGGGLVQPGNSLRLSCAASGFTFSSFGMSWVRQAPGKGLE | 1469 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-116 | EVQLVESGGGLVQPGNSLRLSCAASGFTFSSFGMSWVRQAPGKGLE | 1470 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; scFv | AGSSGGSGGLSGRSDAGSPLGLAGSGGGSDIQMTQSPSSLSASVGDR | |
| Vl-linker-Vh | VTITCHASQNIYVWLNWYQQKPGKAPKLLIYKASNLHTGVPSRFSGS | |
| | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-117 | EVQLVESGGGLVQPGGSLRLSCAASGSTFYTAVMGWVRQAPGKGL | 1471 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; scFv | DWCPRDLWVHCFAGSSGGSGGLSGRSDAGSPLGLAGSGGGSQVQL | |
| Vh-linker-Vl | VQSGAEVKKPGASVKVSCKASGYTFTSYYIHWVRQAPGQGLEWIGS | |
| | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-118 | EVQLVESGGGLVQPGGSLRLSCAASGSTFYTAVMGWVRQAPGKGL | 1472 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-119 | EVQLVESGGGLVQPGNSLRLSCAASGFTFSSFGMSWVRQAPGKGLE | 1473 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-120 | EVQLVESGGGLVQPGNSLRLSCAASGFTFSSFGMSWVRQAPGKGLE | 1474 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-121 | EVQLVESGGGLVQPGGSLRLSCAASGSTFYTAVMGWVRQAPGKGL | 1475 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-122 | EVQLVESGGGLVQPGGSLRLSCAASGSTFYTAVMGWVRQAPGKGL | 1476 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-123 | EVQLVESGGGLVQPGNSLRLSCAASGFTFSSFGMSWVRQAPGKGLE | 1477 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-124 | EVQLVESGGGLVQPGNSLRLSCAASGFTFSSFGMSWVRQAPGKGLE | 1478 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-125 | EVQLVESGGGLVQPGGSLRLSCAASGSTFYTAVMGWVRQAPGKGL | 1479 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-126 | EVQLVESGGGLVQPGGSLRLSCAASGSTFYTAVMGWVRQAPGKGL | 1480 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-127 | EVQLVESGGGLVQPGNSLRLSCAASGFTFSSFGMSWVRQAPGKGLE | 1481 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-128 | EVQLVESGGGLVQPGNSLRLSCAASGFTFSSFGMSWVRQAPGKGLE | 1482 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-129 | EVQLVESGGGLVQPGGSLRLSCAASGSTFYTAVMGWVRQAPGKGL | 1483 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-130 | EVQLVESGGGLVQPGGSLRLSCAASGSTFYTAVMGWVRQAPGKGL | 1484 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-131 | EVQLVESGGGLVQPGNSLRLSCAASGFTFSSFGMSWVRQAPGKGLE | 1485 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-132 | EVQLVESGGGLVQPGNSLRLSCAASGFTFSSFGMSWVRQAPGKGLE | 1486 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-133 | EVQLVESGGGLVQPGGSLRLSCAASGSTFYTAVMGWVRQAPGKGL | 1487 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-134 | EVQLVESGGGLVQPGGSLRLSCAASGSTFYTAVMGWVRQAPGKGL | 1488 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-135 | EVQLVESGGGLVQPGNSLRLSCAASGFTFSSFGMSWVRQAPGKGLE | 1489 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-136 | EVQLVESGGGLVQPGNSLRLSCAASGFTFSSFGMSWVRQAPGKGLE | 1490 |
| HE-4; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSQVQLVQSGAEVKKPGAS | |
| scFv Vh- | VKVSCKASGYTFTSYYIHWVRQAPGQGLEWIGSIYPGNVNTNYNEK | |
| linker-Vl | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-137 | EVQLVESGGGLVQPGGSLRLSCAASGSTFYTAVMGWVRQAPGKGL | 1491 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-138 | EVQLVESGGGLVQPGGSLRLSCAASGSTFYTAVMGWVRQAPGKGL | 1492 |
| HE-1; | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSDIQMT | |
| scFv Vl- | QSPSSLSASVGDRVTITCHASQNIYVWLNWYQQKPGKAPKLLIYKAS | |
| linker-Vh | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-139 | EVQLVESGGGLVQPGNSLRLSCAASGFTFSSFGMSWVRQAPGKGLE | 1493 |
| HE-1; | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-140 | EVQLVESGGGLVQPGNSLRLSCAASGFTFSSFGMSWVRQAPGKGLE | 1494 |
| HE-4: | WVSSISGSGSDTLYADSVKGRFTISRDNAKTTLYLQMNSLRPEDTAV | |
| Peptide-1 | YYCTIGGSLSRSSQGTLVTVSSGGGGSGGGSGGMDWCPRDLWVHCF | |
| mask; CD28 | AGSSGGSGGLSGRSDAGSPLGLAGSGGGSDIQMTQSPSSLSASVGDR | |
| scFv Vl- | VTITCHASQNIYVWLNWYQQKPGKAPKLLIYKASNLHTGVPSRFSGS | |
| linker-Vh | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-141 | EVQLVESGGGLVQPGGSLRLSCAASGSTFYTAVMGWVRQAPGKGL | 1495 |
| HE-1: | EWVAAIRWTALTTSYADSVKGRFTISRDGAKTTLYLQMNSLRPEDT | |
| Peptide-1 | AVYYCAARGTLGLFTTADSYDYWGQGTLVTVSSGGGGSGGGSGGM | |
| mask; CD28 | DWCPRDLWVHCFAGSSGGSGGLSGRSDAGSPLGLAGSGGGSQVQL | |
| scFv Vh- | VQSGAEVKKPGASVKVSCKASGYTFTSYYIHWVRQAPGQGLEWIGS | |
| linker-Vl | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-142 | EVQLVESGGGLVQPGGSLRLSCAASGSTFYTAVMGWVRQAPGKGL | 1496 |
| HE-1; Peptide | EWVAAIRWTALTTSYADSVKGRFTISRDGAKTTLYLQMNSLRPEDT | |
| 1 mask; CD28 | AVYYCAARGTLGLFTTADSYDYWGQGTLVTVSSGGGGSGGGSGGM | |
| scFv Vh- | DWCPRDLWVHCFAGSSGGSGGLSGRSDAGSPLGLAGSGGGSQVQL | |
| linker-Vl | VQSGAEVKKPGASVKVSCKASGYTFTSYYIHWVRQAPGQGLEWIGS | |
| | IYPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-143 | EVQLVESGGGLVQPGNSLRLSCAASGFTFSSFGMSWVRQAPGKGLE | 1497 |
| HE-4; Peptide | WVSSISGSGSDTLYADSVKGRFTISRDNAKTTLYLQMNSLRPEDTAV | |
| 1 mask; scFv- | YYCTIGGSLSRSSQGTLVTVSSGGGGSGGGSGGMDWCPRDLWVHCF | |
| Vh-linker-Vl | AGSSGGSGGLSGRSDAGSPLGLAGSGGGSQVQLVQSGAEVKKPGAS | |
| | VKVSCKASGYTFTSYYIHWVRQAPGQGLEWIGSIYPGNVNTNYNEK | |
| | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-144 | EVQLVESGGGLVQPGNSLRLSCAASGFTFSSFGMSWVRQAPGKGLE | 1498 |
| HE-4; Peptide | WVSSISGSGSDTLYADSVKGRFTISRDNAKTTLYLQMNSLRPEDTAV | |
| 1 mask; scFv- | YYCTIGGSLSRSSQGTLVTVSSGGGGSGGGSGGMDWCPRDLWVHCF | |
| Vh-linker-Vl | AGSSGGSGGLSGRSDAGSPLGLAGSGGGSQVQLVQSGAEVKKPGAS | |
| | VKVSCKASGYTFTSYYIHWVRQAPGQGLEWIGSIYPGNVNTNYNEK | |
| | FKDRATLTVDTSISTAYMELSRLRSDDTAVYFCTRSHYGLDWNFDV | |
| | WGQGTTVTVSSGGGGSGGGGSGGGGSDIQMTQSPSSLSASVGDRVT | |
| | ITCHASQNIYVWLNWYQQKPGKAPKLLIYKASNLHTGVPSRFSGSGS | |
| | GTDFTLTISSLQPEDFATYYCQQGQTYPYTFGGGTKVEIK | |
| Ab-145 | EVQLVESGGGLVQPGGSLRLSCAASGSTFYTAVMGWVRQAPGKGL | 1499 |
| HE-1; Peptide | EWVAAIRWTALTTSYADSVKGRFTISRDGAKTTLYLQMNSLRPEDT | |
| 1 mask; scFv | AVYYCAARGTLGLFTTADSYDYWGQGTLVTVSSGGGGSGGGSGGM | |
| Vl-linker-Vh | DWCPRDLWVHCFAGSSGGSGGLSGRSDAGSPLGLAGSGGGSDIQMT | |
| | QSPSSLSASVGDRVTITCHASQNIYVWLNWYQQKPGKAPKLLIYKAS | |
| | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-146 | EVQLVESGGGLVQPGGSLRLSCAASGSTFYTAVMGWVRQAPGKGL | 1500 |
| HE-1; Peptide | EWVAAIRWTALTTSYADSVKGRFTISRDGAKTTLYLQMNSLRPEDT | |
| 1 mask; scFv | AVYYCAARGTLGLFTTADSYDYWGQGTLVTVSSGGGGSGGGSGGM | |
| Vl-linker-Vh | DWCPRDLWVHCFAGSSGGSGGLSGRSDAGSPLGLAGSGGGSDIQMT | |
| | QSPSSLSASVGDRVTITCHASQNIYVWLNWYQQKPGKAPKLLIYKAS | |
| | NLHTGVPSRFSGSGSGTDFTLTISSLQPEDFATYYCQQGQTYPYTFGG | |
| | GTKVEIKGGGGSGGGGSGGGGSQVQLVQSGAEVKKPGASVKVSCK | |
| | ASGYTFTSYYIHWVRQAPGQGLEWIGSIYPGNVNTNYNEKFKDRAT | |
| | LTVDTSISTAYMELSRLRSDDTAVYFCTRSHYGLDWNFDVWGQGTT | |
| | VTVSS | |
| Ab-147 | EVQLVESGGGLVQPGNSLRLSCAASGFTFSSFGMSWVRQAPGKGLE | 1501 |
| HE-4; Peptide | WVSSISGSGSDTLYADSVKGRFTISRDNAKTTLYLQMNSLRPEDTAV | |
| 1 mask; scFv | YYCTIGGSLSRSSQGTLVTVSSGGGGSGGGSGGMDWCPRDLWVHCF | |
| Vl-linker-Vh | AGSSGGSGGLSGRSDAGSPLGLAGSGGGSDIQMTQSPSSLSASVGDR | |
| | VTITCHASQNIYVWLNWYQQKPGKAPKLLIYKASNLHTGVPSRFSGS | |
| | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-148 | EVQLVESGGGLVQPGNSLRLSCAASGFTFSSFGMSWVRQAPGKGLE | 1502 |
| HE-4; Peptide | WVSSISGSGSDTLYADSVKGRFTISRDNAKTTLYLQMNSLRPEDTAV | |
| 1 mask; scFv | YYCTIGGSLSRSSQGTLVTVSSGGGGSGGGSGGMDWCPRDLWVHCF | |
| Vl-linker-Vh | AGSSGGSGGLSGRSDAGSPLGLAGSGGGSDIQMTQSPSSLSASVGDR | |
| | VTITCHASQNIYVWLNWYQQKPGKAPKLLIYKASNLHTGVPSRFSGS | |
| | GSGTDFTLTISSLQPEDFATYYCQQGQTYPYTFGGGTKVEIKGGGGS | |
| | GGGGSGGGGSQVQLVQSGAEVKKPGASVKVSCKASGYTFTSYYIH | |
| | WVRQAPGQGLEWIGSIYPGNVNTNYNEKFKDRATLTVDTSISTAYM | |
| | ELSRLRSDDTAVYFCTRSHYGLDWNFDVWGQGTTVTVSS | |
| Ab-149 | EVQLVESGGGLVQPGGSLRLSCAASGSTFYTAVMGWVRQAPGKGL | 1503 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| Vh-linker-Vl | CEGHLPEYLAKCLGSSGGSGGLSGRSDAGSPLGLAGSGGGSQVQLV | |
| | QSGAEVKKPGASVKVSCKASGYTFTSYYIHWVRQAPGQGLEWIGSI | |
| | YPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-150 | EVQLVESGGGLVQPGGSLRLSCAASGSTFYTAVMGWVRQAPGKGL | 1504 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| Vh-linker-Vl | CEGHLPEYLAKCLGSSGGSGGLSGRSDAGSPLGLAGSGGGSQVQLV | |
| | QSGAEVKKPGASVKVSCKASGYTFTSYYIHWVRQAPGQGLEWIGSI | |
| | YPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGGGT | |
| | KVEIK | |
| Ab-151 | EVQLVESGGGLVQPGGSLRLSCAASGSTFYTAVMGWVRQAPGKGL | 1505 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| disulfide Vh- | CEGHLPEYLAKCLGSSGGSGGLSGRSDAGSPLGLAGSGGGSQVQLV | |
| linker-Vl | QSGAEVKKPGASVKVSCKASGYTFTSYYIHWVRQAPGQCLEWIGSI | |
| | YPGNVNTNYNEKFKDRATLTVDTSISTAYMELSRLRSDDTAVYFCT | |
| | RSHYGLDWNFDVWGQGTTVTVSSGGGGSGGGGSGGGGSDIQMTQS | |
| | PSSLSASVGDRVTITCHASQNIYVWLNWYQQKPGKAPKLLIYKASNL | |
| | HTGVPSRFSGSGSGTDFTLTISSLQPEDFATYYCQQGQTYPYTFGCGT | |
| | KVEIK | |
| Ab-152 | EVQLVESGGGLVQPGGSLRLSCAASGSTFYTAVMGWVRQAPGKGL | 1506 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| Vl-linker-Vh | CEGHLPEYLAKCLGSSGGSGGLSGRSDAGSPLGLAGSGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIKGGGGSGGGGSGGGGSQVQLVQSGAEVKKPGASVKVSCKA | |
| | SGYTFTSYYIHWVRQAPGQGLEWIGSIYPGNVNTNYNEKFKDRATL | |
| | TVDTSISTAYMELSRLRSDDTAVYFCTRSHYGLDWNFDVWGQGTTV | |
| | TVSS | |
| Ab-153 | EVQLVESGGGLVQPGGSLRLSCAASGSTFYTAVMGWVRQAPGKGL | 1507 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| Vl-linker-Vh | CEGHLPEYLAKCLGSSGGSGGLSGRSDAGSPLGLAGSGGGSDIQMTQ | |
| | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGGG | |
| | TKVEIKGGGGSGGGGSGGGGSQVQLVQSGAEVKKPGASVKVSCKA | |
| | SGYTFTSYYIHWVRQAPGQGLEWIGSIYPGNVNTNYNEKFKDRATL | |
| | TVDTSISTAYMELSRLRSDDTAVYFCTRSHYGLDWNFDVWGQGTTV | |
| | TVSS | |
| Ab-154 | EVQLVESGGGLVQPGGSLRLSCAASGSTFYTAVMGWVRQAPGKGL | 1508 |
| HE-1; Peptide | EWVAAIRWTALTTSYADSVKGRFTISRDGAKTTLYLQMNSLRPEDT | |
| 79 mask; scFv | AVYYCAARGTLGLFTTADSYDYWGQGTLVTVSSGGGGSGGGSGGY | |
| disulfide Vl- | CEGHLPEYLAKCLGSSGGSGGLSGRSDAGSPLGLAGSGGGSDIQMTQ | |
| linker-Vh | SPSSLSASVGDRVTITCHASQNIYVWLNWYQQKPGKAPKLLIYKASN | |
| | LHTGVPSRFSGSGSGTDFTLTISSLQPEDFATYYCQQGQTYPYTFGCG | |
| | TKVEIKGGGGSGGGGSGGGGSQVQLVQSGAEVKKPGASVKVSCKA | |
| | SGYTFTSYYIHWVRQAPGQCLEWIGSIYPGNVNTNYNEKFKDRATLT | |
| | VDTSISTAYMELSRLRSDDTAVYFCTRSHYGLDWNFDVWGQGTTVT | |
| | VSS | |

### Modified Amino Acids

In some embodiments, the isolated polypeptide or polypeptide complex comprises a modified amino acid, a non-natural amino acid, a modified non-natural amino acid, or a combination thereof. In some embodiments, the non-natural amino acid comprises a D-amino acid. In some embodiments, the modified amino acid or modified non-natural amino acid comprises a post-translational modification.

### Pharmaceutical Compositions

Disclosed herein are pharmaceutical compositions comprising: (i) the polypeptide or polypeptide complex according to any embodiment disclosed herein, and (ii) a pharmaceutically acceptable excipient.

Disclosed herein are pharmaceutical compositions comprising an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Disclosed herein are pharmaceutical compositions comprising an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T.

Disclosed herein are pharmaceutical compositions comprising an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Disclosed herein, in some embodiments, are pharmaceutical compositions comprising an isolated polypeptide or polypeptide complex comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A.

Disclosed herein, in some embodiments, are pharmaceutical compositions comprising an isolated polypeptide or polypeptide complex comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A.

Disclosed herein, in some embodiments, are pharmaceutical compositions comprising an isolated polypeptide or polypeptide complex comprising a CD28 binding domain that is linked to a peptide that impairs binding of the CD28 binding domain to CD28, wherein the peptide comprises an amino acid sequence according to any one of SEQ ID NOs: 120-201 and 204-1392.

Disclosed herein, in some embodiments, are pharmaceutical compositions comprising an isolated polypeptide or polypeptide complex comprising a CD28 binding domain that is linked to a peptide that impair binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservatives, emulsifying agents and dispersing agents. Prevention of the action of microorganisms may be ensured by the inclusion of various antibacterial and antifungal agents.

The pharmaceutical composition may be in any suitable form, depending upon the desired method of administration. It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Isolated Recombinant Nucleic Acid Molecules

Disclosed herein are isolated recombinant nucleic acid molecules encoding an isolated polypeptide or polypeptide complex of any one of the embodiments disclosed herein.

Disclosed herein, in some embodiments, are isolated recombinant nucleic acid molecules encoding an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Disclosed herein, in some embodiments, are isolated recombinant nucleic acid molecules encoding an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T.

Disclosed herein, in some embodiments, are isolated recombinant nucleic acid molecules encoding an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Disclosed herein, in some embodiments, are isolated recombinant nucleic acid molecules encoding an isolated polypeptide or polypeptide complex comprising a peptide that impairs binding of a CD28 binding domain to CD28 expressed on an effector cell, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A.

Disclosed herein, in some embodiments, are isolated recombinant nucleic acid molecules encoding an isolated polypeptide or polypeptide complex comprising a peptide that impairs binding of a CD28 binding domain to CD28 expressed on an effector cell, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A.

Disclosed herein, in some embodiments, are isolated recombinant nucleic acid molecules encoding an isolated polypeptide or polypeptide complex comprising a peptide that impairs binding of a CD28 binding domain to CD28 expressed on an effector cell, wherein the peptide comprises an amino acid sequence according to any one of SEQ ID NOs: 120-201 and 204-1392.

Disclosed herein, in some embodiments, are isolated recombinant nucleic acid molecules encoding an isolated polypeptide or polypeptide complex comprising a peptide that impairs binding of a CD28 binding domain to CD28 expressed on an effector cell, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

### Methods of Treatment

Disclosed herein, in some embodiments, are methods of treating cancer in a subject in need thereof comprising administering to the subject an isolated polypeptide or polypeptide complex of any one of the embodiments disclosed herein.

Disclosed herein, in some embodiments, are methods of treating cancer in a subject in need thereof comprising administering to the subject an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Disclosed herein, in some embodiments, are methods of treating cancer in a subject in need thereof comprising administering to the subject an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T.

Disclosed herein, in some embodiments, are methods of treating cancer in a subject in need thereof comprising administering to the subject an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Disclosed herein, in some embodiments, are methods of treating cancer in a subject in need thereof comprising administering to the subject an isolated polypeptide or polypeptide complex comprising a peptide that impairs binding of a CD28 binding domain to CD28 expressed on an effector cell, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A.

Disclosed herein, in some embodiments, are methods of treating cancer in a subject in need thereof comprising administering to the subject an isolated polypeptide or polypeptide complex comprising a peptide that impairs binding of a CD28 binding domain to CD28 expressed on an effector cell, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A.

Disclosed herein, in some embodiments, are methods of treating cancer in a subject in need thereof comprising administering to the subject an isolated polypeptide or polypeptide complex comprising a peptide that impairs binding of a CD28 binding domain to CD28 expressed on an effector cell, wherein the peptide comprises an amino acid sequence according to any one of SEQ ID NOs: 120-201 and 204-1392.

Disclosed herein, in some embodiments, are methods of treating cancer in a subject in need thereof comprising administering to the subject an isolated polypeptide or polypeptide complex comprising a peptide that impairs binding of a CD28 binding domain to CD28 expressed on an effector cell, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

### Production of Antibodies

In some embodiments, the antibodies or antigen binding fragments thereof as described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt- cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1). In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

Disclosed herein, in some embodiments, are vectors comprising an isolated recombinant nucleic acid molecule encoding an isolated polypeptide or polypeptide complex of any one of the embodiments disclosed herein.

Disclosed herein, in some embodiments, are vectors comprising an isolated recombinant nucleic acid molecule encoding an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Disclosed herein, in some embodiments, are vectors comprising an isolated recombinant nucleic acid molecule encoding an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T.

Disclosed herein, in some embodiments, are vectors comprising an isolated recombinant nucleic acid molecule encoding an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Disclosed herein, in some embodiments, are vectors comprising an isolated recombinant nucleic acid molecule encoding an isolated polypeptide or polypeptide complex comprising an isolated polypeptide or polypeptide complex comprising a peptide that impairs binding of a CD28 binding domain to CD28 expressed on an effector cell, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A.

Disclosed herein, in some embodiments, are vectors comprising an isolated recombinant nucleic acid molecule encoding an isolated polypeptide or polypeptide complex comprising a peptide that impairs binding of a CD28 binding domain to CD28 expressed on an effector cell, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A.

Disclosed herein, in some embodiments, are vectors comprising an isolated recombinant nucleic acid encoding an isolated polypeptide or polypeptide complex comprising a peptide that impairs binding of a CD28 binding domain to CD28 expressed on an effector cell, wherein the peptide comprises an amino acid sequence according to any one of SEQ ID NOs: 120-201 and 204-1392.

Disclosed herein, in some embodiments, are vectors comprising an isolated recombinant nucleic acid encoding an isolated polypeptide or polypeptide complex comprising a peptide that impairs binding of a CD28 binding domain to CD28 expressed on an effector cell, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

In some embodiments, vectors include any suitable vectors derived from either eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

Disclosed herein, in some embodiments, are host cells comprising an isolated polypeptide or polypeptide complex of any one of the embodiments disclosed herein.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Disclosed herein, in some embodiments, are host cells comprising an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein X₁ is selected from A, R, N, D, Q, E, G, H, I, L, K, F, P, S, T, W, Y, and V; X₂ is selected from D, P, and Y; X₃ is selected from P and Q; X₄ is selected from R, L, and I; X₅ is selected from D, H, Y, and I; X₆ is selected from L, S, and R; X₇ is selected from W and G; X₈ is selected from V, A, D, H, L, and N; X₉ is selected from H, L, E, and D; X₁₀ is selected from F, Y, and L; and X₁₁ is selected from A, N, H, and T.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein Z₁ is selected from Y, H, and A; Z₂ is selected from D, E, A, Q, S, V, M, H, L, F, T, W, and N; Z₃ is selected from G, E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence according to any one of SEQ ID NOs: 120-201 and 204-1392.

Disclosed herein, in some embodiments, are host cells comprising an isolated polypeptide or polypeptide complex comprising a CD28 binding domain that is linked to a peptide that impairs binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast. Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division. Exemplary insect host cells include, but are not limited to, Drosophila S2 cells, Sf9 cells, Sf21 cells, High Five™ cells, and expresSF+® cells. In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an isolate polypeptide or polypeptide complex of any embodiment disclosed herein. The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EMBODIMENTS

Embodiment 1 comprises an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

Embodiment 2 comprises the isolated polypeptide or polypeptide complex of embodiment 1, wherein the peptide comprises an amino acid sequence selected from any one of SEQ ID NOs: 1-26.

Embodiment 3 comprises the isolated polypeptide or polypeptide complex of embodiment 1, wherein the peptide consists of an amino acid sequence selected from any one of SEQ ID NOs: 1-26.

Embodiment 4 comprises the isolated polypeptide or polypeptide complex of embodiment 1, wherein the peptide comprises the amino acid sequence of SEQ ID NO: 1.

Embodiment 5 comprises the isolated polypeptide or polypeptide complex of embodiment 1, wherein the peptide consists of the amino acid sequence of SEQ ID NO: 1.

Embodiment 6 comprises the isolated polypeptide or polypeptide complex of embodiment 1, wherein the peptide comprises an amino acid sequence selected from any one of SEQ ID NOs: 2-19 and 22- 26 or the peptide comprises 1, 2, or 3 amino acid substitutions, additions, or deletions relative to an amino acid sequence selected from any one of SEQ ID NOs: 2-19 and 22-26.

Embodiment 7 comprises the isolated polypeptide or polypeptide complex of embodiment 1, wherein the peptide comprises an amino acid sequence selected from any one of SEQ ID NOs: 2-19 and 22-26.

Embodiment 8 comprises the isolated polypeptide or polypeptide complex of embodiment 1, wherein the peptide comprises an amino acid sequence selected from any one of SEQ ID NOs: 2-19.

Embodiment 9 comprises the isolated polypeptide or polypeptide complex of embodiment 1, wherein the peptide consists of an amino acid sequence selected from any one of SEQ ID NOs: 2-19.

Embodiment 10 comprises the isolated polypeptide or polypeptide complex of embodiment 1, wherein the peptide comprises an amino acid sequence selected from any one of SEQ ID NOs: 2-9 and 12-18.

Embodiment 11 comprises the isolated polypeptide or polypeptide complex of embodiment 1, wherein the peptide consists of an amino acid sequence selected from any one of SEQ ID NOs: 2-9 and 12-18.

Embodiment 12 comprises the isolated polypeptide or polypeptide complex of embodiment 1, wherein the peptide comprises an amino acid sequence selected from any one of SEQ ID NOs: 3, 6-7, 9-13, and 16-19.

Embodiment 13 comprises the isolated polypeptide or polypeptide complex of embodiment 1, wherein the peptide comprises an amino acid sequence selected from any one of SEQ ID NOs: 3, 6, 11-12, and 16-17.

Embodiment 14 comprises the isolated polypeptide or polypeptide complex of embodiment 1, wherein the peptide comprises an amino acid sequence selected from any one of SEQ ID NOs: 22-26.

Embodiment 15 comprises the isolated polypeptide or polypeptide complex of embodiment 1, wherein the peptide consists of an amino acid sequence selected from any one of SEQ ID NOs: 22-26.

Embodiment 16 comprises the isolated polypeptide or polypeptide complex of embodiment 1, wherein the peptide comprises an amino acid sequence selected from any one of SEQ ID NOs: 22-24 and 26.

Embodiment 17 comprises the isolated polypeptide or polypeptide complex of embodiment 1, wherein the peptide comprises an amino acid sequence selected from any one of SEQ ID Nos: 22-23 and 26.

Embodiment 18 comprises the isolated polypeptide or polypeptide complex of embodiment 1, wherein the peptide comprises the amino acid sequence of SEQ ID NO: 23.

Embodiment 19 comprises the isolated polypeptide or polypeptide complex of embodiment 1, wherein the peptide consists of the amino acid sequence of SEQ ID NO: 23.

Embodiment 20 comprises an isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein
X₁ is selected from A, R, N, D, Q, E, G, H, I, L, K, F, P, S, T, W, Y, and V;
X₂ is selected from D, P, and Y;
X₃ is selected from P and Q;
X₄ is selected from R, L, and I;
X₅ is selected from D, H, Y, and I;
X₆ is selected from L, S, and R;
X₇ is selected from W and G;
X₈ is selected from V, A, D, H, L, and N;
X₉ is selected from H, L, E, and D;
X₁₀ is selected from F, Y, and L; and
X₁₁ is selected from A, N, H, and T.

Embodiment 21 comprises the isolated polypeptide or polypeptide complex of embodiment 20, wherein:
X₁ is selected from A, R, N, D, Q, E, G, H, K, F, P, S, T, W, and Y.

Embodiment 22 comprises the isolated polypeptide or polypeptide complex of embodiment 20, wherein:
X₁ is selected from R, Q, E, H, I, L, K, F, T, W, Y, and V;
X₂ is D;
X₃ is P;
X₄ is R;
X₅ is D;
X₆ is L;
X₇ is W;
X₈ is V;
X₉ is H;
X₁₀ is F; and
X₁₁ is A.

Embodiment 23 comprises the isolated polypeptide or polypeptide complex of embodiment 20, wherein
X₁ is selected from R, Q, E, L, K, T, and W;
X₂ is D;
X₃ is P;
X₄ is R;
X₅ is D;
X₆ is L;
X₇ is W;
X₈ is V;
X₉ is H;
X₁₀ is F; and
X₁₁ is A.

Embodiment 24 comprises the isolated polypeptide or polypeptide complex of embodiment 20, wherein:
X₁ is L or I;
X₂ is D or Y;
X₃ is P;
X₄ is R, L, or I;
X₅ is H, Y, or D;
X₆ is L or R;
X₇ is W;
X₈ is A, D, H, or N;
X₉ is L, E, or D;
X₁₀ is F or Y; and
X₁₁ is N or H.

Embodiment 25 comprises the isolated polypeptide or polypeptide complex of embodiment 20, wherein:
X₁ is L or I;
X₂ is D or Y;
X₃ is P;
X₄ is R or I;
X₅ is H, Y, or D;
X₆ is L or R;
X₇ is W;
X₈ is A, D, or N;
X₉ is L, E, or D;
X₁₀ is F or Y; and
X₁₁ is N.

Embodiment 26 comprises an isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

Embodiment 27 comprises the isolated polypeptide or polypeptide complex of embodiment 26, wherein the peptide comprises an amino acid sequence selected from any one of SEQ ID NOs: 2-26.

Embodiment 28 comprises the isolated polypeptide or polypeptide complex of embodiment 26, wherein the peptide consists of an amino acid sequence selected from any one of SEQ ID NOs: 2-26.

Embodiment 29 comprises an isolated polypeptide or polypeptide complex comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein:
Z₁ is selected from Y, H, and A;
Z₂ is selected from D, E, A, Q, S, V, M, H, L, F, T, W, and N;
Z₃ is selected from G, E, S, and A;
Z₄ is selected from H, L, W, and A;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, Q, and A;
Z₉ is selected from M, I, L, R, V, K, F, Q, Y, W, and A;
Z₁₀ is selected from A, Q, S, W, E, L, G, D, T, M, N, V, H, R, Y, K, P, F, and I;
Z₁₁ is selected from K, Q, N, H, E, P, and A; and
Z₁₂ is selected from L, M, I, F, V, Y, Q, D, T, and A.

Embodiment 30 comprises the isolated polypeptide or polypeptide complex of embodiment 29, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 31 comprises the isolated polypeptide or polypeptide complex of embodiments 29 or 30, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 32 comprises the isolated polypeptide or polypeptide complex of any one of embodiments 29 to 31, wherein:
Z₁ is Y;
Z₂ is selected from D, E, A, and Q;
Z₃ is G;
Z₄ is H;
Z₅ is selected from L, S, A, T, I, V, and M;
Z₆ is selected from P, A, E, Q, S, L, W, G, V, and D;
Z₇ is selected from E, Q, I, M, V, A, and L;
Z₈ is selected from Y and H;
Z₉ is selected from M, I, and L;
Z₁₀ is selected from A, Q, S, W, E, L, G, D, T, M, and N;
Z₁₁ is K; and
Z₁₂ is selected from L and M.

Embodiment 33 comprises the isolated polypeptide or polypeptide complex of any one of embodiments 29-32, wherein:
Z₁ is Y;
Z₂ is selected from D, E, and A;
Z₃ is G;
Z₄ is H;
Z₅ is selected from L, S, A, and T;
Z₆ is selected from P, A, E, Q, S, L, and W;
Z₇ is selected from E, Q, I, and M;
Z₈ is Y;
Z₉ is selected from M, I, and L;
Z₁₀ is selected from A, Q, S, W, E, L, G, and D;
Z₁₁ is K; and
Z₁₂ is L.

Embodiment 34 comprises the isolated polypeptide or polypeptide complex of any one of embodiments 29 to 33, wherein:
Z₁ is Y;
Z₂ is selected from D and E;
Z₃ is G;
Z₄ is H;
Z₅ is selected from L, S, and A;
Z₆ is selected from P, A, E, Q, S, and L;
Z₇ is selected from E, Q, and I;
Z₈ is Y;
Z₉ is selected from M, I, and L;
Z₁₀ is selected from A, Q, S, W, E, L, G, and D;
Z₁₁ is K; and
Z₁₂ is L.

Embodiment 35 comprises the isolated polypeptide or polypeptide complex of embodiment 29, wherein the peptide comprises the amino acid sequence of SEQ ID NO: 132.

Embodiment 36 comprises the isolated polypeptide or polypeptide complex of embodiment 29, wherein the peptide consists of the amino acid sequence of SEQ ID NO: 132.

Embodiment 37 comprises the isolated polypeptide or polypeptide complex of embodiment 29, wherein the peptide comprises an amino acid sequence according to any one of SEQ ID NOs: 162-181.

Embodiment 38 comprises the isolated polypeptide or polypeptide complex of embodiment 29, wherein the peptide comprises the amino acid sequence according to SEQ ID NO: 172.

Embodiment 39 comprises the isolated polypeptide or polypeptide complex of embodiment 29, wherein the peptide comprises an amino acid sequence according to any one of SEQ ID NOs: 152 to 161.

Embodiment 40 comprises an isolated polypeptide or polypeptide complex comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein:
U₁ is selected from T, S, L, M, W, Q, P, R, G, N, I, Y, D, E, V, H, and A;
U₂ is selected from A, S, T, K, E, R, G, N, W, and D;
U₃ is selected from A, E, P, D, G, W, Q, V, and F;
U₄ is selected from G, V, F, D, E, R, and A;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, I, and A;
U₈ is selected from P, S, T, A, E, K, Q, N, V, G, H, L, D, Y, I, M, and R;
U₉ is selected from W, L, and A;
U₁₀ is selected from P, S, R, L, E, and A;
U₁₁ is selected from L, A, T, M, V, W, Y, E, Q, I, F, S, P, K, R, D, H, N, and G; and
U₁₂ is selected from L, F, V, M, I, Y, E, W, and A.

Embodiment 41 comprises the isolated polypeptide or polypeptide complex of embodiment 40, wherein:
U₁ is selected from T, S, L, M, W, Q, P, R, G, N, I, Y, D, E, V, and H;
U₂ is selected from A, S, T, K, E, R, G, N, W, and D;
U₃ is selected from A, E, P, D, G, W, Q, V, and F;
U₄ is selected from G, V, F, D, E, and R;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, and I;
U₈ is selected from P, S, T, A, E, K, Q, N, V, G, H, L, D, Y, I, M, and R;
U₉ is selected from W, L, and A;
U₁₀ is selected from P, S, R, L, and E;
U₁₁ is selected from L, A, T, M, V, W, Y, E, Q, I, F, S, P, K, R, D, H, N, and G; and
U₁₂ is selected from L, F, V, M, I, Y, E, and W.

Embodiment 42 comprises the isolated polypeptide or polypeptide complex of embodiment 40 or 41, wherein:
U₁ is selected from T, S, L, M, and W;
U₂ is selected from A, S, and T;
U₃ is selected from A and E;
U₄ is selected from G, V, and F;
U₅ is selected from A, F, and V;
U₆ is selected from T, S, H, M, A, and N;
U₇ is selected from Y, W, and F;
U₈ is selected from P, S, T, A, E, K, Q, N, and V;
U₉ is selected from W and L;
U₁₀ is selected from P and S;
U₁₁ is selected from L, A, T, M, V, W, Y, E, Q, I, F, and S; and
U₁₂ is selected from L, F, V, and M.

Embodiment 43 comprises the isolated polypeptide or polypeptide complex of any one of embodiments 40 to 42, wherein:
U₁ is selected from T and S;
U₂ is selected from A and S;
U₃ is A;
U₄ is selected from G and V;
U₅ is selected from A and F;
U₆ is selected from T, S, and H;
U₇ is selected from Y and W;
U₈ is selected from P, S, T, and A;
U₉ is W;
U₁₀ is P;
U₁₁ is selected from L, A, T, M, V, W, and Y; and
U₁₂ is selected from L and F.

Embodiment 44 comprises the isolated polypeptide or polypeptide complex of embodiment 40, wherein the peptide comprises the amino acid sequence of SEQ ID NO: 137.

Embodiment 45 comprises the isolated polypeptide or polypeptide complex of embodiment 40, wherein the peptide consists of the amino acid sequence of SEQ ID NO: 137.

Embodiment 46 comprises the isolated polypeptide or polypeptide complex of embodiment 40, wherein the peptide comprises an amino acid sequence according to any one of SEQ ID NOs: 144 to 151.

Embodiment 47 comprises the isolated polypeptide or polypeptide complex of embodiment 40, wherein the peptide comprises an amino acid sequence according to any one of SEQ ID NOs: 182-201.

Embodiment 48 comprises an isolated polypeptide or polypeptide complex comprising a CD28 binding domain that is linked to a peptide that impairs binding of the CD28 binding domain to CD28, wherein the peptide comprises an amino acid sequence according to any one of SEQ ID NOs: 120-201 and 204-1392.

Embodiment 49 comprises an isolated polypeptide or polypeptide complex comprising a CD28 binding domain that is linked to a peptide that impair binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein:
X₁ is selected from M, I, L, and V;
X₂ is selected from D, H, N, A, F, S, T, Y, and V;
X₃ is selected from W, L, and F;
X₄ is selected from P, A, and L;
X₅ is selected from R, T, I, M, S, K, L, V, W, F, A, P, and D;
X₆ is selected from E, D, Y, H, S, F, A, N, T, I, P, and V;
X₇ is selected from L, M, R, S, Q, and H;
X₈ is selected from W and Q;
X₉ is selected from H, N, D, A, S, Y, T, F, V, L, and I;
X₁₀ is selected from E, V, L, D, Y, R, Q, H, F, K, A, M, and N;
X₁₁ is selected from F, Y, L, W, and V; and
X₁₂ is selected from N, A, F, S, Y, H, D, T, and L.

Embodiment 50 comprises the isolated polypeptide or polypeptide complex of embodiment 49, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y.

Embodiment 51 comprises the isolated polypeptide or polypeptide complex of embodiment 49, wherein X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

Embodiment 52 comprises the isolated polypeptide or polypeptide complex of any one of the preceding embodiments, wherein the CD28 binding domain comprises an anti-CD28 antibody.

Embodiment 53 comprises the isolated polypeptide or polypeptide complex of embodiment 52, wherein the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise the amino acid sequences of HC-CDR1: SEQ ID NO: 27, HC-CDR2: SEQ ID NO: 28, and HC-CDR3: SEQ ID NO: 29, and the anti-CD28 antibody comprises an anti-CD28 light chain variable domain that comprises complementarity determining regions (CDRs): LC-CDR1, LC-CDR2, and LC-CDR3, wherein the LC-CDR1, the LC-CDR2, and the LC-CDR3 comprise the amino acid sequences of LC-CDR1: SEQ ID NO: 30, LC-CDR2: (KAS), and LC-CDR3: SEQ ID: 32.

Embodiment 54 comprises the isolated polypeptide or polypeptide complex of embodiment 53, wherein the anti-CD28 heavy chain variable domain comprises an amino acid sequence with at least 80%, 85%, 90%, 95%, 98%, 99%, or 100% sequence identity to SEQ ID NO: 33.

Embodiment 55 comprises the isolated polypeptide or polypeptide complex of embodiment 53 or 54, wherein the anti-CD28 light chain variable domain comprises an amino acid sequence with at least 80%, 85%, 90%, 95%, 98%, 99%, or 100% sequence identity to SEQ ID NO: 34.

Embodiment 56 comprises the isolated polypeptide or polypeptide complex of any one of embodiments 52 to 55, wherein the anti-CD28 antibody comprises a single chain variable fragment (scFv), a single domain antibody, a Fab, or a Fab′.

Embodiment 57 comprises the isolated polypeptide or polypeptide complex of embodiment 56, wherein the anti-CD28 antibody comprises the scFv.

Embodiment 58 comprises the isolated polypeptide or polypeptide complex of embodiment 56, wherein the anti-CD28 antibody comprises the scFv, and the scFv comprises an amino acid sequence with at least 80%, 85%, 90%, 95%, 98%, 99%, or 100% sequence identity to SEQ ID NO: 35.

Embodiment 59 comprises the isolated polypeptide or polypeptide complex of embodiment 56, wherein the anti-CD28 antibody comprises the scFv, and the scFv comprises an amino acid sequence with at least 80%, 85%, 90%, 95%, 98%, 99%, or 100% sequence identity to SEQ ID NO: 67.

Embodiment 60 comprises the isolated polypeptide or polypeptide complex of embodiment 56, wherein the anti-CD28 antibody comprises the scFv, and the scFv comprises an amino acid sequence with at least 80%, 85%, 90%, 95%, 98%, 99%, or 100% sequence identity to SEQ ID NO: 68.

Embodiment 61 comprises the isolated polypeptide or polypeptide complex of embodiment 56, wherein the anti-CD28 antibody comprises the scFv, and the scFv comprises an amino acid sequence with at least 80%, 85%, 90%, 95%, 98%, 99%, or 100% sequence identity to SEQ ID NO: 69.

Embodiment 62 comprises the isolated polypeptide or polypeptide complex of embodiment 56, wherein the anti-CD28 antibody comprises the Fab, wherein the Fab comprises an immunoglobulin light chain with at least 80%, 85%, 90%, 95%, 98%, 99%, or 100% sequence identity to SEQ ID NO: 202, and wherein the Fab comprises an immunoglobulin heavy chain with 80%, 85%, 90%, 95%, 98%, 99%, or 100% sequence identity to SEQ ID NO: 203.

Embodiment 63 comprises the isolated polypeptide or polypeptide complex of any one of the preceding embodiments, wherein the isolated polypeptide or polypeptide complex is human or humanized.

Embodiment 64 comprises the isolated polypeptide or polypeptide complex of any one of the preceding embodiments, wherein the peptide is connected to the CD28 binding domain in a configuration according to A₁-L₁-P₁ wherein A₁ comprises the CD28 binding domain, P₁ comprises the peptide that impairs binding of the CD28 binding domain to CD28, and L₁ comprises a cleavable linker.

Embodiment 65 comprises the isolated polypeptide or polypeptide complex of embodiment 64, wherein P₁ is connected N-terminal to the cleavable linker and A₁ is connected C-terminal to the cleavable linker.

Embodiment 66 comprises the isolated polypeptide or polypeptide complex of embodiment 64, wherein P₁ is connected C-terminal to the cleavable linker and A₁ is connected N-terminal to the cleavable linker.

Embodiment 67 comprises the isolated polypeptide or polypeptide complex of any one of embodiments 64-66, wherein P₁ is bound to A₁ through ionic interactions, electrostatic interactions, hydrophobic interactions, Pi-stacking interactions, H-bonding interactions, or a combination thereof.

Embodiment 68 comprises the isolated polypeptide or polypeptide complex of any one of embodiments 64-67, wherein P₁ is bound to A₁ at or near an antigen binding site.

Embodiment 69 comprises the isolated polypeptide of polypeptide complex any one of embodiments 64-68, wherein P₁ becomes unbound from A₁ when L₁ is cleaved by a tumor specific protease thereby exposing A₁ to CD28.

Embodiment 70 comprises the isolated polypeptide or polypeptide complex of any one of embodiments 64-69, wherein P₁ has less than 75% sequence identity to CD28.

Embodiment 71 comprises the isolated polypeptide or polypeptide complex of any one of embodiments 64-70, wherein P₁ comprises a de novo amino acid sequence that shares less than 10% sequence identity to CD28.

Embodiment 72 comprises the isolated polypeptide or polypeptide complex of any one of embodiments 64-71, wherein P₁ comprises a modified amino acid, or a modified non-natural amino acid, or a combination thereof.

Embodiment 73 comprises the isolated polypeptide or polypeptide complex of any one of embodiments 64-72, wherein P₁ does not comprise albumin or an albumin fragment.

Embodiment 74 comprises the isolated polypeptide or polypeptide complex of any one of embodiments 64-73, wherein P₁ does not comprise an albumin binding domain.

### L₁

Embodiment 75 comprises the isolated polypeptide or polypeptide complex of any one of embodiments 64-74, wherein L₁ is bound to the N-terminus of A₁.

Embodiment 76 comprises the isolated polypeptide or polypeptide complex of any one of embodiments 64-74, wherein L₁ is bound to the C-terminus of A₁.

Embodiment 77 comprises the isolated polypeptide or polypeptide complex of any one of embodiments 64-76, wherein L₁ is a peptide sequence having at least 5 to no more than 50 amino acids.

Embodiment 78 comprises the isolated polypeptide or polypeptide complex of any one of embodiments 64-76, wherein L₁ is a peptide sequence having at least 10 to no more than 30 amino acids.

Embodiment 79 comprises the isolated polypeptide or polypeptide complex of any one of embodiments 64-76, wherein L₁ is a peptide sequence having at least 10 amino acids.

Embodiment 80 comprises the isolated polypeptide or polypeptide complex of any one of embodiments 64-76, wherein L₁ is a peptide sequence having at least 18 amino acids.

Embodiment 81 comprises the isolated polypeptide or polypeptide complex of any one of embodiments 64-76, wherein L₁ is a peptide sequence having at least 26 amino acids.

Embodiment 82 comprises the isolated polypeptide or polypeptide complex of any one of embodiments 64-81, wherein L₁ comprises a formula comprising (G₂S)ₙ, wherein n is an integer from 1 to 3 (SEQ ID NO: 1993).

Embodiment 83 comprises the isolated polypeptide or polypeptide complex of any one of embodiments 64-81, wherein L₁ comprises a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1.

Embodiment 84 comprises the isolated polypeptide or polypeptide complex of any one of embodiments 64-81, wherein L₁ comprises an amino acid sequence selected from any one of SEQ ID NOs: 36-65.

Embodiment 85 comprises the isolated polypeptide or polypeptide complex of any one of embodiments 64-81, wherein L₁ comprises the amino acid sequence of SEQ ID NO: 40.

Embodiment 86 comprises the isolated polypeptide or polypeptide complex of any one of embodiments 64-81, wherein L₁ comprises the amino acid sequence of SEQ ID NO: 65.

Embodiment 87 comprises the isolated polypeptide or polypeptide complex of any one of embodiments 64-86, wherein L₁ is a substrate for a tumor specific protease.

Embodiment 88 comprises the isolated polypeptide or polypeptide complex of any one of embodiments 64-87, wherein P₁ becomes unbound from A₁ when L₁ is cleaved by the tumor specific protease thereby exposing A₁ to CD28.

Embodiment 89 comprises the isolated polypeptide or polypeptide complex of embodiments 87 or 88, wherein the tumor specific protease is selected from the group consisting of metalloprotease, serine protease, cysteine protease, threonine protease, and aspartic protease.

Embodiment 90 comprises the isolated polypeptide or polypeptide complex of any one of embodiments 64-89, wherein L₁ comprises a urokinase cleavable amino acid sequence, a matriptase cleavable amino acid sequence, or a matrix metalloprotease cleavable amino acid sequence.

Embodiment 91 comprises the isolated polypeptide or polypeptide complex of any one of the preceding embodiments, wherein the isolated polypeptide or polypeptide complex comprises an amino acid sequence with at least 80%, 85%, 90%, 95%, 99%, or 100% sequence identity to any one of SEQ ID NOs: 74-78, 80, 82, 84-85, 88, 90, and 1393-1450.

Embodiment 92 comprises the isolated polypeptide or polypeptide complex of any one of the preceding embodiments, wherein the isolated polypeptide or polypeptide complex comprises an amino acid sequence according to any one of SEQ ID NOs: 74-78, 80, 82, 84-85, 88, 90, and 1393-1450.

Embodiment 93 comprises the isolated polypeptide or polypeptide complex of any one of the preceding embodiments, wherein the isolated polypeptide or polypeptide complex comprises an amino acid sequence with at least 80%, 85%, 90%, 95%, 99%, or 100% sequence identity to any one of SEQ ID NOs: 1445-1450.

Embodiment 94 comprises the isolated polypeptide or polypeptide complex of any one of the preceding embodiments, wherein the isolated polypeptide or polypeptide complex comprises an amino acid sequence with at least 80%, 90%, 95%, 99%, or 100% identity to any one of SEQ ID NOs: 1393-1450.

Embodiment 95 comprises the isolated polypeptide or polypeptide complex of any one of the preceding embodiments, wherein the isolated polypeptide or polypeptide complex further comprises a half-life extending molecule, H₁.

Embodiment 96 comprises the isolated polypeptide or polypeptide complex of embodiment 95, wherein H₁ comprises a polymer.

Embodiment 97 comprises the isolated polypeptide or polypeptide complex of embodiment 96, wherein the polymer is polyethylene glycol (PEG).

Embodiment 98 comprises the isolated polypeptide or polypeptide complex of embodiment 95, wherein H₁ comprises albumin.

Embodiment 99 comprises the isolated polypeptide or polypeptide complex of embodiment 95, wherein H₁ comprises an Fc domain.

Embodiment 100 comprises the isolated polypeptide or polypeptide complex of embodiment 98, wherein the albumin is serum albumin.

Embodiment 101 comprises the isolated polypeptide or polypeptide complex of embodiment 98, wherein the albumin is human serum albumin.

Embodiment 102 comprises the isolated polypeptide or polypeptide complex of embodiment 95, wherein H₁ comprises a polypeptide, a ligand, or a small molecule.

Embodiment 103 comprises the isolated polypeptide or polypeptide complex of embodiment 102, wherein the polypeptide, the ligand or the small molecule binds serum protein or a fragment thereof, a circulating immunoglobulin or a fragment thereof, or CD35/CR1.

Embodiment 104 comprises the isolated polypeptide or polypeptide complex of embodiment 103, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 105 comprises the isolated polypeptide or polypeptide complex of embodiment 103, wherein the circulating immunoglobulin molecule comprises IgGl, IgG2, IgG3, IgG4, slgA, IgM or IgD.

Embodiment 106 comprises the isolated polypeptide or polypeptide complex of embodiment 103, wherein the serum protein is albumin.

Embodiment 107 comprises the isolated polypeptide or polypeptide complex of embodiments 102 or 103, wherein the polypeptide is an antibody.

Embodiment 108 comprises the isolated polypeptide or polypeptide complex of embodiment 107, wherein the antibody comprises a single domain antibody, a single chain variable fragment, or a Fab.

Embodiment 109 comprises the isolated polypeptide or polypeptide complex of embodiment 108, wherein the single domain antibody comprises a single domain antibody that binds to albumin.

Embodiment 110 comprises the isolated polypeptide or polypeptide complex of embodiment 107 or 108, wherein the single domain antibody is a human or humanized antibody.

Embodiment 111 comprises the isolated polypeptide or polypeptide complex of embodiment 108, wherein the single domain antibody is 645gH1gL1.

Embodiment 112 comprises the isolated polypeptide or polypeptide complex of embodiment 108, wherein the single domain antibody is 645dsgH5gL4.

Embodiment 113 comprises the isolated polypeptide or polypeptide complex of embodiment 108, wherein the single domain antibody is 23-13-A01-sc02.

Embodiment 114 comprises the isolated polypeptide or polypeptide complex of embodiment 108, wherein the single domain antibody is A10m3 or a fragment thereof.

Embodiment 115 comprises the isolated polypeptide or polypeptide complex of embodiment 108, wherein the single domain antibody is DOM7r-31.

Embodiment 116 comprises the isolated polypeptide or polypeptide complex of embodiment 108, wherein the single domain antibody is DOM7h-11-15.

Embodiment 117 comprises the isolated polypeptide or polypeptide complex of embodiment 108, wherein the single domain antibody is Alb-1, Alb-8, or Alb-23.

Embodiment 118 comprises the isolated polypeptide or polypeptide complex of embodiment 108, wherein the single domain antibody is 10G or 10E.

Embodiment 119 comprises the isolated polypeptide or polypeptide complex of embodiment 108, wherein the single domain antibody is SA21.

Embodiment 120 comprises the isolated polypeptide or polypeptide complex of embodiment 108, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 92, HC-CDR2: SEQ ID NO: 93, and HC-CDR3: SEQ ID NO: 94.

Embodiment 121 comprises the isolated polypeptide or polypeptide complex of embodiment 108, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 96, HC-CDR2: SEQ ID NO: 97, and HC-CDR3: SEQ ID NO: 98.

Embodiment 122 comprises the isolated polypeptide or polypeptide complex of embodiment 108, wherein the single domain antibody comprises an amino acid sequence with at least 80%, 85%, 90%, 95%, 99%, or 100% sequence identity to SEQ ID NO: 95.

Embodiment 123 comprises the isolated polypeptide or polypeptide complex of embodiment 108, wherein the single domain antibody comprises the amino acid sequence of SEQ ID NO: 95.

Embodiment 124 comprises the isolated polypeptide or polypeptide complex of embodiment 108, wherein the single domain antibody comprises an amino acid sequence with at least 80%, 85%, 90%, 95%, 99%, or 100% sequence identity to SEQ ID NO: 118.

Embodiment 125 comprises the isolated polypeptide or polypeptide complex of embodiment 108, wherein the single domain antibody comprises the amino acid sequence of SEQ ID NO: 118.

Embodiment 126 comprises the isolated polypeptide or polypeptide complex of embodiment 108, wherein the single domain antibody comprises an amino acid sequence with at least 80%, 85%, 90%, 95%, 99%, or 100% sequence identity to SEQ ID NO: 1509.

Embodiment 127 comprises the isolated polypeptide or polypeptide complex of embodiment 108, wherein the single domain antibody comprises the amino acid sequence of SEQ ID NO: 1509.

Embodiment 128 comprises the isolated polypeptide or polypeptide complex of any one of the preceding embodiments, wherein the polypeptide or polypeptide complex comprises a modified amino acid, a non-natural amino acid, a modified non-natural amino acid, or a combination thereof.

Embodiment 129 comprises the isolated polypeptide or polypeptide complex of embodiment 128, wherein the modified amino acid or modified non-natural amino acid comprises a post-translational modification.

Embodiment 130 comprises the isolated polypeptide or polypeptide complex of any one of embodiments 95-129, wherein H₁ comprises a linking moiety (L₂) that connects H₁ to P₁.

Embodiment 131 comprises the isolated polypeptide or polypeptide complex of embodiment 130, wherein L₂ is a peptide sequence having at least 5 to no more than 50 amino acids.

Embodiment 132 comprises the isolated polypeptide or polypeptide complex of embodiment 130, wherein L₂ is a peptide sequence having at least 10 to no more than 30 amino acids.

Embodiment 133 comprises the isolated polypeptide or polypeptide complex of embodiment 130, wherein L₂ is a peptide sequence having at least 10 amino acids.

Embodiment 134 comprises the isolated polypeptide or polypeptide complex of embodiment 130, wherein L₂ is a peptide sequence having at least 18 amino acids.

Embodiment 135 comprises the isolated polypeptide or polypeptide complex of embodiment 130, wherein L₂ has a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1.

Embodiment 136 comprises the isolated polypeptide or polypeptide complex of embodiment 130, wherein L₂ comprises an amino acid sequence according to GGGGSGGGS (SEQ ID NO: 1998).

Embodiment 137 comprises the isolated polypeptide or polypeptide complex of any one of the preceding embodiments, wherein the isolated polypeptide or polypeptide complex comprises an amino acid sequence with at least 80%, 85%, 90%, 95%, 99%, or 100% sequence identity to any one of SEQ ID NOs: 100-104, 106, 108, 110-111, 114, 116, and 1451-1502

Embodiment 138 comprises the isolated polypeptide or polypeptide complex of any one of the preceding embodiments, wherein the isolated polypeptide or polypeptide complex comprises an amino acid sequence according to any one of SEQ ID NOs: 100-104, 106, 108, 110-111, 114, 116, and 1451-1502.

Embodiment 139 comprises the isolated polypeptide or polypeptide complex of any one of the preceding embodiments, wherein the isolated polypeptide or polypeptide complex comprises an amino acid sequence with at least 80%, 85%, 90%, 95%, 99%, or 100% sequence identity to any one of SEQ ID NOs: 1503-1508.

Embodiment 140 comprises the isolated polypeptide or polypeptide complex of any one of the preceding embodiments, wherein the isolated polypeptide or polypeptide complex comprises an amino acid sequence according to any one of SEQ ID NOs: 1503-1508.

Embodiment 141 comprises a pharmaceutical composition comprising:
(i) the isolated polypeptide or polypeptide complex of any one of the preceding embodiments; and
(ii) a pharmaceutically acceptable excipient.

Embodiment 142 comprises an isolated recombinant nucleic acid molecule encoding an isolated polypeptide or polypeptide complex of any one of embodiments 1 to 140.

Embodiment 143 comprises a vector comprising the isolated recombinant nucleic acid molecule of embodiment 142.

Embodiment 144 comprises a host cell comprising the isolated recombinant nucleic acid molecule of embodiment 142.

Embodiment 145 comprises a method of treating cancer in a subject in need thereof comprising administering to the subject the isolated polypeptide or polypeptide complex of any one of embodiments 1 to 142.

### EXAMPLES

### Example 1: Anti-CD28 scFV Binding to Peptide Masks

### Equilibrium Binding Via ELISA

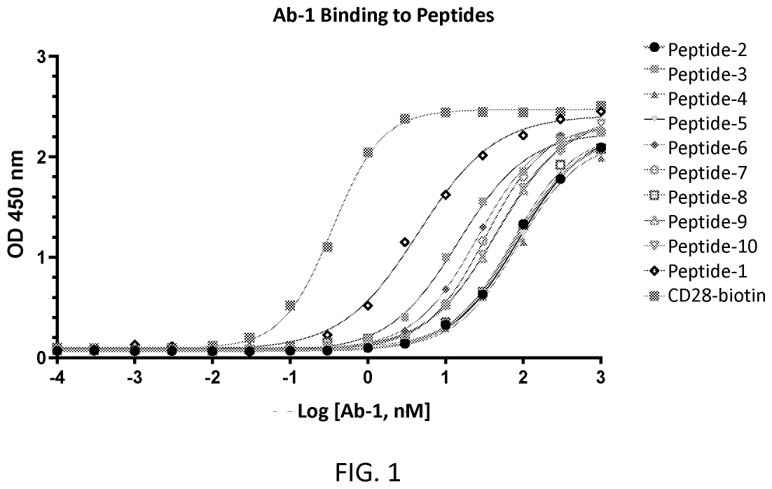

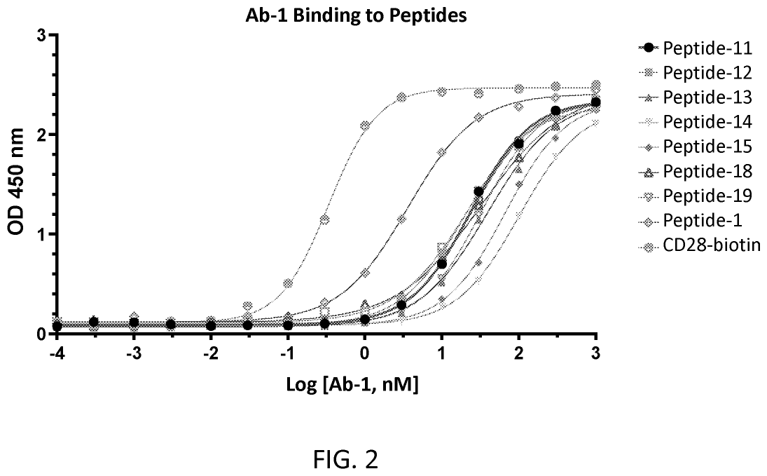

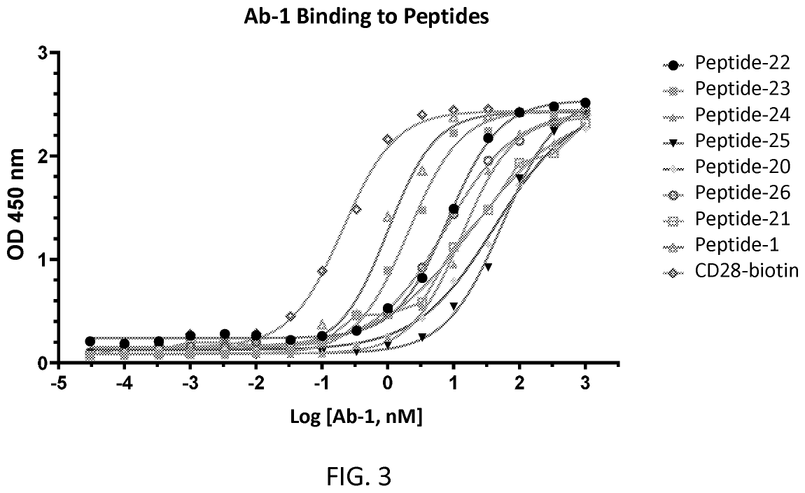

Peptides of Table 1 were evaluated for their ability to bind to human anti-CD28 scFv in a standard enzyme linked immunosorbent assay (ELISA) format. Specifically, peptides were evaluated for their ability to bind polypeptide sequence Ab-1 which is a bispecific antibody comprising the anti-CD28 scFv sequence of SEQ ID NO: 35 connected to a second antibody Fab by a GGGGS linker (SEQ ID NO: 37) (see Table 11). Briefly, biotinylated peptides or CD28-biotin were captured on neutravidin coated plates. Ab-1 diluted in buffer was then added to the peptide captured plates. Bound antibody was detected using a standard horse radish peroxidase conjugate secondary antibody. The concentration of Ab-1 required to achieve 50% maximal signal (EC₅₀) was calculated using Graphpad Prism software. Binding curves for Ab-1 binding to peptides are shown in FIGS. 1-3, and the corresponding EC₅₀s for Ab-1 binding to the peptides are shown in Tables 12-13.

TABLE 11
Exemplary antibody sequence (Ab-1) with a CD28 binding domain and a
second target binding domain.

| | | SEQ |
|---|---|---|
| Construct | Amino Acid Sequence | ID |
| Description | (N to C) | NO: |
| Ab-1 HC | QVQLVQSGAEVKKPGASVKVSCKASGYTFTSYYIHWVRQAPGQGL | 70 |
| | EWIGSIYPGNVNTNYNEKFKDRATLTVDTSISTAYMELSRLRSDDTA | |
| | VYFCTRSHYGLDWNFDVWGQGTTVTVSSGGGGSGGGGSGGGGSDI | |
| | QMTQSPSSLSASVGDRVTITCHASQNIYVWLNWYQQKPGKAPKLLI | |
| | YKASNLHTGVPSRFSGSGSGTDFTLTISSLQPEDFATYYCQQGQTYPY | |
| | TFGGGTKVEIKGGGGSQVQLVQSGAEVKKPGSSVKVSCKTSGDTFS | |
| | TYAISWVRQAPGQGLEWMGGIIPIFGKAHYAQKFQGRVTITADESTS | |
| | TAYMELSSLRSEDTAVYFCARKFHFVSGSPFGMDVWGQGTTVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVD | |
| | KKVEPKSC | |
| Ab-1 LC | EIVLTQSPATLSLSPGERATLSCRASQSVSSYLAWYQQKPGQAPRLLI | 71 |
| | YDASNRATGIPARFSGSGSGTDFTLTISSLEPEDFAVYYCQQRSNWPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| Ab-2 LC | EIVLTQSPATLSLSPGERATLSCRASQSVSSYLAWYQQKPGQAPRLLI | 72 |
| G44C, G235C | YDASNRATGIPARFSGSGSGTDFTLTISSLEPEDFAVYYCQQRSNWPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| Ab-2 HC | QVQLVQSGAEVKKPGASVKVSCKASGYTFTSYYIHWVRQAPGQCLE | 73 |
| G44C, G235C | WIGSIYPGNVNTNYNEKFKDRATLTVDTSISTAYMELSRLRSDDTAV | |
| | YFCTRSHYGLDWNFDVWGQGTTVTVSSGGGGSGGGGSGGGGSDIQ | |
| | MTQSPSSLSASVGDRVTITCHASQNIYVWLNWYQQKPGKAPKLLIY | |
| | KASNLHTGVPSRFSGSGSGTDFTLTISSLQPEDFATYYCQQGQTYPYT | |
| | FGCGTKVEIKGGGGSQVQLVQSGAEVKKPGSSVKVSCKTSGDTFST | |
| | YAISWVRQAPGQGLEWMGGIIPIFGKAHYAQKFQGRVTITADESTST | |
| | AYMELSSLRSEDTAVYFCARKFHFVSGSPFGMDVWGQGTTVTVSSA | |
| | STKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVD | |
| | KKVEPKSC | |

### Kinetic Binding Via BLI

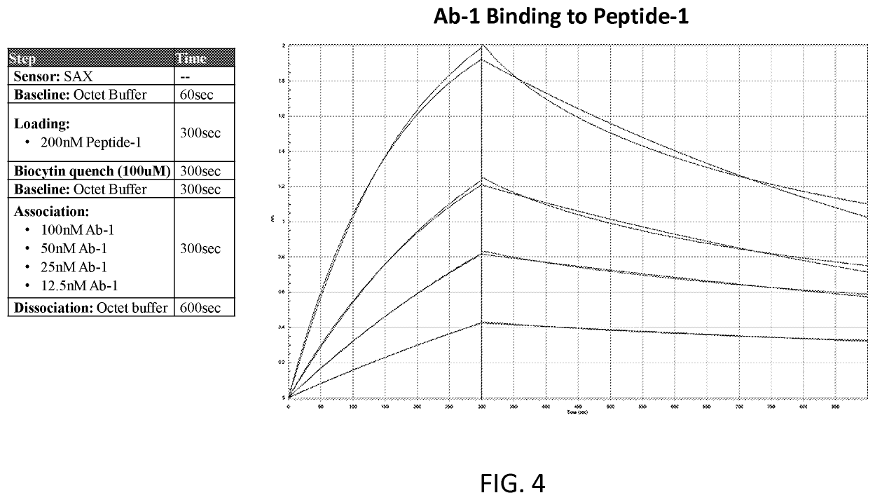

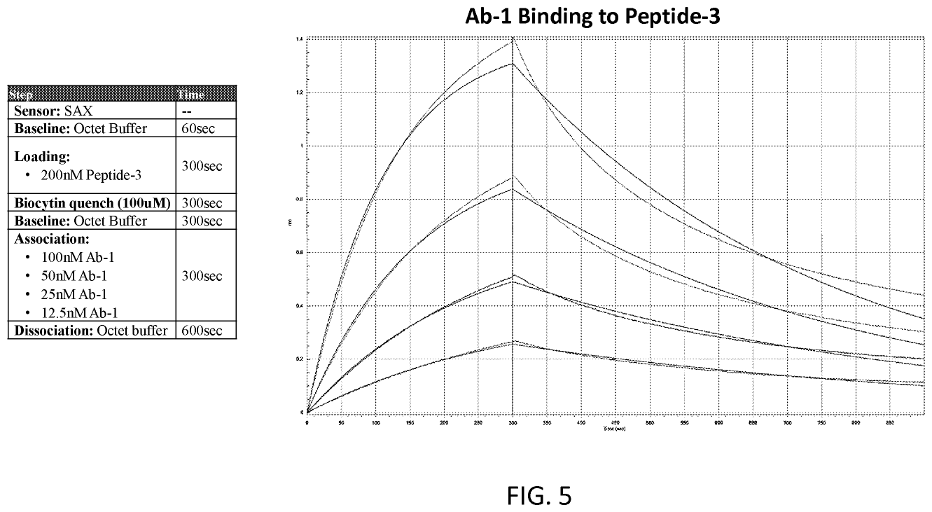

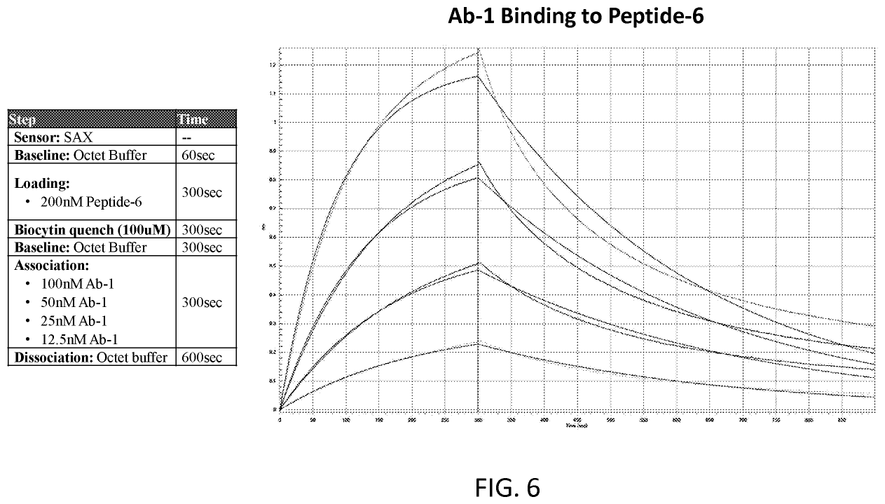

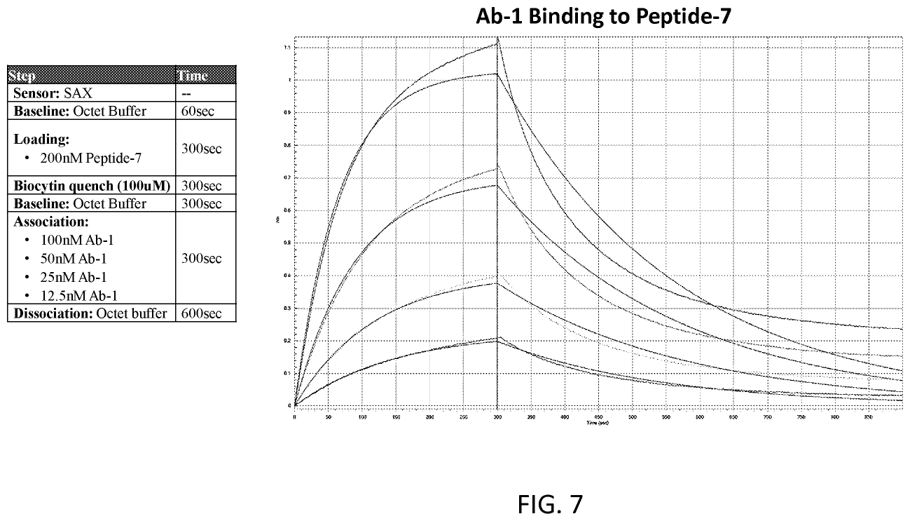

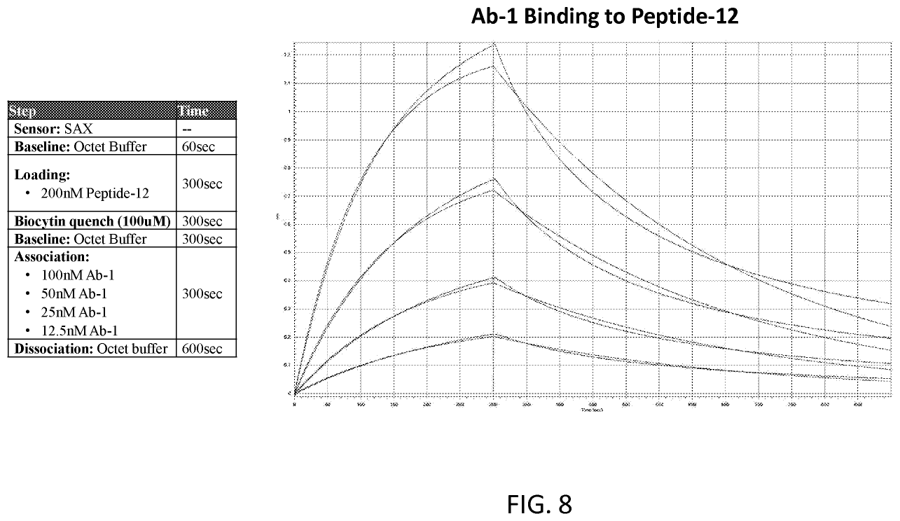

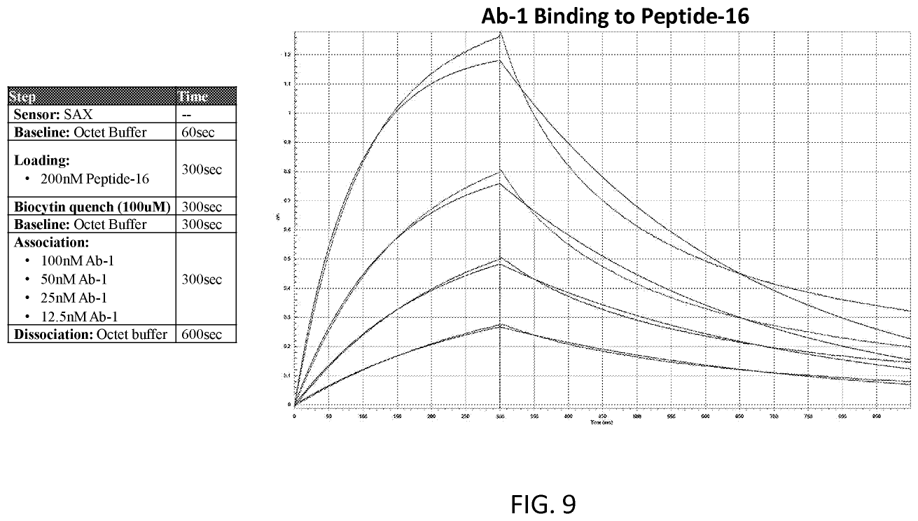

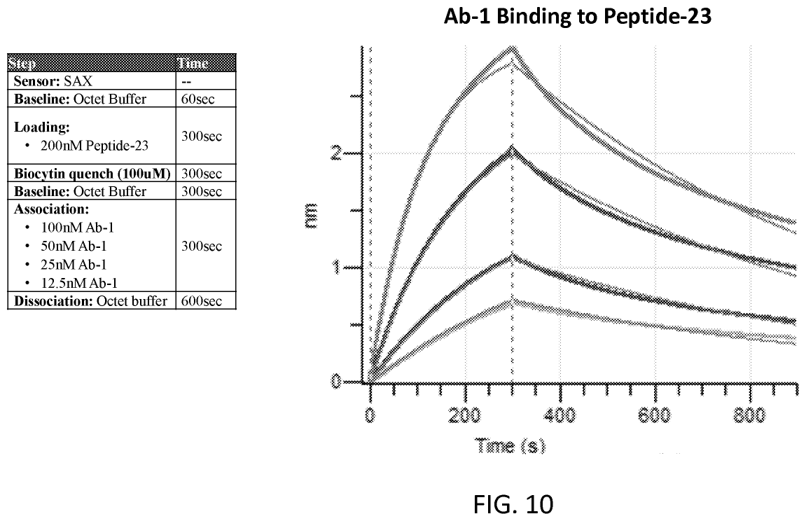

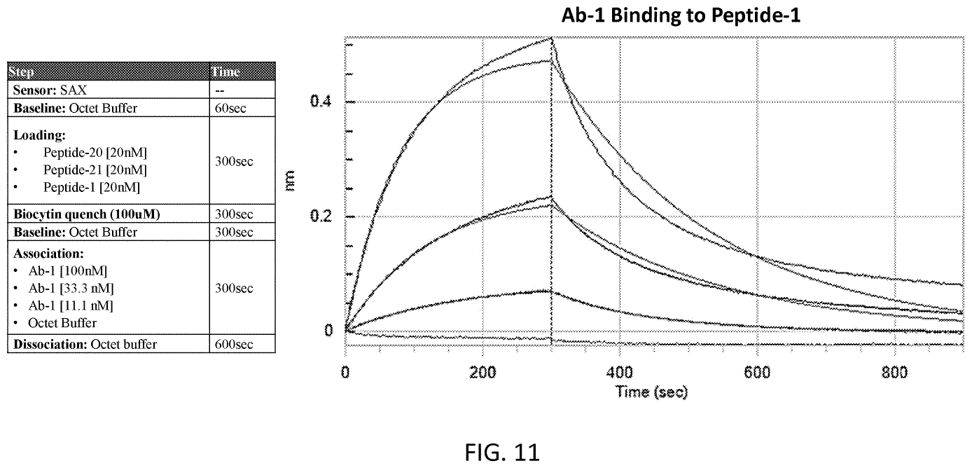

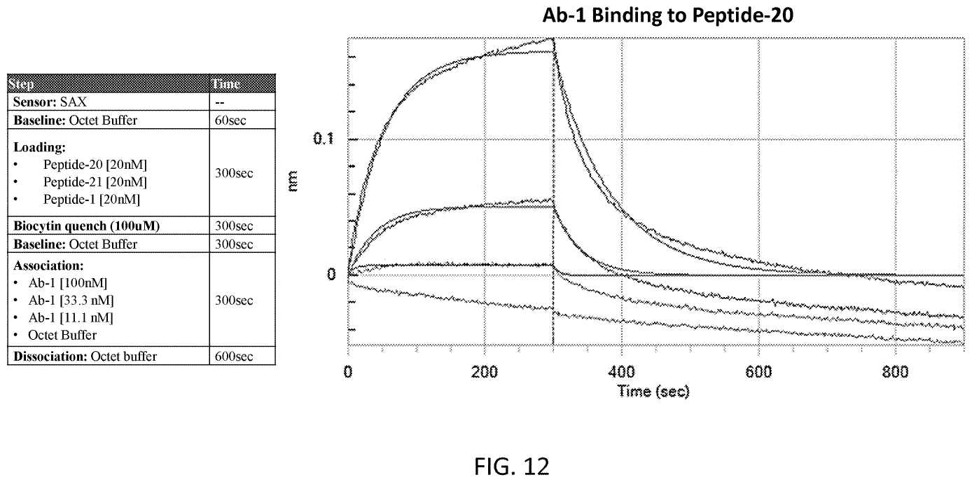

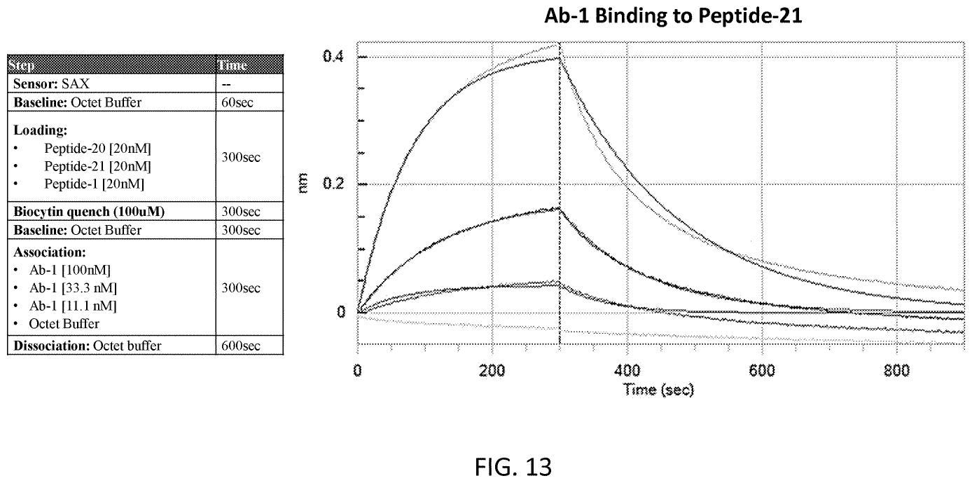

Kinetic binding of the peptides of Table 1 to Ab-1 was evaluated using biolayer interferometry (BLI). Briefly, biotinylated peptides were loaded onto a streptavidin coated Octet® SAX biosensor, quenched in biocytin, and baselined in buffer. Ab-1 was titrated in solution and associated onto the peptide loaded sensor. After a short association period, sensors were transferred into buffer and the dissociation of bound Ab-1 was measured. Association and dissociation signals were recorded in real time and analyzed using a 1:1 binding model within the instrument software. Analysis using a 1:1 binding model enabled the calculation of the on and off rate constants as well as affinity, K_{D}. Binding sensorgrams as well as the timing and steps used for the experiments are shown in FIGS. 4-13. K_{D} and half-life (t_{1/2}) values obtained from the kinetic binding measurements are provided in Tables 12-13. Octet signals and t_{1/2} values when using 200 or 20 nanomolar (nM) peptide and 100 nM Ab-1 are provided in Tables 14-15.

### Binding Inhibition by Peptide Masks

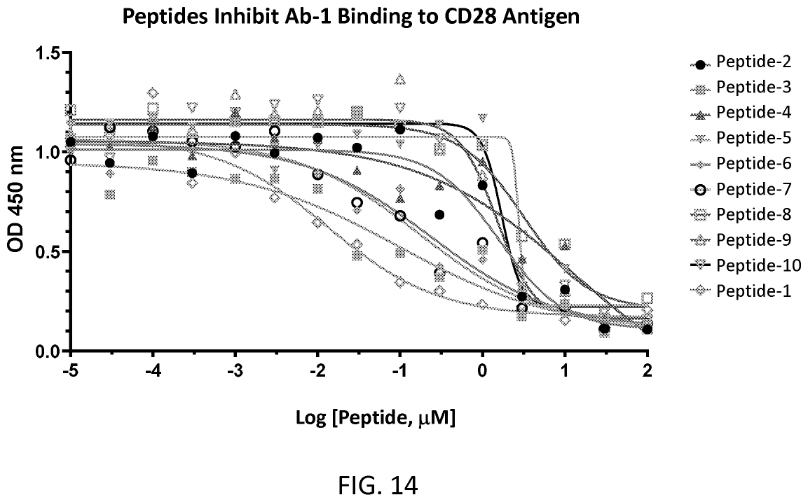

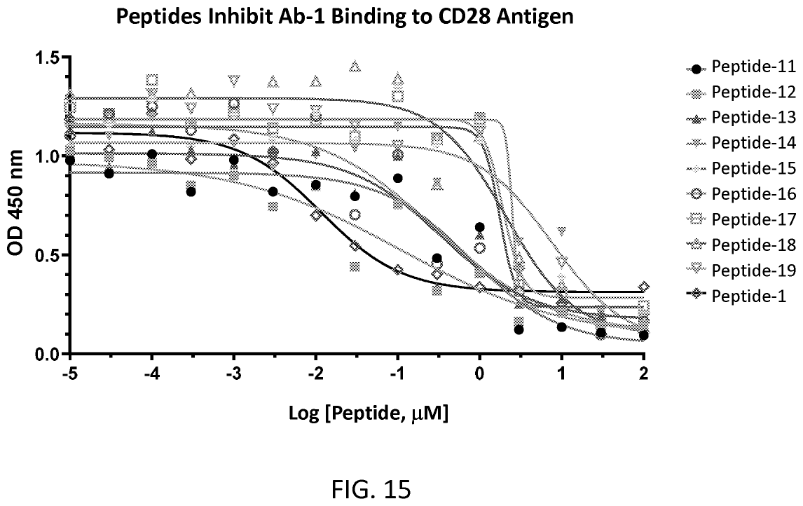

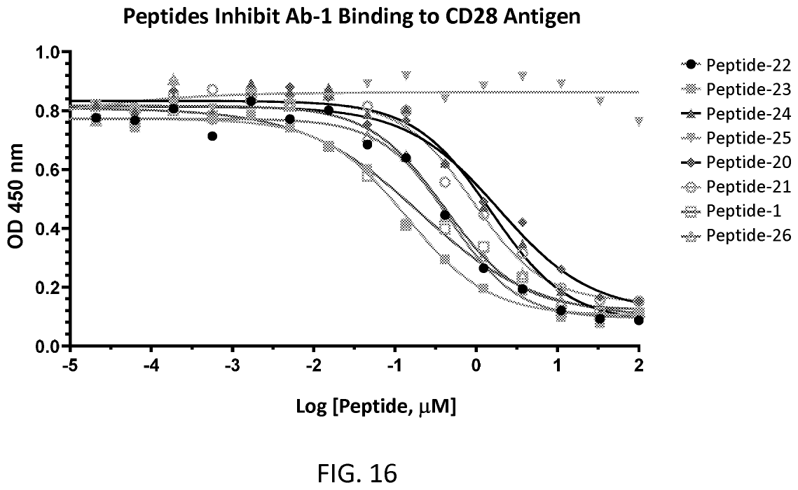

The peptides of Table 1 were further evaluated for their ability to inhibit anti-CD28 scFv harbored in Ab-1 from binding to the CD28 antigen in a standard ELISA format. Briefly, biotinylated CD28 antigen was captured on neutravidin coated plates. Ab-1 fixed at 1 nanomolar (nM) concentration was pre-incubated with 0-100 micromolar (μM) titrated peptides. After a short pre-incubation period, the mixture of titrated peptide with fixed Ab-1 were added to the CD28 antigen coated plates. After a short incubation on the plates, bound Ab-1 was detected with a standard horse radish peroxidase conjugated secondary antibody. The concentration of peptide required to reduce the maximum signal by 50% (IC₅₀) was calculated in Graphpad Prism software. FIGS. 14-16 illustrate inhibition of Ab-1 binding to CD28 by the peptide masks as measured by ELISA. IC₅₀s are provided in Tables 12-13.

TABLE 12

| | Binding | Competition | | |
|---|---|---|---|---|
| | ELISA | ELISA | K_{D}, | t_{1/2}, |
| Peptide | EC₅₀, nM | IC₅₀, μM | nM | min |
| CD28-biotin | 0.3 | | | |
| Peptide-1 | 4.0 | 0.06 | 15.1 | 15.5 |
| Peptide-2 | 75.9 | 2.00 | | |
| Peptide-3 | 14.6 | 0.17 | 20.3 | 6.2 |
| Peptide-4 | 84.3 | 5.03 | | |
| Peptide-5 | 86.8 | 4.46 | | |
| Peptide-6 | 25.7 | 0.33 | 24.6 | 4.2 |
| Peptide-7 | 32.8 | 0.3 | 23.2 | 3.1 |
| Peptide-8 | 73.9 | 7.06 | | |
| Peptide-9 | 45.4 | 2.18 | | |
| Peptide-10 | 44.9 | 2.66 | | |
| Peptide-11 | 23.4 | 0.63 | | |
| Peptide-12 | 19.7 | 0.19 | 26.1 | 4.5 |
| Peptide-13 | 39.3 | 0.78 | | |
| Peptide-14 | 101.6 | 8.34 | | |
| Peptide-15 | 66.5 | 2.63 | | |
| Peptide-16 | 24.0 | 0.59 | 25.5 | 4.7 |
| Peptide-17 | 21.7 | 2.80 | | |
| Peptide-18 | 32.2 | 3.25 | | |
| Peptide-19 | 32.5 | 4.42 | | |

TABLE 13

| | Binding | Competition | | |
|---|---|---|---|---|
| | ELISA | ELISA | K_{D}, | t_{1/2}, |
| Peptide | EC₅₀, nM | IC₅₀, μM | nM | min |
| CD28-biotin | 0.2 | | | |
| Peptide-1 | 1.0 | 0.2 | | |
| Peptide-20 | 41.7 | 1.89 | | |
| Peptide-21 | 19.6 | 0.91 | | |
| Peptide-22 | 8.2 | 0.47 | | |
| Peptide-23 | 2.1 | 0.14 | 16.0 | 9.1 |
| Peptide-24 | 13.3 | 1.52 | | |
| Peptide-25 | 54.9 | >100 | | |
| Peptide-26 | 7.6 | 0.41 | | |

TABLE 14
Kinetic Binding Data (200 nM peptide; 100 nM Ab-1)

| | Octet | t_{1/2} |
|---|---|---|
| Peptide | signal | (min) |
| Peptide-1 | 2.0349 | 9.8 |
| Peptide-2 | 0.7003 | 3.1 |
| Peptide-3 | 1.2187 | 4.9 |
| Peptide-4 | 0.4345 | 4.0 |
| Peptide-5 | 0.4608 | 4.2 |
| Peptide-6 | 1.1165 | 3.6 |
| Peptide-7 | 1.0208 | 3.0 |
| Peptide-8 | 0.4787 | 3.9 |
| Peptide-9 | 0.7365 | 3.0 |
| Peptide-10 | 0.8453 | 3.1 |
| Peptide-11 | 1.3654 | 4.5 |
| Peptide-12 | 1.1326 | 4.0 |
| Peptide-13 | 1.0086 | 3.3 |
| Peptide-14 | 0.3275 | 3.4 |
| Peptide-15 | 0.5506 | 2.9 |
| Peptide-16 | 1.3362 | 4.2 |
| Peptide-17 | 1.1498 | 4.1 |
| Peptide-18 | 0.8489 | 2.9 |
| Peptide-19 | 0.9193 | 2.9 |

TABLE 15
Kinetic Binding Data (100 nM Ab-1)

| | Octet signal | Octet signal |
|---|---|---|
| Peptide | (200 nM peptide) | (20 nM peptide) |
| Peptide-1 | 1.8035 | 0.5088 |
| Peptide-20 | | 0.1733 |
| Peptide-21 | | 0.4179 |
| Peptide-22 | 0.6966 | |
| Peptide-23 | 1.524 | |
| Peptide-24 | 0.8384 | |
| Peptide-25 | 0.1661 | |
| Peptide-26 | 0.7053 | |

### Example 2: Anti-CD28 Fab and Anti-CD28 scFv (Disulfide) Binding to Peptide Masks

### Equilibrium Binding Via ELISA

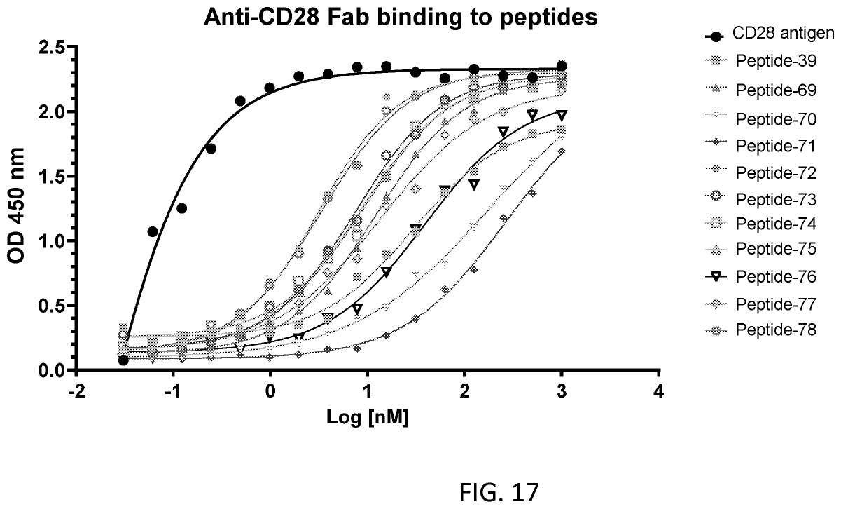

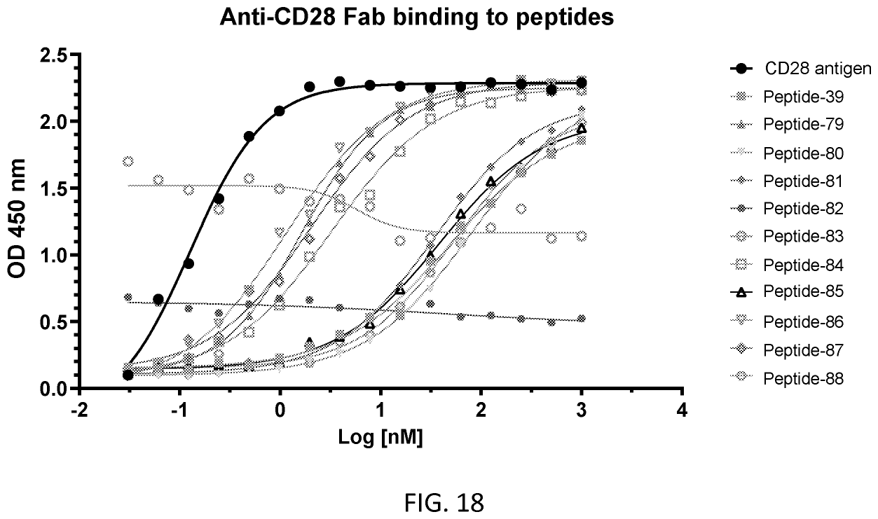

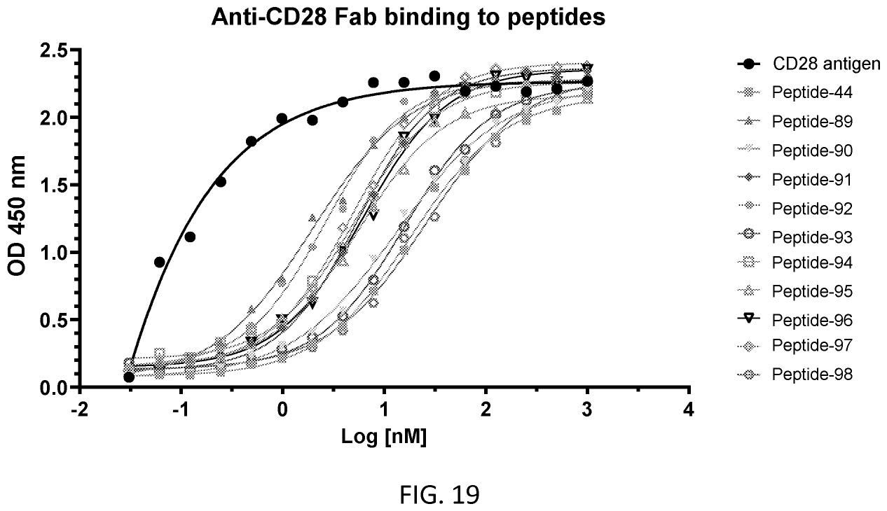

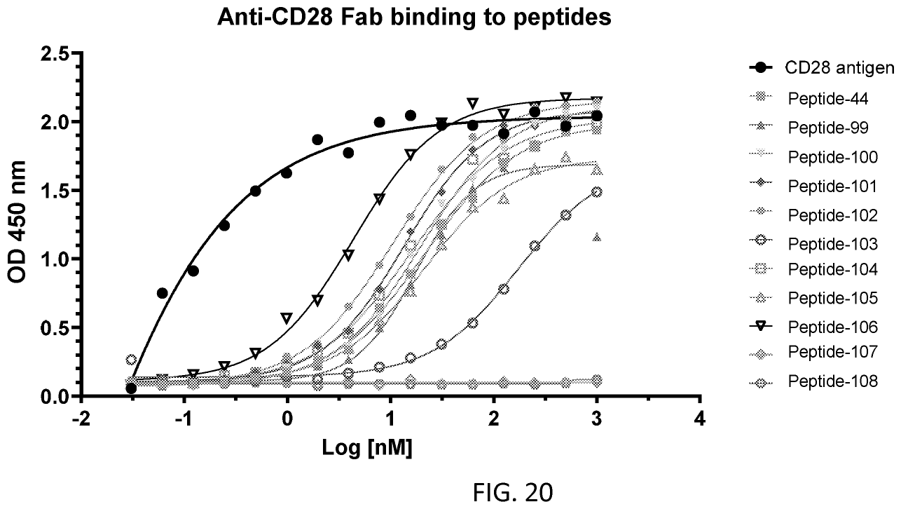

Peptides were evaluated for their ability to bind to anti-CD28 Fab (SEQ ID NOs: 202, 203) or anti-CD28 scFv (disulfide) (SEQ ID NO: 67) in a standard ELISA format as described above. Exemplary binding curves for anti-CD28 binding to peptides are shown in FIGS. 17-20. EC₅₀s are shown in Tables 16-20.

### Binding Inhibition by Peptide Masks

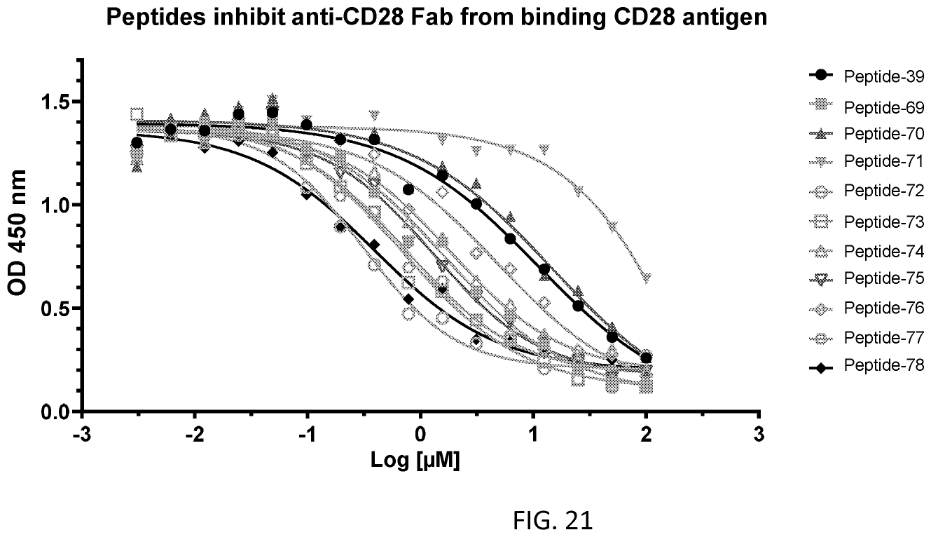

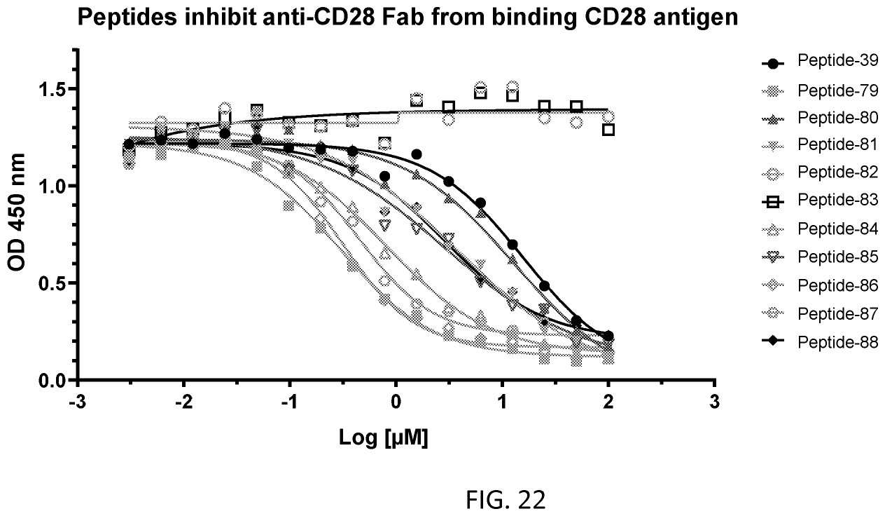

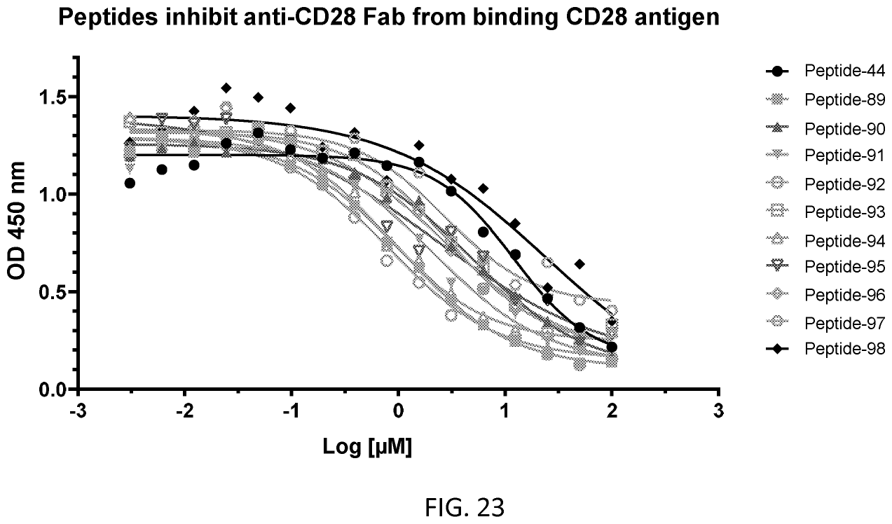

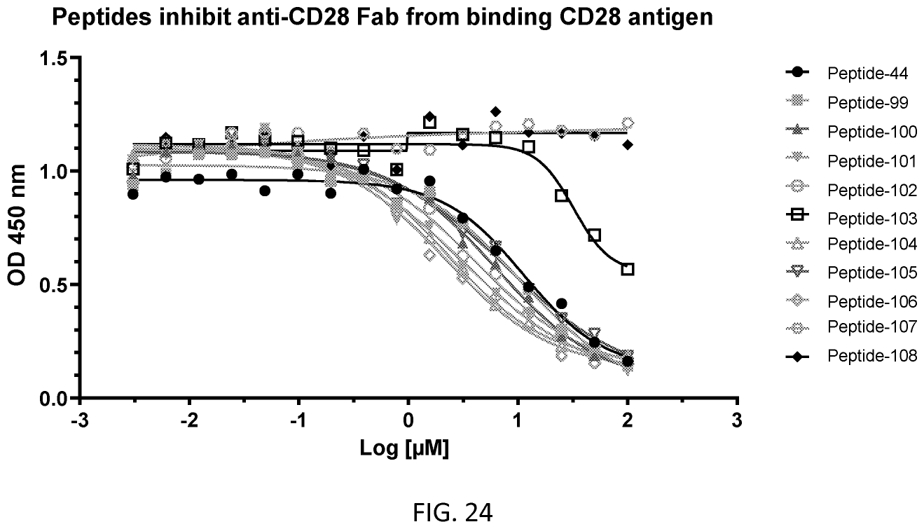

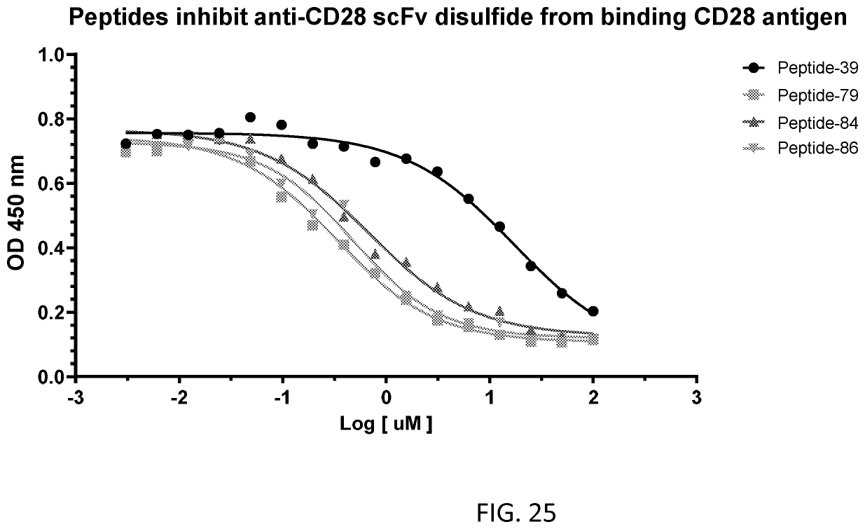

Peptides were further evaluated for their ability to inhibit the anti-CD28 Fab (SEQ ID NOs: 202, 203) or to inhibit anti-CD28 scFv (disulfide) (SEQ ID NO: 67) from binding to plate-bound CD28 antigen by competition ELISA experiments as described above. The anti-CD28 Fab was fixed at a concentration of 1 nM and incubated with titrated 0-100 μM peptides. The anti-CD28 scFv was fixed at a concentration of 0.3 nM and were incubated with titrated 0-100 μM peptides. FIGS. 21-24 illustrate inhibition of anti-CD28 Fab binding to CD28 by the peptide masks. FIG. 25 illustrates inhibition of anti-CD28 scFv (disulfide) (SEQ ID NO: 35) binding to CD28 by peptide masks. IC₅₀s are provided in Tables 16-21.

### Kinetic Binding Via BLI

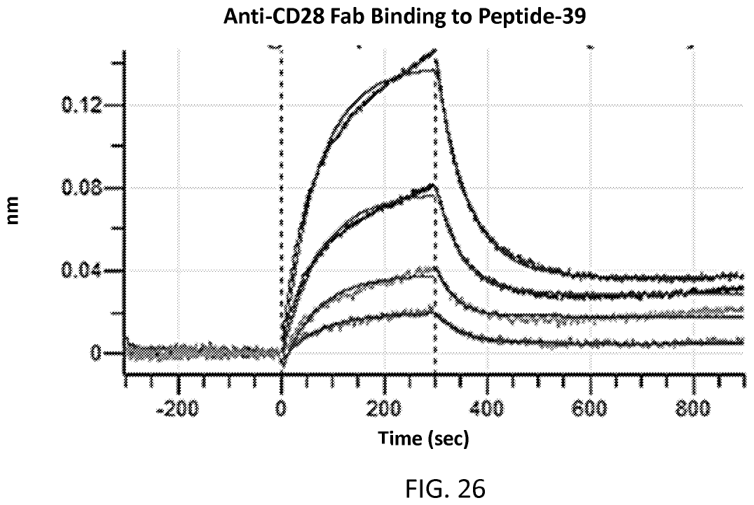

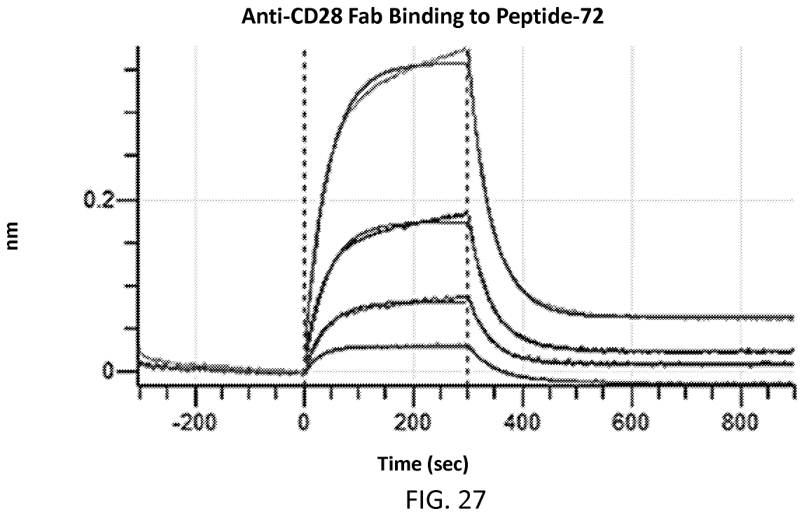

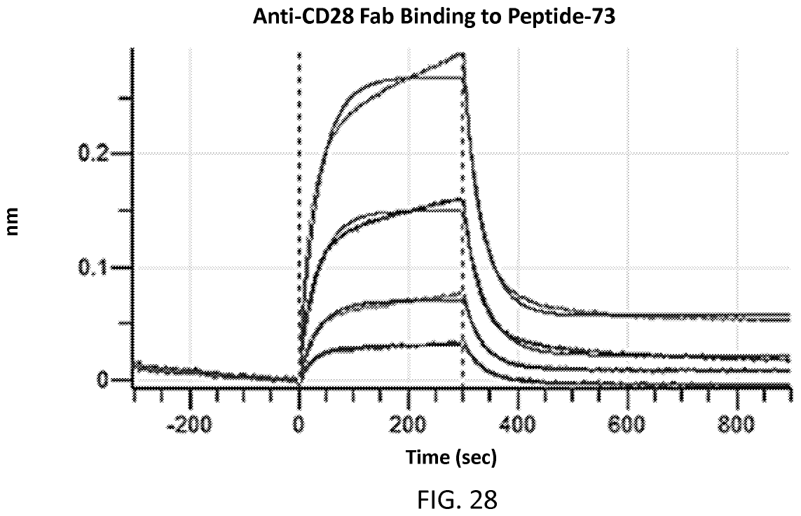

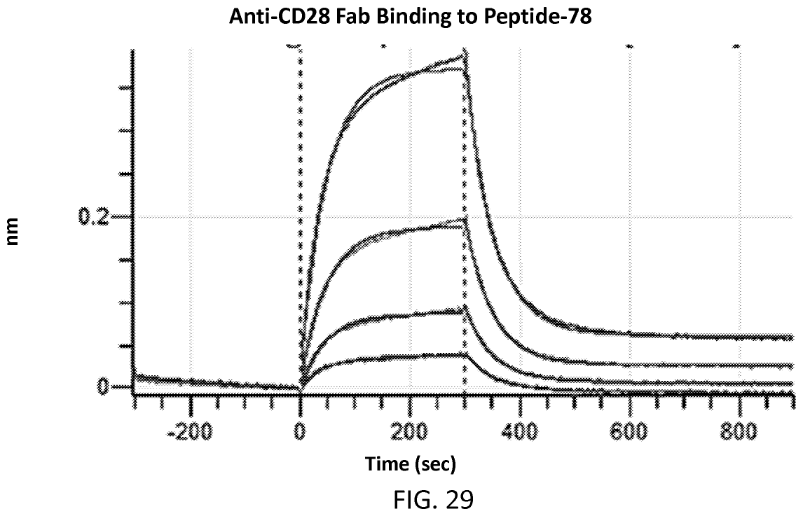

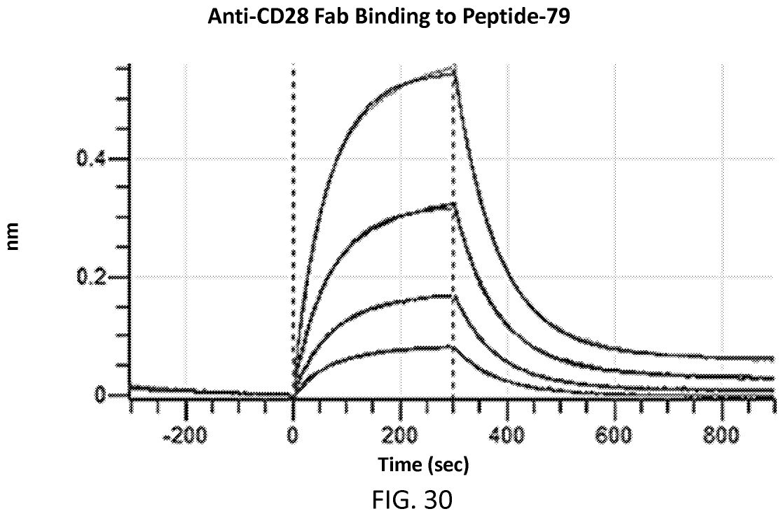

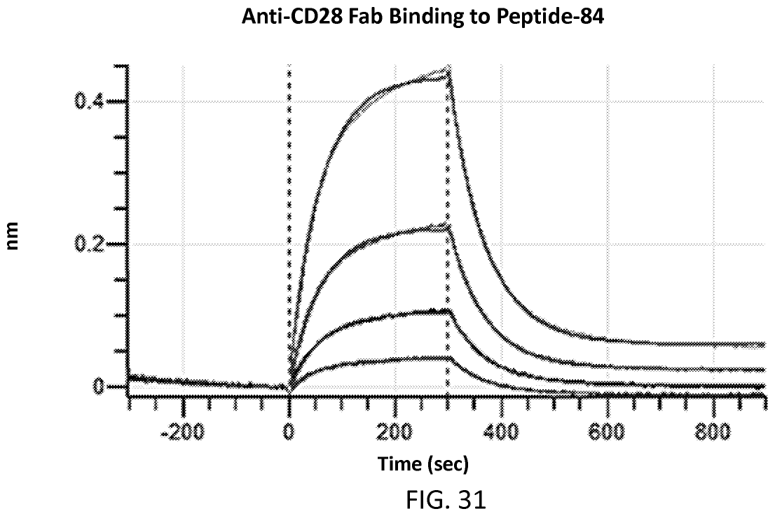

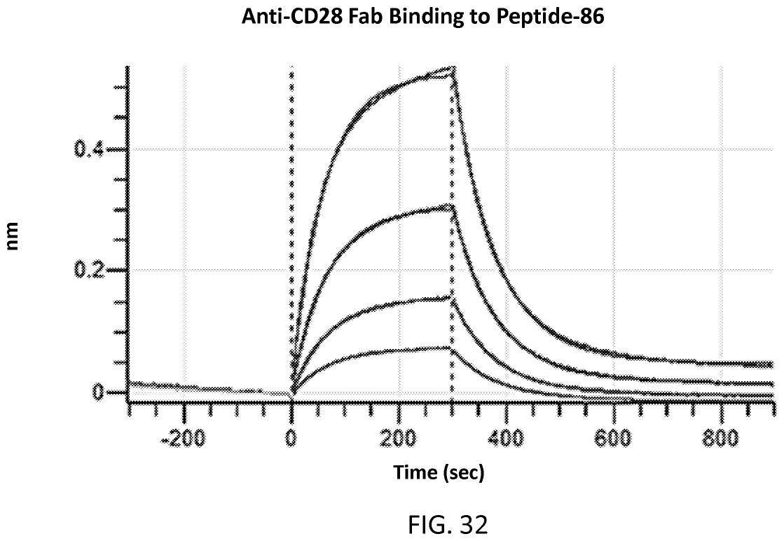

Kinetic binding of the peptides to anti-CD28 Fab (SEQ ID NOs: 202, 203) was evaluated using BLI as described above. FIGS. 26-32 show BLI titration data for binding of various peptides to the anti-CD28 Fab. KD, kₒₙ, k_{dis}, and t_{1/2} values from the BLI data are provided in Table 22.

TABLE 16

anti-CD28 Fab

| | Binding | Competition |
|---|---|---|
| | ELISA | ELISA |
| Peptide | EC₅₀ nM | IC₅₀ μM |
| Peptide-1 | 490.0 | >100 |
| Peptide-27 | >1000 | |
| Peptide-28 | >1000 | |
| Peptide-29 | >1000 | |
| Peptide-30 | >1000 | |
| Peptide-31 | 97 | >100 |
| Peptide-32 | >1000 | |
| Peptide-33 | >1000 | |
| Peptide-34 | >1000 | |
| Peptide-35 | >1000 | |
| Peptide-36 | 337 | 26.82 |
| Peptide-37 | >1000 | >100 |
| Peptide-38 | >1000 | |
| Peptide-39 | 49 | 13.77 |
| Peptide-40 | >1000 | |
| Peptide-41 | >1000 | |
| Peptide-42 | >1000 | |
| Peptide-43 | >1000 | |
| Peptide-44 | 54 | 2.39 |
| Peptide-45 | >1000 | |
| Peptide-46 | >1000 | |
| Peptide-47 | | 43.59 |
| Peptide-48 | | 7.59 |
| Peptide-49 | | 69.10 |
| Peptide-50 | | 7.04 |

TABLE 17

anti-CD28 Fab

| | Binding | Competition |
|---|---|---|
| | ELISA | ELISA |
| Peptide | EC₅₀ nM | IC₅₀ μM |
| Peptide-44 | 18.9 | 11.78 |
| Peptide-89 | 2.1 | 0.89 |
| Peptide-90 | 13.1 | 3.61 |
| Peptide-91 | 5.3 | 2.19 |
| Peptide-92 | 2.6 | 0.76 |
| Peptide-93 | 15.7 | 2.22 |
| Peptide-94 | 4.5 | 0.62 |
| Peptide-95 | 5.6 | 2.56 |
| Peptide-96 | 5.6 | 3.04 |
| Peptide-97 | 4.4 | 7.70 |
| Peptide-98 | 24.3 | 16.12 |
| Peptide-99 | 25.8 | 8.15 |
| Peptide-100 | 19.0 | 5.67 |
| Peptide-101 | 12.8 | 2.66 |
| Peptide-102 | 10.6 | 4.76 |
| Peptide-103 | 112.4 | 80.52 |
| Peptide-104 | 15.6 | 2.22 |
| Peptide-105 | 20.5 | 8.42 |
| Peptide-106 | 4.2 | 2.20 |
| Peptide-107 | >1000 | >100 |
| Peptide-108 | >1000 | >100 |

TABLE 18

| | anti-CD28 Fab | | |
|---|---|---|---|
| | Binding | Competition | |
| | ELISA | ELISA | Off rate |
| Peptide | EC₅₀ nM | IC₅₀ μM | t½ (min) |
| Peptide-39 | 30.6 | 9.76 | 0.7 |
| Peptide-69 | 11.0 | 1.53 | |
| Peptide-70 | 65.8 | 13.33 | |
| Peptide-71 | 127.7 | 77.71 | |
| Peptide-72 | 3.1 | 0.36 | 0.5 |
| Peptide-73 | 7.1 | 0.48 | 0.4 |
| Peptide-74 | 7.8 | 2.22 | |
| Peptide-75 | 8.1 | 1.32 | |
| Peptide-76 | 28.1 | 4.07 | |
| Peptide-77 | 12.1 | 0.83 | |
| Peptide-78 | 3.4 | 0.49 | 0.6 |
| Peptide-79 | 1.7 | 0.30 | 1.0 |
| Peptide-80 | 59.4 | 9.66 | |
| Peptide-81 | 30.5 | 3.18 | |
| Peptide-82 | >1000 | >100 | |
| Peptide-83 | >1000 | >100 | |
| Peptide-84 | 3.2 | 0.35 | 0.8 |
| Peptide-85 | 32.5 | 2.90 | |
| Peptide-86 | 1.3 | 0.37 | 0.9 |
| Peptide-87 | 2.1 | 0.63 | 0.9 |
| Peptide-88 | 44.9 | 4.12 | |

TABLE 19

anti-CD28 scFv (disulfide)

| | Binding | Competition |
|---|---|---|
| | ELISA | ELISA |
| Peptide | EC₅₀ nM | IC₅₀ μM |
| Peptide-1 | 98.5 | 61.53 |
| Peptide-27 | >1000 | |
| Peptide-28 | >1000 | |
| Peptide-29 | >1000 | |
| Peptide-30 | >1000 | |
| Peptide-31 | 73.17 | >100 |
| Peptide-32 | >1000 | |
| Peptide-33 | >1000 | |
| Peptide-34 | >1000 | |
| Peptide-35 | >1000 | |
| Peptide-36 | >1000 | >100 |
| Peptide-37 | >1000 | >100 |
| Peptide-38 | >1000 | |
| Peptide-39 | 155.90 | >100 |
| Peptide-40 | >1000 | |
| Peptide-41 | >1000 | |
| Peptide-42 | >1000 | |
| Peptide-43 | >1000 | |
| Peptide-44 | >1000 | >100 |
| Peptide-45 | >1000 | |
| Peptide-46 | >1000 | |

TABLE 20

| | anti-CD28 Fab |
|---|---|
| | Competition |
| | ELISA |
| Peptide | IC₅₀ μM |
| Peptide-44 | 7.89 |
| Peptide-51 | 20.98 |
| Peptide-52 | >100 |
| Peptide-53 | 7.42 |
| Peptide-54 | >100 |
| Peptide-55 | >100 |
| Peptide-56 | >100 |
| Peptide-57 | 12.95 |
| Peptide-58 | 76.43 |
| Peptide-39 | 12.56 |
| Peptide-59 | >100 |
| Peptide-60 | >100 |
| Peptide-61 | >100 |
| Peptide-62 | 3.16 |
| Peptide-63 | 12.29 |
| Peptide-64 | >100 |
| Peptide-65 | 102.00 |
| Peptide-66 | 5.49 |
| Peptide-67 | >100 |
| Peptide-68 | 98.64 |

TABLE 21

| | anti-CD28 scFv |
|---|---|
| | (disulfide) |
| | Competition |
| | ELISA |
| Peptide | IC₅₀ μM |
| Peptide-44 | 31.18 |
| Peptide-51 | 67.05 |
| Peptide-52 | >100 |
| Peptide-53 | 36.18 |
| Peptide-54 | >100 |
| Peptide-55 | >100 |
| Peptide-56 | >100 |
| Peptide-57 | 56.91 |
| Peptide-58 | >100 |
| Peptide-39 | 10.99 |
| | 14.64 |
| Peptide-59 | >100 |
| Peptide-60 | >100 |
| Peptide-61 | >100 |
| Peptide-62 | 1.91 |
| Peptide-63 | 10.48 |
| Peptide-64 | >100 |
| Peptide-65 | >100 |
| Peptide-66 | 7.09 |
| Peptide-67 | >100 |
| Peptide-68 | 76.41 |
| Peptide-79 | 0.35 |
| Peptide-84 | 0.78 |
| Peptide-86 | 0.48 |

TABLE 22

| Peptide | K_{D} (M) | kₒₙ (1/Ms) | k_{dis} (1/s) | t½ (min) |
|---|---|---|---|---|
| Peptide-39 | 2.02E−06 | 1.00E+04 | 2.02E−02 | 0.6 |
| Peptide-72 | 9.26E−07 | 2.58E+04 | 2.39E−02 | 0.5 |
| Peptide-73 | 8.31E−07 | 3.37E+04 | 2.80E−02 | 0.4 |
| Peptide-78 | 1.41E−07 | 1.35E+05 | 1.91E−02 | 0.6 |
| Peptide-79 | 4.47E−08 | 2.64E+05 | 1.18E−02 | 1.0 |
| Peptide-84 | 1.10E−07 | 1.25E+05 | 1.38E−02 | 0.8 |
| Peptide-86 | 4.02E−08 | 2.86E+05 | 1.15E−02 | 1.0 |

### Example 3: Sequence Activity Relationships—CD28 Masks

Sequence activity relationships (SAR) were established for peptide-39 and peptide-44 which were found to exhibit strong binding to the anti-CD28 Fab. The sequence activity relationships were established by mutating individual residues within each peptide to alanine and measuring the influence of each mutation on the ability of the peptide to inhibit the ability of the anti-CD28 Fab from binding to CD28 by competition ELISA experiments as described above in Example 1. IC₅₀s for binding inhibition by the alanine scanning sequences are provided in Tables 23-24.

TABLE 23
Competition Binding ELISA - Alanine Scanning Sequences of Peptide-39

| | | | Competition ELISA |
|---|---|---|---|
| Peptide | SEQ ID NO: | Sequence (N to C) | IC₅₀ (mM) |
| Peptide-39 | 132 | YCAGHAWQYINKCL | 12.56 |
| Peptide-59 | 152 | ACAGHAWQYINKCL | >100 |
| Peptide-60 | 153 | YCAAHAWQYINKCL | >100 |
| Peptide-61 | 154 | YCAGAAWQYINKCL | >100 |
| Peptide-62 | 155 | YCAGHAAQYINKCL | 3.16 |
| Peptide-63 | 156 | YCAGHAWAYINKCL | 12.29 |
| Peptide-64 | 157 | YCAGHAWQAINKCL | >100 |
| Peptide-65 | 158 | YCAGHAWQYANKCL | 102.00 |
| Peptide-66 | 159 | YCAGHAWQYIAKCL | 5.49 |
| Peptide-67 | 160 | YCAGHAWQYINACL | >100 |
| Peptide-68 | 161 | YCAGHAWQYINKCA | 98.64 |

TABLE 24
Competition Binding ELISA - Alanine Scanning Sequences of Peptide-44

| | | | Competition ELISA |
|---|---|---|---|
| Peptide | SEQ ID NO: | Sequence (N to C) | IC₅₀ (mM) |
| Peptide-44 | 137 | SCAAGASYAWPYCL | 7.89 |
| Peptide-51 | 144 | ACAAGASYAWPYCL | 20.98 |
| Peptide-52 | 145 | SCAAAASYAWPYCL | >100 |
| Peptide-53 | 146 | SCAAGAAYAWPYCL | 7.42 |
| Peptide-54 | 147 | SCAAGASAAWPYCL | >100 |
| Peptide-55 | 148 | SCAAGASYAAPYCL | >100 |
| Peptide-56 | 149 | SCAAGASYAWAYCL | >100 |
| Peptide-57 | 150 | SCAAGASYAWPACL | 12.95 |
| Peptide-58 | 151 | SCAAGASYAWPYCA | 76.43 |

### Example 4: Optimized Phage Library Construction of Peptide-39

Using the peptide sequence activity relationships of Example 3, DNA oligo libraries were constructed where codons encoding critical residues within peptide-39 were minimally mutated, and codons encoding non-critical residues that were tolerated in the alanine scanning mutation studies (e.g., Trp-7, Gln-8, and Asn-11) were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 5: Panning of the Peptide-39 Optimized Phage Library

Once the phage optimization libraries were completed, phage libraries were bio-panned using CD28 Fab loaded beads. Multiple rounds of panning were performed where bacteriophage was allowed to bind to CD28 Fab loaded beads, washed, eluted, and amplified. After panning, phage infected bacteria were plated out and colonies picked into 96 well blocks. Clonal phage was then amplified and separated from bacterial cells via centrifugation. Phage containing supernatants were tested in binding ELISAs against CD28 Fab coated plates in the presence or absence of saturating concentrations of CD28 antigen. Phage able to bind CD28 Fab were selected for sequence analysis if the binding signal was reduced in the presence of CD28.

### Example 6: Panning ELISAs—CD28 Fab Peptides

Clonal phage were harvested as crude supernatants and screened via standard ELISAs. Briefly, biotinylated CD28 Fab was captured on neutravidin coated plates. Prior to the addition of clonal phage, wells were incubated with blocking buffer and CD28 antigen or blocking buffer alone. Without washing or aspirating, clonal phage supernatants were then added to the wells and incubated for a short time. Wells were then washed followed by detection of bound phage using a horse radish conjugated anti-M13 antibody. Clonal phage of interest were then sent for sequence analysis.

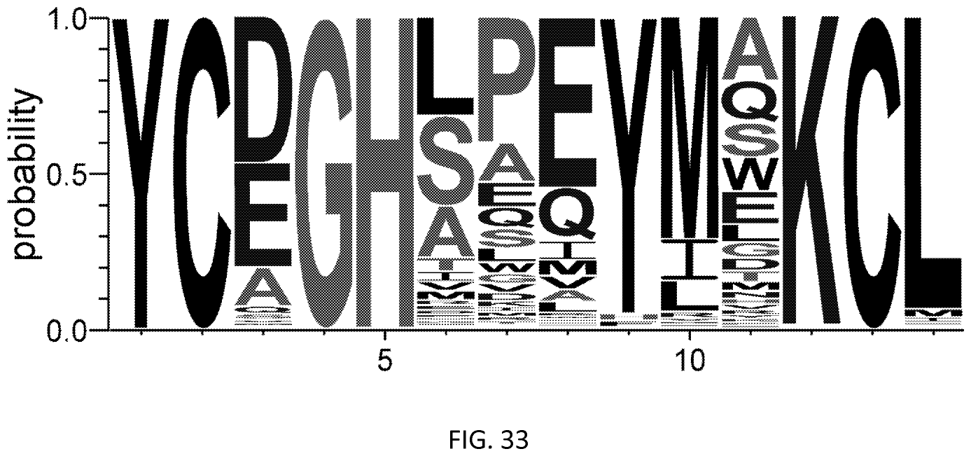

Phage panning results of peptide-39 library sequences are shown in Table 25. 886 clonal phage sequences were identified. A consensus sequence was calculated from all the sequences of Table 25 using WebLogo 3.7.12. The consensus sequence is shown in FIG. 33.

TABLE 25
Phage Panning Results of Peptide-39 Library Sequences.

| | | | | | | | | | | | | | | | Phase binding ELISA | | | |
|---|---|---|---|---|---|---|---|---|---|---|---|---|---|---|---|---|---|---|
| | | | | | | | | | | | | | | | | Anti- | Anti- | |
| | | | | | | | | | | | | | | | | CD28 | CD28 | |
| | | | | | | | | | | | | | | | | Fab | Fab | |
| | | | | | | | | | | | | | | | | (SEQ | signal | |
| | | | | | | | | | | | | | | | | ID | in the | |
| | | | | | | | | | | | | | | | | NOs: | pre- | |
| | | | | | | | | | | | | | | | Back- | 202, | sence | SEQ |
| | Amino acid position sequence | | | | | | | | | | | | | | ground | 203) | of | ID |
| Phage ID | 1 | 2 | 3 | 4 | 5 | 6 | 7 | 8 | 9 | 10 | 11 | 12 | 13 | 14 | signal | signal | CD28 | NO: |
| Phage-13 | Y | C | A | G | H | A | W | Q | Y | I | N | K | C | L | 0.06 | 0.64 | 0.07 | 132 |
| Phage-1 | — | W | S | C | F | Q | I | P | F | T | C | F | Y | F | 0.18 | 1.44 | 0.27 | 120 |
| Phage-2 | W | E | C | L | K | Y | T | E | A | N | A | C | T | Y | 0.05 | 1.55 | 0.07 | 121 |
| Phage-3 | F | M | C | L | P | Q | N | P | H | P | L | C | F | K | 0.06 | 0.84 | 0.06 | 122 |
| Phage-4 | P | D | C | Q | T | I | L | F | K | P | W | C | V | R | 0.05 | 2.09 | 0.06 | 123 |
| Phage-5 | A | — | D | T | Y | F | A | G | M | — | — | E | — | F | 0.07 | 0.92 | 0.10 | 124 |
| Phage-6 | G | — | Q | Y | P | W | A | F | D | A | M | S | — | P | 0.05 | 2.38 | 0.07 | 125 |
| Phage-7 | W | — | W | D | Y | K | L | W | V | P | T | Q | — | P | 0.11 | 2.27 | 0.14 | 126 |
| Phage-8 | W | — | W | D | Y | R | E | W | V | P | M | T | — | P | 0.06 | 2.30 | 0.08 | 127 |
| Phage-9 | E | — | H | W | S | M | — | A | V | G | H | A | — | F | 0.06 | 1.77 | 0.07 | 128 |
| Phage-10 | — | — | — | — | — | S | P | I | N | — | G | — | — | — | 0.05 | 1.49 | 0.06 | 129 |
| Phage-11 | W | — | W | D | Y | K | L | W | V | P | M | T | — | P | 0.11 | 2.51 | 0.11 | 130 |
| Phage-12 | D | — | W | W | M | F | E | S | — | P | G | R | — | T | 0.06 | 1.65 | 0.07 | 131 |
| Phage-14 | W | — | W | Y | Y | R | E | W | V | P | M | T | — | P | 0.06 | 1.85 | 0.07 | 133 |
| Phage-15 | — | W | Y | C | S | P | S | I | V | R | C | V | L | V | 0.06 | 1.51 | 0.09 | 134 |
| Phage-16 | P | D | C | H | T | I | L | F | K | P | W | C | V | R | 0.07 | 1.00 | 0.06 | 135 |
| Phage-17 | W | I | C | D | K | S | G | S | I | M | L | C | R | A | 0.06 | 0.91 | 0.06 | 136 |
| Phage-18 | S | — | — | A | G | — | S | Y | A | W | P | Y | — | — | 0.05 | 1.49 | 0.05 | 137 |
| Phage-19 | D | — | P | W | L | S | G | F | S | R | P | L | L | C | 0.09 | 1.27 | 0.07 | 138 |
| Phage-20 | — | — | — | — | M | S | T | I | Q | L | W | L | K | C | 0.06 | 2.16 | 0.09 | 139 |
| Phage-21 | — | — | — | — | — | M | P | — | — | E | V | — | — | — | 0.07 | 0.22 | 0.06 | 140 |
| Phage-22 | — | — | — | — | — | T | E | E | H | Y | V | — | — | M | 0.05 | 0.62 | 0.07 | 141 |
| Phage-23 | — | — | M | — | — | S | H | E | H | — | W | — | — | — | 0.06 | 0.87 | 0.07 | 142 |
| Phage-24 | — | — | — | — | — | S | H | E | — | L | A | — | — | E | 0.05 | 0.86 | 0.06 | 143 |
| Phage-25 | — | — | E | — | — | S | P | E | — | — | L | — | — | — | 0.05 | 1.75 | 0.06 | 162 |
| Phage-26 | — | — | E | — | — | R | P | E | — | — | L | — | — | — | 0.06 | 1.27 | 0.06 | 163 |
| Phage-27 | — | — | E | — | — | E | G | I | — | F | W | — | — | — | 0.05 | 2.07 | 0.06 | 164 |
| Phage-28 | — | — | E | — | — | L | P | E | — | — | E | — | — | — | 0.05 | 2.06 | 0.08 | 165 |
| Phage-29 | — | — | E | — | — | — | P | E | — | — | A | — | — | — | 0.05 | 2.15 | 0.09 | 166 |
| Phage-30 | — | — | E | — | — | — | P | E | — | — | R | — | — | — | 0.06 | 1.63 | 0.08 | 167 |
| Phage-31 | — | — | E | — | — | — | P | E | — | — | W | — | — | — | 0.08 | 1.63 | 0.07 | 168 |
| Phage-32 | — | — | E | — | — | S | Q | E | N | — | W | — | — | — | 0.05 | 1.41 | 0.07 | 169 |
| Phage-33 | — | — | E | — | — | L | P | E | — | L | G | — | — | — | 0.06 | 1.23 | 0.08 | 170 |
| Phage-34 | — | — | E | — | — | L | P | E | — | R | E | — | — | — | 0.06 | 1.96 | 0.07 | 171 |
| Phage-35 | — | — | E | — | — | L | P | E | — | L | A | — | — | — | 0.06 | 2.34 | 0.10 | 172 |
| Phage-36 | — | — | E | — | — | S | Q | E | — | L | W | — | — | — | 0.07 | 2.36 | 0.07 | 173 |
| Phage-37 | — | — | E | — | — | S | S | E | — | — | Q | — | — | — | 0.06 | 2.25 | 0.10 | 174 |
| Phage-38 | H | — | S | — | — | H | — | L | Q | V | W | — | — | F | 0.06 | 2.31 | 0.07 | 175 |
| Phage-39 | — | — | W | S | — | S | P | P | F | — | K | — | — | Y | 0.07 | 2.06 | 0.06 | 176 |
| Phage-40 | — | — | E | — | — | L | P | E | — | R | — | — | — | — | 0.08 | 2.38 | 0.17 | 177 |
| Phage-41 | — | — | E | — | — | L | P | E | — | L | E | — | — | V | 0.07 | 2.11 | 0.06 | 178 |
| Phage-42 | — | — | E | — | — | L | P | E | — | — | Q | — | — | — | 0.06 | 1.90 | 0.08 | 179 |
| Phage-43 | — | — | E | — | — | L | P | E | — | — | W | — | — | — | 0.09 | 2.32 | 0.09 | 180 |
| Phage-44 | — | — | E | — | — | — | P | E | — | — | G | — | — | — | 0.10 | 2.10 | 0.07 | 181 |
| Phage-45 | — | — | D | — | — | S | P | I | — | M | W | — | — | — | 0.08 | 2.47 | 0.09 | 204 |
| Phage-46 | — | — | E | — | — | — | P | E | — | M | A | — | — | — | 0.07 | 2.47 | 0.08 | 205 |
| Phage-47 | — | — | D | — | — | L | P | — | — | M | E | — | — | — | 0.06 | 2.47 | 0.11 | 206 |
| Phage-48 | — | — | — | — | — | — | P | E | — | M | E | — | — | — | 0.06 | 2.45 | 0.09 | 207 |
| Phage-49 | — | — | D | — | — | S | P | E | — | M | W | — | — | — | 0.07 | 2.44 | 0.09 | 208 |
| Phage-50 | — | — | D | — | — | L | P | E | — | M | E | — | — | — | 0.08 | 2.44 | 0.10 | 209 |
| Phage-51 | — | — | E | — | — | L | P | — | — | M | W | — | — | — | 0.11 | 2.44 | 0.17 | 210 |
| Phage-52 | — | — | D | — | — | L | P | E | — | M | — | — | — | — | 0.06 | 2.43 | 0.13 | 211 |
| Phage-53 | — | — | — | — | — | L | P | E | — | M | D | — | — | — | 0.07 | 2.42 | 0.12 | 212 |
| Phage-54 | — | — | D | — | — | S | A | E | H | M | A | — | — | — | 0.07 | 2.41 | 0.08 | 213 |
| Phage-55 | — | — | — | — | — | L | P | — | — | M | A | — | — | — | 0.07 | 2.41 | 0.17 | 214 |
| Phage-57 | — | — | E | — | — | L | P | E | — | M | A | — | — | — | 0.07 | 2.41 | 0.14 | 215 |
| Phage-58 | — | — | E | — | — | L | P | E | — | M | F | — | — | — | 0.10 | 2.40 | 0.12 | 216 |
| Phage-59 | — | — | D | — | — | L | P | — | — | M | A | Q | — | — | 0.06 | 2.40 | 0.20 | 217 |
| Phage-60 | — | — | E | — | — | L | P | — | — | M | Q | — | — | — | 0.06 | 2.39 | 0.12 | 218 |
| Phage-61 | — | — | D | — | — | — | P | E | — | M | M | — | — | — | 0.10 | 2.39 | 0.09 | 219 |
| Phage-62 | — | — | E | — | — | L | P | E | — | M | W | — | — | — | 0.07 | 2.39 | 0.08 | 220 |
| Phage-63 | — | — | E | — | — | — | A | E | — | M | A | — | — | — | 0.07 | 2.38 | 0.08 | 221 |
| Phage-64 | — | — | D | — | — | L | P | E | — | L | A | — | — | — | 0.08 | 2.38 | 0.11 | 222 |
| Phage-65 | — | — | — | — | — | M | P | E | — | M | A | — | — | — | 0.10 | 2.38 | 0.11 | 223 |
| Phage-66 | — | — | D | — | — | L | P | E | — | M | S | — | — | M | 0.10 | 2.38 | 0.14 | 224 |
| Phage-67 | — | — | F | — | — | T | A | E | — | M | Q | — | — | — | 0.08 | 2.38 | 0.08 | 225 |
| Phage-68 | — | — | M | — | — | — | P | E | — | M | A | — | — | — | 0.11 | 2.38 | 0.09 | 226 |
| Phage-69 | — | — | D | — | — | L | P | E | — | M | A | — | — | — | 0.07 | 2.37 | 0.13 | 227 |
| Phage-70 | — | — | D | — | — | S | — | E | — | — | G | — | — | — | 0.08 | 2.37 | 0.07 | 228 |
| Phage-71 | — | — | D | — | — | L | P | E | — | Q | E | — | — | — | 0.06 | 2.37 | 0.10 | 229 |
| Phage-72 | — | — | — | — | — | L | P | — | — | M | S | — | — | — | 0.06 | 2.37 | 0.11 | 230 |
| Phage-73 | — | — | D | — | — | L | P | I | — | M | S | Q | — | — | 0.07 | 2.36 | 0.12 | 231 |
| Phage-74 | — | — | F | — | — | S | P | E | — | M | A | — | — | — | 0.06 | 2.35 | 0.07 | 232 |
| Phage-75 | — | — | D | — | — | L | P | E | — | M | S | — | — | — | 0.07 | 2.35 | 0.14 | 233 |
| Phage-76 | — | — | D | — | — | L | P | — | — | M | A | — | — | — | 0.07 | 2.35 | 0.15 | 234 |
| Phage-77 | — | — | Q | — | — | L | P | M | — | M | A | — | — | — | 0.06 | 2.34 | 0.08 | 235 |
| Phage-78 | — | — | D | — | — | L | P | E | — | M | M | — | — | — | 0.07 | 2.34 | 0.11 | 236 |
| Phage-79 | — | — | D | — | — | S | P | M | — | — | L | — | — | — | 0.07 | 2.34 | 0.08 | 237 |
| Phage-80 | — | — | Q | — | — | L | P | M | — | M | E | — | — | — | 0.09 | 2.34 | 0.09 | 238 |
| Phage-81 | — | — | E | — | — | L | P | — | — | M | A | — | — | — | 0.07 | 2.34 | 0.14 | 239 |
| Phage-82 | — | — | D | — | — | S | P | L | — | M | W | — | — | — | 0.06 | 2.34 | 0.08 | 240 |
| Phage-83 | — | — | D | — | — | L | P | M | — | M | Q | — | — | — | 0.06 | 2.33 | 0.11 | 241 |
| Phage-84 | — | — | D | — | — | L | P | I | — | M | S | — | — | — | 0.07 | 2.32 | 0.08 | 242 |
| Phage-85 | — | — | E | — | — | S | — | A | N | L | G | — | — | — | 0.07 | 2.32 | 0.08 | 243 |
| Phage-86 | — | — | D | — | — | L | P | E | — | R | E | — | — | F | 0.07 | 2.32 | 0.07 | 244 |
| Phage-87 | — | — | E | — | — | L | P | E | — | M | L | — | — | — | 0.06 | 2.32 | 0.09 | 245 |
| Phage-88 | — | — | D | — | — | E | P | — | — | M | L | — | — | — | 0.09 | 2.32 | 0.12 | 246 |
| Phage-89 | — | — | E | — | — | S | P | E | — | M | S | — | — | — | 0.07 | 2.31 | 0.07 | 247 |
| Phage-90 | — | — | E | — | — | T | M | E | — | M | W | — | — | — | 0.06 | 2.31 | 0.07 | 248 |
| Phage-91 | — | — | — | — | — | L | P | E | — | M | P | H | — | I | 0.07 | 2.31 | 0.09 | 249 |
| Phage-92 | — | — | D | — | — | S | P | — | — | — | E | — | — | — | 0.07 | 2.31 | 0.07 | 250 |
| Phage-93 | — | — | D | — | — | S | P | E | — | — | L | — | — | — | 0.06 | 2.31 | 0.09 | 251 |
| Phage-94 | — | — | D | — | — | L | D | E | — | M | A | — | — | — | 0.07 | 2.31 | 0.08 | 252 |
| Phage-95 | — | — | D | — | — | T | L | E | — | M | W | — | — | — | 0.07 | 2.31 | 0.13 | 253 |
| Phage-96 | — | — | E | — | — | L | P | L | — | M | W | — | — | — | 0.07 | 2.30 | 0.07 | 254 |
| Phage-97 | — | — | D | — | — | I | P | E | — | M | A | — | — | — | 0.07 | 2.30 | 0.09 | 255 |
| Phage-98 | — | — | — | — | — | S | P | E | H | M | W | — | — | — | 0.06 | 2.30 | 0.06 | 256 |
| Phage-99 | — | — | E | — | — | S | E | E | — | — | S | — | — | — | 0.07 | 2.30 | 0.08 | 257 |
| Phage-100 | — | — | D | — | — | L | P | I | — | M | A | — | — | — | 0.07 | 2.30 | 0.14 | 258 |
| Phage-101 | — | — | D | — | — | — | P | — | — | M | R | — | — | — | 0.05 | 2.30 | 0.07 | 259 |
| Phage-102 | — | — | D | — | — | — | K | E | — | M | W | — | — | — | 0.07 | 2.30 | 0.07 | 260 |
| Phage-103 | — | — | E | — | — | S | S | E | — | M | L | — | — | — | 0.06 | 2.30 | 0.07 | 261 |
| Phage-104 | — | — | E | — | — | — | A | E | — | M | E | — | — | — | 0.07 | 2.30 | 0.11 | 262 |
| Phage-105 | — | — | E | — | — | S | A | — | — | M | S | — | — | — | 0.07 | 2.29 | 0.07 | 263 |
| Phage-106 | — | — | D | — | — | S | R | E | — | — | A | — | — | — | 0.06 | 2.29 | 0.08 | 264 |
| Phage-107 | — | — | D | — | — | L | P | — | — | M | W | — | — | — | 0.07 | 2.29 | 0.12 | 265 |
| Phage-108 | — | — | D | — | — | V | P | E | — | M | A | — | — | — | 0.08 | 2.29 | 0.07 | 266 |
| Phage-109 | — | — | D | — | — | L | P | M | — | M | M | — | — | — | 0.07 | 2.28 | 0.14 | 267 |
| Phage-110 | — | — | Q | — | — | — | P | E | — | M | A | — | — | — | 0.08 | 2.28 | 0.10 | 268 |
| Phage-111 | — | — | D | — | — | S | Q | E | — | M | M | — | — | — | 0.06 | 2.28 | 0.06 | 269 |
| Phage-112 | — | — | S | — | — | L | P | — | — | M | Q | — | — | — | 0.06 | 2.28 | 0.08 | 270 |
| Phage-113 | — | — | D | — | — | S | S | E | — | M | A | — | — | — | 0.07 | 2.28 | 0.09 | 271 |
| Phage-114 | — | — | D | — | — | — | P | — | — | M | Q | — | — | — | 0.07 | 2.28 | 0.11 | 272 |
| Phage-115 | — | — | E | — | — | L | P | I | — | L | E | — | — | M | 0.06 | 2.27 | 0.10 | 273 |
| Phage-116 | — | — | E | — | — | T | P | E | — | M | Q | — | — | — | 0.07 | 2.27 | 0.07 | 274 |
| Phage-117 | — | — | D | — | — | L | P | — | — | M | M | — | — | — | 0.07 | 2.27 | 0.14 | 275 |
| Phage-119 | — | — | D | — | — | S | T | E | — | L | W | — | — | — | 0.06 | 2.26 | 0.06 | 276 |
| Phage-120 | — | — | E | — | — | L | G | I | — | M | Y | — | — | — | 0.07 | 2.26 | 0.14 | 277 |
| Phage-121 | — | — | D | — | — | — | A | E | — | M | A | — | — | — | 0.07 | 2.26 | 0.13 | 278 |
| Phage-122 | — | — | E | — | — | S | Q | E | — | M | S | — | — | — | 0.07 | 2.26 | 0.07 | 279 |
| Phage-123 | — | — | D | — | — | L | P | E | — | L | W | — | — | — | 0.07 | 2.26 | 0.13 | 280 |
| Phage-124 | — | — | E | — | — | L | P | M | — | M | E | — | — | — | 0.07 | 2.25 | 0.10 | 281 |
| Phage-125 | — | — | D | — | — | L | S | E | — | M | E | — | — | — | 0.07 | 2.25 | 0.08 | 282 |
| Phage-126 | — | — | E | — | — | S | P | L | — | M | W | — | — | — | 0.06 | 2.25 | 0.06 | 283 |
| Phage-127 | — | — | E | — | — | S | P | E | — | M | E | — | — | — | 0.06 | 2.24 | 0.07 | 284 |
| Phage-128 | — | — | — | — | — | L | P | E | — | M | E | — | — | — | 0.08 | 2.24 | 0.12 | 285 |
| Phage-129 | — | — | E | — | — | — | — | E | — | M | A | — | — | — | 0.09 | 2.24 | 0.07 | 286 |
| Phage-130 | — | — | Q | — | — | L | P | E | — | M | — | — | — | — | 0.06 | 2.24 | 0.08 | 287 |
| Phage-131 | — | — | D | — | — | S | Q | E | — | M | A | — | — | — | 0.05 | 2.24 | 0.08 | 288 |
| Phage-132 | — | — | Q | — | — | L | P | I | — | M | E | — | — | — | 0.06 | 2.24 | 0.09 | 289 |
| Phage-133 | — | — | D | — | — | L | P | L | — | L | A | — | — | — | 0.05 | 2.23 | 0.09 | 290 |
| Phage-134 | — | — | D | — | — | L | P | — | — | M | — | — | — | — | 0.05 | 2.23 | 0.10 | 291 |
| Phage-135 | — | — | E | — | — | I | P | M | — | M | W | — | — | — | 0.06 | 2.23 | 0.08 | 292 |
| Phage-136 | — | — | E | — | — | L | P | E | — | M | Q | — | — | M | 0.06 | 2.23 | 0.08 | 293 |
| Phage-137 | — | — | D | — | — | S | A | E | — | M | A | — | — | — | 0.08 | 2.23 | 0.07 | 294 |
| Phage-138 | — | — | E | — | — | S | P | E | — | M | Y | — | — | — | 0.06 | 2.23 | 0.10 | 295 |
| Phage-139 | — | — | D | — | — | S | M | E | — | M | S | — | — | — | 0.08 | 2.22 | 0.09 | 296 |
| Phage-140 | — | — | E | — | — | L | P | M | — | M | D | — | — | — | 0.06 | 2.22 | 0.07 | 297 |
| Phage-141 | — | — | D | — | — | R | G | E | — | L | Q | — | — | — | 0.07 | 2.22 | 0.09 | 298 |
| Phage-142 | — | — | E | — | — | S | P | — | — | M | W | — | — | — | 0.08 | 2.22 | 0.12 | 299 |
| Phage-143 | — | — | E | — | — | — | A | E | — | M | T | — | — | — | 0.07 | 2.22 | 0.08 | 300 |
| Phage-144 | — | — | E | — | — | L | P | — | — | M | K | — | — | — | 0.08 | 2.22 | 0.10 | 301 |
| Phage-145 | — | — | D | — | — | I | E | E | — | R | A | — | — | — | 0.06 | 2.21 | 0.07 | 302 |
| Phage-146 | — | — | E | — | — | — | P | E | — | M | L | — | — | — | 0.06 | 2.21 | 0.07 | 303 |
| Phage-148 | — | — | D | — | — | — | P | E | — | M | E | — | — | M | 0.08 | 2.21 | 0.07 | 304 |
| Phage-149 | — | — | E | — | — | T | V | E | — | M | A | — | — | — | 0.08 | 2.20 | 0.08 | 305 |
| Phage-150 | — | — | D | — | — | K | A | E | — | M | D | — | — | — | 0.07 | 2.20 | 0.07 | 306 |
| Phage-151 | — | — | D | — | — | — | P | E | — | M | L | — | — | — | 0.05 | 2.20 | 0.07 | 307 |
| Phage-152 | — | — | D | — | — | — | P | D | — | M | L | — | — | — | 0.05 | 2.20 | 0.09 | 308 |
| Phage-153 | — | — | D | — | — | L | P | E | — | L | P | — | — | — | 0.07 | 2.19 | 0.09 | 309 |
| Phage-154 | — | — | D | — | — | M | P | E | — | — | A | — | — | — | 0.07 | 2.19 | 0.08 | 310 |
| Phage-155 | — | — | D | — | — | L | P | E | — | L | S | — | — | — | 0.07 | 2.19 | 0.07 | 311 |
| Phage-156 | — | — | — | — | — | L | P | E | — | F | L | — | — | — | 0.07 | 2.19 | 0.08 | 312 |
| Phage-157 | — | — | D | — | — | S | P | L | — | — | W | — | — | — | 0.07 | 2.19 | 0.09 | 313 |
| Phage-158 | — | — | D | — | — | L | P | E | — | M | H | — | — | — | 0.05 | 2.19 | 0.09 | 314 |
| Phage-159 | — | — | D | — | — | L | P | E | — | — | E | — | — | — | 0.06 | 2.19 | 0.07 | 315 |
| Phage-160 | — | — | D | — | — | L | P | — | — | K | A | — | — | — | 0.07 | 2.19 | 0.09 | 316 |
| Phage-161 | — | — | D | — | — | — | V | E | — | M | A | — | — | — | 0.07 | 2.18 | 0.10 | 317 |
| Phage-162 | — | — | D | — | — | S | I | E | — | R | W | — | — | — | 0.06 | 2.18 | 0.06 | 318 |
| Phage-163 | — | — | E | — | — | — | E | E | — | M | Q | — | — | — | 0.06 | 2.18 | 0.06 | 319 |
| Phage-164 | — | — | D | — | — | — | P | E | — | — | Y | — | — | — | 0.06 | 2.18 | 0.06 | 320 |
| Phage-165 | — | — | D | — | — | L | E | E | — | K | Q | — | — | — | 0.07 | 2.18 | 0.08 | 321 |
| Phage-166 | — | — | M | — | — | L | P | — | — | M | Q | — | — | — | 0.09 | 2.18 | 0.11 | 322 |
| Phage-167 | — | — | D | — | — | T | Q | E | — | — | A | — | — | — | 0.05 | 2.17 | 0.06 | 323 |
| Phage-168 | — | — | D | — | — | T | Q | E | — | M | A | — | — | — | 0.08 | 2.17 | 0.07 | 324 |
| Phage-169 | — | — | E | — | — | L | P | V | — | M | W | — | — | — | 0.07 | 2.17 | 0.09 | 325 |
| Phage-170 | — | — | D | — | — | M | P | E | — | M | W | — | — | — | 0.07 | 2.17 | 0.09 | 326 |
| Phage-171 | — | — | — | — | — | S | P | E | — | M | T | — | — | — | 0.05 | 2.17 | 0.09 | 327 |
| Phage-172 | — | — | — | — | — | — | P | E | — | — | Q | — | — | — | 0.06 | 2.17 | 0.09 | 328 |
| Phage-173 | — | — | D | — | — | V | A | E | — | R | D | — | — | — | 0.09 | 2.17 | 0.07 | 329 |
| Phage-174 | — | — | — | — | — | — | P | E | — | M | Q | — | — | — | 0.06 | 2.16 | 0.07 | 330 |
| Phage-175 | — | — | D | — | — | S | N | E | — | M | A | — | — | — | 0.06 | 2.16 | 0.07 | 331 |
| Phage-176 | — | — | E | — | — | L | P | — | — | L | Q | — | — | — | 0.07 | 2.16 | 0.09 | 332 |
| Phage-177 | — | — | D | — | — | V | A | E | — | M | A | — | — | — | 0.15 | 2.16 | 0.07 | 333 |
| Phage-178 | — | — | D | — | — | L | P | E | — | M | W | — | — | I | 0.07 | 2.15 | 0.07 | 334 |
| Phage-179 | — | — | D | — | — | S | A | E | — | M | W | — | — | — | 0.06 | 2.15 | 0.08 | 335 |
| Phage-180 | — | — | D | — | — | S | L | E | — | M | A | — | — | — | 0.05 | 2.15 | 0.06 | 336 |
| Phage-181 | — | — | E | — | — | L | P | E | — | M | S | — | — | — | 0.06 | 2.15 | 0.07 | 337 |
| Phage-182 | — | — | — | — | — | L | P | E | — | M | Q | — | — | — | 0.17 | 2.15 | 0.09 | 338 |
| Phage-183 | — | — | D | — | — | L | G | I | — | M | W | — | — | — | 0.06 | 2.14 | 0.08 | 339 |
| Phage-184 | — | — | E | — | — | S | A | E | — | M | A | — | — | — | 0.05 | 2.14 | 0.06 | 340 |
| Phage-185 | — | — | D | — | — | S | D | E | — | M | A | — | — | — | 0.06 | 2.14 | 0.07 | 341 |
| Phage-186 | — | — | E | — | — | S | L | E | — | — | G | — | — | — | 0.06 | 2.14 | 0.06 | 342 |
| Phage-187 | — | — | D | — | — | Q | P | E | — | M | A | — | — | — | 0.07 | 2.14 | 0.07 | 343 |
| Phage-188 | — | — | — | — | — | L | P | E | — | — | S | — | — | — | 0.06 | 2.14 | 0.08 | 344 |
| Phage-189 | — | — | D | — | — | I | P | I | — | M | S | — | — | — | 0.07 | 2.13 | 0.08 | 345 |
| Phage-190 | — | — | E | — | — | L | P | — | — | L | A | — | — | — | 0.07 | 2.12 | 0.10 | 346 |
| Phage-191 | — | — | — | — | — | L | P | L | — | M | E | — | — | — | 0.06 | 2.12 | 0.06 | 347 |
| Phage-192 | — | — | D | — | — | S | A | E | — | M | M | — | — | — | 0.08 | 2.12 | 0.08 | 348 |
| Phage-193 | — | — | E | — | — | L | P | — | — | — | P | — | — | — | 0.07 | 2.12 | 0.07 | 349 |
| Phage-194 | — | — | D | — | — | — | P | — | — | M | L | — | — | — | 0.07 | 2.12 | 0.09 | 350 |
| Phage-195 | — | — | D | — | — | L | P | V | — | M | A | — | — | — | 0.07 | 2.11 | 0.09 | 351 |
| Phage-196 | — | — | M | — | — | S | E | E | — | M | Q | — | — | — | 0.07 | 2.11 | 0.07 | 352 |
| Phage-197 | — | — | D | — | — | L | P | E | — | M | H | — | — | — | 0.08 | 2.11 | 0.11 | 353 |
| Phage-198 | — | — | D | — | — | L | E | E | — | M | A | — | — | — | 0.07 | 2.11 | 0.09 | 354 |
| Phage-199 | — | — | D | — | — | M | P | E | — | M | L | — | — | — | 0.07 | 2.11 | 0.09 | 355 |
| Phage-200 | — | — | S | — | — | — | P | I | — | M | A | — | — | — | 0.08 | 2.10 | 0.07 | 356 |
| Phage-201 | — | — | — | — | — | T | Q | E | — | M | S | — | — | — | 0.06 | 2.10 | 0.05 | 357 |
| Phage-202 | — | — | E | — | — | S | P | — | — | M | Q | — | — | — | 0.06 | 2.10 | 0.06 | 358 |
| Phage-203 | — | — | D | — | — | V | P | — | — | M | A | — | — | — | 0.08 | 2.10 | 0.07 | 359 |
| Phage-204 | — | — | D | — | — | S | A | E | — | M | S | — | — | — | 0.06 | 2.10 | 0.06 | 360 |
| Phage-205 | — | — | Q | — | — | L | P | — | — | — | A | — | — | — | 0.06 | 2.09 | 0.08 | 361 |
| Phage-206 | — | — | — | — | — | L | P | — | — | M | E | — | — | M | 0.06 | 2.09 | 0.08 | 362 |
| Phage-207 | — | — | — | — | — | L | P | E | H | — | G | — | — | — | 0.05 | 2.09 | 0.07 | 363 |
| Phage-208 | — | — | E | — | — | T | A | E | — | L | Q | — | — | — | 0.07 | 2.09 | 0.10 | 364 |
| Phage-209 | — | — | — | — | — | S | P | E | — | M | G | — | — | M | 0.07 | 2.09 | 0.06 | 365 |
| Phage-210 | — | — | S | — | — | L | P | E | — | M | E | — | — | — | 0.08 | 2.09 | 0.09 | 366 |
| Phage-211 | — | — | — | — | — | L | E | I | — | M | Q | — | — | — | 0.07 | 2.09 | 0.08 | 367 |
| Phage-212 | — | — | M | — | — | L | P | — | — | M | E | — | — | — | 0.06 | 2.08 | 0.08 | 368 |
| Phage-213 | — | — | E | — | — | S | A | E | — | M | W | — | — | — | 0.06 | 2.08 | 0.09 | 369 |
| Phage-214 | — | — | E | — | — | — | P | E | H | M | A | — | — | — | 0.08 | 2.08 | 0.10 | 370 |
| Phage-215 | — | — | D | — | — | — | E | — | — | M | E | — | — | — | 0.06 | 2.08 | 0.07 | 371 |
| Phage-216 | — | — | E | — | — | S | P | E | — | M | S | H | — | F | 0.06 | 2.08 | 0.09 | 372 |
| Phage-217 | — | — | E | — | — | S | M | — | H | M | A | — | — | — | 0.06 | 2.07 | 0.08 | 373 |
| Phage-218 | — | — | D | — | — | — | Q | E | F | M | S | — | — | — | 0.07 | 2.07 | 0.12 | 374 |
| Phage-219 | — | — | — | — | — | L | P | E | — | — | D | — | — | — | 0.06 | 2.07 | 0.08 | 375 |
| Phage-220 | — | — | E | — | — | — | P | — | — | — | Q | — | — | — | 0.06 | 2.07 | 0.07 | 376 |
| Phage-221 | — | — | D | — | — | E | L | R | — | M | A | — | — | — | 0.07 | 2.07 | 0.07 | 377 |
| Phage-222 | — | — | D | — | — | L | P | A | — | M | A | — | — | — | 0.07 | 2.06 | 0.12 | 378 |
| Phage-223 | — | — | E | — | — | S | D | E | — | M | L | — | — | — | 0.07 | 2.06 | 0.07 | 379 |
| Phage-224 | — | — | E | — | — | — | P | E | — | M | M | — | — | I | 0.05 | 2.06 | 0.06 | 380 |
| Phage-225 | — | — | E | — | — | — | D | I | — | M | Q | — | — | — | 0.06 | 2.06 | 0.07 | 381 |
| Phage-226 | — | — | E | — | — | S | A | E | — | M | — | — | — | — | 0.06 | 2.06 | 0.07 | 382 |
| Phage-227 | — | — | D | — | — | — | P | — | — | M | G | — | — | — | 0.08 | 2.06 | 0.08 | 383 |
| Phage-228 | — | — | E | — | — | L | P | I | — | M | D | — | — | — | 0.07 | 2.06 | 0.08 | 384 |
| Phage-229 | — | — | D | — | — | — | I | E | — | M | Q | — | — | — | 0.08 | 2.05 | 0.07 | 385 |
| Phage-230 | — | — | — | — | — | I | E | E | — | M | W | — | — | — | 0.06 | 2.05 | 0.06 | 386 |
| Phage-231 | — | — | Q | — | — | M | P | E | — | M | D | — | — | — | 0.08 | 2.05 | 0.07 | 387 |
| Phage-232 | — | — | D | — | — | — | P | E | — | V | M | — | — | — | 0.08 | 2.05 | 0.10 | 388 |
| Phage-233 | — | — | D | — | — | V | G | E | — | M | Q | — | — | — | 0.10 | 2.05 | 0.08 | 389 |
| Phage-234 | — | — | D | — | — | S | A | — | — | M | H | — | — | — | 0.06 | 2.05 | 0.06 | 390 |
| Phage-235 | — | — | E | — | — | — | P | E | — | M | Y | — | — | — | 0.05 | 2.05 | 0.07 | 391 |
| Phage-236 | — | — | D | — | — | L | P | E | — | Q | M | — | — | — | 0.06 | 2.05 | 0.08 | 392 |
| Phage-237 | — | — | D | — | — | — | S | E | — | M | M | — | — | — | 0.06 | 2.05 | 0.06 | 393 |
| Phage-238 | — | — | D | — | — | L | I | E | — | M | E | — | — | — | 0.06 | 2.04 | 0.06 | 394 |
| Phage-239 | — | — | E | — | — | — | I | — | — | M | S | — | — | — | 0.06 | 2.04 | 0.06 | 395 |
| Phage-240 | — | — | E | — | — | — | P | — | H | M | D | — | — | — | 0.06 | 2.04 | 0.08 | 396 |
| Phage-241 | — | — | E | — | — | S | P | — | — | M | S | — | — | — | 0.06 | 2.04 | 0.07 | 397 |
| Phage-242 | — | — | D | — | — | F | R | E | — | M | Q | — | — | — | 0.08 | 2.04 | 0.06 | 398 |
| Phage-243 | — | — | L | — | — | — | P | — | — | — | Q | — | — | — | 0.08 | 2.04 | 0.14 | 399 |
| Phage-244 | — | — | D | — | — | S | S | E | — | M | W | — | — | — | 0.06 | 2.03 | 0.06 | 400 |
| Phage-245 | — | — | S | — | — | L | P | E | — | M | S | — | — | — | 0.06 | 2.03 | 0.09 | 401 |
| Phage-246 | — | — | E | — | — | — | P | — | — | — | A | — | — | — | 0.06 | 2.03 | 0.08 | 402 |
| Phage-247 | — | — | D | — | — | S | P | M | — | — | G | — | — | — | 0.08 | 2.03 | 0.07 | 403 |
| Phage-248 | — | — | — | — | — | L | P | E | — | M | M | — | — | — | 0.05 | 2.03 | 0.11 | 404 |
| Phage-249 | — | — | E | — | — | R | P | E | — | M | Q | — | — | — | 0.06 | 2.03 | 0.11 | 405 |
| Phage-250 | — | — | E | — | — | — | R | E | H | M | A | — | — | — | 0.07 | 2.02 | 0.08 | 406 |
| Phage-251 | — | — | E | — | — | S | P | E | — | M | T | — | — | — | 0.09 | 2.02 | 0.08 | 407 |
| Phage-252 | — | — | E | — | — | S | I | — | — | M | A | — | — | — | 0.08 | 2.02 | 0.06 | 408 |
| Phage-253 | — | — | E | — | — | S | L | E | — | M | G | — | — | — | 0.07 | 2.02 | 0.09 | 409 |
| Phage-254 | — | — | D | — | — | — | P | E | — | M | E | — | — | — | 0.08 | 2.01 | 0.09 | 410 |
| Phage-255 | — | — | D | — | — | M | G | V | — | Y | W | — | — | — | 0.07 | 2.01 | 0.09 | 411 |
| Phage-256 | — | — | D | — | — | E | A | — | — | M | S | — | — | — | 0.08 | 2.01 | 0.09 | 412 |
| Phage-257 | — | — | D | — | — | S | H | E | — | M | E | — | — | — | 0.05 | 2.01 | 0.06 | 413 |
| Phage-258 | — | — | Q | — | — | L | P | E | — | M | S | — | — | — | 0.06 | 2.01 | 0.06 | 414 |
| Phage-259 | — | — | E | — | — | L | S | — | — | M | Q | — | — | — | 0.11 | 2.00 | 0.09 | 415 |
| Phage-260 | — | — | D | — | — | S | P | I | — | M | S | — | — | — | 0.06 | 2.00 | 0.09 | 416 |
| Phage-261 | — | — | D | — | — | S | D | — | — | M | W | — | — | — | 0.09 | 2.00 | 0.11 | 417 |
| Phage-262 | — | — | D | — | — | L | P | — | — | M | S | — | — | — | 0.07 | 2.00 | 0.10 | 418 |
| Phage-263 | — | — | E | — | — | — | A | E | — | M | M | — | — | — | 0.05 | 1.99 | 0.07 | 419 |
| Phage-264 | — | — | — | — | — | L | P | M | — | M | H | — | — | — | 0.13 | 1.99 | 0.18 | 420 |
| Phage-265 | — | — | D | — | — | S | Q | E | — | — | A | — | — | — | 0.07 | 1.99 | 0.07 | 421 |
| Phage-266 | — | — | D | — | — | M | P | — | — | M | S | — | — | — | 0.06 | 1.98 | 0.08 | 422 |
| Phage-267 | — | — | V | — | — | L | P | — | — | M | E | — | — | — | 0.06 | 1.98 | 0.08 | 423 |
| Phage-268 | — | — | D | — | — | L | G | I | — | M | T | — | — | — | 0.06 | 1.98 | 0.08 | 424 |
| Phage-269 | — | — | D | — | — | L | — | E | — | M | D | — | — | — | 0.05 | 1.97 | 0.06 | 425 |
| Phage-270 | — | — | E | — | — | L | P | M | — | F | L | — | — | M | 0.06 | 1.97 | 0.06 | 426 |
| Phage-271 | — | — | D | — | — | L | P | I | — | M | T | Q | — | — | 0.06 | 1.97 | 0.08 | 427 |
| Phage-272 | — | — | E | — | — | L | A | E | — | M | D | — | — | — | 0.07 | 1.96 | 0.07 | 428 |
| Phage-273 | — | — | D | — | — | L | E | — | — | R | A | — | — | — | 0.06 | 1.96 | 0.08 | 429 |
| Phage-274 | — | — | D | — | — | L | P | E | — | L | G | — | — | — | 0.07 | 1.96 | 0.08 | 430 |
| Phage-275 | — | — | — | — | — | S | V | E | — | M | A | — | — | — | 0.07 | 1.96 | 0.08 | 431 |
| Phage-276 | — | — | D | — | — | S | A | L | — | M | W | — | — | — | 0.11 | 1.96 | 0.08 | 432 |
| Phage-277 | — | — | D | — | — | S | S | E | — | M | I | — | — | — | 0.06 | 1.95 | 0.06 | 433 |
| Phage-278 | — | — | D | — | — | L | I | E | — | M | Y | — | — | — | 0.08 | 1.95 | 0.09 | 434 |
| Phage-279 | — | — | D | — | — | S | A | E | — | M | Q | — | — | — | 0.06 | 1.95 | 0.07 | 435 |
| Phage-280 | — | — | D | — | — | L | V | E | — | M | S | — | — | — | 0.09 | 1.95 | 0.08 | 436 |
| Phage-281 | — | — | E | — | — | L | S | — | — | M | E | — | — | — | 0.06 | 1.95 | 0.08 | 437 |
| Phage-282 | — | — | D | — | — | S | P | E | H | — | R | — | — | — | 0.08 | 1.94 | 0.11 | 438 |
| Phage-283 | — | — | — | — | — | L | E | I | — | M | — | — | — | — | 0.05 | 1.94 | 0.07 | 439 |
| Phage-284 | — | — | E | — | — | L | S | E | — | M | A | — | — | — | 0.06 | 1.94 | 0.20 | 440 |
| Phage-285 | — | — | E | — | — | L | P | I | — | L | S | — | — | — | 0.07 | 1.94 | 0.08 | 441 |
| Phage-286 | — | — | D | — | — | — | S | E | — | M | W | — | — | — | 0.06 | 1.94 | 0.06 | 442 |
| Phage-287 | — | — | S | — | — | — | P | E | — | M | A | — | — | — | 0.05 | 1.94 | 0.06 | 443 |
| Phage-288 | — | — | E | — | — | R | A | E | — | M | E | — | — | — | 0.06 | 1.93 | 0.06 | 444 |
| Phage-289 | — | — | D | — | — | S | P | D | — | — | W | — | — | — | 0.07 | 1.93 | 0.10 | 445 |
| Phage-290 | — | — | M | — | — | — | P | E | — | M | E | — | — | — | 0.06 | 1.93 | 0.07 | 446 |
| Phage-291 | — | — | M | — | — | Q | P | E | — | M | Q | — | — | — | 0.08 | 1.93 | 0.08 | 447 |
| Phage-292 | — | — | D | — | — | M | P | E | — | M | E | — | — | — | 0.08 | 1.93 | 0.09 | 448 |
| Phage-293 | — | — | D | — | — | S | A | E | — | L | A | — | — | — | 0.07 | 1.93 | 0.08 | 449 |
| Phage-294 | — | — | D | — | — | S | P | E | — | M | T | — | — | — | 0.06 | 1.93 | 0.07 | 450 |
| Phage-295 | — | — | E | — | — | S | M | E | — | M | A | — | — | — | 0.07 | 1.92 | 0.07 | 451 |
| Phage-296 | — | — | — | — | — | M | P | E | — | — | D | — | — | — | 0.05 | 1.92 | 0.06 | 452 |
| Phage-297 | — | — | — | — | — | S | P | E | F | M | E | — | — | — | 0.06 | 1.91 | 0.06 | 453 |
| Phage-298 | — | — | D | — | — | L | N | E | — | M | E | — | — | — | 0.05 | 1.91 | 0.06 | 454 |
| Phage-299 | — | — | D | — | — | L | K | E | — | Q | E | — | — | — | 0.07 | 1.91 | 0.09 | 455 |
| Phage-300 | — | — | E | — | — | L | A | — | — | M | W | — | — | — | 0.08 | 1.91 | 0.12 | 456 |
| Phage-301 | — | — | E | — | — | L | A | V | — | M | Q | — | — | — | 0.08 | 1.91 | 0.10 | 457 |
| Phage-302 | — | — | E | — | — | S | S | E | — | M | E | — | — | — | 0.05 | 1.91 | 0.06 | 458 |
| Phage-303 | — | — | D | — | — | L | D | I | — | L | A | — | — | — | 0.07 | 1.91 | 0.08 | 459 |
| Phage-304 | — | — | E | — | — | S | E | E | — | M | S | — | — | — | 0.06 | 1.91 | 0.07 | 460 |
| Phage-305 | — | — | D | — | — | S | Q | E | — | M | W | — | — | — | 0.06 | 1.90 | 0.09 | 461 |
| Phage-306 | — | — | D | — | — | — | P | E | — | M | K | — | — | — | 0.05 | 1.90 | 0.06 | 462 |
| Phage-307 | — | — | D | — | — | S | K | E | — | M | H | — | — | — | 0.07 | 1.90 | 0.07 | 463 |
| Phage-308 | — | — | D | — | — | E | P | — | — | L | E | — | — | — | 0.08 | 1.90 | 0.08 | 464 |
| Phage-309 | — | — | D | — | — | — | E | E | — | M | S | — | — | — | 0.05 | 1.90 | 0.09 | 465 |
| Phage-310 | — | — | D | — | — | V | E | E | — | L | Y | — | — | — | 0.05 | 1.90 | 0.06 | 466 |
| Phage-311 | — | — | — | — | — | L | E | E | — | M | D | — | — | — | 0.05 | 1.90 | 0.06 | 467 |
| Phage-312 | — | — | — | — | — | — | P | — | — | M | E | — | — | — | 0.08 | 1.90 | 0.08 | 468 |
| Phage-313 | — | — | — | — | — | L | P | — | — | — | A | — | — | — | 0.07 | 1.90 | 0.09 | 469 |
| Phage-314 | — | — | D | — | — | L | K | E | — | M | A | — | — | — | 0.05 | 1.90 | 0.07 | 470 |
| Phage-315 | — | — | D | — | — | R | P | E | — | M | E | — | — | — | 0.05 | 1.90 | 0.06 | 471 |
| Phage-316 | — | — | D | — | — | L | P | E | — | M | G | — | — | — | 0.06 | 1.89 | 0.11 | 472 |
| Phage-317 | — | — | E | — | — | L | Q | E | — | M | E | — | — | — | 0.06 | 1.89 | 0.10 | 473 |
| Phage-318 | — | — | E | — | — | I | P | E | — | M | A | — | — | — | 0.06 | 1.89 | 0.06 | 474 |
| Phage-319 | — | — | D | — | — | S | P | E | — | M | P | — | — | — | 0.06 | 1.89 | 0.08 | 475 |
| Phage-320 | — | — | D | — | — | L | A | E | — | M | P | — | — | — | 0.08 | 1.89 | 0.07 | 476 |
| Phage-321 | — | — | E | — | — | S | P | E | — | M | W | — | — | — | 0.05 | 1.89 | 0.09 | 477 |
| Phage-322 | — | — | D | — | — | I | D | E | — | L | Q | — | — | — | 0.06 | 1.88 | 0.06 | 478 |
| Phage-323 | — | — | E | — | — | V | A | E | — | M | L | — | — | — | 0.05 | 1.88 | 0.05 | 479 |
| Phage-324 | — | — | E | — | — | L | S | E | — | M | S | — | — | — | 0.07 | 1.88 | 0.09 | 480 |
| Phage-325 | — | — | E | — | — | M | S | E | — | M | A | — | — | — | 0.08 | 1.88 | 0.11 | 481 |
| Phage-326 | — | — | D | — | — | S | — | E | — | — | Q | — | — | — | 0.06 | 1.88 | 0.07 | 482 |
| Phage-327 | — | — | E | — | — | S | L | E | — | M | A | — | — | — | 0.07 | 1.88 | 0.08 | 483 |
| Phage-328 | — | — | E | — | — | S | Y | E | — | M | W | — | — | — | 0.06 | 1.88 | 0.08 | 484 |
| Phage-329 | — | — | E | — | — | L | P | E | — | M | M | — | — | — | 0.06 | 1.88 | 0.27 | 485 |
| Phage-330 | — | — | D | — | — | L | P | E | — | M | R | — | — | — | 0.07 | 1.88 | 0.08 | 486 |
| Phage-331 | — | — | E | — | — | S | P | L | — | M | S | — | — | — | 0.06 | 1.87 | 0.09 | 487 |
| Phage-332 | — | — | E | — | — | S | — | E | — | — | G | — | — | — | 0.08 | 1.87 | 0.07 | 488 |
| Phage-333 | — | — | D | — | — | S | A | L | — | M | S | — | — | — | 0.06 | 1.87 | 0.08 | 489 |
| Phage-334 | — | — | D | — | — | T | G | V | — | F | Y | — | — | — | 0.07 | 1.87 | 0.08 | 490 |
| Phage-335 | — | — | E | — | — | T | S | E | — | M | — | — | — | — | 0.06 | 1.87 | 0.06 | 491 |
| Phage-336 | — | — | E | — | — | R | S | E | — | M | A | — | — | — | 0.06 | 1.86 | 0.06 | 492 |
| Phage-337 | — | — | D | — | — | L | A | M | — | M | D | — | — | — | 0.05 | 1.86 | 0.08 | 493 |
| Phage-338 | — | — | D | — | — | M | P | E | — | L | A | — | — | — | 0.06 | 1.86 | 0.07 | 494 |
| Phage-339 | — | — | E | — | — | — | A | E | — | M | S | — | — | — | 0.08 | 1.86 | 0.07 | 495 |
| Phage-340 | — | — | — | — | — | S | S | E | — | M | W | — | — | — | 0.08 | 1.85 | 0.07 | 496 |
| Phage-341 | — | — | D | — | — | S | — | A | — | M | W | — | — | — | 0.11 | 1.85 | 0.13 | 497 |
| Phage-342 | — | — | D | — | — | S | A | A | — | — | M | — | — | — | 0.06 | 1.84 | 0.09 | 498 |
| Phage-343 | — | — | D | — | — | S | L | — | — | L | G | — | — | — | 0.06 | 1.84 | 0.09 | 499 |
| Phage-344 | — | — | E | — | — | S | Q | E | — | M | Q | — | — | — | 0.05 | 1.84 | 0.05 | 500 |
| Phage-345 | — | — | — | — | — | L | P | E | — | L | S | — | — | — | 0.07 | 1.84 | 0.07 | 501 |
| Phage-346 | — | — | E | — | — | L | Q | I | — | M | S | — | — | — | 0.10 | 1.84 | 0.14 | 502 |
| Phage-347 | — | — | — | — | — | — | P | E | — | M | F | — | — | M | 0.06 | 1.83 | 0.06 | 503 |
| Phage-348 | — | — | E | — | — | R | P | E | — | M | A | — | — | — | 0.06 | 1.83 | 0.07 | 504 |
| Phage-349 | — | — | E | — | — | L | P | E | — | M | R | — | — | — | 0.05 | 1.83 | 0.06 | 505 |
| Phage-350 | — | — | D | — | — | S | P | A | — | M | A | — | — | — | 0.09 | 1.83 | 0.10 | 506 |
| Phage-351 | — | — | D | — | — | L | P | M | — | F | A | — | — | M | 0.06 | 1.83 | 0.06 | 507 |
| Phage-352 | — | — | D | — | — | S | S | E | — | — | V | — | — | — | 0.08 | 1.83 | 0.06 | 508 |
| Phage-353 | — | — | E | — | — | — | A | E | — | M | W | — | — | — | 0.09 | 1.83 | 0.07 | 509 |
| Phage-354 | — | — | E | — | — | S | A | E | — | — | F | — | — | — | 0.07 | 1.83 | 0.05 | 510 |
| Phage-355 | — | — | E | — | — | S | P | M | — | M | Q | — | — | M | 0.07 | 1.83 | 0.06 | 511 |
| Phage-356 | — | — | D | — | — | S | A | E | — | M | E | — | — | I | 0.06 | 1.83 | 0.07 | 512 |
| Phage-357 | — | — | D | — | — | L | P | L | — | M | H | — | — | — | 0.05 | 1.82 | 0.07 | 513 |
| Phage-358 | — | — | E | — | — | — | P | — | — | L | — | — | — | — | 0.09 | 1.82 | 0.08 | 514 |
| Phage-359 | — | — | D | — | — | — | R | E | — | M | T | — | — | — | 0.06 | 1.82 | 0.06 | 515 |
| Phage-360 | — | — | D | — | — | T | E | E | — | M | D | — | — | — | 0.07 | 1.82 | 0.09 | 516 |
| Phage-361 | — | — | D | — | — | S | S | M | — | M | A | — | — | — | 0.05 | 1.82 | 0.06 | 517 |
| Phage-362 | — | — | — | — | — | S | S | E | — | — | W | — | — | — | 0.06 | 1.82 | 0.06 | 518 |
| Phage-363 | — | — | E | — | — | S | A | E | F | M | T | — | — | — | 0.06 | 1.81 | 0.06 | 519 |
| Phage-364 | — | — | Q | — | — | S | A | E | — | M | M | — | — | — | 0.06 | 1.81 | 0.07 | 520 |
| Phage-365 | — | — | — | — | — | L | P | — | — | — | — | — | — | — | 0.05 | 1.81 | 0.06 | 521 |
| Phage-366 | — | — | D | — | — | K | A | E | — | M | E | — | — | — | 0.11 | 1.81 | 0.09 | 522 |
| Phage-367 | — | — | — | — | — | L | P | M | — | M | A | — | — | — | 0.05 | 1.80 | 0.08 | 523 |
| Phage-368 | — | — | D | — | — | T | R | E | — | — | D | — | — | — | 0.06 | 1.80 | 0.08 | 524 |
| Phage-369 | — | — | V | — | — | — | P | E | F | M | A | — | — | — | 0.07 | 1.80 | 0.10 | 525 |
| Phage-370 | — | — | — | — | — | — | E | — | — | M | A | — | — | — | 0.07 | 1.80 | 0.08 | 526 |
| Phage-371 | — | — | D | — | — | L | S | E | — | M | F | — | — | — | 0.05 | 1.79 | 0.10 | 527 |
| Phage-372 | — | — | D | — | — | L | G | V | — | M | A | — | — | — | 0.05 | 1.79 | 0.07 | 528 |
| Phage-373 | — | — | D | — | — | T | S | E | — | — | T | — | — | — | 0.07 | 1.79 | 0.07 | 529 |
| Phage-374 | — | — | D | — | — | S | P | E | — | — | A | — | — | — | 0.08 | 1.79 | 0.23 | 530 |
| Phage-375 | — | — | D | — | — | L | A | E | — | M | V | — | — | — | 0.08 | 1.79 | 0.08 | 531 |
| Phage-376 | — | — | D | — | — | I | P | M | — | M | A | — | — | — | 0.06 | 1.79 | 0.08 | 532 |
| Phage-377 | — | — | E | — | — | L | P | A | — | M | Q | — | — | — | 0.06 | 1.78 | 0.07 | 533 |
| Phage-378 | — | — | E | — | — | H | P | I | — | M | A | — | — | — | 0.06 | 1.78 | 0.09 | 534 |
| Phage-379 | — | — | — | — | — | V | P | E | — | M | A | — | — | — | 0.06 | 1.78 | 0.07 | 535 |
| Phage-380 | — | — | D | — | — | S | A | E | — | — | T | — | — | — | 0.06 | 1.77 | 0.06 | 536 |
| Phage-381 | — | — | E | — | — | L | D | E | — | M | W | — | — | — | 0.06 | 1.77 | 0.06 | 537 |
| Phage-382 | — | — | D | — | L | — | S | E | — | M | S | — | — | — | 0.05 | 1.77 | 0.05 | 538 |
| Phage-383 | — | — | D | — | — | S | S | E | — | — | A | — | — | — | 0.09 | 1.77 | 0.07 | 539 |
| Phage-384 | — | — | E | — | — | S | P | — | — | V | Q | — | — | — | 0.05 | 1.77 | 0.06 | 540 |
| Phage-385 | — | — | E | — | — | S | P | E | — | M | R | — | — | — | 0.07 | 1.77 | 0.08 | 541 |
| Phage-386 | — | — | D | — | — | L | D | E | — | M | E | — | — | — | 0.06 | 1.76 | 0.08 | 542 |
| Phage-387 | — | — | — | — | — | L | P | E | — | M | L | — | — | — | 0.07 | 1.76 | 0.09 | 543 |
| Phage-388 | — | — | E | — | — | L | P | I | — | M | S | — | — | — | 0.07 | 1.76 | 0.09 | 544 |
| Phage-389 | — | — | V | — | — | L | Q | E | — | M | E | — | — | — | 0.06 | 1.76 | 0.06 | 545 |
| Phage-390 | — | — | D | — | — | S | P | — | — | M | Q | — | — | — | 0.06 | 1.76 | 0.07 | 546 |
| Phage-391 | — | — | D | — | — | I | P | E | — | L | A | — | — | — | 0.05 | 1.76 | 0.06 | 547 |
| Phage-392 | — | — | D | — | — | S | Q | E | — | M | L | — | — | — | 0.05 | 1.76 | 0.05 | 548 |
| Phage-393 | — | — | D | — | — | V | E | P | — | L | S | — | — | — | 0.07 | 1.76 | 0.10 | 549 |
| Phage-394 | — | — | D | — | — | K | P | — | — | M | E | — | — | — | 0.06 | 1.76 | 0.08 | 550 |
| Phage-395 | — | — | D | — | — | I | G | E | — | M | V | — | — | — | 0.06 | 1.76 | 0.66 | 551 |
| Phage-396 | — | — | E | — | — | L | A | — | — | M | — | — | — | — | 0.08 | 1.76 | 0.07 | 552 |
| Phage-397 | — | — | D | — | — | V | Q | E | — | M | Q | — | — | — | 0.06 | 1.76 | 0.07 | 553 |
| Phage-398 | — | — | H | — | — | L | Q | E | — | M | A | — | — | — | 0.08 | 1.76 | 0.07 | 554 |
| Phage-399 | — | — | D | — | — | S | P | E | — | M | — | — | — | — | 0.06 | 1.75 | 0.06 | 555 |
| Phage-400 | — | — | D | — | — | — | P | V | — | M | A | — | — | — | 0.06 | 1.75 | 0.06 | 556 |
| Phage-401 | — | — | E | — | — | S | A | E | — | M | L | — | — | — | 0.07 | 1.75 | 0.07 | 557 |
| Phage-402 | — | — | D | — | — | H | I | E | — | L | A | — | — | — | 0.07 | 1.75 | 0.10 | 558 |
| Phage-403 | — | — | S | — | — | L | P | E | — | M | Q | — | — | — | 0.06 | 1.75 | 0.08 | 559 |
| Phage-404 | — | — | E | — | — | L | P | E | — | M | Q | — | — | — | 0.07 | 1.75 | 0.07 | 560 |
| Phage-405 | — | — | D | — | — | S | L | — | — | M | D | — | — | — | 0.06 | 1.75 | 0.06 | 561 |
| Phage-406 | — | — | D | — | — | Y | P | E | — | M | S | — | — | — | 0.07 | 1.75 | 0.06 | 562 |
| Phage-407 | — | — | D | — | — | — | S | E | — | M | A | — | — | — | 0.06 | 1.75 | 0.09 | 563 |
| Phage-408 | — | — | D | — | — | — | E | K | — | M | Q | — | — | — | 0.06 | 1.74 | 0.06 | 564 |
| Phage-409 | — | — | E | — | — | — | I | E | — | L | A | — | — | — | 0.05 | 1.74 | 0.07 | 565 |
| Phage-410 | — | — | E | — | — | S | A | E | — | M | F | — | — | — | 0.06 | 1.73 | 0.07 | 566 |
| Phage-411 | — | — | S | — | — | S | A | E | — | M | L | — | — | — | 0.07 | 1.73 | 0.08 | 567 |
| Phage-412 | — | — | E | — | — | L | P | D | — | M | Q | — | — | — | 0.06 | 1.73 | 0.07 | 568 |
| Phage-413 | — | — | D | — | — | L | E | E | — | M | V | — | — | M | 0.06 | 1.73 | 0.06 | 569 |
| Phage-414 | — | — | E | — | — | L | Q | T | — | M | A | — | — | — | 0.08 | 1.73 | 0.07 | 570 |
| Phage-415 | — | — | — | — | — | S | Q | E | H | — | Q | — | — | — | 0.07 | 1.73 | 0.09 | 571 |
| Phage-416 | — | — | D | — | — | L | P | E | — | — | F | — | — | — | 0.05 | 1.73 | 0.09 | 572 |
| Phage-417 | — | — | S | — | — | L | P | E | — | M | G | — | — | — | 0.06 | 1.73 | 0.10 | 573 |
| Phage-418 | — | — | D | — | — | — | E | E | — | L | W | — | — | — | 0.05 | 1.73 | 0.06 | 574 |
| Phage-419 | — | — | — | — | — | S | A | E | — | M | A | — | — | — | 0.05 | 1.73 | 0.07 | 575 |
| Phage-420 | — | — | D | — | — | S | G | V | — | M | W | — | — | — | 0.08 | 1.73 | 0.08 | 576 |
| Phage-421 | — | — | D | — | — | T | Q | A | — | — | Q | — | — | — | 0.05 | 1.73 | 0.06 | 577 |
| Phage-422 | — | — | D | — | — | — | I | — | — | — | G | — | — | — | 0.06 | 1.72 | 0.09 | 578 |
| Phage-423 | — | — | D | — | — | — | A | E | F | M | M | — | — | — | 0.05 | 1.72 | 0.06 | 579 |
| Phage-424 | — | — | D | — | — | L | S | E | — | M | A | — | — | — | 0.07 | 1.72 | 0.08 | 580 |
| Phage-425 | — | — | E | — | — | S | E | M | — | M | K | — | — | — | 0.08 | 1.72 | 0.07 | 581 |
| Phage-426 | — | — | D | — | — | L | Q | I | — | L | W | — | — | — | 0.06 | 1.72 | 0.07 | 582 |
| Phage-427 | — | — | Q | — | — | L | P | V | — | M | E | — | — | — | 0.06 | 1.72 | 0.06 | 583 |
| Phage-428 | — | — | D | — | — | — | A | E | — | M | D | — | — | — | 0.05 | 1.72 | 0.07 | 584 |
| Phage-429 | — | — | E | — | — | S | V | — | — | M | Q | — | — | — | 0.06 | 1.71 | 0.06 | 585 |
| Phage-430 | — | — | — | — | — | L | P | E | — | L | D | — | — | — | 0.06 | 1.71 | 0.07 | 586 |
| Phage-431 | — | — | D | — | — | S | R | E | — | M | V | — | — | — | 0.06 | 1.71 | 0.06 | 587 |
| Phage-432 | — | — | D | — | — | L | V | E | — | R | G | — | — | — | 0.06 | 1.71 | 0.07 | 588 |
| Phage-433 | — | — | — | — | — | S | A | E | — | M | W | — | — | — | 0.06 | 1.71 | 0.11 | 589 |
| Phage-434 | — | — | D | — | — | L | Q | I | — | M | H | — | — | — | 0.07 | 1.71 | 0.10 | 590 |
| Phage-435 | — | — | — | — | — | S | M | E | — | M | A | — | — | — | 0.06 | 1.71 | 0.07 | 591 |
| Phage-436 | — | — | D | — | — | L | P | E | — | F | L | — | — | — | 0.09 | 1.71 | 0.11 | 592 |
| Phage-437 | — | — | D | — | — | S | L | A | — | — | A | — | — | — | 0.05 | 1.70 | 0.06 | 593 |
| Phage-438 | — | — | — | — | — | T | Q | E | — | M | Y | — | — | — | 0.06 | 1.70 | 0.05 | 594 |
| Phage-439 | — | — | E | — | — | — | Q | E | — | M | S | — | — | — | 0.05 | 1.70 | 0.08 | 595 |
| Phage-440 | — | — | E | — | — | L | P | — | — | F | M | — | — | — | 0.06 | 1.70 | 0.07 | 596 |
| Phage-441 | — | — | D | — | — | S | — | E | — | M | E | — | — | — | 0.05 | 1.70 | 0.12 | 597 |
| Phage-442 | — | — | — | — | — | S | P | L | — | M | L | — | — | — | 0.06 | 1.70 | 0.10 | 598 |
| Phage-443 | — | — | E | — | — | S | A | E | — | M | Q | — | — | — | 0.08 | 1.70 | 0.29 | 599 |
| Phage-444 | — | — | D | — | — | L | P | V | — | L | E | — | — | — | 0.05 | 1.69 | 0.06 | 600 |
| Phage-445 | — | — | — | — | — | L | Q | E | — | M | Q | — | — | — | 0.06 | 1.69 | 0.07 | 601 |
| Phage-446 | — | — | D | — | — | — | I | E | — | M | A | — | — | — | 0.17 | 1.69 | 0.08 | 602 |
| Phage-447 | — | — | E | — | — | K | P | E | — | M | Q | — | — | — | 0.06 | 1.69 | 0.06 | 603 |
| Phage-448 | — | — | D | — | — | — | — | E | — | M | T | — | — | — | 0.10 | 1.69 | 0.35 | 604 |
| Phage-449 | — | — | E | — | — | S | P | W | — | M | — | — | — | — | 0.06 | 1.68 | 0.06 | 605 |
| Phage-450 | — | — | D | — | — | S | P | A | — | — | W | — | — | — | 0.08 | 1.68 | 0.08 | 606 |
| Phage-451 | — | — | D | — | — | S | E | — | — | M | S | — | — | — | 0.06 | 1.68 | 0.07 | 607 |
| Phage-452 | — | — | D | — | — | — | K | E | — | M | D | — | — | — | 0.08 | 1.68 | 0.08 | 608 |
| Phage-453 | — | — | D | — | — | S | T | E | — | M | E | — | — | — | 0.06 | 1.68 | 0.08 | 609 |
| Phage-454 | — | — | D | — | — | S | S | L | — | M | H | — | — | — | 0.06 | 1.67 | 0.07 | 610 |
| Phage-455 | — | — | D | — | — | S | — | — | — | — | S | — | — | — | 0.07 | 1.67 | 0.05 | 611 |
| Phage-456 | — | — | E | — | — | — | P | — | — | M | — | — | — | — | 0.08 | 1.67 | 0.08 | 612 |
| Phage-457 | — | — | D | — | — | — | P | — | W | M | A | — | — | — | 0.08 | 1.67 | 0.10 | 613 |
| Phage-458 | — | — | D | — | — | S | R | E | — | M | S | — | — | — | 0.07 | 1.67 | 0.10 | 614 |
| Phage-459 | — | — | D | — | — | — | A | E | — | M | A | — | — | V | 0.06 | 1.67 | 0.07 | 615 |
| Phage-460 | — | — | E | — | — | — | Q | E | — | M | — | — | — | — | 0.06 | 1.67 | 0.06 | 616 |
| Phage-461 | — | — | E | — | — | — | P | — | — | M | T | — | — | — | 0.06 | 1.67 | 0.08 | 617 |
| Phage-462 | — | — | D | — | — | S | A | D | — | M | A | — | — | — | 0.06 | 1.67 | 0.07 | 618 |
| Phage-463 | — | — | D | — | — | I | G | L | — | L | — | — | — | — | 0.07 | 1.66 | 0.06 | 619 |
| Phage-464 | — | — | E | — | — | L | P | M | — | M | I | — | — | — | 0.07 | 1.66 | 0.09 | 620 |
| Phage-465 | — | — | E | — | — | L | P | — | — | M | R | P | — | — | 0.06 | 1.66 | 0.08 | 621 |
| Phage-466 | — | — | E | — | — | L | S | — | — | M | S | — | — | — | 0.05 | 1.66 | 0.07 | 622 |
| Phage-467 | — | — | D | — | — | S | P | E | — | — | G | — | — | — | 0.06 | 1.66 | 0.07 | 623 |
| Phage-468 | — | — | — | — | — | L | P | E | F | M | D | — | — | — | 0.06 | 1.66 | 0.06 | 624 |
| Phage-469 | — | — | D | — | — | S | R | E | — | M | — | — | — | — | 0.05 | 1.66 | 0.06 | 625 |
| Phage-470 | — | — | D | — | — | D | V | E | — | M | A | — | — | — | 0.07 | 1.66 | 0.07 | 626 |
| Phage-471 | — | — | D | — | — | S | E | V | — | M | A | — | — | — | 0.07 | 1.66 | 0.06 | 627 |
| Phage-472 | — | — | D | — | — | S | L | L | — | L | S | — | — | — | 0.07 | 1.65 | 0.07 | 628 |
| Phage-473 | — | — | E | — | — | L | A | V | — | M | E | — | — | — | 0.08 | 1.65 | 0.12 | 629 |
| Phage-474 | — | — | E | — | — | S | L | M | — | M | W | — | — | — | 0.06 | 1.65 | 0.08 | 630 |
| Phage-475 | — | — | — | — | — | — | A | E | — | M | T | — | — | — | 0.06 | 1.65 | 0.07 | 631 |
| Phage-476 | — | — | D | — | — | S | V | E | — | M | E | — | — | — | 0.07 | 1.65 | 0.09 | 632 |
| Phage-477 | — | — | D | — | — | — | Q | E | — | R | L | — | — | — | 0.07 | 1.65 | 0.07 | 633 |
| Phage-478 | — | — | D | — | — | S | A | E | — | L | L | — | — | — | 0.07 | 1.65 | 0.07 | 634 |
| Phage-479 | — | — | D | — | — | G | P | — | — | — | Q | — | — | — | 0.06 | 1.64 | 0.07 | 635 |
| Phage-480 | — | — | E | — | — | I | A | E | — | M | Q | — | — | — | 0.06 | 1.64 | 0.13 | 636 |
| Phage-481 | — | — | E | — | — | S | A | — | — | M | H | — | — | — | 0.05 | 1.64 | 0.06 | 637 |
| Phage-482 | — | — | E | — | — | T | A | E | H | M | Q | — | — | — | 0.05 | 1.64 | 0.05 | 638 |
| Phage-483 | — | — | — | — | — | V | A | E | — | M | A | — | — | — | 0.06 | 1.64 | 0.06 | 639 |
| Phage-484 | — | — | D | — | — | — | T | E | — | M | Q | — | — | — | 0.05 | 1.64 | 0.10 | 640 |
| Phage-485 | — | — | E | — | — | M | — | E | — | M | A | — | — | — | 0.08 | 1.64 | 0.13 | 641 |
| Phage-486 | — | — | D | — | — | D | V | E | — | M | S | — | — | — | 0.06 | 1.64 | 0.05 | 642 |
| Phage-487 | — | — | — | — | — | S | L | E | — | M | Q | — | — | — | 0.06 | 1.63 | 0.07 | 643 |
| Phage-488 | — | — | E | — | — | D | L | I | — | M | W | — | — | — | 0.06 | 1.63 | 0.06 | 644 |
| Phage-489 | — | — | — | — | — | — | P | E | — | M | S | — | — | — | 0.05 | 1.63 | 0.05 | 645 |
| Phage-490 | — | — | E | — | — | S | S | E | — | M | W | — | — | — | 0.07 | 1.63 | 0.09 | 646 |
| Phage-491 | — | — | D | — | — | L | D | V | — | R | A | — | — | — | 0.07 | 1.63 | 0.09 | 647 |
| Phage-492 | — | — | E | — | — | L | E | — | — | M | Q | — | — | — | 0.05 | 1.63 | 0.06 | 648 |
| Phage-493 | — | — | E | — | — | S | — | V | — | M | A | — | — | — | 0.09 | 1.63 | 0.07 | 649 |
| Phage-494 | — | — | D | — | — | S | L | E | — | — | G | — | — | — | 0.06 | 1.63 | 0.06 | 650 |
| Phage-495 | — | — | D | — | — | S | E | A | F | M | Q | — | — | — | 0.07 | 1.63 | 0.07 | 651 |
| Phage-496 | — | — | — | — | — | L | P | E | — | M | G | — | — | — | 0.05 | 1.63 | 0.07 | 652 |
| Phage-497 | — | — | — | — | — | L | A | I | — | M | S | — | — | — | 0.08 | 1.63 | 0.08 | 653 |
| Phage-498 | — | — | D | — | — | L | S | E | — | M | S | — | — | — | 0.05 | 1.62 | 0.06 | 654 |
| Phage-499 | — | — | E | — | — | V | T | E | — | M | Q | — | — | — | 0.07 | 1.62 | 0.08 | 655 |
| Phage-500 | — | — | D | — | — | — | Q | E | — | M | D | — | — | — | 0.06 | 1.62 | 0.08 | 656 |
| Phage-501 | — | — | F | — | — | — | P | — | — | — | M | — | — | — | 0.06 | 1.62 | 0.06 | 657 |
| Phage-502 | — | — | D | — | — | E | L | K | — | M | L | — | — | — | 0.05 | 1.62 | 0.08 | 658 |
| Phage-503 | — | — | D | — | — | L | L | E | — | K | A | — | — | — | 0.07 | 1.62 | 0.07 | 659 |
| Phage-504 | — | — | E | — | — | L | F | E | — | M | A | — | — | — | 0.08 | 1.62 | 0.07 | 660 |
| Phage-505 | — | — | D | — | — | S | P | E | — | M | L | — | — | — | 0.07 | 1.61 | 0.07 | 661 |
| Phage-506 | — | — | — | — | — | S | P | — | — | M | E | — | — | — | 0.06 | 1.61 | 0.06 | 662 |
| Phage-507 | — | — | D | — | — | S | V | K | — | M | A | — | — | — | 0.07 | 1.61 | 0.06 | 663 |
| Phage-508 | — | — | S | — | — | L | P | E | — | M | W | — | — | — | 0.07 | 1.61 | 0.10 | 664 |
| Phage-509 | — | — | D | — | — | S | V | E | — | K | L | — | — | M | 0.05 | 1.61 | 0.06 | 665 |
| Phage-510 | — | — | D | — | — | — | Q | E | — | M | V | — | — | — | 0.05 | 1.61 | 0.06 | 666 |
| Phage-511 | — | — | — | — | — | S | P | E | — | M | Q | — | — | — | 0.06 | 1.60 | 0.08 | 667 |
| Phage-512 | — | — | E | — | — | L | P | L | — | M | D | — | — | — | 0.07 | 1.60 | 0.08 | 668 |
| Phage-513 | — | — | E | — | — | S | A | A | — | M | Q | — | — | — | 0.07 | 1.60 | 0.09 | 669 |
| Phage-514 | — | — | E | — | — | M | P | E | — | L | Q | — | — | — | 0.05 | 1.60 | 0.10 | 670 |
| Phage-515 | — | — | D | — | — | E | L | — | — | M | A | — | — | — | 0.07 | 1.60 | 0.08 | 671 |
| Phage-516 | — | — | D | — | — | R | L | E | — | L | E | — | — | — | 0.06 | 1.60 | 0.06 | 672 |
| Phage-517 | — | — | — | — | — | L | D | — | — | — | E | — | — | — | 0.06 | 1.60 | 0.10 | 673 |
| Phage-518 | — | — | D | — | — | — | Q | E | F | — | W | — | — | — | 0.06 | 1.60 | 0.10 | 674 |
| Phage-519 | — | — | D | — | — | S | E | A | — | M | W | — | — | — | 0.07 | 1.59 | 0.07 | 675 |
| Phage-520 | — | — | E | — | — | — | D | E | — | M | W | — | — | — | 0.07 | 1.59 | 0.06 | 676 |
| Phage-521 | — | — | E | — | — | S | V | E | — | — | G | — | — | — | 0.06 | 1.59 | 0.07 | 677 |
| Phage-522 | — | — | D | — | — | R | V | E | — | M | A | — | — | — | 0.05 | 1.58 | 0.09 | 678 |
| Phage-523 | — | — | E | — | — | S | — | E | H | M | E | — | — | — | 0.07 | 1.58 | 0.06 | 679 |
| Phage-524 | — | — | — | — | — | S | S | E | — | — | P | — | — | — | 0.06 | 1.58 | 0.09 | 680 |
| Phage-525 | — | — | D | — | — | L | P | — | — | L | Q | — | — | T | 0.07 | 1.58 | 0.07 | 681 |
| Phage-526 | — | — | D | — | — | — | K | E | — | — | Q | — | — | — | 0.06 | 1.58 | 0.06 | 682 |
| Phage-527 | — | — | E | — | — | L | P | — | — | M | V | — | — | — | 0.07 | 1.58 | 0.07 | 683 |
| Phage-528 | — | — | Q | — | — | — | D | E | — | M | A | — | — | — | 0.07 | 1.58 | 0.08 | 684 |
| Phage-529 | — | — | D | — | — | — | E | E | — | L | L | — | — | — | 0.05 | 1.57 | 0.05 | 685 |
| Phage-530 | — | — | E | — | — | L | G | I | — | M | T | — | — | — | 0.06 | 1.57 | 0.08 | 686 |
| Phage-531 | — | — | D | — | — | S | — | — | — | M | W | — | — | I | 0.06 | 1.57 | 0.09 | 687 |
| Phage-532 | — | — | E | — | — | S | E | E | F | M | S | — | — | — | 0.05 | 1.57 | 0.08 | 688 |
| Phage-533 | — | — | D | — | — | L | P | M | — | M | K | — | — | — | 0.08 | 1.57 | 0.10 | 689 |
| Phage-534 | — | — | Q | — | — | L | P | — | — | V | E | — | — | — | 0.08 | 1.57 | 0.08 | 690 |
| Phage-535 | — | — | E | — | — | L | D | M | — | V | W | — | — | — | 0.06 | 1.56 | 0.08 | 691 |
| Phage-536 | — | — | D | — | — | S | Q | E | — | M | A | H | — | — | 0.06 | 1.56 | 0.07 | 692 |
| Phage-537 | — | — | D | — | — | S | P | L | — | M | V | — | — | — | 0.05 | 1.56 | 0.06 | 693 |
| Phage-538 | — | — | D | — | — | F | L | T | — | M | T | — | — | — | 0.06 | 1.56 | 0.07 | 694 |
| Phage-539 | — | — | — | — | — | S | E | E | — | M | D | — | — | — | 0.05 | 1.55 | 0.05 | 695 |
| Phage-540 | — | — | D | — | — | S | E | V | — | M | S | — | — | — | 0.06 | 1.55 | 0.06 | 696 |
| Phage-541 | — | — | E | — | — | T | L | E | — | M | W | — | — | — | 0.07 | 1.55 | 0.33 | 697 |
| Phage-542 | — | — | D | — | — | S | Q | A | H | M | Q | — | — | — | 0.06 | 1.55 | 0.08 | 698 |
| Phage-543 | — | — | — | — | — | D | Q | — | — | M | E | — | — | — | 0.14 | 1.55 | 0.23 | 699 |
| Phage-544 | — | — | D | — | — | L | P | V | — | M | M | — | — | — | 0.06 | 1.55 | 0.07 | 700 |
| Phage-545 | — | — | — | — | — | N | A | — | — | M | S | — | — | — | 0.06 | 1.55 | 0.05 | 701 |
| Phage-546 | — | — | D | — | — | T | S | E | — | M | T | — | — | — | 0.06 | 1.54 | 0.06 | 702 |
| Phage-547 | — | — | E | — | — | — | P | V | — | M | F | — | — | — | 0.05 | 1.54 | 0.08 | 703 |
| Phage-548 | — | — | E | — | — | L | G | — | — | M | E | — | — | — | 0.06 | 1.54 | 0.08 | 704 |
| Phage-549 | — | — | D | — | — | N | G | V | — | W | W | — | — | I | 0.06 | 1.53 | 0.07 | 705 |
| Phage-550 | — | — | D | — | — | | P | E | — | L | T | — | — | — | 0.08 | 1.53 | 0.06 | 706 |
| Phage-551 | — | — | E | — | — | S | I | — | — | M | T | — | — | — | 0.06 | 1.53 | 0.13 | 707 |
| Phage-552 | — | — | D | — | — | L | Q | I | — | M | G | — | — | — | 0.05 | 1.53 | 0.06 | 708 |
| Phage-553 | — | — | D | — | — | S | V | E | — | L | D | — | — | — | 0.05 | 1.53 | 0.08 | 709 |
| Phage-554 | — | — | D | — | — | — | — | — | — | M | W | — | — | — | 0.09 | 1.53 | 0.07 | 710 |
| Phage-555 | — | — | D | — | — | L | P | E | — | L | A | — | — | — | 0.05 | 1.52 | 0.06 | 711 |
| Phage-556 | — | — | D | — | — | L | A | V | — | M | A | — | — | — | 0.07 | 1.52 | 0.08 | 712 |
| Phage-557 | — | — | — | — | — | L | P | V | — | M | D | — | — | — | 0.09 | 1.52 | 0.08 | 713 |
| Phage-558 | — | — | E | — | — | S | A | M | — | — | W | — | — | — | 0.09 | 1.52 | 0.10 | 714 |
| Phage-559 | — | — | — | — | — | L | E | E | — | M | W | — | — | — | 0.07 | 1.51 | 0.08 | 715 |
| Phage-560 | — | — | D | — | — | S | F | L | — | M | A | — | — | — | 0.06 | 1.51 | 0.06 | 716 |
| Phage-561 | — | — | D | — | — | S | A | E | — | L | S | — | — | — | 0.07 | 1.51 | 0.07 | 717 |
| Phage-562 | — | — | E | — | — | M | P | 1 | — | M | E | — | — | — | 0.06 | 1.51 | 0.06 | 718 |
| Phage-563 | — | — | E | — | — | S | L | M | — | M | A | — | — | — | 0.06 | 1.50 | 0.06 | 719 |
| Phage-564 | — | — | D | — | — | S | Q | E | — | M | E | — | — | — | 0.06 | 1.50 | 0.06 | 720 |
| Phage-565 | — | — | D | — | — | K | A | E | — | M | Q | — | — | — | 0.09 | 1.50 | 0.06 | 721 |
| Phage-566 | — | — | D | — | — | S | P | — | — | — | Q | — | — | — | 0.07 | 1.50 | 0.07 | 722 |
| Phage-567 | — | — | S | — | — | L | P | — | — | L | E | — | — | — | 0.11 | 1.50 | 0.31 | 723 |
| Phage-568 | — | — | D | — | — | H | K | E | — | L | L | — | — | — | 0.05 | 1.49 | 0.06 | 724 |
| Phage-569 | — | — | D | — | — | L | A | L | H | M | K | — | — | — | 0.08 | 1.49 | 0.08 | 725 |
| Phage-570 | — | — | E | — | — | I | M | — | — | M | A | Q | — | — | 0.06 | 1.49 | 0.06 | 726 |
| Phage-571 | — | — | D | — | — | L | P | I | — | Q | A | — | — | — | 0.06 | 1.49 | 0.10 | 727 |
| Phage-572 | — | — | D | — | — | S | E | — | — | — | A | — | — | — | 0.06 | 1.49 | 0.08 | 728 |
| Phage-573 | — | — | D | — | — | L | P | E | — | — | T | — | — | — | 0.06 | 1.49 | 0.06 | 729 |
| Phage-574 | — | — | E | — | — | R | P | E | — | M | Q | — | — | V | 0.06 | 1.48 | 0.09 | 730 |
| Phage-575 | — | — | — | — | — | T | V | E | N | M | A | — | — | — | 0.07 | 1.48 | 0.07 | 731 |
| Phage-576 | — | — | E | — | — | V | L | E | — | R | G | — | — | D | 0.07 | 1.48 | 0.09 | 732 |
| Phage-577 | — | — | D | — | — | V | A | E | — | R | H | — | — | — | 0.05 | 1.47 | 0.06 | 733 |
| Phage-578 | — | — | E | — | — | L | D | — | — | M | T | — | — | — | 0.06 | 1.47 | 0.08 | 734 |
| Phage-579 | — | — | — | — | — | T | Q | E | — | — | E | — | — | — | 0.06 | 1.47 | 0.06 | 735 |
| Phage-580 | — | — | D | — | — | L | A | I | — | — | G | — | — | — | 0.07 | 1.47 | 0.08 | 736 |
| Phage-581 | — | — | D | — | — | L | E | I | — | M | S | — | — | — | 0.08 | 1.46 | 0.08 | 737 |
| Phage-582 | — | — | D | — | — | L | L | E | — | M | S | — | — | — | 0.06 | 1.46 | 0.09 | 738 |
| Phage-583 | — | — | D | — | — | S | L | E | — | M | V | — | — | — | 0.06 | 1.46 | 0.06 | 739 |
| Phage-584 | — | — | E | — | — | S | L | E | H | — | A | — | — | — | 0.05 | 1.46 | 0.07 | 740 |
| Phage-585 | — | — | E | — | — | T | P | V | — | M | P | — | — | — | 0.08 | 1.46 | 0.06 | 741 |
| Phage-586 | — | — | Q | — | — | I | E | P | — | L | A | — | — | — | 0.06 | 1.46 | 0.05 | 742 |
| Phage-587 | — | — | D | — | — | L | A | I | — | M | W | — | — | — | 0.07 | 1.45 | 0.09 | 743 |
| Phage-588 | — | — | D | — | — | — | E | E | — | M | A | — | — | — | 0.06 | 1.45 | 0.06 | 744 |
| Phage-589 | — | — | D | — | — | S | L | — | — | M | S | — | — | — | 0.06 | 1.45 | 0.06 | 745 |
| Phage-590 | — | — | E | — | — | L | P | — | — | M | T | — | — | I | 0.07 | 1.44 | 0.08 | 746 |
| Phage-591 | — | — | — | — | — | L | P | — | — | — | Q | — | — | — | 0.06 | 1.44 | 0.07 | 747 |
| Phage-592 | — | — | E | — | — | L | A | I | — | Q | K | — | — | — | 0.06 | 1.44 | 0.06 | 748 |
| Phage-593 | — | — | E | — | — | — | V | E | — | M | A | — | — | — | 0.05 | 1.44 | 0.07 | 749 |
| Phage-594 | — | — | F | — | — | L | P | I | — | M | E | — | — | — | 0.10 | 1.44 | 0.07 | 750 |
| Phage-595 | — | — | F | — | — | S | M | E | — | — | Q | — | — | — | 0.08 | 1.44 | 0.07 | 751 |
| Phage-596 | — | — | E | — | — | S | Q | E | — | M | A | — | — | — | 0.05 | 1.44 | 0.06 | 752 |
| Phage-597 | — | — | D | — | — | L | P | E | — | M | — | Q | — | — | 0.05 | 1.44 | 0.07 | 753 |
| Phage-598 | — | — | D | — | — | N | F | — | — | M | E | — | — | — | 0.07 | 1.44 | 0.06 | 754 |
| Phage-599 | — | — | V | — | — | L | P | — | — | M | Q | — | — | — | 0.07 | 1.43 | 0.06 | 755 |
| Phage-600 | — | — | D | — | — | L | P | E | — | — | A | — | — | Q | 0.06 | 1.43 | 0.07 | 756 |
| Phage-601 | — | — | D | — | — | S | L | E | — | — | E | — | — | — | 0.06 | 1.43 | 0.06 | 757 |
| Phage-602 | — | — | L | — | — | S | — | P | — | — | H | — | — | Y | 0.05 | 1.43 | 0.07 | 758 |
| Phage-603 | — | — | D | — | — | L | D | E | — | M | L | — | — | — | 0.06 | 1.43 | 0.06 | 759 |
| Phage-604 | — | — | D | — | — | T | P | E | — | M | W | — | — | — | 0.06 | 1.43 | 0.06 | 760 |
| Phage-605 | — | — | E | — | — | S | Q | — | — | M | Q | — | — | — | 0.06 | 1.42 | 0.08 | 761 |
| Phage-606 | — | — | D | — | — | S | P | A | F | — | Q | — | — | — | 0.07 | 1.42 | 0.08 | 762 |
| Phage-607 | — | — | D | — | — | S | L | E | — | — | D | — | — | M | 0.07 | 1.42 | 0.09 | 763 |
| Phage-608 | — | — | Q | — | — | S | P | N | — | — | T | — | — | F | 0.06 | 1.41 | 0.06 | 764 |
| Phage-609 | — | — | D | — | — | — | P | E | — | L | G | — | — | — | 0.06 | 1.41 | 0.06 | 765 |
| Phage-610 | — | — | — | — | — | — | E | E | — | L | E | — | — | — | 0.06 | 1.41 | 0.15 | 766 |
| Phage-611 | — | — | D | — | — | S | H | E | — | M | Q | — | — | — | 0.06 | 1.41 | 0.06 | 767 |
| Phage-612 | — | — | — | — | — | S | A | E | — | M | S | — | — | — | 0.06 | 1.41 | 0.07 | 768 |
| Phage-613 | — | — | D | — | — | L | M | E | — | K | A | — | — | — | 0.06 | 1.41 | 0.06 | 769 |
| Phage-614 | — | — | E | — | — | S | A | E | — | — | A | — | — | — | 0.07 | 1.41 | 0.09 | 770 |
| Phage-615 | — | — | D | — | — | S | P | E | H | M | G | — | — | | 0.06 | 1.40 | 0.09 | 771 |
| Phage-616 | — | — | D | — | — | D | A | R | — | M | E | — | — | — | 0.07 | 1.40 | 0.06 | 772 |
| Phage-617 | — | — | D | — | — | S | P | E | — | M | Q | — | — | — | 0.07 | 1.40 | 0.53 | 773 |
| Phage-618 | — | — | N | — | — | L | P | E | — | M | S | — | — | I | 0.06 | 1.40 | 0.07 | 774 |
| Phage-619 | — | — | S | — | — | L | P | E | — | M | D | — | — | — | 0.05 | 1.39 | 0.07 | 775 |
| Phage-620 | — | — | D | — | — | S | L | E | — | M | L | — | — | — | 0.05 | 1.39 | 0.07 | 776 |
| Phage-621 | — | — | E | — | — | H | P | W | — | V | Q | — | — | — | 0.07 | 1.39 | 0.07 | 777 |
| Phage-622 | — | — | D | — | — | N | — | E | — | M | E | — | — | | 0.05 | 1.38 | 0.05 | 778 |
| Phage-623 | — | — | D | — | — | — | E | — | — | M | S | — | — | — | 0.07 | 1.38 | 0.07 | 779 |
| Phage-624 | — | — | D | — | L | F | Q | L | — | M | M | — | — | — | 0.07 | 1.38 | 0.07 | 780 |
| Phage-625 | — | — | D | — | — | — | P | E | — | — | Q | — | — | — | 0.07 | 1.38 | 0.06 | 781 |
| Phage-626 | — | — | E | — | — | L | D | E | — | L | Q | — | — | — | 0.06 | 1.38 | 0.07 | 782 |
| Phage-627 | — | — | D | — | — | L | Q | E | — | M | Q | — | — | — | 0.06 | 1.38 | 0.06 | 783 |
| Phage-628 | — | — | D | — | — | Q | P | — | — | M | S | — | — | — | 0.07 | 1.38 | 0.09 | 784 |
| Phage-629 | — | — | D | — | — | L | S | E | — | — | G | — | — | — | 0.06 | 1.37 | 0.06 | 785 |
| Phage-630 | — | — | E | — | — | S | Q | L | — | M | A | — | — | — | 0.07 | 1.37 | 0.07 | 786 |
| Phage-631 | — | — | D | — | — | S | E | T | — | L | W | — | — | — | 0.07 | 1.37 | 0.10 | 787 |
| Phage-632 | — | — | E | — | — | L | P | A | — | M | G | — | — | I | 0.07 | 1.37 | 0.09 | 788 |
| Phage-633 | — | — | E | — | — | L | P | F | — | M | I | — | — | I | 0.06 | 1.36 | 0.10 | 789 |
| Phage-634 | — | — | — | — | — | T | S | E | — | M | S | — | — | — | 0.06 | 1.36 | 0.07 | 790 |
| Phage-635 | — | — | D | — | — | M | K | E | — | M | L | — | — | — | 0.05 | 1.36 | 0.06 | 791 |
| Phage-636 | — | — | D | — | — | V | K | E | — | M | Y | — | — | — | 0.06 | 1.36 | 0.06 | 792 |
| Phage-637 | — | — | D | — | — | L | A | L | — | M | W | — | — | — | 0.11 | 1.36 | 0.17 | 793 |
| Phage-638 | — | — | D | — | — | L | S | I | — | M | T | — | — | — | 0.06 | 1.36 | 0.07 | 794 |
| Phage-639 | — | — | D | — | — | M | V | — | — | M | A | — | — | — | 0.06 | 1.36 | 0.07 | 795 |
| Phage-640 | — | — | D | — | — | S | E | E | F | M | W | — | — | — | 0.07 | 1.35 | 0.06 | 796 |
| Phage-641 | — | — | E | — | — | M | E | K | — | M | E | — | — | — | 0.07 | 1.35 | 0.11 | 797 |
| Phage-642 | — | — | D | — | — | L | P | R | — | M | A | — | — | — | 0.06 | 1.35 | 0.10 | 798 |
| Phage-643 | — | — | D | — | — | L | P | E | — | M | Q | — | — | — | 0.09 | 1.35 | 0.08 | 799 |
| Phage-644 | — | — | D | — | — | L | L | — | — | M | H | — | — | — | 0.10 | 1.35 | 0.07 | 800 |
| Phage-645 | — | — | D | — | — | I | P | L | — | — | Q | — | — | — | 0.07 | 1.34 | 0.06 | 801 |
| Phage-646 | — | — | D | — | — | S | K | E | — | M | A | — | — | — | 0.07 | 1.34 | 0.06 | 802 |
| Phage-647 | — | — | E | — | — | S | P | E | — | M | H | — | — | — | 0.07 | 1.34 | 0.09 | 803 |
| Phage-648 | — | — | D | — | — | S | E | A | — | — | E | — | — | — | 0.08 | 1.34 | 0.06 | 804 |
| Phage-649 | — | — | — | — | — | V | D | I | — | L | W | — | — | — | 0.07 | 1.34 | 0.06 | 805 |
| Phage-650 | — | — | — | — | — | — | S | E | — | M | A | — | — | — | 0.07 | 1.33 | 0.08 | 806 |
| Phage-651 | — | — | D | — | — | — | P | — | — | M | R | — | — | — | 0.08 | 1.33 | 0.08 | 807 |
| Phage-652 | — | — | D | — | — | L | P | L | — | M | Q | — | — | F | 0.06 | 1.33 | 0.10 | 808 |
| Phage-653 | — | — | — | — | — | L | P | M | — | M | Q | — | — | — | 0.06 | 1.33 | 0.08 | 809 |
| Phage-654 | — | — | S | E | — | Q | I | I | F | — | Q | — | — | — | 0.07 | 1.32 | 0.08 | 810 |
| Phage-655 | — | — | E | — | — | L | E | I | — | L | A | — | — | — | 0.06 | 1.32 | 0.07 | 811 |
| Phage-656 | — | — | D | — | — | H | A | E | F | — | G | — | — | — | 0.06 | 1.32 | 0.06 | 812 |
| Phage-657 | — | — | — | — | — | — | L | E | — | M | G | — | — | — | 0.07 | 1.32 | 0.06 | 813 |
| Phage-658 | — | — | E | — | — | L | P | E | — | M | I | — | — | — | 0.06 | 1.32 | 0.06 | 814 |
| Phage-659 | — | — | E | — | — | L | P | A | — | M | W | — | — | — | 0.06 | 1.31 | 0.08 | 815 |
| Phage-660 | — | — | E | — | — | L | P | M | — | M | Q | — | — | | 0.07 | 1.31 | 0.08 | 816 |
| Phage-661 | — | — | D | — | — | I | P | E | — | M | T | — | — | — | 0.06 | 1.31 | 0.06 | 817 |
| Phage-662 | — | — | D | — | — | S | A | E | — | — | A | — | — | — | 0.05 | 1.31 | 0.05 | 818 |
| Phage-663 | — | — | E | — | — | S | A | V | — | M | S | — | — | — | 0.06 | 1.31 | 0.06 | 819 |
| Phage-664 | — | — | E | — | — | S | L | E | — | M | M | — | — | V | 0.06 | 1.30 | 0.07 | 820 |
| Phage-665 | — | — | D | — | — | — | P | — | — | M | A | — | — | I | 0.06 | 1.30 | 0.06 | 821 |
| Phage-666 | — | — | Q | — | — | S | V | E | H | M | A | — | — | — | 0.06 | 1.30 | 0.06 | 822 |
| Phage-667 | — | — | — | — | — | M | P | I | — | M | T | — | — | — | 0.06 | 1.30 | 0.08 | 823 |
| Phage-668 | — | — | D | — | — | R | A | E | — | M | L | — | — | — | 0.06 | 1.29 | 0.08 | 824 |
| Phage-669 | — | — | E | — | — | L | V | — | — | M | Q | — | — | — | 0.06 | 1.28 | 0.06 | 825 |
| Phage-670 | — | — | M | — | — | L | P | I | — | M | E | — | — | — | 0.05 | 1.28 | 0.05 | 826 |
| Phage-671 | — | — | E | — | — | L | P | R | — | M | L | — | — | — | 0.06 | 1.28 | 0.30 | 827 |
| Phage-672 | — | — | D | — | — | L | E | V | — | M | D | — | — | — | 0.13 | 1.28 | 0.07 | 828 |
| Phage-673 | — | — | D | — | — | S | P | A | — | L | Q | — | — | — | 0.07 | 1.28 | 0.06 | 829 |
| Phage-674 | — | — | E | — | — | S | S | — | W | M | W | — | — | — | 0.06 | 1.27 | 0.09 | 830 |
| Phage-675 | — | — | E | — | — | F | P | L | — | M | — | — | — | — | 0.06 | 1.27 | 0.08 | 831 |
| Phage-676 | — | — | E | — | — | S | T | E | — | M | A | — | — | — | 0.08 | 1.27 | 0.06 | 832 |
| Phage-677 | — | — | E | — | — | Q | P | A | — | M | E | — | — | — | 0.05 | 1.27 | 0.06 | 833 |
| Phage-678 | — | — | D | — | — | — | E | E | — | R | M | — | — | — | 0.05 | 1.27 | 0.08 | 834 |
| Phage-679 | — | — | D | — | — | L | P | V | — | M | T | — | — | — | 0.07 | 1.27 | 0.07 | 835 |
| Phage-680 | — | — | D | — | — | S | S | V | — | M | L | — | — | — | 0.08 | 1.26 | 0.06 | 836 |
| Phage-681 | — | — | E | — | — | H | R | I | — | M | Q | — | — | — | 0.05 | 1.26 | 0.07 | 837 |
| Phage-682 | — | — | E | — | — | S | E | E | — | M | A | — | — | — | 0.06 | 1.26 | 0.07 | 838 |
| Phage-683 | — | — | E | — | — | M | S | I | — | M | Q | — | — | — | 0.07 | 1.25 | 0.07 | 839 |
| Phage-684 | — | — | E | — | — | L | Q | E | H | M | A | — | — | — | 0.06 | 1.25 | 0.05 | 840 |
| Phage-685 | — | — | — | — | L | F | E | E | — | M | S | — | — | — | 0.06 | 1.25 | 0.06 | 841 |
| Phage-686 | — | — | E | — | — | L | P | M | — | M | T | — | — | — | 0.05 | 1.25 | 0.06 | 842 |
| Phage-687 | — | — | D | — | — | S | E | — | — | M | A | — | — | — | 0.06 | 1.24 | 0.06 | 843 |
| Phage-688 | — | — | E | — | — | T | Q | E | — | M | Q | — | — | — | 0.07 | 1.24 | 0.07 | 844 |
| Phage-689 | — | — | — | — | — | H | A | E | — | M | A | — | — | — | 0.06 | 1.24 | 0.08 | 845 |
| Phage-690 | — | — | E | — | — | L | A | E | — | M | — | — | — | — | 0.06 | 1.24 | 0.07 | 846 |
| Phage-691 | — | — | D | — | — | V | E | E | — | M | T | — | — | — | 0.06 | 1.24 | 0.07 | 847 |
| Phage-692 | — | — | D | — | — | L | E | — | — | M | L | — | — | — | 0.07 | 1.24 | 0.09 | 848 |
| Phage-693 | — | — | E | — | — | I | G | I | — | M | S | — | — | — | 0.07 | 1.23 | 0.07 | 849 |
| Phage-694 | — | — | E | — | — | S | T | E | — | — | Q | — | — | — | 0.06 | 1.23 | 0.09 | 850 |
| Phage-695 | — | — | D | — | — | S | M | M | — | M | Q | — | — | — | 0.06 | 1.23 | 0.06 | 851 |
| Phage-696 | — | — | E | — | — | S | A | E | H | M | P | — | — | — | 0.07 | 1.23 | 0.09 | 852 |
| Phage-697 | — | — | V | — | — | Y | P | E | — | M | E | — | — | — | 0.06 | 1.22 | 0.07 | 853 |
| Phage-698 | — | — | D | — | — | E | A | R | — | M | Q | — | — | — | 0.09 | 1.21 | 0.07 | 854 |
| Phage-699 | — | — | D | — | — | S | Q | E | — | M | S | — | — | — | 0.06 | 1.21 | 0.06 | 855 |
| Phage-700 | — | — | H | — | — | L | P | — | — | M | S | — | — | M | 0.07 | 1.21 | 0.06 | 856 |
| Phage-701 | — | — | — | — | — | L | D | I | — | L | A | — | — | — | 0.06 | 1.21 | 0.07 | 857 |
| Phage-702 | — | — | — | — | — | — | P | E | — | — | S | — | — | — | 0.08 | 1.21 | 0.07 | 858 |
| Phage-703 | — | — | D | — | — | S | — | E | — | L | G | — | — | — | 0.05 | 1.20 | 0.05 | 859 |
| Phage-705 | — | — | E | — | — | — | A | E | — | L | W | — | — | — | 0.05 | 1.18 | 0.09 | 860 |
| Phage-706 | — | — | D | — | — | R | P | E | — | M | S | — | — | — | 0.05 | 1.18 | 0.07 | 861 |
| Phage-707 | — | — | E | — | — | M | P | E | — | L | A | — | — | — | 0.06 | 1.18 | 0.07 | 862 |
| Phage-708 | — | — | E | — | — | — | G | V | — | M | A | — | — | — | 0.06 | 1.17 | 0.05 | 863 |
| Phage-709 | — | — | E | — | — | S | Q | T | — | M | W | — | — | — | 0.07 | 1.17 | 0.08 | 864 |
| Phage-710 | — | — | — | — | — | S | S | E | — | — | G | — | — | — | 0.05 | 1.17 | 0.06 | 865 |
| Phage-711 | — | — | E | — | — | S | Q | — | — | M | W | — | — | — | 0.05 | 1.17 | 0.05 | 866 |
| Phage-712 | — | — | D | — | — | L | P | D | — | M | A | — | — | — | 0.06 | 1.17 | 0.07 | 867 |
| Phage-713 | — | — | E | — | — | — | E | — | — | M | E | — | — | — | 0.07 | 1.17 | 0.08 | 868 |
| Phage-714 | — | — | E | — | — | L | K | E | — | M | Q | — | — | — | 0.06 | 1.17 | 0.07 | 869 |
| Phage-715 | — | — | E | — | — | S | Y | — | — | M | Q | — | — | — | 0.06 | 1.16 | 0.06 | 870 |
| Phage-716 | — | — | E | — | — | R | A | E | — | M | S | — | — | — | 0.07 | 1.16 | 0.09 | 871 |
| Phage-717 | — | — | D | — | — | S | — | I | — | M | V | — | — | — | 0.06 | 1.16 | 0.06 | 872 |
| Phage-718 | — | — | D | — | — | L | S | V | — | M | S | — | — | — | 0.06 | 1.15 | 0.07 | 873 |
| Phage-719 | — | — | E | — | — | L | P | — | — | M | S | — | — | — | 0.06 | 1.15 | 0.09 | 874 |
| Phage-720 | — | — | E | — | — | V | A | E | — | R | A | — | — | — | 0.06 | 1.15 | 0.09 | 875 |
| Phage-721 | — | — | Q | — | — | L | P | — | — | — | E | — | — | — | 0.06 | 1.14 | 0.06 | 876 |
| Phage-722 | — | — | E | — | — | — | A | A | — | M | G | — | — | — | 0.06 | 1.14 | 0.08 | 877 |
| Phage-723 | — | — | D | — | — | E | Q | — | — | M | E | — | — | — | 0.06 | 1.14 | 0.06 | 878 |
| Phage-724 | — | — | D | — | — | L | Q | M | — | M | M | — | — | — | 0.08 | 1.14 | 0.07 | 879 |
| Phage-725 | — | — | E | — | — | L | Y | E | — | M | W | — | — | — | 0.06 | 1.13 | 0.07 | 880 |
| Phage-726 | — | — | V | — | — | S | E | — | — | M | A | — | — | — | 0.07 | 1.13 | 0.06 | 881 |
| Phage-727 | — | — | D | — | — | E | A | E | — | M | W | — | — | — | 0.09 | 1.13 | 0.10 | 882 |
| Phage-728 | — | — | E | — | — | — | E | — | — | M | K | — | — | — | 0.06 | 1.12 | 0.07 | 883 |
| Phage-729 | — | — | D | — | — | V | V | E | — | M | S | — | — | — | 0.09 | 1.12 | 0.08 | 884 |
| Phage-730 | — | — | — | — | — | L | P | — | — | M | H | — | — | — | 0.06 | 1.11 | 0.08 | 885 |
| Phage-731 | — | — | D | — | — | I | — | P | W | — | E | — | — | — | 0.06 | 1.11 | 0.11 | 886 |
| Phage-732 | — | — | E | — | — | S | S | L | — | L | E | — | — | — | 0.07 | 1.11 | 0.08 | 887 |
| Phage-733 | — | — | D | — | — | — | M | E | — | M | E | — | — | — | 0.07 | 1.11 | 0.08 | 888 |
| Phage-734 | — | — | E | — | — | L | — | E | — | L | A | — | — | — | 0.06 | 1.10 | 0.05 | 889 |
| Phage-735 | — | — | E | — | — | M | G | E | — | M | L | — | — | — | 0.07 | 1.10 | 0.06 | 890 |
| Phage-736 | — | — | D | — | — | T | K | E | — | M | W | — | — | — | 0.06 | 1.10 | 0.06 | 891 |
| Phage-737 | — | — | — | — | — | — | E | E | — | M | L | — | — | — | 0.05 | 1.09 | 0.07 | 892 |
| Phage-738 | — | — | E | — | — | S | A | E | — | — | G | — | — | — | 0.07 | 1.08 | 0.07 | 893 |
| Phage-739 | — | — | V | — | — | L | P | E | — | M | L | E | — | M | 0.06 | 1.08 | 0.07 | 894 |
| Phage-740 | — | — | D | — | — | S | F | H | — | M | A | — | — | — | 0.06 | 1.08 | 0.06 | 895 |
| Phage-741 | — | — | E | — | — | S | E | M | — | M | S | — | — | — | 0.06 | 1.07 | 0.09 | 896 |
| Phage-742 | — | — | D | — | — | — | N | A | — | — | W | — | — | — | 0.06 | 1.07 | 0.09 | 897 |
| Phage-743 | — | — | E | — | — | I | P | M | — | M | L | — | — | M | 0.06 | 1.06 | 0.09 | 898 |
| Phage-744 | — | — | E | — | — | — | Q | E | — | M | A | — | — | — | 0.07 | 1.06 | 0.08 | 899 |
| Phage-745 | — | — | D | — | — | — | K | V | — | — | W | — | — | — | 0.06 | 1.06 | 0.08 | 900 |
| Phage-746 | — | — | E | — | — | I | P | A | — | M | A | — | — | — | 0.06 | 1.06 | 0.08 | 901 |
| Phage-747 | — | — | D | — | — | — | P | I | — | M | Q | — | — | — | 0.06 | 1.06 | 0.06 | 902 |
| Phage-748 | — | — | E | — | — | L | — | E | — | M | K | — | — | — | 0.07 | 1.06 | 0.07 | 903 |
| Phage-749 | — | — | E | — | — | D | V | — | — | M | Q | — | — | — | 0.07 | 1.06 | 0.08 | 904 |
| Phage-750 | — | — | E | — | — | S | S | E | — | L | G | — | — | — | 0.05 | 1.05 | 0.09 | 905 |
| Phage-751 | — | — | D | — | — | L | P | R | — | M | E | — | — | M | 0.06 | 1.05 | 0.06 | 906 |
| Phage-752 | — | — | D | — | — | S | S | A | — | — | D | — | — | — | 0.09 | 1.05 | 0.06 | 907 |
| Phage-753 | — | — | E | — | — | L | P | S | — | L | A | — | — | — | 0.06 | 1.05 | 0.06 | 908 |
| Phage-754 | — | — | D | — | — | — | P | E | F | — | R | — | — | — | 0.07 | 1.05 | 0.07 | 909 |
| Phage-755 | — | — | E | — | — | — | P | — | — | M | S | — | — | — | 0.06 | 1.04 | 0.08 | 910 |
| Phage-756 | — | — | D | — | — | D | A | R | — | M | T | — | — | — | 0.06 | 1.04 | 0.09 | 911 |
| Phage-757 | — | — | E | — | — | S | V | E | — | — | L | — | — | — | 0.11 | 1.04 | 0.06 | 912 |
| Phage-758 | — | — | E | — | — | S | S | A | — | M | R | — | — | — | 0.06 | 1.04 | 0.06 | 913 |
| Phage-759 | — | — | D | — | — | L | A | E | — | — | Q | — | — | — | 0.06 | 1.04 | 0.08 | 914 |
| Phage-760 | — | — | — | — | — | S | S | E | — | L | Q | — | — | — | 0.07 | 1.03 | 0.09 | 915 |
| Phage-761 | — | — | — | — | — | S | Q | V | — | M | W | — | — | — | 0.05 | 1.02 | 0.06 | 916 |
| Phage-762 | — | — | E | — | — | I | E | — | — | M | R | — | — | — | 0.06 | 1.01 | 0.06 | 917 |
| Phage-763 | — | — | F | — | — | — | P | E | — | L | L | — | — | — | 0.08 | 1.01 | 0.08 | 918 |
| Phage-764 | — | — | E | — | — | L | R | E | — | M | L | — | — | — | 0.06 | 1.01 | 0.07 | 919 |
| Phage-765 | — | — | E | — | — | S | S | V | — | L | W | — | — | — | 0.05 | 1.00 | 0.05 | 920 |
| Phage-766 | — | — | E | — | — | S | — | I | — | — | A | — | — | — | 0.05 | 1.00 | 0.07 | 921 |
| Phage-767 | — | — | D | — | — | L | E | V | — | M | S | — | — | — | 0.06 | 1.00 | 0.09 | 922 |
| Phage-768 | — | — | E | — | — | R | P | E | — | M | A | N | — | — | 0.05 | 1.00 | 0.06 | 923 |
| Phage-769 | — | — | E | — | — | I | E | P | — | M | Q | — | — | — | 0.05 | 0.99 | 0.06 | 924 |
| Phage-770 | — | — | D | — | — | K | P | E | — | M | D | — | — | — | 0.06 | 0.99 | 0.07 | 925 |
| Phage-771 | — | — | D | — | — | V | G | A | — | M | W | — | — | — | 0.05 | 0.99 | 0.06 | 926 |
| Phage-772 | — | — | E | — | — | S | A | I | — | — | A | — | — | M | 0.09 | 0.98 | 0.11 | 927 |
| Phage-773 | — | — | F | — | — | F | P | E | — | M | A | — | — | — | 0.07 | 0.98 | 0.07 | 928 |
| Phage-774 | — | — | D | — | — | T | G | M | — | — | S | — | — | — | 0.06 | 0.98 | 0.06 | 929 |
| Phage-775 | — | — | S | — | — | S | — | A | — | — | G | — | — | — | 0.08 | 0.98 | 0.07 | 930 |
| Phage-776 | — | — | — | — | — | — | A | E | — | M | W | — | — | — | 0.05 | 0.97 | 0.06 | 931 |
| Phage-777 | — | — | Q | — | — | F | P | — | — | M | A | — | — | — | 0.05 | 0.97 | 0.07 | 932 |
| Phage-778 | — | — | E | — | — | V | P | E | — | L | V | — | — | — | 0.07 | 0.96 | 0.06 | 933 |
| Phage-779 | — | — | D | — | — | S | K | E | — | M | S | — | — | — | 0.06 | 0.96 | 0.07 | 934 |
| Phage-780 | — | — | D | — | — | L | P | E | — | M | L | — | — | — | 0.06 | 0.96 | 0.09 | 935 |
| Phage-781 | — | — | — | — | — | L | D | V | — | M | S | — | — | — | 0.08 | 0.96 | 0.09 | 936 |
| Phage-784 | — | — | — | — | — | T | P | E | — | L | D | — | — | — | 0.07 | 0.95 | 0.06 | 937 |
| Phage-785 | — | — | E | — | — | D | I | R | — | M | M | — | — | — | 0.08 | 0.95 | 0.08 | 938 |
| Phage-786 | — | — | D | — | — | E | E | R | — | M | A | — | — | — | 0.06 | 0.94 | 0.06 | 939 |
| Phage-787 | — | — | S | — | — | V | P | E | — | M | E | — | — | — | 0.07 | 0.94 | 0.06 | 940 |
| Phage-788 | — | — | D | — | — | I | P | — | — | V | E | — | — | — | 0.06 | 0.94 | 0.08 | 941 |
| Phage-789 | — | — | — | — | — | — | E | I | — | M | Q | — | — | — | 0.05 | 0.93 | 0.05 | 942 |
| Phage-790 | — | — | E | — | — | — | H | E | — | M | E | — | — | V | 0.06 | 0.93 | 0.07 | 943 |
| Phage-791 | — | — | D | — | — | H | A | E | — | M | W | — | — | — | 0.05 | 0.93 | 0.06 | 944 |
| Phage-792 | — | — | E | — | — | T | Q | A | — | L | A | — | — | — | 0.07 | 0.93 | 0.10 | 945 |
| Phage-793 | — | — | — | — | — | S | A | — | — | M | W | — | — | — | 0.05 | 0.92 | 0.07 | 946 |
| Phage-794 | — | — | D | — | — | S | F | E | — | M | A | — | — | — | 0.06 | 0.92 | 0.06 | 947 |
| Phage-795 | — | — | D | — | — | S | A | E | — | L | G | — | — | — | 0.07 | 0.92 | 0.07 | 948 |
| Phage-796 | — | — | V | — | — | S | P | E | — | — | W | — | — | — | 0.07 | 0.92 | 0.06 | 949 |
| Phage-797 | — | — | D | — | — | E | L | M | — | M | E | — | — | — | 0.05 | 0.92 | 0.06 | 950 |
| Phage-798 | — | — | — | — | — | L | E | M | — | M | S | — | — | — | 0.06 | 0.91 | 0.07 | 951 |
| Phage-799 | — | — | D | — | — | I | K | E | — | M | S | — | — | — | 0.05 | 0.91 | 0.08 | 952 |
| Phage-800 | — | — | D | — | — | L | L | M | — | M | E | — | — | — | 0.05 | 0.91 | 0.06 | 953 |
| Phage-801 | — | — | E | — | — | D | — | M | — | M | E | — | — | — | 0.07 | 0.91 | 0.07 | 954 |
| Phage-802 | — | — | E | — | — | S | Q | E | — | K | A | — | — | F | 0.07 | 0.90 | 0.07 | 955 |
| Phage-803 | — | — | D | — | — | V | L | M | — | L | A | — | — | — | 0.07 | 0.90 | 0.09 | 956 |
| Phage-804 | — | — | E | — | — | S | S | A | — | M | A | — | — | — | 0.15 | 0.90 | 0.07 | 957 |
| Phage-805 | — | — | E | — | — | L | A | A | — | M | Y | — | — | — | 0.05 | 0.89 | 0.07 | 958 |
| Phage-806 | — | — | D | — | — | S | T | L | — | M | Q | — | — | — | 0.07 | 0.88 | 0.06 | 959 |
| Phage-807 | — | — | D | — | — | T | E | A | — | M | S | — | — | — | 0.07 | 0.88 | 0.06 | 960 |
| Phage-808 | — | — | E | — | — | I | S | E | — | M | G | — | — | — | 0.07 | 0.88 | 0.05 | 961 |
| Phage-809 | — | — | D | — | — | F | T | W | — | M | A | — | — | — | 0.07 | 0.87 | 0.08 | 962 |
| Phage-810 | — | — | D | — | — | I | A | E | — | R | S | — | — | — | 0.11 | 0.87 | 0.07 | 963 |
| Phage-811 | — | — | E | — | — | L | S | E | — | — | G | — | — | — | 0.06 | 0.87 | 0.06 | 964 |
| Phage-812 | — | — | D | — | — | T | A | — | — | M | W | — | — | — | 0.07 | 0.87 | 0.07 | 965 |
| Phage-813 | — | — | — | — | — | S | L | — | — | L | G | — | — | Y | 0.06 | 0.86 | 0.06 | 966 |
| Phage-814 | — | — | — | — | — | — | A | I | — | M | A | — | — | — | 0.06 | 0.85 | 0.07 | 967 |
| Phage-815 | — | — | D | — | — | S | M | T | — | M | A | — | — | I | 0.06 | 0.84 | 0.06 | 968 |
| Phage-816 | — | — | D | — | — | K | — | E | — | L | Q | — | — | — | 0.06 | 0.84 | 0.06 | 969 |
| Phage-817 | — | — | D | — | — | D | P | — | — | — | R | — | — | I | 0.08 | 0.83 | 0.06 | 970 |
| Phage-818 | — | — | E | — | — | L | P | M | — | M | Q | — | — | M | 0.05 | 0.82 | 0.07 | 971 |
| Phage-819 | — | — | D | — | — | S | G | E | — | M | S | — | — | — | 0.06 | 0.82 | 0.06 | 972 |
| Phage-820 | — | — | E | — | — | — | R | E | — | M | D | — | — | — | 0.07 | 0.80 | 0.07 | 973 |
| Phage-821 | — | — | D | — | — | S | M | E | — | M | A | — | — | — | 0.05 | 0.80 | 0.07 | 974 |
| Phage-822 | — | — | D | — | — | S | P | — | — | L | W | — | — | — | 0.06 | 0.80 | 0.05 | 975 |
| Phage-823 | — | — | — | — | — | S | A | E | — | — | Q | — | — | — | 0.08 | 0.80 | 0.08 | 976 |
| Phage-824 | — | — | — | — | — | T | Q | E | — | M | L | — | — | — | 0.07 | 0.78 | 0.06 | 977 |
| Phage-825 | — | — | Q | — | — | S | V | — | — | M | A | — | — | — | 0.06 | 0.77 | 0.06 | 978 |
| Phage-826 | — | — | E | — | — | S | S | E | — | M | V | — | — | — | 0.06 | 0.77 | 0.06 | 979 |
| Phage-827 | — | — | D | — | — | T | G | V | — | K | W | — | — | I | 0.07 | 0.76 | 0.06 | 980 |
| Phage-828 | — | — | — | — | — | I | G | E | — | M | D | — | — | — | 0.06 | 0.75 | 0.58 | 981 |
| Phage-829 | — | — | D | — | — | — | G | I | — | V | L | — | — | — | 0.06 | 0.75 | 0.08 | 982 |
| Phage-830 | — | — | D | — | — | N | P | V | — | L | A | — | — | — | 0.05 | 0.74 | 0.08 | 983 |
| Phage-831 | — | — | E | — | — | — | Q | — | — | M | L | — | — | — | 0.06 | 0.74 | 0.05 | 984 |
| Phage-832 | — | — | — | — | — | S | V | — | — | M | Y | — | — | — | 0.05 | 0.74 | 0.06 | 985 |
| Phage-833 | — | — | E | — | — | F | G | M | — | M | S | — | — | — | 0.06 | 0.73 | 0.07 | 986 |
| Phage-834 | — | — | — | — | — | M | P | — | — | M | S | — | — | — | 0.07 | 0.72 | 0.05 | 987 |
| Phage-835 | — | — | Q | — | — | — | L | — | — | L | Q | — | — | — | 0.07 | 0.71 | 0.08 | 988 |
| Phage-836 | — | — | — | — | — | L | P | — | — | V | S | — | — | — | 0.06 | 0.70 | 0.06 | 989 |
| Phage-837 | — | — | E | — | — | L | P | W | — | M | S | — | — | — | 0.06 | 0.69 | 0.09 | 990 |
| Phage-838 | — | — | D | — | — | H | L | E | — | — | D | — | — | — | 0.06 | 0.69 | 0.06 | 991 |
| Phage-839 | — | — | D | — | — | L | P | R | F | M | V | — | — | — | 0.05 | 0.69 | 0.07 | 992 |
| Phage-840 | — | — | Q | — | — | — | P | W | — | M | V | — | — | — | 0.07 | 0.69 | 0.08 | 993 |
| Phage-841 | — | — | E | — | — | M | S | E | — | M | R | — | — | — | 0.07 | 0.69 | 0.10 | 994 |
| Phage-842 | — | — | V | — | — | L | P | L | — | M | P | N | — | — | 0.09 | 0.68 | 0.11 | 995 |
| Phage-843 | — | — | D | — | — | E | P | — | — | M | T | — | — | — | 0.07 | 0.68 | 0.10 | 996 |
| Phage-844 | — | — | E | — | — | L | P | — | — | L | A | — | — | Y | 0.07 | 0.67 | 0.08 | 997 |
| Phage-845 | — | — | D | — | — | Y | G | A | — | — | L | — | — | I | 0.05 | 0.67 | 0.06 | 998 |
| Phage-846 | — | — | E | — | — | — | A | V | — | M | H | — | — | — | 0.14 | 0.67 | 0.09 | 999 |
| Phage-847 | — | — | D | — | — | M | D | A | — | M | S | — | — | — | 0.05 | 0.65 | 0.06 | 1000 |
| Phage-848 | — | — | M | — | — | S | A | E | — | M | G | — | — | — | 0.06 | 0.64 | 0.06 | 1001 |
| Phage-849 | — | — | D | — | — | — | — | W | — | M | E | — | — | — | 0.05 | 0.64 | 0.06 | 1002 |
| Phage-850 | — | — | D | — | — | — | A | W | — | L | Q | — | — | — | 0.06 | 0.64 | 0.07 | 1003 |
| Phage-851 | — | — | E | — | — | — | Q | M | — | M | L | — | — | — | 0.07 | 0.64 | 0.06 | 1004 |
| Phage-852 | — | — | E | — | — | S | R | — | — | M | E | — | — | M | 0.05 | 0.63 | 0.33 | 1005 |
| Phage-853 | — | — | S | — | — | L | P | M | — | M | A | — | — | — | 0.06 | 0.62 | 0.13 | 1006 |
| Phage-854 | — | — | E | — | — | L | A | V | H | M | S | — | — | — | 0.06 | 0.62 | 0.06 | 1007 |
| Phage-855 | — | — | D | — | — | I | E | M | — | M | S | — | — | M | 0.06 | 0.61 | 0.06 | 1008 |
| Phage-856 | — | — | I | — | — | — | L | E | — | M | Q | — | — | M | 0.07 | 0.61 | 0.08 | 1009 |
| Phage-857 | — | — | E | — | — | I | G | D | — | — | A | — | — | — | 0.05 | 0.61 | 0.36 | 1010 |
| Phage-858 | — | — | Q | — | — | S | E | L | — | M | S | — | — | — | 0.05 | 0.60 | 0.08 | 1011 |
| Phage-859 | — | — | D | — | — | T | P | — | F | R | A | — | — | — | 0.06 | 0.59 | 0.07 | 1012 |
| Phage-860 | — | — | E | — | — | L | Q | E | — | M | G | — | — | — | 0.05 | 0.58 | 0.25 | 1013 |
| Phage-861 | — | — | D | — | — | V | T | E | — | M | A | — | — | — | 0.06 | 0.58 | 0.07 | 1014 |
| Phage-862 | — | — | D | — | — | I | M | P | — | L | D | — | — | — | 0.06 | 0.57 | 0.05 | 1015 |
| Phage-863 | — | — | E | — | — | K | K | E | — | M | L | — | — | — | 0.07 | 0.56 | 0.08 | 1016 |
| Phage-864 | — | — | E | — | — | V | E | L | — | R | Q | — | — | — | 0.06 | 0.56 | 0.06 | 1017 |
| Phage-865 | — | — | D | — | — | L | E | T | — | M | Q | — | — | — | 0.05 | 0.53 | 0.08 | 1018 |
| Phage-866 | — | — | Q | — | — | L | P | I | — | — | Q | — | — | — | 0.06 | 0.51 | 0.07 | 1019 |
| Phage-867 | — | — | E | — | — | L | D | E | — | L | Q | — | — | M | 0.07 | 0.51 | 0.08 | 1020 |
| Phage-868 | — | — | — | — | — | T | T | E | — | — | V | — | — | — | 0.06 | 0.50 | 0.05 | 1021 |
| Phage-869 | — | — | E | — | — | V | P | — | — | M | G | — | — | — | 0.08 | 0.50 | 0.08 | 1022 |
| Phage-870 | — | — | D | — | — | — | A | M | — | M | S | — | — | — | 0.07 | 0.50 | 0.07 | 1023 |
| Phage-871 | — | — | E | — | — | L | G | L | — | M | K | — | — | — | 0.06 | 0.49 | 0.06 | 1024 |
| Phage-872 | — | — | — | — | — | — | Q | E | — | M | R | — | — | — | 0.07 | 0.49 | 0.07 | 1025 |
| Phage-873 | — | — | — | — | — | I | P | — | — | L | — | — | — | — | 0.07 | 0.49 | 0.07 | 1026 |
| Phage-874 | — | — | D | — | — | S | T | E | — | M | W | — | — | — | 0.05 | 0.49 | 0.06 | 1027 |
| Phage-875 | — | — | E | — | — | I | K | D | — | M | T | — | — | — | 0.06 | 0.47 | 0.08 | 1028 |
| Phage-876 | — | — | — | — | — | D | A | E | — | M | Q | — | — | — | 0.07 | 0.46 | 0.06 | 1029 |
| Phage-877 | — | — | E | — | — | M | Q | W | — | M | A | — | — | — | 0.08 | 0.45 | 0.09 | 1030 |
| Phage-878 | — | — | E | — | — | — | P | V | — | M | Q | — | — | — | 0.07 | 0.44 | 0.08 | 1031 |
| Phage-879 | — | — | D | — | — | D | E | — | — | L | M | — | — | — | 0.09 | 0.44 | 0.07 | 1032 |
| Phage-880 | — | — | D | — | — | L | P | E | — | M | E | — | — | — | 0.08 | 0.43 | 0.07 | 1033 |
| Phage-881 | — | — | — | — | — | — | S | E | — | L | Q | — | — | — | 0.05 | 0.43 | 0.06 | 1034 |
| Phage-882 | — | — | D | — | — | S | Q | I | — | M | S | — | — | — | 0.05 | 0.42 | 0.06 | 1035 |
| Phage-883 | — | — | D | — | — | W | P | G | — | L | W | — | — | — | 0.07 | 0.42 | 0.06 | 1036 |
| Phage-884 | — | — | T | — | W | Q | V | A | — | M | Q | N | — | F | 0.06 | 0.42 | 0.06 | 1037 |
| Phage-885 | — | — | — | — | — | L | D | E | — | M | E | — | — | — | 0.07 | 0.40 | 0.07 | 1038 |
| Phage-886 | — | — | D | — | — | T | — | M | — | K | Q | — | — | — | 0.06 | 0.40 | 0.08 | 1039 |

(—) indicates the same amino acid position as in Peptide-39 corresponding position.

### Example 7: Optimized Phage Library Construction of Peptide-44

Using the peptide sequence activity relationships of Example 3, DNA oligo libraries were constructed where codons encoding critical residues within peptide-44 were minimally mutated, and codons encoding non-critical residues that were tolerated in the alanine scanning mutation studies (e.g., Ser-1, Gly-5, Ser-7, Tyr-8, Try-12) were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 8: Panning of the Peptide-44 Optimized Phage Library

Once the phage optimization libraries were completed, phage libraries were bio-panned using CD28 Fab loaded beads. Multiple rounds of panning were performed where bacteriophage was allowed to bind to CD28 Fab loaded beads, washed, eluted, and amplified. After panning, phage infected bacteria were plated out and colonies picked into 96 well blocks. Clonal phage was then amplified and separated from bacterial cells via centrifugation. Phage containing supernatants were tested in binding ELISAs against CD28 Fab coated plates in the presence or absence of saturating concentrations of CD28 antigen. Phage able to bind CD28 Fab were selected for sequence analysis if the binding signal was reduced in the presence of CD28.

### Example 9: Panning ELISAs—CD28 Fab Peptides

Clonal phage were harvested as crude supernatants and screened via standard ELISAs. Briefly, biotinylated CD28 Fab was captured on neutravidin coated plates. Prior to the addition of clonal phage, wells were incubated with blocking buffer and CD28 antigen or blocking buffer alone. Without washing or aspirating, clonal phage supernatants were then added to the wells and incubated for a short time. Wells were then washed followed by detection of bound phage using a horse radish conjugated anti-M13 antibody. Clonal phage of interest were then sent for sequence analysis.

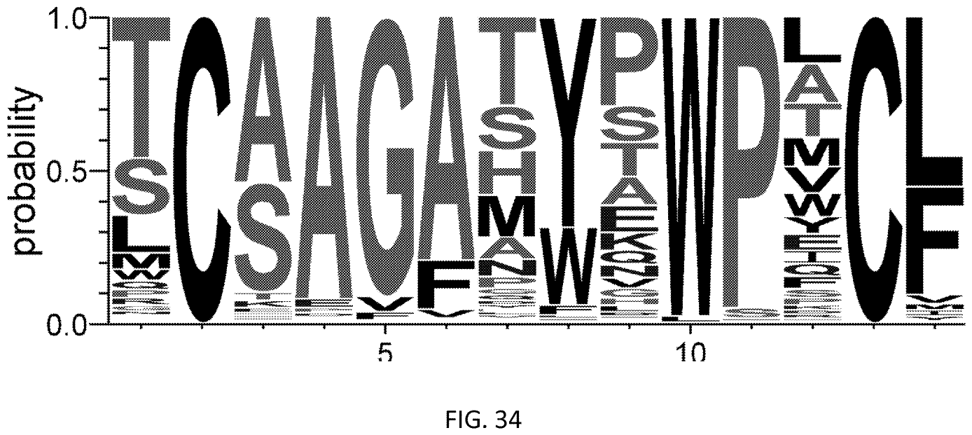

Phage panning results of peptide-44 library sequences are shown in Table 26. 397 clonal phage sequences were identified. A consensus sequence was calculated from all the sequences of Table 26 using WebLogo 3.7.12. The consensus sequence is shown in FIG. 34.

TABLE 26
Phage Panning Results of Peptide-44 Library Sequences.

| | | | | | | | | | | | | | | | Phage binding ELISA | | | |
|---|---|---|---|---|---|---|---|---|---|---|---|---|---|---|---|---|---|---|
| | | | | | | | | | | | | | | | | Anti- | Anti- | |
| | | | | | | | | | | | | | | | | CD28 | CD28 | |
| | | | | | | | | | | | | | | | | Fab | Fab | |
| | | | | | | | | | | | | | | | | (SEQ | signal | |
| | | | | | | | | | | | | | | | | ID | in the | |
| | | | | | | | | | | | | | | | | NOs: | pre- | |
| | | | | | | | | | | | | | | | Back- | 202, | sence | SEQ |
| Phage | Amino acid position sequence | | | | | | | | | | | | | | ground | 203) | of | ID |
| ID | 1 | 2 | 3 | 4 | 5 | 6 | 7 | 8 | 9 | 10 | 11 | 12 | 13 | 14 | signal | signal | CD28 | NO: |
| Phage- | S | C | A | A | G | A | S | Y | A | W | P | Y | C | L | 0.05 | 1.49 | 0.05 | 137 |
| 18 | | | | | | | | | | | | | | | | | | |
| Phage- | Y | W | S | C | F | Q | I | P | F | T | C | F | Y | F | 0.18 | 1.44 | 0.27 | 120 |
| 1 | | | | | | | | | | | | | | | | | | |
| Phage- | W | E | C | L | K | Y | T | E | — | N | A | C | T | Y | 0.05 | 1.55 | 0.07 | 121 |
| 2 | | | | | | | | | | | | | | | | | | |
| Phage- | F | M | C | L | P | Q | N | P | H | P | L | C | F | K | 0.06 | 0.84 | 0.06 | 122 |
| 3 | | | | | | | | | | | | | | | | | | |
| Phage- | P | D | C | Q | T | I | L | F | K | P | W | C | V | R | 0.05 | 2.09 | 0.06 | 123 |
| 4 | | | | | | | | | | | | | | | | | | |
| Phage- | A | — | D | T | Y | F | A | G | M | I | N | E | — | F | 0.07 | 0.92 | 0.10 | 124 |
| 5 | | | | | | | | | | | | | | | | | | |
| Phage- | G | — | Q | Y | P | W | A | F | D | A | M | S | — | P | 0.05 | 2.38 | 0.07 | 125 |
| 6 | | | | | | | | | | | | | | | | | | |
| Phage- | W | — | W | D | Y | K | L | W | V | P | T | Q | — | P | 0.11 | 2.27 | 0.14 | 126 |
| 7 | | | | | | | | | | | | | | | | | | |
| Phage- | W | — | W | D | Y | R | E | W | V | P | M | T | — | P | 0.06 | 2.30 | 0.08 | 127 |
| 8 | | | | | | | | | | | | | | | | | | |
| Phage- | E | — | H | W | S | M | W | A | V | G | H | A | — | F | 0.06 | 1.77 | 0.07 | 128 |
| 9 | | | | | | | | | | | | | | | | | | |
| Phage- | Y | — | — | G | H | S | P | I | N | I | G | K | — | — | 0.05 | 1.49 | 0.06 | 129 |
| 10 | | | | | | | | | | | | | | | | | | |
| Phage- | W | — | W | D | Y | K | L | W | V | P | M | T | — | P | 0.11 | 2.51 | 0.11 | 130 |
| 11 | | | | | | | | | | | | | | | | | | |
| Phage- | D | — | W | W | M | F | E | S | Y | P | G | R | — | I | 0.06 | 1.65 | 0.07 | 131 |
| 12 | | | | | | | | | | | | | | | | | | |
| Phage- | Y | — | — | G | H | — | W | Q | Y | I | N | K | — | — | 0.06 | 0.64 | 0.07 | 132 |
| 13 | | | | | | | | | | | | | | | | | | |
| Phage- | W | — | W | Y | Y | R | E | W | V | P | M | T | — | P | 0.06 | 1.85 | 0.07 | 133 |
| 14 | | | | | | | | | | | | | | | | | | |
| Phage- | Y | W | Y | C | S | P | S | I | V | R | C | V | L | V | 0.06 | 1.51 | 0.09 | 134 |
| 15 | | | | | | | | | | | | | | | | | | |
| Phage- | P | D | C | H | T | I | L | F | K | P | W | C | V | R | 0.07 | 1.00 | 0.06 | 135 |
| 16 | | | | | | | | | | | | | | | | | | |
| Phage- | W | I | C | D | K | S | G | S | I | M | L | C | R | A | 0.06 | 0.91 | 0.06 | 136 |
| 17 | | | | | | | | | | | | | | | | | | |
| Phage- | D | — | P | W | L | S | G | F | S | R | — | L | L | C | 0.09 | 1.27 | 0.07 | 138 |
| 19 | | | | | | | | | | | | | | | | | | |
| Phage- | Y | — | — | G | M | S | T | I | Q | L | W | L | K | C | 0.06 | 2.16 | 0.09 | 139 |
| 20 | | | | | | | | | | | | | | | | | | |
| Phage- | Y | — | — | G | H | M | P | Q | Y | E | V | K | — | — | 0.07 | 0.22 | 0.06 | 140 |
| 21 | | | | | | | | | | | | | | | | | | |
| Phage- | Y | — | — | G | H | T | E | E | H | Y | V | K | — | M | 0.05 | 0.62 | 0.07 | 141 |
| 22 | | | | | | | | | | | | | | | | | | |
| Phage- | Y | — | M | G | H | S | H | E | H | I | W | K | — | — | 0.06 | 0.87 | 0.07 | 142 |
| 23 | | | | | | | | | | | | | | | | | | |
| Phage- | Y | — | — | G | H | S | H | E | Y | L | A | K | — | E | 0.05 | 0.86 | 0.06 | 143 |
| 24 | | | | | | | | | | | | | | | | | | |
| Phage- | T | — | S | — | — | — | T | W | P | — | — | I | — | — | 0.08 | 2.00 | 0.07 | 182 |
| 887 | | | | | | | | | | | | | | | | | | |
| Phage- | L | — | — | — | — | — | — | W | P | — | — | V | — | — | 0.06 | 2.16 | 0.08 | 183 |
| 888 | | | | | | | | | | | | | | | | | | |
| Phage- | T | — | S | — | — | — | — | — | P | — | — | L | — | — | 0.07 | 2.38 | 0.06 | 184 |
| 889 | | | | | | | | | | | | | | | | | | |
| Phage- | T | — | — | — | — | — | A | W | P | — | — | Q | — | — | 0.06 | 2.23 | 0.08 | 185 |
| 890 | | | | | | | | | | | | | | | | | | |
| Phage- | T | — | S | — | — | — | T | — | T | — | — | K | — | F | 0.06 | 1.87 | 0.06 | 186 |
| 891 | | | | | | | | | | | | | | | | | | |
| Phage- | T | — | — | — | — | — | T | W | S | — | — | A | — | F | 0.07 | 2.23 | 0.07 | 187 |
| 892 | | | | | | | | | | | | | | | | | | |
| Phage- | T | — | — | — | — | — | A | W | G | — | — | L | — | F | 0.08 | 2.30 | 0.07 | 188 |
| 893 | | | | | | | | | | | | | | | | | | |
| Phage- | L | — | — | — | — | — | T | — | P | — | — | L | — | — | 0.05 | 2.39 | 0.07 | 189 |
| 894 | | | | | | | | | | | | | | | | | | |
| Phage- | T | — | S | — | — | — | A | — | P | — | — | L | — | — | 0.09 | 2.36 | 0.08 | 190 |
| 895 | | | | | | | | | | | | | | | | | | |
| Phage- | T | — | S | — | — | — | A | — | S | — | — | A | — | F | 0.07 | 1.89 | 0.15 | 191 |
| 896 | | | | | | | | | | | | | | | | | | |
| Phage- | L | — | — | — | — | — | T | — | P | — | — | T | — | — | 0.06 | 1.71 | 0.06 | 192 |
| 897 | | | | | | | | | | | | | | | | | | |
| Phage- | — | — | S | — | — | — | H | — | P | — | — | Q | — | F | 0.06 | 1.16 | 0.06 | 193 |
| 898 | | | | | | | | | | | | | | | | | | |
| Phage- | T | — | S | — | — | — | T | — | E | — | — | R | — | — | 0.06 | 1.84 | 0.06 | 194 |
| 899 | | | | | | | | | | | | | | | | | | |
| Phage- | T | — | S | — | — | — | H | — | P | — | — | I | — | — | 0.06 | 1.78 | 0.07 | 195 |
| 900 | | | | | | | | | | | | | | | | | | |
| Phage- | — | — | S | — | — | — | A | — | Y | — | — | W | — | — | 0.09 | 1.90 | 0.09 | 196 |
| 901 | | | | | | | | | | | | | | | | | | |
| Phage- | T | — | S | — | — | — | T | W | — | — | — | L | — | F | 0.06 | 2.31 | 0.09 | 197 |
| 902 | | | | | | | | | | | | | | | | | | |
| Phage- | T | — | — | — | — | — | T | — | L | — | — | T | — | — | 0.06 | 1.68 | 0.06 | 198 |
| 903 | | | | | | | | | | | | | | | | | | |
| Phage- | T | — | — | — | — | — | H | — | P | — | — | A | — | — | 0.10 | 1.92 | 0.07 | 199 |
| 904 | | | | | | | | | | | | | | | | | | |
| Phage- | L | — | — | — | — | F | T | — | P | — | — | L | — | — | 0.05 | 1.73 | 0.21 | 200 |
| 905 | | | | | | | | | | | | | | | | | | |
| Phage- | L | — | K | P | — | V | P | — | K | — | — | T | — | — | 0.06 | 1.86 | 0.07 | 201 |
| 906 | | | | | | | | | | | | | | | | | | |
| Phage- | T | — | S | — | — | — | T | W | P | — | — | I | — | — | 0.09 | 2.41 | 0.11 | 1040 |
| 907 | | | | | | | | | | | | | | | | | | |
| Phage- | L | — | S | — | — | — | M | W | P | — | — | T | — | — | 0.08 | 2.39 | 0.08 | 1041 |
| 908 | | | | | | | | | | | | | | | | | | |
| Phage- | L | — | S | — | — | — | M | — | S | — | — | T | — | — | 0.07 | 2.39 | 0.12 | 1042 |
| 909 | | | | | | | | | | | | | | | | | | |
| Phage- | T | — | S | — | — | — | T | W | P | — | — | I | — | — | 0.06 | 2.35 | 0.08 | 1043 |
| 910 | | | | | | | | | | | | | | | | | | |
| Phage- | T | — | — | — | — | — | M | W | — | — | — | V | — | — | 0.10 | 2.32 | 0.09 | 1044 |
| 911 | | | | | | | | | | | | | | | | | | |
| Phage- | M | — | S | — | — | — | M | — | K | — | — | L | — | — | 0.05 | 2.32 | 0.05 | 1045 |
| 912 | | | | | | | | | | | | | | | | | | |
| Phage- | T | — | S | — | — | — | T | — | — | — | — | F | — | — | 0.06 | 2.31 | 0.06 | 1046 |
| 913 | | | | | | | | | | | | | | | | | | |
| Phage- | M | — | — | — | — | — | T | — | P | — | — | A | — | — | 0.07 | 2.30 | 0.06 | 1047 |
| 914 | | | | | | | | | | | | | | | | | | |
| Phage- | — | — | — | — | — | — | M | W | P | — | — | V | — | V | 0.07 | 2.29 | 0.13 | 1048 |
| 915 | | | | | | | | | | | | | | | | | | |
| Phage- | T | — | S | — | — | — | — | — | P | — | — | L | — | — | 0.06 | 2.29 | 0.08 | 1049 |
| 916 | | | | | | | | | | | | | | | | | | |
| Phage- | — | — | K | P | — | V | A | N | Y | — | — | W | — | — | 0.08 | 2.29 | 0.10 | 1050 |
| 917 | | | | | | | | | | | | | | | | | | |
| Phage- | M | — | S | — | — | — | M | — | S | — | — | L | — | — | 0.10 | 2.29 | 0.08 | 1051 |
| 918 | | | | | | | | | | | | | | | | | | |
| Phage- | L | — | — | — | — | — | T | — | P | — | — | F | — | — | 0.06 | 2.28 | 0.07 | 1052 |
| 919 | | | | | | | | | | | | | | | | | | |
| Phage- | M | — | — | — | — | — | N | — | P | — | — | Q | — | — | 0.06 | 2.28 | 0.08 | 1053 |
| 920 | | | | | | | | | | | | | | | | | | |
| Phage- | T | — | S | — | — | — | T | W | S | — | — | M | — | — | 0.07 | 2.26 | 0.09 | 1054 |
| 921 | | | | | | | | | | | | | | | | | | |
| Phage- | L | — | S | — | — | — | M | — | P | — | — | T | — | — | 0.09 | 2.25 | 0.07 | 1055 |
| 922 | | | | | | | | | | | | | | | | | | |
| Phage- | T | — | — | — | — | — | H | — | S | — | — | A | — | F | 0.14 | 2.25 | 0.07 | 1056 |
| 923 | | | | | | | | | | | | | | | | | | |
| Phage- | T | — | — | — | — | — | T | — | P | — | — | S | — | — | 0.06 | 2.25 | 0.06 | 1057 |
| 924 | | | | | | | | | | | | | | | | | | |
| Phage- | R | — | — | — | — | — | — | — | P | — | — | P | — | — | 0.06 | 2.25 | 0.05 | 1058 |
| 925 | | | | | | | | | | | | | | | | | | |
| Phage- | T | — | K | — | — | — | T | — | L | — | — | F | — | — | 0.08 | 2.23 | 0.08 | 1059 |
| 926 | | | | | | | | | | | | | | | | | | |
| Phage- | M | — | — | — | — | F | H | — | P | — | — | A | — | — | 0.06 | 2.23 | 0.10 | 1060 |
| 927 | | | | | | | | | | | | | | | | | | |
| Phage- | T | — | — | — | — | — | N | W | E | — | — | W | — | F | 0.08 | 2.22 | 0.08 | 1061 |
| 928 | | | | | | | | | | | | | | | | | | |
| Phage- | T | — | — | — | — | — | T | — | — | — | — | — | — | V | 0.10 | 2.21 | 0.09 | 1062 |
| 929 | | | | | | | | | | | | | | | | | | |
| Phage- | T | — | — | — | — | — | A | — | P | — | — | L | — | F | 0.12 | 2.18 | 0.11 | 1063 |
| 930 | | | | | | | | | | | | | | | | | | |
| Phage- | T | — | S | — | — | — | — | N | K | L | S | L | — | — | 0.05 | 2.18 | 0.06 | 1064 |
| 931 | | | | | | | | | | | | | | | | | | |
| Phage- | M | — | S | — | — | — | N | W | P | — | — | L | — | F | 0.06 | 2.18 | 0.07 | 1065 |
| 932 | | | | | | | | | | | | | | | | | | |
| Phage- | R | — | S | — | — | F | H | — | P | L | S | L | — | — | 0.08 | 2.18 | 0.06 | 1066 |
| 933 | | | | | | | | | | | | | | | | | | |
| Phage- | N | — | S | — | — | — | T | — | P | — | — | L | — | — | 0.06 | 2.17 | 0.07 | 1067 |
| 934 | | | | | | | | | | | | | | | | | | |
| Phage- | — | — | — | — | — | — | M | — | S | — | — | T | — | — | 0.08 | 2.16 | 0.08 | 1068 |
| 935 | | | | | | | | | | | | | | | | | | |
| Phage- | N | — | S | — | — | — | Q | W | P | — | — | I | — | — | 0.07 | 2.15 | 0.08 | 1069 |
| 936 | | | | | | | | | | | | | | | | | | |
| Phage- | L | — | — | — | — | — | H | — | P | — | — | Q | — | — | 0.07 | 2.15 | 0.07 | 1070 |
| 937 | | | | | | | | | | | | | | | | | | |
| Phage- | T | — | — | — | — | — | H | W | P | — | — | T | — | F | 0.08 | 2.15 | 0.07 | 1071 |
| 938 | | | | | | | | | | | | | | | | | | |
| Phage- | T | — | S | — | — | — | T | — | T | — | S | K | — | F | 0.05 | 2.15 | 0.06 | 1072 |
| 939 | | | | | | | | | | | | | | | | | | |
| Phage- | L | — | N | — | — | — | T | — | S | — | — | T | — | — | 0.05 | 2.14 | 0.06 | 1073 |
| 940 | | | | | | | | | | | | | | | | | | |
| Phage- | T | — | S | — | — | — | T | — | T | — | — | W | — | F | 0.08 | 2.14 | 0.07 | 1074 |
| 941 | | | | | | | | | | | | | | | | | | |
| Phage- | W | — | K | P | — | — | P | — | K | — | — | P | — | F | 0.05 | 2.14 | 0.07 | 1075 |
| 942 | | | | | | | | | | | | | | | | | | |
| Phage- | T | — | — | — | — | — | M | — | T | — | — | L | — | F | 0.06 | 2.14 | 0.09 | 1076 |
| 943 | | | | | | | | | | | | | | | | | | |
| Phage- | T | — | S | — | — | — | T | W | T | — | — | Q | — | — | 0.07 | 2.14 | 0.08 | 1077 |
| 944 | | | | | | | | | | | | | | | | | | |
| Phage- | L | — | — | — | — | — | T | — | — | — | — | L | — | — | 0.08 | 2.13 | 0.07 | 1078 |
| 945 | | | | | | | | | | | | | | | | | | |
| Phage- | M | — | — | E | F | F | N | — | P | — | — | L | — | — | 0.07 | 2.11 | 0.07 | 1079 |
| 946 | | | | | | | | | | | | | | | | | | |
| Phage- | T | — | — | — | — | — | T | — | E | — | — | A | — | — | 0.05 | 2.09 | 0.07 | 1080 |
| 947 | | | | | | | | | | | | | | | | | | |
| Phage- | T | — | S | — | — | — | Y | — | T | — | — | — | — | F | 0.07 | 2.09 | 0.08 | 1081 |
| 948 | | | | | | | | | | | | | | | | | | |
| Phage- | T | — | — | — | V | F | — | — | G | — | — | E | — | F | 0.06 | 2.09 | 0.07 | 1082 |
| 949 | | | | | | | | | | | | | | | | | | |
| Phage- | — | — | S | — | — | — | M | — | T | — | — | T | — | — | 0.09 | 2.09 | 0.06 | 1083 |
| 950 | | | | | | | | | | | | | | | | | | |
| Phage- | Q | — | — | — | — | — | — | — | P | — | — | M | — | — | 0.07 | 2.08 | 0.08 | 1084 |
| 951 | | | | | | | | | | | | | | | | | | |
| Phage- | Y | — | — | — | — | — | T | — | P | — | — | L | — | — | 0.06 | 2.08 | 0.08 | 1085 |
| 952 | | | | | | | | | | | | | | | | | | |
| Phage- | T | — | — | — | — | — | T | — | — | — | — | E | — | — | 0.05 | 2.08 | 0.11 | 1086 |
| 953 | | | | | | | | | | | | | | | | | | |
| Phage- | T | — | S | E | — | — | T | — | T | — | — | W | — | F | 0.06 | 2.05 | 0.06 | 1087 |
| 954 | | | | | | | | | | | | | | | | | | |
| Phage- | T | — | — | — | — | — | H | — | L | — | — | W | — | M | 0.10 | 2.05 | 0.08 | 1088 |
| 955 | | | | | | | | | | | | | | | | | | |
| Phage- | T | — | S | — | — | — | T | — | V | — | — | Q | — | F | 0.08 | 2.05 | 0.07 | 1089 |
| 956 | | | | | | | | | | | | | | | | | | |
| Phage- | G | — | — | — | — | — | T | — | P | — | — | S | — | — | 0.06 | 2.05 | 0.06 | 1090 |
| 957 | | | | | | | | | | | | | | | | | | |
| Phage- | T | — | — | — | — | — | T | — | K | — | — | A | — | — | 0.07 | 2.05 | 0.09 | 1091 |
| 958 | | | | | | | | | | | | | | | | | | |
| Phage- | M | — | S | — | — | — | M | — | P | — | — | T | — | — | 0.06 | 2.04 | 0.08 | 1092 |
| 959 | | | | | | | | | | | | | | | | | | |
| Phage- | T | — | — | — | — | — | — | — | — | — | — | W | — | — | 0.07 | 2.03 | 0.08 | 1093 |
| 960 | | | | | | | | | | | | | | | | | | |
| Phage- | T | — | S | — | — | — | — | W | — | — | — | W | — | F | 0.07 | 2.03 | 0.08 | 1094 |
| 961 | | | | | | | | | | | | | | | | | | |
| Phage- | T | — | — | — | — | — | M | W | H | — | — | V | — | — | 0.07 | 2.03 | 0.09 | 1095 |
| 962 | | | | | | | | | | | | | | | | | | |
| Phage- | T | — | S | — | — | F | H | — | E | — | — | R | — | — | 0.05 | 2.02 | 0.05 | 1096 |
| 963 | | | | | | | | | | | | | | | | | | |
| Phage- | L | — | — | — | — | F | K | — | P | — | — | L | — | — | 0.07 | 2.02 | 0.07 | 1097 |
| 964 | | | | | | | | | | | | | | | | | | |
| Phage- | L | — | E | E | F | — | D | — | P | — | — | M | — | — | 0.05 | 2.01 | 0.06 | 1098 |
| 965 | | | | | | | | | | | | | | | | | | |
| Phage- | T | — | — | — | — | — | T | — | S | — | — | M | — | F | 0.06 | 2.00 | 0.07 | 1099 |
| 966 | | | | | | | | | | | | | | | | | | |
| Phage- | L | — | — | — | — | — | — | — | P | — | — | T | — | — | 0.09 | 2.00 | 0.10 | 1100 |
| 967 | | | | | | | | | | | | | | | | | | |
| Phage- | H | — | — | — | — | — | H | — | P | — | — | A | — | — | 0.07 | 2.00 | 0.12 | 1101 |
| 968 | | | | | | | | | | | | | | | | | | |
| Phage- | T | — | — | — | — | — | N | — | P | — | — | — | — | — | 0.07 | 1.99 | 0.14 | 1102 |
| 969 | | | | | | | | | | | | | | | | | | |
| Phage- | M | — | — | — | — | — | T | — | P | — | — | L | — | — | 0.06 | 1.99 | 0.07 | 1103 |
| 970 | | | | | | | | | | | | | | | | | | |
| Phage- | T | — | — | — | — | — | T | — | S | — | — | W | — | F | 0.09 | 1.99 | 0.07 | 1104 |
| 971 | | | | | | | | | | | | | | | | | | |
| Phage- | T | — | — | — | — | — | H | — | T | — | — | E | — | F | 0.07 | 1.99 | 0.07 | 1105 |
| 972 | | | | | | | | | | | | | | | | | | |
| Phage- | — | — | — | — | V | F | — | — | S | — | — | M | — | F | 0.06 | 1.99 | 0.06 | 1106 |
| 973 | | | | | | | | | | | | | | | | | | |
| Phage- | — | — | — | — | — | — | T | — | S | — | — | M | — | — | 0.07 | 1.99 | 0.09 | 1107 |
| 974 | | | | | | | | | | | | | | | | | | |
| Phage- | L | — | — | E | F | F | N | — | P | — | — | L | — | — | 0.06 | 1.98 | 0.06 | 1108 |
| 975 | | | | | | | | | | | | | | | | | | |
| Phage- | Q | — | S | — | — | — | A | W | P | — | — | F | — | — | 0.07 | 1.97 | 0.08 | 1109 |
| 976 | | | | | | | | | | | | | | | | | | |
| Phage- | M | — | — | — | — | — | H | — | T | — | — | T | — | — | 0.10 | 1.97 | 0.07 | 1110 |
| 977 | | | | | | | | | | | | | | | | | | |
| Phage- | T | — | S | — | — | — | T | — | E | — | — | W | — | M | 0.06 | 1.97 | 0.07 | 1111 |
| 978 | | | | | | | | | | | | | | | | | | |
| Phage- | M | — | S | — | — | — | N | — | P | — | — | W | — | — | 0.07 | 1.97 | 0.08 | 1112 |
| 979 | | | | | | | | | | | | | | | | | | |
| Phage- | T | — | — | — | — | — | T | — | E | — | — | — | — | F | 0.08 | 1.96 | 0.06 | 1113 |
| 980 | | | | | | | | | | | | | | | | | | |
| Phage- | W | — | — | — | V | F | — | — | T | — | S | P | — | — | 0.06 | 1.96 | 0.07 | 1114 |
| 981 | | | | | | | | | | | | | | | | | | |
| Phage- | T | — | — | — | — | — | A | — | T | — | — | A | — | F | 0.06 | 1.96 | 0.06 | 1115 |
| 982 | | | | | | | | | | | | | | | | | | |
| Phage- | T | — | — | — | — | — | T | — | E | — | — | T | — | — | 0.08 | 1.96 | 0.08 | 1116 |
| 983 | | | | | | | | | | | | | | | | | | |
| Phage- | L | — | K | P | — | V | P | N | S | — | — | T | — | — | 0.06 | 1.95 | 0.06 | 1117 |
| 984 | | | | | | | | | | | | | | | | | | |
| Phage- | T | — | S | — | — | — | H | — | P | — | — | D | — | I | 0.06 | 1.95 | 0.07 | 1118 |
| 985 | | | | | | | | | | | | | | | | | | |
| Phage- | P | — | — | — | — | — | M | W | P | — | — | V | — | — | 0.05 | 1.95 | 0.06 | 1119 |
| 986 | | | | | | | | | | | | | | | | | | |
| Phage- | L | — | N | — | — | — | M | — | K | — | — | T | — | — | 0.07 | 1.94 | 0.06 | 1120 |
| 987 | | | | | | | | | | | | | | | | | | |
| Phage- | M | — | S | — | — | — | M | — | P | — | — | L | — | — | 0.07 | 1.93 | 0.07 | 1121 |
| 988 | | | | | | | | | | | | | | | | | | |
| Phage- | T | — | S | — | — | — | M | W | T | — | — | — | — | F | 0.09 | 1.93 | 0.08 | 1122 |
| 989 | | | | | | | | | | | | | | | | | | |
| Phage- | T | — | — | — | — | — | Y | W | T | — | — | A | — | F | 0.09 | 1.93 | 0.09 | 1123 |
| 990 | | | | | | | | | | | | | | | | | | |
| Phage- | T | — | S | — | — | — | G | — | P | — | — | L | — | Y | 0.07 | 1.92 | 0.08 | 1124 |
| 991 | | | | | | | | | | | | | | | | | | |
| Phage- | — | — | S | — | — | — | — | L | Q | L | S | I | — | — | 0.05 | 1.92 | 0.06 | 1125 |
| 992 | | | | | | | | | | | | | | | | | | |
| Phage- | T | — | S | — | — | — | T | W | — | — | — | S | — | F | 0.09 | 1.92 | 0.10 | 1126 |
| 993 | | | | | | | | | | | | | | | | | | |
| Phage- | T | — | S | — | — | — | H | — | V | — | — | M | — | F | 0.07 | 1.91 | 0.17 | 1127 |
| 994 | | | | | | | | | | | | | | | | | | |
| Phage- | T | — | S | — | — | — | T | — | I | — | — | M | — | F | 0.05 | 1.90 | 0.08 | 1128 |
| 995 | | | | | | | | | | | | | | | | | | |
| Phage- | T | — | R | — | — | — | T | — | N | — | — | F | — | — | 0.05 | 1.90 | 0.07 | 1129 |
| 996 | | | | | | | | | | | | | | | | | | |
| Phage- | T | — | S | — | — | — | T | W | P | — | — | I | — | — | 0.07 | 1.89 | 0.08 | 1130 |
| 997 | | | | | | | | | | | | | | | | | | |
| Phage- | T | — | — | — | — | — | — | — | P | — | — | A | — | — | 0.10 | 1.89 | 0.09 | 1131 |
| 998 | | | | | | | | | | | | | | | | | | |
| Phage- | — | — | — | — | V | F | — | F | P | — | S | P | — | — | 0.06 | 1.87 | 0.08 | 1132 |
| 999 | | | | | | | | | | | | | | | | | | |
| Phage- | T | — | S | — | — | F | — | — | S | — | — | A | — | F | 0.05 | 1.86 | 0.07 | 1133 |
| 1000 | | | | | | | | | | | | | | | | | | |
| Phage- | T | — | T | — | — | F | — | — | N | — | — | M | — | F | 0.06 | 1.85 | 0.06 | 1134 |
| 1001 | | | | | | | | | | | | | | | | | | |
| Phage- | T | — | S | — | — | — | T | — | — | — | — | A | — | Y | 0.11 | 1.84 | 0.14 | 1135 |
| 1002 | | | | | | | | | | | | | | | | | | |
| Phage- | T | — | S | — | — | — | T | — | S | — | — | Q | — | — | 0.06 | 1.84 | 0.07 | 1136 |
| 1003 | | | | | | | | | | | | | | | | | | |
| Phage- | T | — | S | — | — | — | Y | — | — | — | — | V | — | — | 0.09 | 1.84 | 0.08 | 1137 |
| 1004 | | | | | | | | | | | | | | | | | | |
| Phage- | L | — | R | — | — | — | M | W | P | — | — | T | — | — | 0.07 | 1.83 | 0.06 | 1138 |
| 1005 | | | | | | | | | | | | | | | | | | |
| Phage- | T | — | S | — | — | — | T | F | H | — | — | Q | — | F | 0.07 | 1.83 | 0.08 | 1139 |
| 1006 | | | | | | | | | | | | | | | | | | |
| Phage- | T | — | S | — | — | — | H | — | E | — | — | — | — | — | 0.08 | 1.82 | 0.11 | 1140 |
| 1007 | | | | | | | | | | | | | | | | | | |
| Phage- | Q | — | — | — | — | — | — | — | P | — | — | V | — | — | 0.06 | 1.82 | 0.07 | 1141 |
| 1008 | | | | | | | | | | | | | | | | | | |
| Phage- | L | — | K | P | — | V | P | — | Q | — | — | T | — | F | 0.06 | 1.82 | 0.05 | 1142 |
| 1009 | | | | | | | | | | | | | | | | | | |
| Phage- | T | — | — | — | — | — | M | W | Q | — | — | V | — | — | 0.06 | 1.81 | 0.06 | 1143 |
| 1010 | | | | | | | | | | | | | | | | | | |
| Phage- | P | — | S | — | — | — | T | — | — | — | L | — | — | V | 0.06 | 1.81 | 0.06 | 1144 |
| 1011 | | | | | | | | | | | | | | | | | | |
| Phage- | — | — | — | — | — | — | M | W | P | — | — | V | — | V | 0.05 | 1.80 | 0.06 | 1145 |
| 1012 | | | | | | | | | | | | | | | | | | |
| Phage- | — | — | — | — | — | — | A | — | P | — | — | A | — | — | 0.06 | 1.80 | 0.06 | 1146 |
| 1013 | | | | | | | | | | | | | | | | | | |
| Phage- | Y | — | S | — | — | — | Q | W | P | — | — | I | — | — | 0.09 | 1.79 | 0.08 | 1147 |
| 1014 | | | | | | | | | | | | | | | | | | |
| Phage- | T | — | — | — | — | — | — | — | Q | — | — | L | — | F | 0.07 | 1.79 | 0.07 | 1148 |
| 1015 | | | | | | | | | | | | | | | | | | |
| Phage- | — | — | — | — | — | — | — | — | S | — | — | A | — | F | 0.06 | 1.79 | 0.07 | 1149 |
| 1016 | | | | | | | | | | | | | | | | | | |
| Phage- | — | — | S | — | — | F | H | — | P | — | — | I | — | — | 0.05 | 1.79 | 0.07 | 1150 |
| 1017 | | | | | | | | | | | | | | | | | | |
| Phage- | L | — | K | P | — | V | P | — | Q | — | — | T | — | — | 0.06 | 1.78 | 0.07 | 1151 |
| 1018 | | | | | | | | | | | | | | | | | | |
| Phage- | M | — | T | — | — | — | L | — | N | — | — | T | — | — | 0.06 | 1.78 | 0.09 | 1152 |
| 1019 | | | | | | | | | | | | | | | | | | |
| Phage- | T | — | — | — | — | — | H | — | P | — | — | — | — | — | 0.06 | 1.77 | 0.05 | 1153 |
| 1020 | | | | | | | | | | | | | | | | | | |
| Phage- | W | — | — | E | F | F | N | — | H | — | — | L | — | F | 0.07 | 1.77 | 0.06 | 1154 |
| 1021 | | | | | | | | | | | | | | | | | | |
| Phage- | T | — | S | — | — | F | — | L | Q | L | S | I | — | — | 0.06 | 1.77 | 0.06 | 1155 |
| 1022 | | | | | | | | | | | | | | | | | | |
| Phage- | — | — | — | — | — | — | — | — | P | — | — | Q | — | — | 0.07 | 1.77 | 0.11 | 1156 |
| 1023 | | | | | | | | | | | | | | | | | | |
| Phage- | L | — | T | — | — | L | A | — | N | — | — | T | — | — | 0.05 | 1.77 | 0.05 | 1157 |
| 1024 | | | | | | | | | | | | | | | | | | |
| Phage- | G | — | — | — | — | — | H | — | P | — | — | F | — | — | 0.06 | 1.76 | 0.06 | 1158 |
| 1025 | | | | | | | | | | | | | | | | | | |
| Phage- | T | — | — | — | — | — | V | W | E | — | — | M | — | F | 0.07 | 1.76 | 0.07 | 1159 |
| 1026 | | | | | | | | | | | | | | | | | | |
| Phage- | T | — | — | — | — | — | N | — | P | — | — | L | — | — | 0.05 | 1.75 | 0.08 | 1160 |
| 1027 | | | | | | | | | | | | | | | | | | |
| Phage- | M | — | — | — | — | — | M | — | — | — | — | T | — | — | 0.06 | 1.75 | 0.09 | 1161 |
| 1028 | | | | | | | | | | | | | | | | | | |
| Phage- | T | — | — | — | — | — | — | F | P | — | — | W | — | — | 0.06 | 1.75 | 0.06 | 1162 |
| 1029 | | | | | | | | | | | | | | | | | | |
| Phage- | — | — | — | — | V | F | P | — | P | — | — | P | — | F | 0.05 | 1.75 | 0.06 | 1163 |
| 1030 | | | | | | | | | | | | | | | | | | |
| Phage- | T | — | S | — | — | — | M | — | P | — | — | W | — | V | 0.06 | 1.75 | 0.08 | 1164 |
| 1031 | | | | | | | | | | | | | | | | | | |
| Phage- | W | — | — | — | V | F | — | — | S | — | — | L | — | — | 0.05 | 1.75 | 0.06 | 1165 |
| 1032 | | | | | | | | | | | | | | | | | | |
| Phage- | G | — | — | — | — | F | H | — | P | — | — | L | — | — | 0.05 | 1.74 | 0.08 | 1166 |
| 1033 | | | | | | | | | | | | | | | | | | |
| Phage- | L | — | E | E | F | F | H | — | S | — | — | V | — | — | 0.06 | 1.74 | 0.06 | 1167 |
| 1034 | | | | | | | | | | | | | | | | | | |
| Phage- | T | — | — | — | — | — | A | W | P | — | — | H | — | — | 0.05 | 1.73 | 0.06 | 1168 |
| 1035 | | | | | | | | | | | | | | | | | | |
| Phage- | Q | — | — | — | — | — | M | W | T | — | — | A | — | F | 0.06 | 1.73 | 0.07 | 1169 |
| 1036 | | | | | | | | | | | | | | | | | | |
| Phage- | M | — | S | — | — | — | T | — | P | — | — | M | — | — | 0.07 | 1.73 | 0.06 | 1170 |
| 1037 | | | | | | | | | | | | | | | | | | |
| Phage- | T | — | — | — | — | — | T | — | P | — | — | S | — | — | 0.06 | 1.73 | 0.05 | 1171 |
| 1038 | | | | | | | | | | | | | | | | | | |
| Phage- | T | — | — | — | — | — | V | W | G | — | — | L | — | F | 0.06 | 1.73 | 0.06 | 1172 |
| 1039 | | | | | | | | | | | | | | | | | | |
| Phage- | T | — | — | — | — | — | H | — | — | — | — | E | — | F | 0.06 | 1.72 | 0.06 | 1173 |
| 1040 | | | | | | | | | | | | | | | | | | |
| Phage- | R | — | R | — | — | F | — | — | N | — | — | — | — | M | 0.05 | 1.72 | 0.06 | 1174 |
| 1041 | | | | | | | | | | | | | | | | | | |
| Phage- | W | — | — | — | — | — | T | W | P | — | — | L | — | I | 0.14 | 1.70 | 0.17 | 1175 |
| 1042 | | | | | | | | | | | | | | | | | | |
| Phage- | R | — | — | — | — | V | M | — | D | — | — | V | — | — | 0.07 | 1.70 | 0.07 | 1176 |
| 1043 | | | | | | | | | | | | | | | | | | |
| Phage- | T | — | S | — | — | — | H | W | T | — | — | L | — | F | 0.06 | 1.69 | 0.06 | 1177 |
| 1044 | | | | | | | | | | | | | | | | | | |
| Phage- | T | — | S | — | — | — | H | F | — | — | — | M | — | F | 0.06 | 1.69 | 0.07 | 1178 |
| 1045 | | | | | | | | | | | | | | | | | | |
| Phage- | — | — | — | — | — | — | H | — | V | — | — | F | — | F | 0.07 | 1.68 | 0.08 | 1179 |
| 1046 | | | | | | | | | | | | | | | | | | |
| Phage- | T | — | S | — | — | — | T | — | N | — | — | W | — | F | 0.06 | 1.68 | 0.05 | 1180 |
| 1047 | | | | | | | | | | | | | | | | | | |
| Phage- | T | — | S | — | — | — | — | — | P | — | — | V | — | — | 0.06 | 1.67 | 0.07 | 1181 |
| 1048 | | | | | | | | | | | | | | | | | | |
| Phage- | W | — | — | — | — | — | M | — | P | — | — | L | — | — | 0.08 | 1.67 | 0.06 | 1182 |
| 1049 | | | | | | | | | | | | | | | | | | |
| Phage- | L | — | N | — | — | — | P | — | K | — | — | T | — | — | 0.06 | 1.66 | 0.07 | 1183 |
| 1050 | | | | | | | | | | | | | | | | | | |
| Phage- | L | — | S | — | — | — | M | — | P | — | — | L | — | Y | 0.06 | 1.66 | 0.08 | 1184 |
| 1051 | | | | | | | | | | | | | | | | | | |
| Phage- | T | — | — | — | — | — | M | W | V | — | — | T | — | — | 0.06 | 1.66 | 0.07 | 1185 |
| 1052 | | | | | | | | | | | | | | | | | | |
| Phage- | L | — | — | — | — | — | N | — | P | — | — | A | — | — | 0.06 | 1.66 | 0.07 | 1186 |
| 1053 | | | | | | | | | | | | | | | | | | |
| Phage- | — | — | — | — | — | — | T | — | T | — | — | M | — | I | 0.07 | 1.66 | 0.07 | 1187 |
| 1054 | | | | | | | | | | | | | | | | | | |
| Phage- | — | — | — | — | — | L | — | — | P | — | — | D | — | — | 0.05 | 1.65 | 0.07 | 1188 |
| 1055 | | | | | | | | | | | | | | | | | | |
| Phage- | T | — | — | — | — | — | G | W | — | — | — | F | — | F | 0.07 | 1.65 | 0.09 | 1189 |
| 1056 | | | | | | | | | | | | | | | | | | |
| Phage- | M | — | S | — | — | — | T | — | P | — | — | — | — | — | 0.06 | 1.64 | 0.10 | 1190 |
| 1057 | | | | | | | | | | | | | | | | | | |
| Phage- | L | — | S | — | — | — | T | — | P | — | — | W | — | M | 0.06 | 1.64 | 0.06 | 1191 |
| 1058 | | | | | | | | | | | | | | | | | | |
| Phage- | D | — | W | G | D | — | P | — | P | — | R | V | — | — | 0.06 | 1.64 | 0.07 | 1192 |
| 1059 | | | | | | | | | | | | | | | | | | |
| Phage- | W | — | T | — | — | F | — | — | K | L | S | I | — | — | 0.06 | 1.63 | 0.06 | 1193 |
| 1060 | | | | | | | | | | | | | | | | | | |
| Phage- | L | — | S | — | — | — | M | — | Q | — | — | T | — | — | 0.06 | 1.63 | 0.06 | 1194 |
| 1061 | | | | | | | | | | | | | | | | | | |
| Phage- | T | — | S | — | — | — | N | — | E | — | — | W | — | F | 0.06 | 1.62 | 0.06 | 1195 |
| 1062 | | | | | | | | | | | | | | | | | | |
| Phage- | T | — | — | — | — | — | H | — | E | — | — | M | — | Y | 0.08 | 1.61 | 0.12 | 1196 |
| 1063 | | | | | | | | | | | | | | | | | | |
| Phage- | — | — | S | — | V | F | — | — | — | — | — | T | — | — | 0.05 | 1.61 | 0.08 | 1197 |
| 1064 | | | | | | | | | | | | | | | | | | |
| Phage- | T | — | — | — | — | — | T | W | P | — | — | S | — | — | 0.06 | 1.61 | 0.08 | 1198 |
| 1065 | | | | | | | | | | | | | | | | | | |
| Phage- | T | — | — | — | — | — | H | W | I | — | — | W | — | E | 0.07 | 1.61 | 0.07 | 1199 |
| 1066 | | | | | | | | | | | | | | | | | | |
| Phage- | T | — | — | — | — | — | G | W | Q | — | — | M | — | F | 0.10 | 1.60 | 0.11 | 1200 |
| 1067 | | | | | | | | | | | | | | | | | | |
| Phage- | — | — | — | — | — | — | T | — | E | — | — | R | — | — | 0.06 | 1.60 | 0.07 | 1201 |
| 1068 | | | | | | | | | | | | | | | | | | |
| Phage- | G | — | — | — | — | — | N | — | P | — | — | L | — | — | 0.06 | 1.59 | 0.05 | 1202 |
| 1069 | | | | | | | | | | | | | | | | | | |
| Phage- | T | — | — | — | — | — | Y | — | — | — | — | M | — | — | 0.06 | 1.59 | 0.07 | 1203 |
| 1070 | | | | | | | | | | | | | | | | | | |
| Phage- | — | — | — | — | — | — | P | — | P | — | — | E | — | — | 0.06 | 1.59 | 0.07 | 1204 |
| 1071 | | | | | | | | | | | | | | | | | | |
| Phage- | T | — | S | — | — | — | A | — | — | — | — | — | — | — | 0.10 | 1.59 | 0.07 | 1205 |
| 1072 | | | | | | | | | | | | | | | | | | |
| Phage- | L | — | — | — | — | — | A | — | P | — | — | M | — | — | 0.06 | 1.59 | 0.06 | 1206 |
| 1073 | | | | | | | | | | | | | | | | | | |
| Phage- | — | — | — | — | — | — | T | — | E | — | — | I | — | — | 0.05 | 1.59 | 0.05 | 1207 |
| 1074 | | | | | | | | | | | | | | | | | | |
| Phage- | — | — | — | — | — | — | M | W | P | — | — | V | — | V | 0.05 | 1.59 | 0.07 | 1208 |
| 1075 | | | | | | | | | | | | | | | | | | |
| Phage- | — | — | — | — | — | — | Q | — | G | — | — | M | — | — | 0.07 | 1.59 | 0.05 | 1209 |
| 1076 | | | | | | | | | | | | | | | | | | |
| Phage- | W | — | — | — | — | — | — | — | P | — | — | W | — | — | 0.06 | 1.58 | 0.05 | 1210 |
| 1077 | | | | | | | | | | | | | | | | | | |
| Phage- | T | — | T | — | V | F | P | — | E | — | — | R | — | — | 0.06 | 1.57 | 0.07 | 1211 |
| 1078 | | | | | | | | | | | | | | | | | | |
| Phage- | — | — | — | — | — | — | T | F | H | — | — | M | — | F | 0.07 | 1.57 | 0.09 | 1212 |
| 1079 | | | | | | | | | | | | | | | | | | |
| Phage- | T | — | S | — | — | F | — | — | K | — | — | W | — | — | 0.05 | 1.57 | 0.06 | 1213 |
| 1080 | | | | | | | | | | | | | | | | | | |
| Phage- | T | — | — | — | — | — | M | W | T | — | — | E | — | — | 0.07 | 1.57 | 0.07 | 1214 |
| 1081 | | | | | | | | | | | | | | | | | | |
| Phage- | T | — | — | — | — | — | T | — | P | — | — | E | — | M | 0.05 | 1.57 | 0.06 | 1215 |
| 1082 | | | | | | | | | | | | | | | | | | |
| Phage- | L | — | — | — | — | F | N | — | P | — | — | L | — | — | 0.05 | 1.57 | 0.05 | 1216 |
| 1083 | | | | | | | | | | | | | | | | | | |
| Phage- | M | — | T | — | — | — | T | — | P | — | — | A | — | — | 0.08 | 1.56 | 0.07 | 1217 |
| 1084 | | | | | | | | | | | | | | | | | | |
| Phage- | — | — | S | — | — | — | A | W | P | — | — | W | — | — | 0.09 | 1.55 | 0.08 | 1218 |
| 1085 | | | | | | | | | | | | | | | | | | |
| Phage- | L | — | — | E | — | F | D | — | S | — | — | M | — | — | 0.06 | 1.55 | 0.08 | 1219 |
| 1086 | | | | | | | | | | | | | | | | | | |
| Phage- | T | — | S | — | — | — | T | — | S | — | — | F | — | F | 0.07 | 1.55 | 0.07 | 1220 |
| 1087 | | | | | | | | | | | | | | | | | | |
| Phage- | — | — | K | — | — | — | P | — | Y | — | — | G | — | F | 0.07 | 1.54 | 0.08 | 1221 |
| 1088 | | | | | | | | | | | | | | | | | | |
| Phage- | T | — | S | — | V | F | — | — | T | — | S | N | — | F | 0.05 | 1.54 | 0.06 | 1222 |
| 1089 | | | | | | | | | | | | | | | | | | |
| Phage- | Q | — | — | — | — | — | M | W | T | — | — | A | — | F | 0.06 | 1.54 | 0.06 | 1223 |
| 1090 | | | | | | | | | | | | | | | | | | |
| Phage- | T | — | — | — | — | — | A | W | E | — | — | I | — | F | 0.06 | 1.54 | 0.06 | 1224 |
| 1091 | | | | | | | | | | | | | | | | | | |
| Phage- | T | — | — | — | — | — | M | W | N | — | — | Q | — | Y | 0.07 | 1.54 | 0.07 | 1225 |
| 1092 | | | | | | | | | | | | | | | | | | |
| Phage- | — | — | S | — | — | — | M | — | P | — | — | M | — | — | 0.06 | 1.53 | 0.06 | 1226 |
| 1093 | | | | | | | | | | | | | | | | | | |
| Phage- | T | — | — | — | — | — | — | W | E | — | — | L | — | F | 0.06 | 1.52 | 0.08 | 1227 |
| 1094 | | | | | | | | | | | | | | | | | | |
| Phage- | T | — | S | — | — | — | T | — | V | — | — | D | — | F | 0.06 | 1.52 | 0.09 | 1228 |
| 1095 | | | | | | | | | | | | | | | | | | |
| Phage- | T | — | E | — | V | F | I | — | S | — | — | F | — | — | 0.09 | 1.52 | 0.08 | 1229 |
| 1096 | | | | | | | | | | | | | | | | | | |
| Phage- | L | — | S | — | — | — | M | W | Q | — | — | T | — | — | 0.05 | 1.51 | 0.05 | 1230 |
| 1097 | | | | | | | | | | | | | | | | | | |
| Phage- | L | — | — | E | — | — | D | — | P | — | — | M | — | — | 0.06 | 1.51 | 0.08 | 1231 |
| 1098 | | | | | | | | | | | | | | | | | | |
| Phage- | T | — | — | — | — | — | T | W | D | — | — | L | — | F | 0.07 | 1.51 | 0.06 | 1232 |
| 1099 | | | | | | | | | | | | | | | | | | |
| Phage- | — | — | — | — | — | — | H | — | K | — | — | V | — | I | 0.06 | 1.51 | 0.06 | 1233 |
| 1100 | | | | | | | | | | | | | | | | | | |
| Phage- | I | — | — | — | — | F | I | W | S | — | — | A | — | F | 0.08 | 1.51 | 0.08 | 1234 |
| 1101 | | | | | | | | | | | | | | | | | | |
| Phage- | T | — | — | — | — | — | T | — | S | — | — | D | — | F | 0.08 | 1.51 | 0.06 | 1235 |
| 1102 | | | | | | | | | | | | | | | | | | |
| Phage- | Q | — | — | — | — | — | M | W | T | — | — | V | — | F | 0.09 | 1.50 | 0.07 | 1236 |
| 1103 | | | | | | | | | | | | | | | | | | |
| Phage- | T | — | — | — | — | — | H | — | L | — | — | H | — | — | 0.07 | 1.50 | 0.12 | 1237 |
| 1104 | | | | | | | | | | | | | | | | | | |
| Phage- | P | — | — | — | — | — | M | W | — | — | — | — | — | — | 0.06 | 1.49 | 0.05 | 1238 |
| 1105 | | | | | | | | | | | | | | | | | | |
| Phage- | N | — | S | — | — | — | T | W | P | — | — | M | — | F | 0.08 | 1.49 | 0.10 | 1239 |
| 1106 | | | | | | | | | | | | | | | | | | |
| Phage- | T | — | — | — | — | V | H | F | G | — | — | L | — | F | 0.06 | 1.49 | 0.07 | 1240 |
| 1107 | | | | | | | | | | | | | | | | | | |
| Phage- | T | — | — | — | — | — | T | — | T | — | — | T | — | F | 0.05 | 1.48 | 0.05 | 1241 |
| 1108 | | | | | | | | | | | | | | | | | | |
| Phage- | T | — | — | — | — | — | — | W | E | — | — | A | — | — | 0.05 | 1.48 | 0.08 | 1242 |
| 1109 | | | | | | | | | | | | | | | | | | |
| Phage- | T | — | — | — | — | — | M | — | Q | — | — | E | — | F | 0.05 | 1.48 | 0.06 | 1243 |
| 1110 | | | | | | | | | | | | | | | | | | |
| Phage- | T | — | — | — | — | — | H | — | T | — | — | T | — | — | 0.06 | 1.47 | 0.07 | 1244 |
| 1111 | | | | | | | | | | | | | | | | | | |
| Phage- | T | — | S | — | — | — | T | — | K | — | — | V | — | — | 0.06 | 1.47 | 0.06 | 1245 |
| 1112 | | | | | | | | | | | | | | | | | | |
| Phage- | — | — | S | — | — | — | T | — | E | — | — | — | — | F | 0.06 | 1.47 | 0.06 | 1246 |
| 1113 | | | | | | | | | | | | | | | | | | |
| Phage- | T | — | — | — | — | — | N | W | H | — | — | W | — | F | 0.08 | 1.46 | 0.15 | 1247 |
| 1114 | | | | | | | | | | | | | | | | | | |
| Phage- | L | — | — | — | — | — | T | — | P | — | — | I | — | — | 0.05 | 1.46 | 0.08 | 1248 |
| 1115 | | | | | | | | | | | | | | | | | | |
| Phage- | — | — | S | — | — | — | L | W | K | — | — | V | — | F | 0.06 | 1.45 | 0.07 | 1249 |
| 1116 | | | | | | | | | | | | | | | | | | |
| Phage- | T | — | S | — | — | F | — | — | K | — | — | W | — | W | 0.05 | 1.45 | 0.06 | 1250 |
| 1117 | | | | | | | | | | | | | | | | | | |
| Phage- | — | — | S | — | — | — | M | — | Q | — | — | L | — | F | 0.06 | 1.44 | 0.09 | 1251 |
| 1118 | | | | | | | | | | | | | | | | | | |
| Phage- | Q | — | — | — | — | — | M | W | T | — | — | A | — | F | 0.06 | 1.44 | 0.06 | 1252 |
| 1119 | | | | | | | | | | | | | | | | | | |
| Phage- | T | — | S | — | — | — | T | — | M | — | — | A | — | F | 0.09 | 1.44 | 0.10 | 1253 |
| 1120 | | | | | | | | | | | | | | | | | | |
| Phage- | T | — | S | — | — | — | T | — | — | — | — | A | — | F | 0.06 | 1.44 | 0.08 | 1254 |
| 1121 | | | | | | | | | | | | | | | | | | |
| Phage- | W | — | G | V | — | M | D | W | R | — | — | E | — | — | 0.06 | 1.44 | 0.16 | 1255 |
| 1122 | | | | | | | | | | | | | | | | | | |
| Phage- | T | — | E | D | — | F | H | F | S | — | — | E | — | F | 0.08 | 1.44 | 0.07 | 1256 |
| 1123 | | | | | | | | | | | | | | | | | | |
| Phage- | Q | — | — | — | — | — | M | W | T | — | — | A | — | F | 0.05 | 1.42 | 0.07 | 1257 |
| 1124 | | | | | | | | | | | | | | | | | | |
| Phage- | T | — | S | — | — | — | T | — | T | — | — | S | — | F | 0.07 | 1.42 | 0.06 | 1258 |
| 1125 | | | | | | | | | | | | | | | | | | |
| Phage- | W | — | D | Q | F | F | H | — | K | — | — | L | — | — | 0.05 | 1.42 | 0.07 | 1259 |
| 1126 | | | | | | | | | | | | | | | | | | |
| Phage- | — | — | — | — | — | — | T | — | V | — | — | M | — | F | 0.06 | 1.42 | 0.07 | 1260 |
| 1127 | | | | | | | | | | | | | | | | | | |
| Phage- | T | — | — | — | — | — | H | — | S | — | — | F | — | M | 0.07 | 1.42 | 0.07 | 1261 |
| 1128 | | | | | | | | | | | | | | | | | | |
| Phage- | — | — | S | — | — | — | — | L | Q | L | S | Q | — | F | 0.06 | 1.41 | 0.05 | 1262 |
| 1129 | | | | | | | | | | | | | | | | | | |
| Phage- | R | — | S | F | R | F | I | T | L | A | E | A | — | V | 0.05 | 1.41 | 0.08 | 1263 |
| 1130 | | | | | | | | | | | | | | | | | | |
| Phage- | T | — | S | — | — | — | H | — | P | — | — | I | — | — | 0.06 | 1.41 | 0.08 | 1264 |
| 1131 | | | | | | | | | | | | | | | | | | |
| Phage- | T | — | — | — | — | — | Q | F | — | — | — | L | — | — | 0.06 | 1.41 | 0.07 | 1265 |
| 1132 | | | | | | | | | | | | | | | | | | |
| Phage- | L | — | S | — | — | F | L | F | Q | — | S | T | — | — | 0.05 | 1.41 | 0.06 | 1266 |
| 1133 | | | | | | | | | | | | | | | | | | |
| Phage- | R | — | — | — | — | F | H | — | S | — | — | N | — | F | 0.05 | 1.41 | 0.06 | 1267 |
| 1134 | | | | | | | | | | | | | | | | | | |
| Phage- | G | — | — | — | — | — | — | — | P | — | — | A | — | — | 0.05 | 1.41 | 0.06 | 1268 |
| 1135 | | | | | | | | | | | | | | | | | | |
| Phage- | P | — | — | — | — | — | M | W | P | — | — | V | — | — | 0.05 | 1.41 | 0.06 | 1269 |
| 1136 | | | | | | | | | | | | | | | | | | |
| Phage- | T | — | S | — | — | — | T | — | S | — | — | A | — | — | 0.05 | 1.41 | 0.06 | 1270 |
| 1137 | | | | | | | | | | | | | | | | | | |
| Phage- | T | — | — | — | — | — | — | W | N | — | — | A | — | E | 0.07 | 1.41 | 0.07 | 1271 |
| 1138 | | | | | | | | | | | | | | | | | | |
| Phage- | L | — | — | — | — | — | H | — | S | — | — | A | — | F | 0.09 | 1.41 | 0.09 | 1272 |
| 1139 | | | | | | | | | | | | | | | | | | |
| Phage- | P | — | S | — | — | V | T | — | — | — | L | — | — | V | 0.05 | 1.41 | 0.06 | 1273 |
| 1140 | | | | | | | | | | | | | | | | | | |
| Phage- | M | — | — | — | — | — | A | W | — | — | — | L | — | — | 0.06 | 1.40 | 0.07 | 1274 |
| 1141 | | | | | | | | | | | | | | | | | | |
| Phage- | T | — | — | — | — | — | — | — | P | — | — | L | — | V | 0.06 | 1.40 | 0.06 | 1275 |
| 1142 | | | | | | | | | | | | | | | | | | |
| Phage- | L | — | — | — | — | F | M | — | E | — | — | L | — | F | 0.05 | 1.40 | 0.06 | 1276 |
| 1143 | | | | | | | | | | | | | | | | | | |
| Phage- | T | — | S | — | — | — | E | — | T | — | — | L | — | F | 0.07 | 1.40 | 0.08 | 1277 |
| 1144 | | | | | | | | | | | | | | | | | | |
| Phage- | — | — | — | — | — | — | A | W | G | — | — | A | — | F | 0.06 | 1.39 | 0.09 | 1278 |
| 1145 | | | | | | | | | | | | | | | | | | |
| Phage- | T | — | — | — | — | — | H | W | L | — | — | V | — | — | 0.06 | 1.39 | 0.08 | 1279 |
| 1146 | | | | | | | | | | | | | | | | | | |
| Phage- | — | — | S | — | F | F | H | F | H | — | — | A | — | — | 0.08 | 1.38 | 0.06 | 1280 |
| 1147 | | | | | | | | | | | | | | | | | | |
| Phage- | T | — | S | — | — | — | N | W | V | — | — | L | — | — | 0.06 | 1.38 | 0.07 | 1281 |
| 1148 | | | | | | | | | | | | | | | | | | |
| Phage- | T | — | S | — | — | — | H | W | V | — | — | — | — | — | 0.07 | 1.37 | 0.09 | 1282 |
| 1149 | | | | | | | | | | | | | | | | | | |
| Phage- | — | — | — | — | — | — | T | — | G | — | — | E | — | F | 0.06 | 1.36 | 0.07 | 1283 |
| 1150 | | | | | | | | | | | | | | | | | | |
| Phage- | — | — | — | — | — | — | T | W | — | — | — | A | — | — | 0.06 | 1.36 | 0.06 | 1284 |
| 1151 | | | | | | | | | | | | | | | | | | |
| Phage- | L | — | S | — | — | — | M | W | E | — | — | L | — | — | 0.06 | 1.36 | 0.07 | 1285 |
| 1152 | | | | | | | | | | | | | | | | | | |
| Phage- | T | — | S | — | — | — | G | — | P | — | — | T | — | — | 0.06 | 1.36 | 0.06 | 1286 |
| 1153 | | | | | | | | | | | | | | | | | | |
| Phage- | T | — | S | — | — | — | A | W | T | — | — | L | — | F | 0.05 | 1.36 | 0.06 | 1287 |
| 1154 | | | | | | | | | | | | | | | | | | |
| Phage- | T | — | S | — | — | — | A | — | E | — | — | E | — | F | 0.06 | 1.36 | 0.06 | 1288 |
| 1155 | | | | | | | | | | | | | | | | | | |
| Phage- | T | — | S | — | — | — | L | W | E | — | — | M | — | F | 0.06 | 1.35 | 0.06 | 1289 |
| 1156 | | | | | | | | | | | | | | | | | | |
| Phage- | P | — | — | — | — | — | H | — | T | — | — | T | — | — | 0.07 | 1.34 | 0.07 | 1290 |
| 1157 | | | | | | | | | | | | | | | | | | |
| Phage- | — | — | S | — | — | — | T | — | T | — | — | A | — | — | 0.08 | 1.34 | 0.07 | 1291 |
| 1158 | | | | | | | | | | | | | | | | | | |
| Phage- | T | — | S | — | — | — | H | — | S | — | — | K | — | F | 0.06 | 1.33 | 0.07 | 1292 |
| 1159 | | | | | | | | | | | | | | | | | | |
| Phage- | — | — | — | — | — | — | H | — | P | — | — | E | — | — | 0.05 | 1.32 | 0.05 | 1293 |
| 1160 | | | | | | | | | | | | | | | | | | |
| Phage- | — | — | — | E | V | F | N | — | V | — | — | F | — | F | 0.07 | 1.32 | 0.07 | 1294 |
| 1161 | | | | | | | | | | | | | | | | | | |
| Phage- | T | — | — | — | — | — | A | — | T | — | — | V | — | F | 0.05 | 1.32 | 0.08 | 1295 |
| 1162 | | | | | | | | | | | | | | | | | | |
| Phage- | — | — | — | — | — | — | M | W | E | — | — | K | — | F | 0.07 | 1.32 | 0.09 | 1296 |
| 1163 | | | | | | | | | | | | | | | | | | |
| Phage- | — | — | — | D | — | — | I | W | E | — | — | W | — | F | 0.06 | 1.32 | 0.07 | 1297 |
| 1164 | | | | | | | | | | | | | | | | | | |
| Phage- | T | — | — | — | — | — | T | — | E | — | — | S | — | — | 0.06 | 1.32 | 0.09 | 1298 |
| 1165 | | | | | | | | | | | | | | | | | | |
| Phage- | — | — | S | — | — | — | M | — | T | — | — | T | — | — | 0.08 | 1.31 | 0.08 | 1299 |
| 1166 | | | | | | | | | | | | | | | | | | |
| Phage- | L | — | — | — | — | — | A | W | E | — | — | M | — | F | 0.08 | 1.30 | 0.06 | 1300 |
| 1167 | | | | | | | | | | | | | | | | | | |
| Phage- | Q | — | S | — | — | — | T | — | — | — | — | F | — | — | 0.07 | 1.29 | 0.08 | 1301 |
| 1168 | | | | | | | | | | | | | | | | | | |
| Phage- | T | — | — | — | — | V | P | — | K | — | — | P | — | — | 0.06 | 1.29 | 0.06 | 1302 |
| 1169 | | | | | | | | | | | | | | | | | | |
| Phage- | — | — | — | — | — | — | T | — | T | — | — | F | — | — | 0.07 | 1.29 | 0.07 | 1303 |
| 1170 | | | | | | | | | | | | | | | | | | |
| Phage- | R | — | — | — | V | F | — | — | T | — | — | P | — | — | 0.06 | 1.29 | 0.07 | 1304 |
| 1171 | | | | | | | | | | | | | | | | | | |
| Phage- | T | — | S | — | — | — | T | — | K | — | — | A | — | F | 0.07 | 1.29 | 0.05 | 1305 |
| 1172 | | | | | | | | | | | | | | | | | | |
| Phage- | L | — | — | — | — | — | Y | — | P | — | — | L | — | F | 0.06 | 1.28 | 0.06 | 1306 |
| 1173 | | | | | | | | | | | | | | | | | | |
| Phage- | T | — | T | — | — | F | — | — | K | — | — | T | — | — | 0.08 | 1.28 | 0.09 | 1307 |
| 1174 | | | | | | | | | | | | | | | | | | |
| Phage- | N | — | S | — | — | — | T | W | P | — | — | M | — | F | 0.05 | 1.27 | 0.06 | 1308 |
| 1175 | | | | | | | | | | | | | | | | | | |
| Phage- | — | — | S | — | — | — | T | — | Q | — | — | — | — | F | 0.08 | 1.26 | 0.06 | 1309 |
| 1176 | | | | | | | | | | | | | | | | | | |
| Phage- | — | — | — | — | — | — | T | — | S | — | — | A | — | — | 0.07 | 1.25 | 0.07 | 1310 |
| 1177 | | | | | | | | | | | | | | | | | | |
| Phage- | N | — | — | — | — | — | M | — | S | — | — | T | — | — | 0.06 | 1.25 | 0.07 | 1311 |
| 1178 | | | | | | | | | | | | | | | | | | |
| Phage- | R | — | — | — | — | — | M | W | T | — | — | A | — | F | 0.16 | 1.25 | 0.06 | 1312 |
| 1179 | | | | | | | | | | | | | | | | | | |
| Phage- | T | — | — | — | — | — | T | — | M | — | — | V | — | — | 0.05 | 1.24 | 0.07 | 1313 |
| 1180 | | | | | | | | | | | | | | | | | | |
| Phage- | — | — | — | — | — | — | T | W | K | — | — | E | — | — | 0.05 | 1.24 | 0.07 | 1314 |
| 1181 | | | | | | | | | | | | | | | | | | |
| Phage- | T | — | — | — | — | — | T | W | L | — | — | W | — | — | 0.06 | 1.23 | 0.07 | 1315 |
| 1182 | | | | | | | | | | | | | | | | | | |
| Phage- | T | — | — | — | — | — | V | W | T | — | — | I | — | F | 0.06 | 1.23 | 0.05 | 1316 |
| 1183 | | | | | | | | | | | | | | | | | | |
| Phage- | T | — | — | D | — | — | F | W | Q | — | — | V | — | — | 0.07 | 1.22 | 0.07 | 1317 |
| 1184 | | | | | | | | | | | | | | | | | | |
| Phage- | T | — | S | — | — | — | — | W | G | — | — | W | — | — | 0.06 | 1.22 | 0.07 | 1318 |
| 1185 | | | | | | | | | | | | | | | | | | |
| Phage- | T | — | S | — | — | — | T | — | S | — | — | — | — | — | 0.06 | 1.22 | 0.07 | 1319 |
| 1186 | | | | | | | | | | | | | | | | | | |
| Phage- | T | — | S | — | — | — | T | — | S | — | — | S | — | F | 0.07 | 1.21 | 0.09 | 1320 |
| 1187 | | | | | | | | | | | | | | | | | | |
| Phage- | L | — | — | — | — | — | T | — | P | — | — | D | — | V | 0.06 | 1.21 | 0.05 | 1321 |
| 1188 | | | | | | | | | | | | | | | | | | |
| Phage- | T | — | — | — | — | — | N | W | P | — | — | E | — | — | 0.07 | 1.21 | 0.08 | 1322 |
| 1189 | | | | | | | | | | | | | | | | | | |
| Phage- | L | — | E | E | F | F | H | — | S | — | — | L | — | — | 0.06 | 1.21 | 0.06 | 1323 |
| 1190 | | | | | | | | | | | | | | | | | | |
| Phage- | P | — | S | D | — | V | A | — | P | — | — | — | — | V | 0.07 | 1.20 | 0.06 | 1324 |
| 1191 | | | | | | | | | | | | | | | | | | |
| Phage- | — | — | — | — | — | — | H | W | S | — | — | V | — | F | 0.06 | 1.20 | 0.08 | 1325 |
| 1192 | | | | | | | | | | | | | | | | | | |
| Phage- | L | — | S | — | — | — | M | — | P | — | — | V | — | — | 0.06 | 1.19 | 0.08 | 1326 |
| 1193 | | | | | | | | | | | | | | | | | | |
| Phage- | — | — | S | — | — | — | T | W | — | — | — | Q | — | F | 0.06 | 1.19 | 0.08 | 1327 |
| 1194 | | | | | | | | | | | | | | | | | | |
| Phage- | — | — | S | — | V | F | — | — | R | — | — | K | — | — | 0.05 | 1.18 | 0.06 | 1328 |
| 1195 | | | | | | | | | | | | | | | | | | |
| Phage- | — | — | S | — | — | — | G | W | — | — | — | M | — | F | 0.06 | 1.16 | 0.08 | 1329 |
| 1196 | | | | | | | | | | | | | | | | | | |
| Phage- | — | — | — | — | — | F | D | — | G | — | — | M | — | — | 0.08 | 1.16 | 0.06 | 1330 |
| 1197 | | | | | | | | | | | | | | | | | | |
| Phage- | — | — | — | D | — | F | N | — | V | — | — | L | — | F | 0.06 | 1.14 | 0.08 | 1331 |
| 1198 | | | | | | | | | | | | | | | | | | |
| Phage- | T | — | — | — | — | — | N | — | S | — | — | A | — | — | 0.06 | 1.14 | 0.08 | 1332 |
| 1199 | | | | | | | | | | | | | | | | | | |
| Phage- | Q | — | — | — | — | — | T | — | P | — | — | V | — | — | 0.08 | 1.13 | 0.09 | 1333 |
| 1200 | | | | | | | | | | | | | | | | | | |
| Phage- | T | — | S | — | — | — | T | — | T | — | — | V | — | F | 0.06 | 1.13 | 0.06 | 1334 |
| 1201 | | | | | | | | | | | | | | | | | | |
| Phage- | T | — | S | — | — | — | H | — | E | — | — | K | — | — | 0.09 | 1.12 | 0.07 | 1335 |
| 1202 | | | | | | | | | | | | | | | | | | |
| Phage- | T | — | S | — | — | — | T | — | S | — | — | L | — | F | 0.07 | 1.12 | 0.09 | 1336 |
| 1203 | | | | | | | | | | | | | | | | | | |
| Phage- | T | — | — | — | — | — | T | — | S | — | — | S | — | F | 0.06 | 1.10 | 0.07 | 1337 |
| 1204 | | | | | | | | | | | | | | | | | | |
| Phage- | T | — | — | — | — | — | N | — | — | — | — | V | — | — | 0.06 | 1.09 | 0.13 | 1338 |
| 1205 | | | | | | | | | | | | | | | | | | |
| Phage- | T | — | — | — | — | — | H | — | E | — | — | Q | — | F | 0.06 | 1.08 | 0.06 | 1339 |
| 1206 | | | | | | | | | | | | | | | | | | |
| Phage- | T | — | — | — | — | — | T | — | N | — | — | W | — | F | 0.07 | 1.07 | 0.07 | 1340 |
| 1207 | | | | | | | | | | | | | | | | | | |
| Phage- | T | — | — | — | — | — | T | W | T | — | — | T | — | M | 0.06 | 1.07 | 0.06 | 1341 |
| 1208 | | | | | | | | | | | | | | | | | | |
| Phage- | P | — | — | — | — | — | Q | W | S | — | — | M | — | — | 0.07 | 1.07 | 0.09 | 1342 |
| 1209 | | | | | | | | | | | | | | | | | | |
| Phage- | — | — | — | — | — | S | T | W | — | — | — | A | — | — | 0.05 | 1.07 | 0.07 | 1343 |
| 1210 | | | | | | | | | | | | | | | | | | |
| Phage- | — | — | — | — | — | F | H | — | E | — | — | Q | — | F | 0.05 | 1.06 | 0.06 | 1344 |
| 1211 | | | | | | | | | | | | | | | | | | |
| Phage- | — | — | T | — | — | F | R | I | H | — | — | M | — | F | 0.06 | 1.06 | 0.12 | 1345 |
| 1212 | | | | | | | | | | | | | | | | | | |
| Phage- | — | — | — | — | — | — | A | — | — | — | — | W | — | — | 0.06 | 1.05 | 0.07 | 1346 |
| 1213 | | | | | | | | | | | | | | | | | | |
| Phage- | M | — | T | — | — | F | — | F | N | — | — | E | — | — | 0.07 | 1.05 | 0.07 | 1347 |
| 1214 | | | | | | | | | | | | | | | | | | |
| Phage- | L | — | D | Q | F | F | H | — | N | — | — | A | — | — | 0.06 | 1.04 | 0.05 | 1348 |
| 1215 | | | | | | | | | | | | | | | | | | |
| Phage- | — | — | — | — | V | F | — | F | N | — | — | E | — | F | 0.07 | 1.01 | 0.07 | 1349 |
| 1216 | | | | | | | | | | | | | | | | | | |
| Phage- | T | — | S | — | — | — | — | — | S | — | — | — | — | M | 0.07 | 1.00 | 0.07 | 1350 |
| 1217 | | | | | | | | | | | | | | | | | | |
| Phage- | W | — | S | E | V | F | — | — | P | — | — | A | — | — | 0.06 | 0.99 | 0.06 | 1351 |
| 1218 | | | | | | | | | | | | | | | | | | |
| Phage- | — | — | T | — | V | F | — | — | — | L | S | P | — | F | 0.05 | 0.99 | 0.05 | 1352 |
| 1219 | | | | | | | | | | | | | | | | | | |
| Phage- | L | — | — | — | V | F | A | — | T | — | — | M | — | F | 0.06 | 0.98 | 0.07 | 1353 |
| 1220 | | | | | | | | | | | | | | | | | | |
| Phage- | P | — | — | — | — | — | H | — | V | — | — | V | — | F | 0.07 | 0.97 | 0.06 | 1354 |
| 1221 | | | | | | | | | | | | | | | | | | |
| Phage- | T | — | S | — | — | — | T | — | K | — | — | T | — | — | 0.05 | 0.97 | 0.07 | 1355 |
| 1222 | | | | | | | | | | | | | | | | | | |
| Phage- | — | — | — | — | — | — | H | W | S | — | — | A | — | — | 0.06 | 0.97 | 0.06 | 1356 |
| 1223 | | | | | | | | | | | | | | | | | | |
| Phage- | M | — | — | — | — | — | M | — | N | — | — | L | — | — | 0.07 | 0.95 | 0.09 | 1357 |
| 1224 | | | | | | | | | | | | | | | | | | |
| Phage- | — | — | — | — | — | — | — | — | S | — | — | L | — | F | 0.06 | 0.95 | 0.08 | 1358 |
| 1225 | | | | | | | | | | | | | | | | | | |
| Phage- | V | — | — | — | — | — | A | — | P | — | — | A | — | — | 0.06 | 0.93 | 0.07 | 1359 |
| 1226 | | | | | | | | | | | | | | | | | | |
| Phage- | T | — | S | — | — | — | H | — | P | — | — | M | — | — | 0.07 | 0.93 | 0.06 | 1360 |
| 1227 | | | | | | | | | | | | | | | | | | |
| Phage- | T | — | — | — | — | — | T | W | V | — | — | T | — | F | 0.07 | 0.92 | 0.07 | 1361 |
| 1228 | | | | | | | | | | | | | | | | | | |
| Phage- | T | — | S | — | — | — | M | — | N | — | — | E | — | F | 0.08 | 0.92 | 0.08 | 1362 |
| 1229 | | | | | | | | | | | | | | | | | | |
| Phage- | T | — | S | — | — | — | M | — | S | — | — | L | — | I | 0.06 | 0.91 | 0.08 | 1363 |
| 1230 | | | | | | | | | | | | | | | | | | |
| Phage- | Q | — | — | — | — | F | — | — | T | — | — | E | — | Y | 0.06 | 0.90 | 0.11 | 1364 |
| 1231 | | | | | | | | | | | | | | | | | | |
| Phage- | W | — | — | — | F | F | H | — | P | — | — | Q | — | F | 0.05 | 0.87 | 0.05 | 1365 |
| 1232 | | | | | | | | | | | | | | | | | | |
| Phage- | R | — | K | — | — | V | P | — | K | — | — | M | — | — | 0.06 | 0.87 | 0.06 | 1366 |
| 1233 | | | | | | | | | | | | | | | | | | |
| Phage- | T | — | — | — | — | — | T | — | E | — | — | S | — | I | 0.07 | 0.86 | 0.09 | 1367 |
| 1234 | | | | | | | | | | | | | | | | | | |
| Phage- | T | — | — | — | — | — | K | — | V | — | — | L | — | F | 0.09 | 0.85 | 0.12 | 1368 |
| 1235 | | | | | | | | | | | | | | | | | | |
| Phage- | L | — | E | — | — | F | N | — | D | — | — | R | — | — | 0.06 | 0.85 | 0.07 | 1369 |
| 1236 | | | | | | | | | | | | | | | | | | |
| Phage- | T | — | — | — | — | — | — | W | D | — | — | F | — | F | 0.07 | 0.82 | 0.08 | 1370 |
| 1237 | | | | | | | | | | | | | | | | | | |
| Phage- | E | — | G | W | E | P | G | Q | H | — | R | V | — | F | 0.06 | 0.82 | 0.06 | 1371 |
| 1238 | | | | | | | | | | | | | | | | | | |
| Phage- | — | — | — | — | — | — | T | — | E | — | — | T | — | — | 0.08 | 0.81 | 0.07 | 1372 |
| 1239 | | | | | | | | | | | | | | | | | | |
| Phage- | — | — | — | — | — | — | T | — | K | — | — | A | — | F | 0.05 | 0.80 | 0.06 | 1373 |
| 1240 | | | | | | | | | | | | | | | | | | |
| Phage- | — | — | S | — | — | — | M | W | L | — | — | E | — | F | 0.05 | 0.80 | 0.06 | 1374 |
| 1241 | | | | | | | | | | | | | | | | | | |
| Phage- | W | — | S | — | V | F | — | — | Q | — | — | P | — | F | 0.06 | 0.78 | 0.06 | 1375 |
| 1242 | | | | | | | | | | | | | | | | | | |
| Phage- | T | — | S | — | — | — | T | — | K | — | — | M | — | F | 0.06 | 0.77 | 0.07 | 1376 |
| 1243 | | | | | | | | | | | | | | | | | | |
| Phage- | T | — | S | — | — | — | T | — | G | — | — | A | — | F | 0.07 | 0.76 | 0.07 | 1377 |
| 1244 | | | | | | | | | | | | | | | | | | |
| Phage- | I | — | — | — | — | — | G | W | P | — | — | W | — | — | 0.08 | 0.75 | 0.10 | 1378 |
| 1245 | | | | | | | | | | | | | | | | | | |
| Phage- | E | — | S | — | — | — | M | W | G | — | — | A | — | F | 0.06 | 0.73 | 0.09 | 1379 |
| 1246 | | | | | | | | | | | | | | | | | | |
| Phage- | T | — | — | — | — | — | T | — | D | — | — | S | — | — | 0.06 | 0.71 | 0.08 | 1380 |
| 1247 | | | | | | | | | | | | | | | | | | |
| Phage- | T | — | — | — | — | — | V | W | Q | — | — | T | — | F | 0.06 | 0.71 | 0.06 | 1381 |
| 1248 | | | | | | | | | | | | | | | | | | |
| Phage- | P | — | S | — | — | — | T | — | — | — | — | — | — | — | 0.06 | 0.71 | 0.09 | 1382 |
| 1249 | | | | | | | | | | | | | | | | | | |
| Phage- | T | — | — | — | — | — | Q | L | E | — | — | W | — | F | 0.09 | 0.70 | 0.07 | 1383 |
| 1250 | | | | | | | | | | | | | | | | | | |
| Phage- | G | — | — | — | — | — | E | — | N | — | — | T | — | — | 0.05 | 0.69 | 0.64 | 1384 |
| 1251 | | | | | | | | | | | | | | | | | | |
| Phage- | T | — | — | — | — | — | A | — | T | — | — | H | — | F | 0.09 | 0.69 | 0.11 | 1385 |
| 1252 | | | | | | | | | | | | | | | | | | |
| Phage- | T | — | S | — | — | — | Q | W | E | — | — | A | — | — | 0.06 | 0.67 | 0.08 | 1386 |
| 1253 | | | | | | | | | | | | | | | | | | |
| Phage- | W | — | S | — | V | F | — | — | Q | — | — | V | — | — | 0.07 | 0.61 | 0.06 | 1387 |
| 1254 | | | | | | | | | | | | | | | | | | |
| Phage- | — | — | S | — | — | — | — | — | — | — | — | M | — | I | 0.06 | 0.59 | 0.06 | 1388 |
| 1255 | | | | | | | | | | | | | | | | | | |
| Phage- | D | — | W | W | D | S | P | — | H | — | R | V | — | — | 0.06 | 0.57 | 0.07 | 1389 |
| 1256 | | | | | | | | | | | | | | | | | | |
| Phage- | — | — | — | — | — | — | G | — | G | — | — | Q | — | — | 0.07 | 0.55 | 0.13 | 1390 |
| 1257 | | | | | | | | | | | | | | | | | | |
| Phage- | — | — | G | G | — | F | H | — | S | — | — | L | — | F | 0.07 | 0.55 | 0.15 | 1391 |
| 1258 | | | | | | | | | | | | | | | | | | |
| Phage- | G | — | — | — | — | F | H | — | T | L | S | T | — | — | 0.05 | 0.42 | 0.05 | 1392 |
| 1259 | | | | | | | | | | | | | | | | | | |

(—) indicates the same amino acid position as in Peptide-44 corresponding position.

### Example 10. Discovery of Peptides that Bind to Anti-CD28 scFv

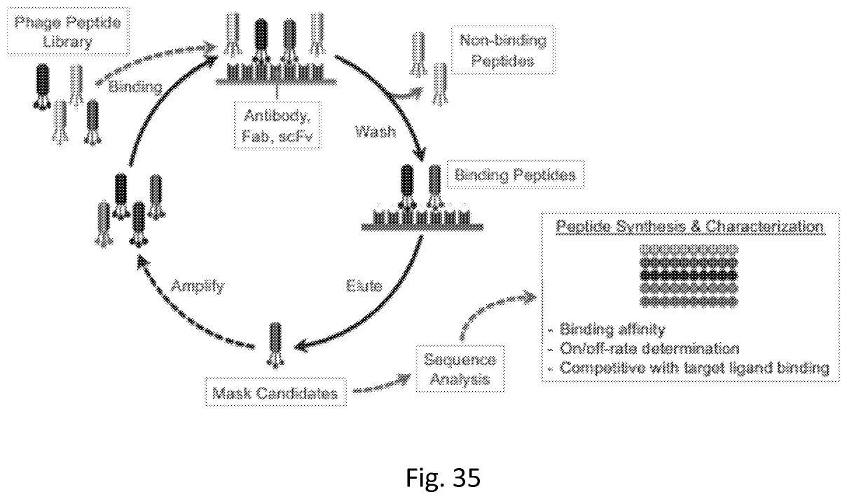

Lead peptides that mask the anti-CD28 scFv according to SEQ ID NO: 35 were identified by phage display according to the method of FIG. 35. Lead hits were then synthesized as peptides and evaluated as described below. Synthetic peptides were evaluated for their ability to bind human anti-CD28 scFv (SEQ ID NO: 35) in a standard enzyme linked immunosorbent assay (ELISA) format. Briefly, biotinylated peptides were captured on neutravidin coated plates. Anti-CD28 scFv diluted in buffer was then added to the peptide captured plates. Bound anti-CD28 scFv was detected using a standard horse radish peroxidase conjugate secondary antibody. The concentration of anti-CD28 scFv required to achieve 50% maximal signal (EC₅₀) was calculated using Graphpad Prism software. Peptides were also evaluated for their ability to inhibit anti-CD28 scFv from binding its cognate antigen, CD28. Briefly, biotinylated CD28 antigen was captured on neutravidin coated plates. Anti-CD28 scFv at 2 nM was pre-incubated with 0-100 μM titrated peptides. After a short pre-incubation period the mixture of titrated peptides with fixed anti-CD28 scFv (2 nM) was added to the CD28 antigen captured plates. After a short incubation on the plates, bound anti-CD28 scFv was detected with a standard horse radish peroxidase conjugated secondary antibody. The concentration of peptide required to reduce the max signal by 50% (IC50) was calculated in Graphpad Prism software.

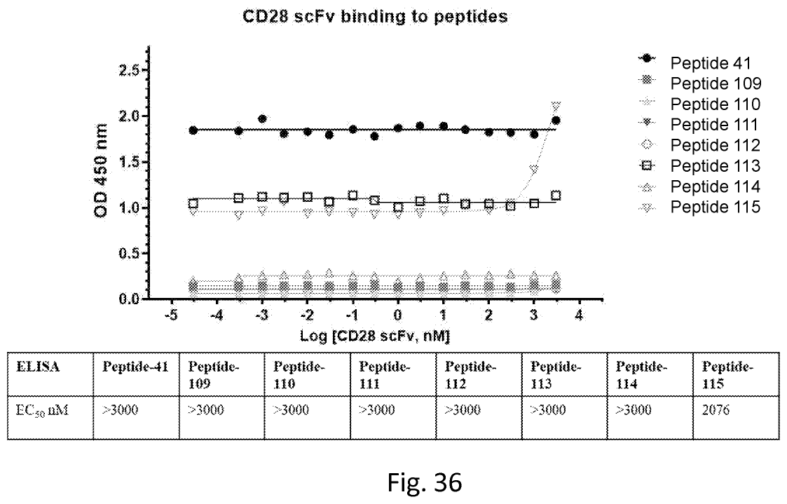

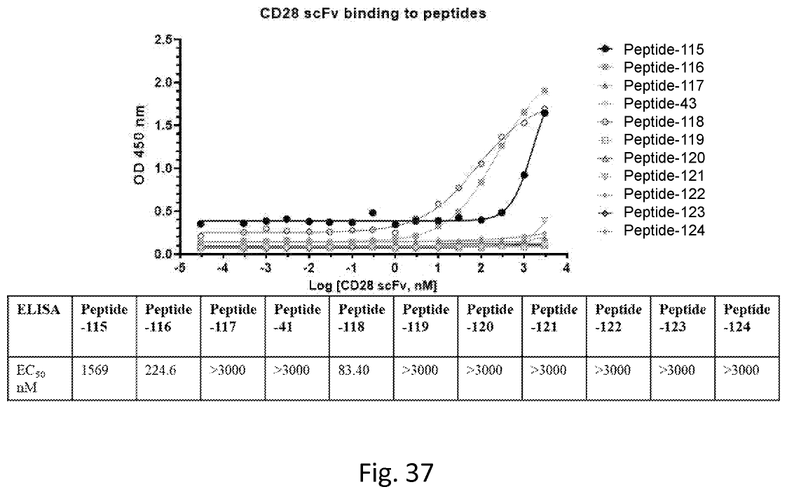

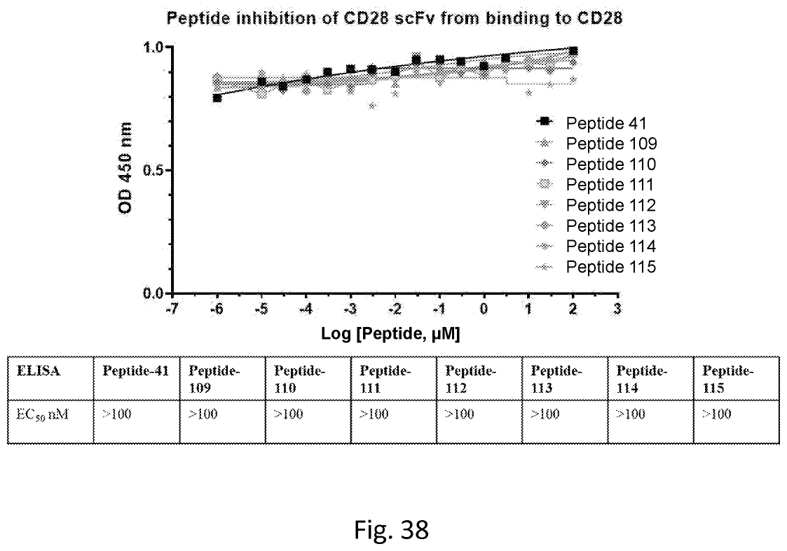

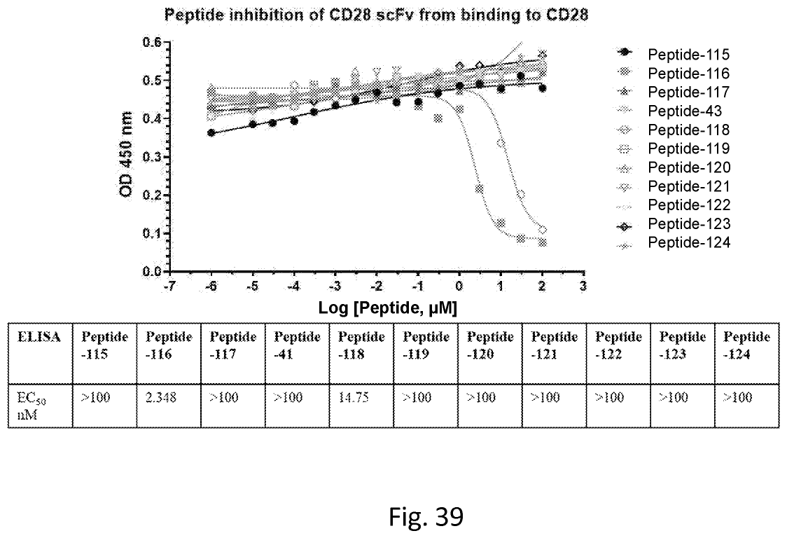

FIG. 36 illustrates anti-CD28 scFv (SEQ ID NO: 35) binding to peptides measured by ELISA. FIG. 37 illustrates anti-CD28 scFv binding to peptides measured by ELISA. FIGS. 38-39 illustrate that peptides inhibit anti-CD28 scFv from binding to CD28 antigen as measured by ELISA.

### Example 11. Kinetic Binding Assays of Anti-CD28 scFv (SEQ ID NO: 35) to Peptides-116 and -118

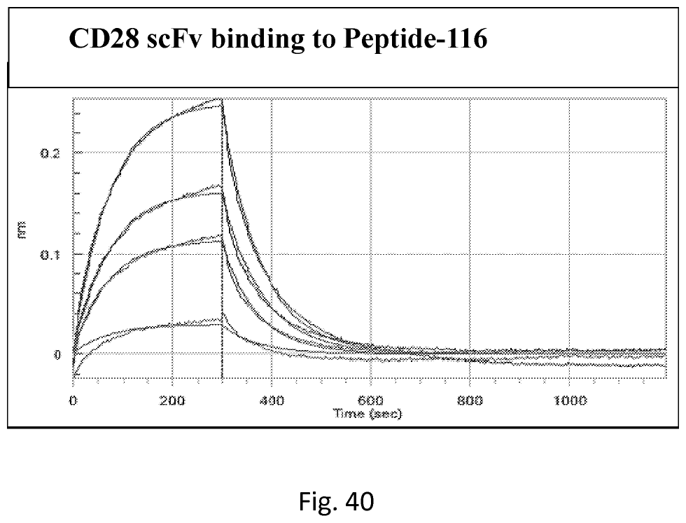

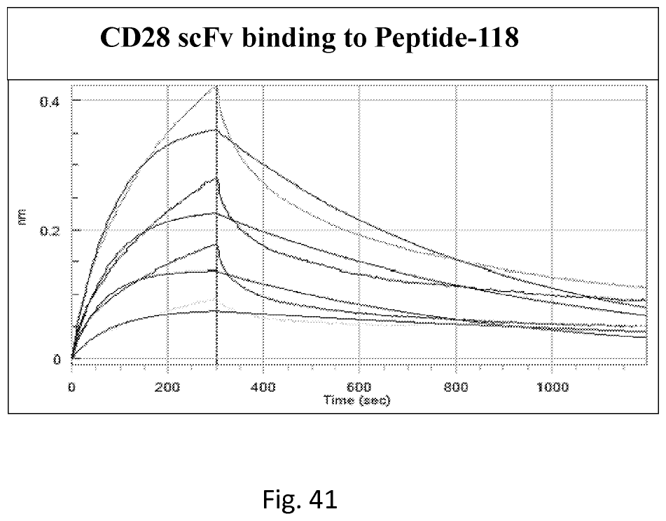

This Example assesses binding of anti-CD28 scFv to Peptide-116 and Peptide-118 in an in vitro kinetic binding assay. Kinetic binding of anti-CD28 scFv to Peptide-116 and Peptide-118 were evaluated by bio-layer interferometry using an Octet RED96 instrument. Briefly, streptavidin biosensors were loaded with biotinylated peptides and baselined in buffer. Anti-CD28 scFv were titrated in solution at 100 nM, 50 nM, 25 nM, and 12.5 nM, then associated onto the peptide loaded sensors. After a short association period, sensors were transferred into buffer and the dissociation of bound anti-CD28 scFv was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. Peptide-116 and peptide-118 kinetic binding sensorgrams are shown in FIGS. 40-41 and are summarized in Tables 27-28.

TABLE 27
Timing and Steps of Assay

| Step | Time |
|---|---|
| Baseline: Buffer | 60 sec |
| Load: | 300 sec |
| 200 nM Peptide-116 | |
| 200 nM Peptide-118 | |
| Baseline: Buffer | 300 sec |
| Association in octet buffer | 300 sec |
| 100 nM CD28 scFv | |
| 50 nM CD28 scFv | |
| 25 nM CD28 scFv | |
| 12.5 nM CD28 scFv | |
| Dissociation: Buffer | 900 sec |

TABLE 28
Binding Kinetics Summary of anti-CD28
scFv to Peptide-116 and Peptide-118

| Binding domain | Peptide | KD (M) | kon(1/Ms) | kdis(1/s) |
|---|---|---|---|---|
| CD28 scFv | Peptide-116 | 3.77E−07 | 3.25E+04 | 1.23E−02 |
| CD28 scFv | Peptide-118 | 1.70E−08 | 9.87E+04 | 1.67E−03 |

### Example 12: Optimized Phage Library Construction—Anti-CD28 scFv (SEQ ID NO: 35) Peptide-116

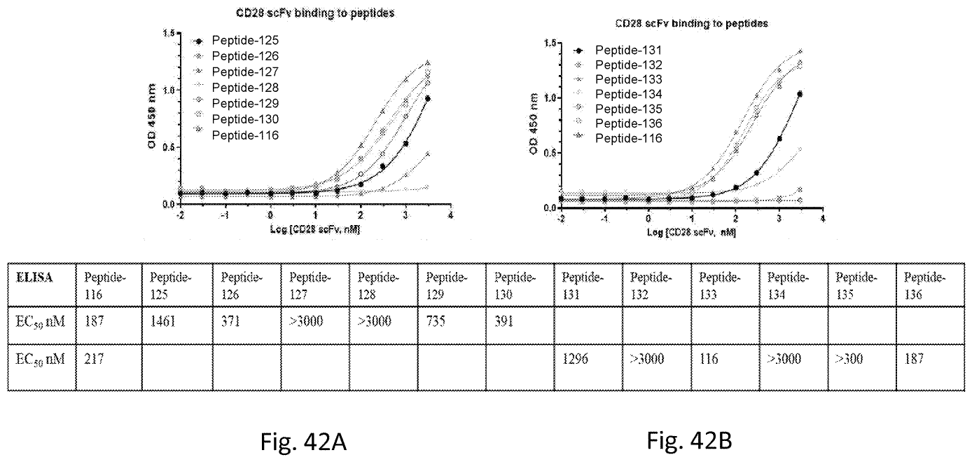

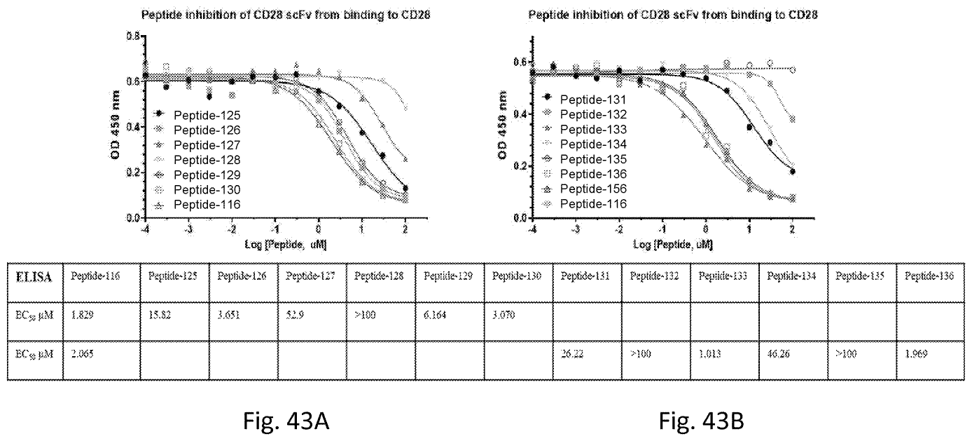

Sequence activity relationships were established for Peptide-116 by mutating each individual residue within the peptide to alanine and measuring binding and inhibition against anti-CD28 scFv. Peptide residues whose alanine mutations significantly weakened binding and inhibition were considered key residues where mutations were not tolerated. Peptide residues whose alanine mutations performed similarly to the non-mutated sequence were considered non-critical sites where mutations were indeed tolerated. Using the peptide sequence activity relationships (SAR), DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field. FIG. 42A and FIG. 42B demonstrate anti-CD28 scFv binding of alanine scanning peptides of Peptide-116 according to the ELISA protocol of Example 10. FIG. 43A and FIG. 43B demonstrate anti-CD28 scFv inhibition of alanine scanning peptides of Peptide-116 according to the ELISA protocol of Example 10.

### Example 13: Panning ELISAs—Anti-CD28 scFv Peptides

Clonal phage were harvested as crude supernatants and screened via standard enzyme linked immunosorbent assays (ELISAs). Briefly, biotinylated anti-CD28 scFv was captured on neutravidin coated plates. Prior to the addition of clonal phage, wells were incubated with blocking buffer and CD28 soluble protein or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest were then sent for sequence analysis.

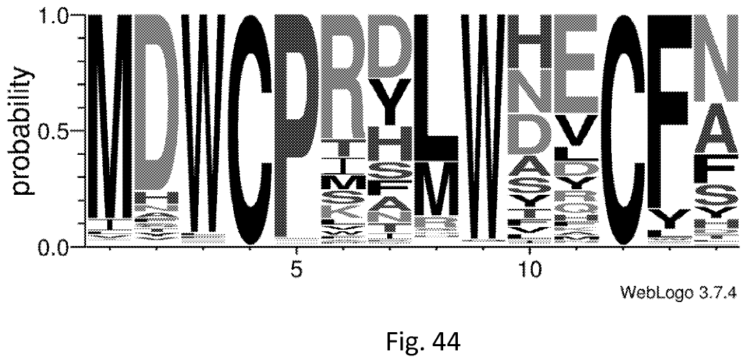

Phage panning results of anti-CD28 scFv Peptide-116 library sequences are shown in Table 29. 453 clonal phage sequences were identified. The consensus sequence calculated from all the sequences of Table 30 is shown in FIG. 44 and was generated using WebLogo 3.7.4.

TABLE 29
Phage panning results of Anti-CD28 scFv Peptide-116 library sequences.

| | | | | | | | | | | | | | | | Phage binding ELISA | | | |
|---|---|---|---|---|---|---|---|---|---|---|---|---|---|---|---|---|---|---|
| | | | | | | | | | | | | | | | | | CD28 | |
| | | | | | | | | | | | | | | | | | scFv | |
| | | | | | | | | | | | | | | | Back- | CD28 | signal in | |
| Phage | Amino acid position sequence | | | | | | | | | | | | | | groud | scFv | presence | SEQ |
| ID | 1 | 2 | 3 | 4 | 5 | 6 | 7 | 8 | 9 | 10 | 11 | 12 | 13 | 14 | signal | signal | of CD28 | ID NO: |
| Phage- | M | D | W | C | P | R | E | R | W | V | D | C | F | F | 0.084 | 2.042 | 0.489 | 1517 |
| 1260 | | | | | | | | | | | | | | | | | | |
| Phage- | — | — | — | — | — | I | D | L | — | N | E | — | — | — | 0.066 | 2.515 | 0.187 | 1558 |
| 1261 | | | | | | | | | | | | | | | | | | |
| Phage- | — | — | — | — | — | I | H | L | — | H | V | — | — | N | 0.082 | 2.526 | 0.167 | 1559 |
| 1262 | | | | | | | | | | | | | | | | | | |
| Phage- | — | — | — | — | — | I | Y | L | — | S | E | — | — | N | 0.075 | 2.635 | 0.513 | 1560 |
| 1263 | | | | | | | | | | | | | | | | | | |
| Phage- | — | N | — | — | — | K | D | I | — | Y | L | — | — | N | 0.073 | 2.625 | 0.168 | 1561 |
| 1264 | | | | | | | | | | | | | | | | | | |
| Phage- | — | — | — | — | — | L | H | M | — | H | E | — | — | S | 0.086 | 2.511 | 0.151 | 1562 |
| 1265 | | | | | | | | | | | | | | | | | | |
| Phage- | — | — | — | — | — | L | Y | L | — | N | E | — | — | N | 0.065 | 2.612 | 0.247 | 1563 |
| 1266 | | | | | | | | | | | | | | | | | | |
| Phage- | — | — | — | — | — | — | D | L | — | D | L | — | — | A | 0.078 | 2.696 | 0.219 | 1564 |
| 1267 | | | | | | | | | | | | | | | | | | |
| Phage- | — | — | — | — | — | — | D | L | — | H | E | — | — | A | 0.063 | 2.71 | 0.277 | 1565 |
| 1268 | | | | | | | | | | | | | | | | | | |
| Phage- | — | — | — | — | — | — | D | L | — | H | L | — | — | S | 0.059 | 2.592 | 0.29 | 1566 |
| 1269 | | | | | | | | | | | | | | | | | | |
| Phage- | — | — | — | — | — | — | D | L | — | S | E | — | — | — | 0.068 | 2.574 | 0.218 | 1567 |
| 1270 | | | | | | | | | | | | | | | | | | |
| Phage- | — | — | — | — | — | — | D | L | — | — | H | — | — | A | 0.062 | 2.554 | 0.179 | 1568 |
| 1271 | | | | | | | | | | | | | | | | | | |
| Phage- | — | — | — | — | — | — | D | M | — | D | E | — | — | A | 0.116 | 2.593 | 0.25 | 1569 |
| 1272 | | | | | | | | | | | | | | | | | | |
| Phage- | — | — | — | — | — | — | D | M | — | S | E | — | — | A | 0.069 | 2.701 | 0.293 | 1570 |
| 1273 | | | | | | | | | | | | | | | | | | |
| Phage- | — | — | — | — | — | — | D | M | — | S | V | — | — | S | 0.062 | 2.619 | 0.207 | 1571 |
| 1274 | | | | | | | | | | | | | | | | | | |
| Phage- | — | — | — | — | — | — | F | M | — | D | E | — | — | N | 0.079 | 2.68 | 0.256 | 1572 |
| 1275 | | | | | | | | | | | | | | | | | | |
| Phage- | — | — | — | — | — | — | H | M | — | N | Y | — | — | A | 0.089 | 2.712 | 0.242 | 1573 |
| 1276 | | | | | | | | | | | | | | | | | | |
| Phage- | — | — | — | — | — | — | S | L | — | H | E | — | — | A | 0.064 | 2.544 | 0.255 | 1574 |
| 1277 | | | | | | | | | | | | | | | | | | |
| Phage- | — | — | — | — | — | — | Y | L | — | H | V | — | — | A | 0.072 | 2.475 | 0.226 | 1575 |
| 1278 | | | | | | | | | | | | | | | | | | |
| Phage- | — | H | — | — | — | V | D | L | — | Y | L | — | Y | N | 0.079 | 2.659 | 0.417 | 1576 |
| 1279 | | | | | | | | | | | | | | | | | | |
| Phage- | — | — | — | — | — | V | H | L | — | S | V | — | — | A | 0.072 | 2.65 | 0.302 | 1577 |
| 1280 | | | | | | | | | | | | | | | | | | |
| Phage- | — | — | — | — | — | M | H | L | — | H | Q | — | — | N | 0.073 | 2.555 | 0.299 | 1578 |
| 1281 | | | | | | | | | | | | | | | | | | |
| Phage- | — | — | — | — | — | I | D | M | — | D | Q | — | — | N | 0.061 | 2.661 | 0.297 | 1579 |
| 1282 | | | | | | | | | | | | | | | | | | |
| Phage- | — | A | — | — | — | — | D | K | — | S | E | — | — | S | 0.062 | 0.853 | 0.147 | 1580 |
| 1283 | | | | | | | | | | | | | | | | | | |
| Phage- | — | — | — | — | — | — | H | L | — | — | H | — | — | N | 0.071 | 2.505 | 0.254 | 1581 |
| 1284 | | | | | | | | | | | | | | | | | | |
| Phage- | — | — | — | — | — | — | A | L | — | H | E | — | — | Y | 0.115 | 2.499 | 0.252 | 1582 |
| 1285 | | | | | | | | | | | | | | | | | | |
| Phage- | — | — | — | — | — | I | A | L | — | A | E | — | — | N | 0.111 | 2.458 | 0.224 | 1583 |
| 1286 | | | | | | | | | | | | | | | | | | |
| Phage- | — | — | — | — | — | — | P | L | — | H | E | — | — | S | 0.078 | 2.346 | 0.121 | 1584 |
| 1287 | | | | | | | | | | | | | | | | | | |
| Phage- | — | H | — | — | — | I | D | L | — | A | E | — | Y | A | 0.082 | 2.278 | 0.225 | 1585 |
| 1288 | | | | | | | | | | | | | | | | | | |
| Phage- | — | A | — | — | — | V | Y | L | — | H | E | — | — | N | 0.094 | 2.273 | 0.351 | 1586 |
| 1289 | | | | | | | | | | | | | | | | | | |
| Phage- | I | — | — | — | — | — | Y | L | — | D | E | — | Y | N | 0.061 | 2.243 | 0.153 | 1587 |
| 1290 | | | | | | | | | | | | | | | | | | |
| Phage- | — | S | — | — | — | I | H | L | — | N | E | — | — | N | 0.083 | 2.057 | 0.284 | 1588 |
| 1291 | | | | | | | | | | | | | | | | | | |
| Phage- | — | — | — | — | — | P | Y | L | — | N | V | — | — | S | 0.063 | 1.639 | 0.1 | 1589 |
| 1292 | | | | | | | | | | | | | | | | | | |
| Phage- | — | — | — | — | — | M | D | L | — | D | Y | — | — | N | 0.071 | 1.635 | 0.107 | 1590 |
| 1293 | | | | | | | | | | | | | | | | | | |
| Phage- | — | — | — | — | — | I | N | L | — | D | E | — | — | S | 0.062 | 1.498 | 0.096 | 1591 |
| 1294 | | | | | | | | | | | | | | | | | | |
| Phage- | — | — | — | — | — | M | H | L | — | N | K | — | — | N | 0.071 | 1.043 | 0.122 | 1592 |
| 1295 | | | | | | | | | | | | | | | | | | |
| Phage- | — | N | — | — | — | — | D | M | — | Y | Q | — | — | N | 0.068 | 1.948 | 0.177 | 1593 |
| 1296 | | | | | | | | | | | | | | | | | | |
| Phage- | Q | — | — | — | — | — | H | M | — | S | F | — | — | H | 0.138 | 0.074 | 0.081 | 1594 |
| 1297 | | | | | | | | | | | | | | | | | | |
| Phage- | — | H | — | — | — | M | D | Q | — | S | N | — | — | N | 0.062 | 0.515 | 0.084 | 1595 |
| 1298 | | | | | | | | | | | | | | | | | | |
| Phage- | — | — | — | — | — | — | H | — | — | — | — | — | — | Y | 0.073 | 0.478 | 0.11 | 1596 |
| 1299 | | | | | | | | | | | | | | | | | | |
| Phage- | — | H | — | — | — | I | D | — | — | A | — | — | — | N | 0.074 | 0.287 | 0.077 | 1597 |
| 1300 | | | | | | | | | | | | | | | | | | |
| Phage- | — | — | — | — | — | — | D | S | — | H | L | — | — | A | 0.073 | 2.446 | 0.147 | 1598 |
| 1301 | | | | | | | | | | | | | | | | | | |
| Phage- | — | — | — | — | — | — | T | L | — | Y | L | — | — | N | 0.508 | 2.593 | 1.198 | 1599 |
| 1302 | | | | | | | | | | | | | | | | | | |
| Phage- | — | V | S | — | — | T | T | M | — | N | R | — | — | N | 0.079 | 1.329 | 0.072 | 1600 |
| 1303 | | | | | | | | | | | | | | | | | | |
| Phage- | — | — | — | — | — | S | Y | L | — | N | E | — | — | — | 0.065 | 1.995 | 0.133 | 1601 |
| 1304 | | | | | | | | | | | | | | | | | | |
| Phage- | — | — | — | — | — | — | A | L | — | A | E | — | — | N | 0.068 | 2.676 | 0.339 | 1602 |
| 1305 | | | | | | | | | | | | | | | | | | |
| Phage- | — | — | — | — | — | M | Y | L | — | N | E | — | — | N | 0.064 | 2.609 | 0.471 | 1603 |
| 1306 | | | | | | | | | | | | | | | | | | |
| Phage- | — | — | — | — | — | — | Y | L | — | N | E | — | — | Y | 0.079 | 2.537 | 0.358 | 1604 |
| 1307 | | | | | | | | | | | | | | | | | | |
| Phage- | — | N | — | — | — | T | A | L | — | H | V | — | — | N | 0.067 | 1.644 | 0.169 | 1605 |
| 1308 | | | | | | | | | | | | | | | | | | |
| Phage- | — | — | — | — | — | M | Y | M | — | Y | E | — | — | A | 0.08 | 2.479 | 0.358 | 1606 |
| 1309 | | | | | | | | | | | | | | | | | | |
| Phage- | — | — | — | — | — | I | H | M | — | A | — | — | — | A | 0.065 | 2.457 | 0.143 | 1607 |
| 1310 | | | | | | | | | | | | | | | | | | |
| Phage- | I | T | — | — | — | S | S | M | — | N | R | — | — | I | 0.065 | 0.379 | 0.11 | 1608 |
| 1311 | | | | | | | | | | | | | | | | | | |
| Phage- | — | — | — | — | — | — | Y | L | — | H | E | — | — | A | 0.071 | 2.628 | 0.429 | 1609 |
| 1312 | | | | | | | | | | | | | | | | | | |
| Phage- | — | F | — | — | — | T | T | M | — | N | R | — | — | T | 0.111 | 0.662 | 0.338 | 1610 |
| 1313 | | | | | | | | | | | | | | | | | | |
| Phage- | — | — | — | — | — | — | A | L | — | H | E | — | — | N | 0.071 | 2.686 | 0.401 | 1611 |
| 1314 | | | | | | | | | | | | | | | | | | |
| Phage- | — | — | — | — | — | — | D | M | — | L | F | — | Y | N | 0.097 | 2.707 | 1.023 | 1612 |
| 1315 | | | | | | | | | | | | | | | | | | |
| Phage- | — | — | — | — | — | — | S | H | — | H | V | — | Y | N | 0.072 | 1.951 | 0.281 | 1613 |
| 1316 | | | | | | | | | | | | | | | | | | |
| Phage- | — | — | — | — | — | — | Y | L | — | S | — | — | — | A | 0.084 | 2.727 | 1.53 | 1614 |
| 1317 | | | | | | | | | | | | | | | | | | |
| Phage- | — | — | — | — | — | F | Y | L | — | D | E | — | — | N | 0.092 | 2.672 | 0.291 | 1615 |
| 1318 | | | | | | | | | | | | | | | | | | |
| Phage- | — | — | — | — | — | — | H | L | — | H | E | — | — | N | 0.07 | 2.654 | 0.704 | 1616 |
| 1319 | | | | | | | | | | | | | | | | | | |
| Phage- | — | — | — | — | — | — | D | L | — | H | E | — | — | S | 0.066 | 2.649 | 0.382 | 1617 |
| 1320 | | | | | | | | | | | | | | | | | | |
| Phage- | — | — | — | — | — | M | Y | L | — | N | E | — | — | S | 0.071 | 2.641 | 0.411 | 1618 |
| 1321 | | | | | | | | | | | | | | | | | | |
| Phage- | — | — | — | — | — | — | F | L | — | S | V | — | — | N | 0.515 | 2.637 | 1.151 | 1619 |
| 1322 | | | | | | | | | | | | | | | | | | |
| Phage- | — | — | — | — | — | — | D | L | — | T | E | — | — | A | 0.06 | 2.634 | 0.151 | 1620 |
| 1323 | | | | | | | | | | | | | | | | | | |
| Phage- | — | — | — | — | — | — | F | L | — | D | E | — | — | N | 0.065 | 2.629 | 0.483 | 1621 |
| 1324 | | | | | | | | | | | | | | | | | | |
| Phage- | — | — | — | — | — | K | Y | L | — | S | V | — | — | — | 0.184 | 2.622 | 0.826 | 1622 |
| 1325 | | | | | | | | | | | | | | | | | | |
| Phage- | — | — | — | — | — | M | D | L | — | Y | Q | — | — | N | 0.063 | 2.617 | 0.187 | 1623 |
| 1326 | | | | | | | | | | | | | | | | | | |
| Phage- | — | — | — | — | — | — | H | L | — | A | E | — | — | — | 0.066 | 2.611 | 0.292 | 1624 |
| 1327 | | | | | | | | | | | | | | | | | | |
| Phage- | — | — | — | — | — | K | D | L | — | Y | L | — | — | A | 0.2 | 2.608 | 0.213 | 1625 |
| 1328 | | | | | | | | | | | | | | | | | | |
| Phage- | — | — | — | — | — | I | H | L | — | H | Y | — | — | N | 0.078 | 2.602 | 0.453 | 1626 |
| 1329 | | | | | | | | | | | | | | | | | | |
| Phage- | — | — | — | — | — | — | A | L | — | N | V | — | — | N | 0.065 | 2.598 | 0.472 | 1627 |
| 1330 | | | | | | | | | | | | | | | | | | |
| Phage- | — | — | — | — | — | I | D | L | — | H | L | — | — | Y | 0.062 | 2.59 | 0.488 | 1628 |
| 1331 | | | | | | | | | | | | | | | | | | |
| Phage- | — | — | — | — | — | — | D | L | — | F | L | — | Y | N | 0.06 | 2.587 | 0.424 | 1629 |
| 1332 | | | | | | | | | | | | | | | | | | |
| Phage- | — | — | — | — | — | — | H | L | — | H | E | — | — | — | 0.364 | 2.583 | 0.373 | 1630 |
| 1333 | | | | | | | | | | | | | | | | | | |
| Phage- | — | — | — | — | — | — | Y | L | — | T | V | — | — | S | 0.115 | 2.582 | 0.372 | 1631 |
| 1334 | | | | | | | | | | | | | | | | | | |
| Phage- | — | — | — | — | — | — | D | L | — | S | L | — | — | Y | 0.065 | 2.573 | 0.246 | 1632 |
| 1335 | | | | | | | | | | | | | | | | | | |
| Phage- | — | — | — | — | — | — | F | L | — | S | E | — | — | N | 0.062 | 2.571 | 0.227 | 1633 |
| 1336 | | | | | | | | | | | | | | | | | | |
| Phage- | — | — | — | — | — | — | T | L | — | A | Y | — | — | N | 0.07 | 2.571 | 0.584 | 1634 |
| 1337 | | | | | | | | | | | | | | | | | | |
| Phage- | — | — | — | — | — | K | D | L | — | D | Y | — | — | A | 0.078 | 2.568 | 0.165 | 1635 |
| 1338 | | | | | | | | | | | | | | | | | | |
| Phage- | — | — | — | — | — | K | Y | L | — | D | V | — | — | N | 0.096 | 2.566 | 0.422 | 1636 |
| 1339 | | | | | | | | | | | | | | | | | | |
| Phage- | — | — | — | — | — | — | Y | L | — | N | M | — | — | H | 0.114 | 2.564 | 0.404 | 1637 |
| 1340 | | | | | | | | | | | | | | | | | | |
| Phage- | — | — | — | — | — | — | Y | L | — | T | E | — | — | N | 0.062 | 2.56 | 0.549 | 1638 |
| 1341 | | | | | | | | | | | | | | | | | | |
| Phage- | — | — | — | — | — | — | S | L | — | H | Y | — | — | A | 0.14 | 2.552 | 0.338 | 1639 |
| 1342 | | | | | | | | | | | | | | | | | | |
| Phage- | — | — | — | — | — | — | Y | L | — | A | E | — | — | Y | 0.063 | 2.552 | 0.354 | 1640 |
| 1343 | | | | | | | | | | | | | | | | | | |
| Phage- | — | — | — | — | — | — | D | L | — | H | E | — | — | N | 0.074 | 2.552 | 0.218 | 1641 |
| 1344 | | | | | | | | | | | | | | | | | | |
| Phage- | — | — | — | — | — | — | D | L | — | D | L | — | — | — | 0.076 | 2.549 | 0.156 | 1642 |
| 1345 | | | | | | | | | | | | | | | | | | |
| Phage- | — | — | — | — | — | — | Y | L | — | N | V | — | — | N | 0.087 | 2.546 | 0.57 | 1643 |
| 1346 | | | | | | | | | | | | | | | | | | |
| Phage- | — | — | — | — | — | I | Y | L | — | D | E | — | — | N | 0.061 | 2.546 | 0.215 | 1644 |
| 1347 | | | | | | | | | | | | | | | | | | |
| Phage- | — | — | — | — | — | — | D | L | — | A | E | — | — | N | 0.061 | 2.537 | 0.207 | 1645 |
| 1348 | | | | | | | | | | | | | | | | | | |
| Phage- | — | — | — | — | — | — | A | L | — | H | E | — | — | T | 0.075 | 2.536 | 0.191 | 1646 |
| 1349 | | | | | | | | | | | | | | | | | | |
| Phage- | — | — | — | — | — | K | N | L | — | H | V | — | — | N | 0.081 | 2.53 | 0.248 | 1647 |
| 1350 | | | | | | | | | | | | | | | | | | |
| Phage- | — | — | — | — | — | — | Y | L | — | D | E | — | — | N | 0.069 | 2.529 | 0.283 | 1648 |
| 1351 | | | | | | | | | | | | | | | | | | |
| Phage- | — | — | — | — | — | F | Y | L | — | N | E | — | — | Y | 0.099 | 2.528 | 0.594 | 1649 |
| 1352 | | | | | | | | | | | | | | | | | | |
| Phage- | — | H | — | — | — | — | A | L | — | D | V | — | Y | N | 0.077 | 2.528 | 0.24 | 1650 |
| 1353 | | | | | | | | | | | | | | | | | | |
| Phage- | — | — | — | — | — | — | D | L | — | N | V | — | — | — | 0.164 | 2.52 | 0.137 | 1651 |
| 1354 | | | | | | | | | | | | | | | | | | |
| Phage- | — | — | — | — | — | — | Y | L | — | F | E | — | — | A | 0.066 | 2.504 | 0.21 | 1652 |
| 1355 | | | | | | | | | | | | | | | | | | |
| Phage- | — | — | — | — | — | — | Y | L | — | H | E | — | — | N | 0.105 | 2.49 | 0.421 | 1653 |
| 1356 | | | | | | | | | | | | | | | | | | |
| Phage- | — | — | — | — | — | — | D | L | — | Y | A | — | — | A | 0.072 | 2.474 | 0.242 | 1654 |
| 1357 | | | | | | | | | | | | | | | | | | |
| Phage- | — | — | — | — | — | — | Y | L | — | F | E | — | — | S | 0.079 | 2.462 | 0.235 | 1655 |
| 1358 | | | | | | | | | | | | | | | | | | |
| Phage- | — | — | — | — | — | — | H | L | — | D | E | — | — | — | 0.074 | 2.459 | 0.663 | 1656 |
| 1359 | | | | | | | | | | | | | | | | | | |
| Phage- | — | — | — | — | — | — | Y | L | — | H | M | — | Y | S | 0.129 | 2.419 | 1.166 | 1657 |
| 1360 | | | | | | | | | | | | | | | | | | |
| Phage- | — | — | — | — | — | — | D | L | — | H | A | — | — | S | 0.074 | 2.414 | 0.173 | 1658 |
| 1361 | | | | | | | | | | | | | | | | | | |
| Phage- | — | — | — | — | — | — | D | L | — | H | V | — | — | — | 0.068 | 2.412 | 0.268 | 1659 |
| 1362 | | | | | | | | | | | | | | | | | | |
| Phage- | — | — | — | — | — | — | D | L | — | D | Q | — | Y | A | 0.059 | 2.406 | 0.201 | 1660 |
| 1363 | | | | | | | | | | | | | | | | | | |
| Phage- | — | — | — | — | — | I | H | L | — | N | E | — | — | A | 0.104 | 2.405 | 0.203 | 1661 |
| 1364 | | | | | | | | | | | | | | | | | | |
| Phage- | — | — | — | — | — | — | P | L | — | D | M | — | — | — | 0.063 | 2.403 | 0.138 | 1662 |
| 1365 | | | | | | | | | | | | | | | | | | |
| Phage- | — | — | — | — | — | V | S | L | — | H | V | — | — | Y | 0.107 | 2.401 | 0.409 | 1663 |
| 1366 | | | | | | | | | | | | | | | | | | |
| Phage- | — | — | — | — | — | — | F | L | — | N | E | — | — | N | 0.072 | 2.4 | 0.503 | 1664 |
| 1367 | | | | | | | | | | | | | | | | | | |
| Phage- | — | — | — | — | — | — | A | L | — | N | E | — | — | A | 0.08 | 2.385 | 0.375 | 1665 |
| 1368 | | | | | | | | | | | | | | | | | | |
| Phage- | — | — | — | — | — | — | D | L | — | I | E | — | — | — | 0.084 | 2.372 | 0.137 | 1666 |
| 1369 | | | | | | | | | | | | | | | | | | |
| Phage- | — | — | — | — | — | S | Y | L | — | T | V | — | — | A | 0.062 | 2.34 | 0.096 | 1667 |
| 1370 | | | | | | | | | | | | | | | | | | |
| Phage- | — | — | — | — | — | — | Y | L | — | D | A | — | — | — | 0.066 | 2.337 | 0.168 | 1668 |
| 1371 | | | | | | | | | | | | | | | | | | |
| Phage- | — | — | — | — | — | — | S | L | — | I | Y | — | — | N | 0.113 | 2.336 | 0.401 | 1669 |
| 1372 | | | | | | | | | | | | | | | | | | |
| Phage- | — | — | — | — | — | T | Y | L | — | F | E | — | — | N | 0.205 | 2.329 | 0.224 | 1670 |
| 1373 | | | | | | | | | | | | | | | | | | |
| Phage- | — | — | — | — | — | — | F | L | — | D | E | — | — | — | 0.064 | 2.284 | 0.246 | 1671 |
| 1374 | | | | | | | | | | | | | | | | | | |
| Phage- | — | — | — | — | — | S | Y | L | — | H | E | — | — | A | 0.096 | 2.284 | 0.198 | 1672 |
| 1375 | | | | | | | | | | | | | | | | | | |
| Phage- | — | — | — | — | — | K | F | L | — | H | E | — | — | S | 0.065 | 2.279 | 0.142 | 1673 |
| 1376 | | | | | | | | | | | | | | | | | | |
| Phage- | — | H | — | — | — | I | Y | L | — | D | E | — | — | N | 0.071 | 2.234 | 0.193 | 1674 |
| 1377 | | | | | | | | | | | | | | | | | | |
| Phage- | — | — | — | — | — | — | Y | L | — | H | E | — | — | H | 0.101 | 2.193 | 0.308 | 1675 |
| 1378 | | | | | | | | | | | | | | | | | | |
| Phage- | — | — | — | — | — | T | N | L | — | H | E | — | — | A | 0.061 | 2.163 | 0.096 | 1676 |
| 1379 | | | | | | | | | | | | | | | | | | |
| Phage- | — | — | — | — | — | — | D | L | — | D | V | — | — | A | 0.096 | 2.158 | 0.28 | 1677 |
| 1380 | | | | | | | | | | | | | | | | | | |
| Phage- | — | — | — | — | — | M | D | L | — | D | V | — | — | N | 0.09 | 2.149 | 0.263 | 1678 |
| 1381 | | | | | | | | | | | | | | | | | | |
| Phage- | — | — | — | — | — | — | S | L | — | N | V | — | — | — | 0.106 | 2.131 | 0.356 | 1679 |
| 1382 | | | | | | | | | | | | | | | | | | |
| Phage- | — | — | — | — | — | V | I | L | — | D | F | — | — | N | 0.066 | 2.03 | 0.173 | 1680 |
| 1383 | | | | | | | | | | | | | | | | | | |
| Phage- | L | — | — | — | — | L | N | L | — | D | L | — | Y | — | 0.062 | 2.028 | 0.095 | 1681 |
| 1384 | | | | | | | | | | | | | | | | | | |
| Phage- | — | — | — | — | — | — | H | L | — | Y | A | — | — | N | 0.061 | 2.012 | 0.159 | 1682 |
| 1385 | | | | | | | | | | | | | | | | | | |
| Phage- | — | — | — | — | — | K | H | L | — | I | E | — | — | A | 0.065 | 1.977 | 0.132 | 1683 |
| 1386 | | | | | | | | | | | | | | | | | | |
| Phage- | — | — | — | — | — | — | H | L | — | S | E | — | — | Y | 0.136 | 1.918 | 0.199 | 1684 |
| 1387 | | | | | | | | | | | | | | | | | | |
| Phage- | — | H | — | — | — | — | D | L | — | — | V | — | — | N | 0.07 | 1.834 | 0.112 | 1685 |
| 1388 | | | | | | | | | | | | | | | | | | |
| Phage- | — | H | — | — | — | E | Y | L | — | N | E | — | — | A | 0.074 | 1.687 | 0.092 | 1686 |
| 1389 | | | | | | | | | | | | | | | | | | |
| Phage- | — | — | — | — | — | — | D | L | — | A | V | — | — | A | 0.07 | 1.669 | 0.089 | 1687 |
| 1390 | | | | | | | | | | | | | | | | | | |
| Phage- | — | — | — | — | — | — | H | L | — | N | V | — | — | S | 0.058 | 1.613 | 0.176 | 1688 |
| 1391 | | | | | | | | | | | | | | | | | | |
| Phage- | — | — | F | — | — | I | S | L | — | H | E | — | — | — | 0.081 | 1.585 | 0.129 | 1689 |
| 1392 | | | | | | | | | | | | | | | | | | |
| Phage- | — | — | — | — | — | K | Y | L | — | D | K | — | — | H | 0.073 | 1.54 | 0.109 | 1690 |
| 1393 | | | | | | | | | | | | | | | | | | |
| Phage- | — | — | — | — | — | — | H | L | — | D | L | — | — | — | 0.093 | 1.395 | 0.182 | 1691 |
| 1394 | | | | | | | | | | | | | | | | | | |
| Phage- | — | — | — | — | — | — | D | L | — | N | V | — | — | A | 0.114 | 1.382 | 0.145 | 1692 |
| 1395 | | | | | | | | | | | | | | | | | | |
| Phage- | — | H | — | — | — | L | H | L | — | N | E | — | Y | H | 0.063 | 1.37 | 0.113 | 1693 |
| 1396 | | | | | | | | | | | | | | | | | | |
| Phage- | — | — | — | — | — | K | H | L | — | H | Q | — | — | H | 0.058 | 1.351 | 0.108 | 1694 |
| 1397 | | | | | | | | | | | | | | | | | | |
| Phage- | — | — | — | — | — | — | S | L | — | S | Y | — | — | H | 0.066 | 1.335 | 0.152 | 1695 |
| 1398 | | | | | | | | | | | | | | | | | | |
| Phage- | — | — | — | — | — | — | Y | L | — | T | E | — | — | — | 0.11 | 1.265 | 0.244 | 1696 |
| 1399 | | | | | | | | | | | | | | | | | | |
| Phage- | — | A | — | — | — | M | N | L | — | D | Q | — | — | — | 0.07 | 1.202 | 0.145 | 1697 |
| 1400 | | | | | | | | | | | | | | | | | | |
| Phage- | — | H | — | — | — | — | A | L | — | H | E | — | — | N | 0.07 | 1.178 | 0.144 | 1698 |
| 1401 | | | | | | | | | | | | | | | | | | |
| Phage- | — | — | — | — | — | — | H | L | — | D | Q | — | — | A | 0.15 | 1.122 | 0.144 | 1699 |
| 1402 | | | | | | | | | | | | | | | | | | |
| Phage- | — | N | — | — | — | T | D | L | — | H | E | — | — | N | 0.087 | 1.093 | 0.095 | 1700 |
| 1403 | | | | | | | | | | | | | | | | | | |
| Phage- | — | F | — | — | — | — | Y | L | — | H | E | — | — | N | 0.086 | 1.078 | 0.175 | 1701 |
| 1404 | | | | | | | | | | | | | | | | | | |
| Phage- | — | — | — | — | — | K | F | L | — | D | L | — | — | A | 0.1 | 1.075 | 0.139 | 1702 |
| 1405 | | | | | | | | | | | | | | | | | | |
| Phage- | — | — | — | — | — | F | Y | L | — | D | E | — | — | L | 0.07 | 1.024 | 0.141 | 1703 |
| 1406 | | | | | | | | | | | | | | | | | | |
| Phage- | — | — | — | — | — | — | H | L | — | D | L | — | — | A | 0.061 | 0.925 | 0.096 | 1704 |
| 1407 | | | | | | | | | | | | | | | | | | |
| Phage- | — | S | — | — | — | Q | D | L | — | H | V | — | — | N | 0.095 | 0.86 | 0.112 | 1705 |
| 1408 | | | | | | | | | | | | | | | | | | |
| Phage- | — | — | — | — | — | K | D | L | — | H | E | — | — | N | 0.073 | 0.762 | 0.1 | 1706 |
| 1409 | | | | | | | | | | | | | | | | | | |
| Phage- | — | — | — | — | — | — | D | L | — | N | V | — | — | N | 0.1 | 0.74 | 0.108 | 1707 |
| 1410 | | | | | | | | | | | | | | | | | | |
| Phage- | — | N | — | — | — | S | D | L | — | H | L | — | — | N | 0.071 | 0.739 | 0.131 | 1708 |
| 1411 | | | | | | | | | | | | | | | | | | |
| Phage- | — | N | — | — | — | S | H | L | — | H | M | — | Y | — | 0.195 | 0.702 | 0.192 | 1709 |
| 1412 | | | | | | | | | | | | | | | | | | |
| Phage- | — | — | — | — | — | P | Y | L | — | Y | E | — | — | A | 0.068 | 0.692 | 0.094 | 1710 |
| 1413 | | | | | | | | | | | | | | | | | | |
| Phage- | — | — | — | — | — | M | N | L | — | S | E | — | — | N | 0.168 | 0.67 | 0.124 | 1711 |
| 1414 | | | | | | | | | | | | | | | | | | |
| Phage- | — | — | — | — | — | K | H | L | — | N | E | — | — | N | 0.063 | 0.663 | 0.105 | 1712 |
| 1415 | | | | | | | | | | | | | | | | | | |
| Phage- | — | — | — | — | — | A | Y | L | — | A | E | — | — | S | 0.082 | 0.64 | 0.12 | 1713 |
| 1416 | | | | | | | | | | | | | | | | | | |
| Phage- | — | — | — | — | — | S | D | L | — | H | E | — | — | H | 0.075 | 0.629 | 0.164 | 1714 |
| 1417 | | | | | | | | | | | | | | | | | | |
| Phage- | — | — | — | — | — | V | S | L | — | D | H | — | — | N | 0.075 | 0.616 | 0.1 | 1715 |
| 1418 | | | | | | | | | | | | | | | | | | |
| Phage- | L | — | — | — | — | — | D | L | — | H | V | — | — | — | 0.176 | 0.61 | 0.105 | 1716 |
| 1419 | | | | | | | | | | | | | | | | | | |
| Phage- | — | — | — | — | — | W | I | L | — | N | E | — | — | N | 0.141 | 0.581 | 0.137 | 1717 |
| 1420 | | | | | | | | | | | | | | | | | | |
| Phage- | — | H | — | — | — | — | Y | L | — | D | E | — | — | N | 0.132 | 0.55 | 0.127 | 1718 |
| 1421 | | | | | | | | | | | | | | | | | | |
| Phage- | — | Y | — | — | — | — | D | L | — | D | V | — | — | N | 0.145 | 0.512 | 0.102 | 1719 |
| 1422 | | | | | | | | | | | | | | | | | | |
| Phage- | — | H | — | — | — | — | S | L | — | N | E | — | Y | — | 0.168 | 0.456 | 0.115 | 1720 |
| 1423 | | | | | | | | | | | | | | | | | | |
| Phage- | I | — | — | — | — | — | D | L | — | A | L | — | — | N | 0.127 | 0.37 | 0.129 | 1721 |
| 1424 | | | | | | | | | | | | | | | | | | |
| Phage- | — | H | — | — | — | I | N | L | — | N | E | — | — | S | 0.081 | 0.316 | 0.098 | 1722 |
| 1425 | | | | | | | | | | | | | | | | | | |
| Phage- | — | E | R | — | — | — | F | L | — | N | E | — | — | N | 0.132 | 0.216 | 0.093 | 1723 |
| 1426 | | | | | | | | | | | | | | | | | | |
| Phage- | Q | — | — | — | — | T | Y | L | — | H | H | — | — | N | 0.101 | 0.123 | 0.122 | 1724 |
| 1427 | | | | | | | | | | | | | | | | | | |
| Phage- | I | G | K | L | T | L | C | L | N | A | — | L | V | I | 0.274 | 0.116 | 0.1 | 1725 |
| 1428 | | | | | | | | | | | | | | | | | | |
| Phage- | — | — | — | — | — | S | Y | L | — | D | Q | — | — | — | 0.1 | 0.083 | 0.079 | 1726 |
| 1429 | | | | | | | | | | | | | | | | | | |
| Phage- | V | — | — | — | — | — | Y | L | — | H | V | — | Y | N | 0.064 | 0.065 | 0.061 | 1727 |
| 1430 | | | | | | | | | | | | | | | | | | |
| Phage- | — | — | — | — | — | — | D | M | — | A | E | — | — | — | 0.099 | 2.687 | 2.259 | 1728 |
| 1431 | | | | | | | | | | | | | | | | | | |
| Phage- | — | — | — | — | — | — | D | M | — | Y | E | — | — | N | 0.099 | 2.564 | 0.235 | 1729 |
| 1432 | | | | | | | | | | | | | | | | | | |
| Phage- | D | V | — | — | — | K | Y | M | — | S | L | — | — | N | 0.101 | 2.529 | 0.418 | 1730 |
| 1433 | | | | | | | | | | | | | | | | | | |
| Phage- | — | — | — | — | — | M | D | M | — | — | N | — | — | N | 0.059 | 2.516 | 0.277 | 1731 |
| 1434 | | | | | | | | | | | | | | | | | | |
| Phage- | — | — | — | — | — | S | D | M | — | H | E | — | Y | A | 0.062 | 2.474 | 0.2 | 1732 |
| 1435 | | | | | | | | | | | | | | | | | | |
| Phage- | — | — | — | — | — | K | H | M | — | F | M | — | — | N | 0.101 | 2.395 | 0.372 | 1733 |
| 1436 | | | | | | | | | | | | | | | | | | |
| Phage- | — | — | — | — | — | — | Y | M | — | Y | Q | — | — | S | 0.079 | 2.364 | 0.357 | 1734 |
| 1437 | | | | | | | | | | | | | | | | | | |
| Phage- | — | — | — | — | — | — | H | M | — | Y | E | — | — | — | 0.129 | 2.342 | 0.298 | 1735 |
| 1438 | | | | | | | | | | | | | | | | | | |
| Phage- | — | — | — | — | — | — | A | M | — | N | H | — | — | N | 0.321 | 2.326 | 0.426 | 1736 |
| 1439 | | | | | | | | | | | | | | | | | | |
| Phage- | — | — | — | — | — | — | N | M | — | A | Q | — | — | A | 0.102 | 2.315 | 0.209 | 1737 |
| 1440 | | | | | | | | | | | | | | | | | | |
| Phage- | — | F | — | — | — | F | D | M | — | H | F | — | — | N | 0.15 | 2.292 | 0.469 | 1738 |
| 1441 | | | | | | | | | | | | | | | | | | |
| Phage- | — | — | — | — | — | — | D | M | — | D | Q | — | — | D | 0.072 | 2.292 | 0.119 | 1739 |
| 1442 | | | | | | | | | | | | | | | | | | |
| Phage- | — | F | — | — | — | M | D | M | — | D | Q | — | — | N | 0.091 | 2.26 | 0.207 | 1740 |
| 1443 | | | | | | | | | | | | | | | | | | |
| Phage- | — | S | — | — | — | — | D | M | — | F | Y | — | Y | A | 0.065 | 2.248 | 0.174 | 1741 |
| 1444 | | | | | | | | | | | | | | | | | | |
| Phage- | — | — | — | — | — | — | H | M | — | N | V | — | — | S | 0.067 | 2.245 | 0.13 | 1742 |
| 1445 | | | | | | | | | | | | | | | | | | |
| Phage- | — | — | — | — | — | T | D | M | — | H | H | — | — | L | 0.061 | 2.201 | 0.126 | 1743 |
| 1446 | | | | | | | | | | | | | | | | | | |
| Phage- | I | H | — | — | — | I | N | M | — | D | K | — | Y | N | 0.06 | 2.08 | 0.203 | 1744 |
| 1447 | | | | | | | | | | | | | | | | | | |
| Phage- | — | — | — | — | — | — | A | M | — | H | E | — | — | Y | 0.069 | 1.915 | 0.129 | 1745 |
| 1448 | | | | | | | | | | | | | | | | | | |
| Phage- | — | — | — | — | — | T | D | M | — | I | V | — | — | A | 0.363 | 1.495 | 0.172 | 1746 |
| 1449 | | | | | | | | | | | | | | | | | | |
| Phage- | I | — | — | — | — | Q | D | M | — | F | Y | — | — | N | 0.069 | 1.416 | 0.16 | 1747 |
| 1450 | | | | | | | | | | | | | | | | | | |
| Phage- | — | — | — | — | — | — | D | M | — | F | E | — | — | A | 0.076 | 1.31 | 0.143 | 1748 |
| 1451 | | | | | | | | | | | | | | | | | | |
| Phage- | — | — | — | — | — | — | N | M | — | I | V | — | — | L | 0.06 | 1.116 | 0.093 | 1749 |
| 1452 | | | | | | | | | | | | | | | | | | |
| Phage- | — | — | — | — | — | — | A | M | — | D | K | — | — | — | 0.073 | 0.985 | 0.094 | 1750 |
| 1453 | | | | | | | | | | | | | | | | | | |
| Phage- | — | N | — | — | — | S | Y | M | — | D | Q | — | — | A | 0.125 | 0.854 | 0.105 | 1751 |
| 1454 | | | | | | | | | | | | | | | | | | |
| Phage- | — | — | — | — | — | T | Y | M | — | S | E | — | — | N | 0.086 | 0.754 | 0.107 | 1752 |
| 1455 | | | | | | | | | | | | | | | | | | |
| Phage- | — | — | — | — | — | — | Y | M | — | N | E | — | — | N | 0.064 | 0.687 | 0.128 | 1753 |
| 1456 | | | | | | | | | | | | | | | | | | |
| Phage- | — | — | — | — | — | M | N | M | — | Y | Q | — | — | N | 0.11 | 0.639 | 0.137 | 1754 |
| 1457 | | | | | | | | | | | | | | | | | | |
| Phage- | — | — | — | — | — | W | D | M | — | D | K | — | — | N | 0.101 | 0.586 | 0.108 | 1755 |
| 1458 | | | | | | | | | | | | | | | | | | |
| Phage- | T | F | G | — | — | T | T | M | — | N | R | — | — | A | 0.117 | 0.181 | 0.068 | 1756 |
| 1459 | | | | | | | | | | | | | | | | | | |
| Phage- | N | Y | — | — | — | S | S | M | — | N | R | — | L | H | 0.295 | 0.158 | 0.088 | 1757 |
| 1460 | | | | | | | | | | | | | | | | | | |
| Phage- | F | — | F | — | — | T | T | M | — | T | Y | — | Q | H | 0.084 | 0.086 | 0.081 | 1758 |
| 1461 | | | | | | | | | | | | | | | | | | |
| Phage- | T | T | — | — | — | T | S | M | — | L | H | — | — | D | 0.096 | 0.074 | 0.072 | 1759 |
| 1462 | | | | | | | | | | | | | | | | | | |
| Phage- | — | — | — | — | — | — | D | Q | — | H | N | — | — | N | 0.075 | 0.2 | 0.078 | 1760 |
| 1463 | | | | | | | | | | | | | | | | | | |
| Phage- | — | — | — | — | — | — | D | — | — | — | — | — | — | — | 0.066 | 1.577 | 0.14 | 1761 |
| 1464 | | | | | | | | | | | | | | | | | | |
| Phage- | — | — | — | — | — | K | D | — | — | N | — | — | Y | — | 0.078 | 1.091 | 0.093 | 1762 |
| 1465 | | | | | | | | | | | | | | | | | | |
| Phage- | — | — | — | — | — | — | D | — | — | A | — | — | — | — | 0.069 | 0.392 | 0.089 | 1763 |
| 1466 | | | | | | | | | | | | | | | | | | |
| Phage- | — | — | — | — | — | — | D | — | — | I | — | — | — | N | 0.218 | 0.339 | 0.131 | 1764 |
| 1467 | | | | | | | | | | | | | | | | | | |
| Phage- | — | — | — | — | — | — | D | — | — | S | — | — | — | N | 0.074 | 0.102 | 0.074 | 1765 |
| 1468 | | | | | | | | | | | | | | | | | | |
| Phage- | I | T | — | — | H | V | I | S | G | L | E | — | W | N | 0.076 | 0.193 | 0.1 | 1766 |
| 1469 | | | | | | | | | | | | | | | | | | |
| Phage- | V | P | — | — | Q | I | I | S | G | L | E | — | L | T | 0.076 | 0.174 | 0.077 | 1767 |
| 1470 | | | | | | | | | | | | | | | | | | |
| Phage- | A | P | — | — | Q | I | I | S | G | L | E | — | L | T | 0.08 | 0.169 | 0.078 | 1768 |
| 1471 | | | | | | | | | | | | | | | | | | |
| Phage- | V | P | — | — | L | I | I | S | G | L | — | — | L | N | 0.455 | 0.107 | 0.09 | 1769 |
| 1472 | | | | | | | | | | | | | | | | | | |
| Phage- | — | — | — | — | A | — | F | V | G | Y | G | — | L | D | 0.948 | 0.065 | 0.074 | 1770 |
| 1473 | | | | | | | | | | | | | | | | | | |
| Phage- | — | T | — | — | — | T | S | F | — | N | R | — | L | D | 0.142 | 0.392 | 0.405 | 1771 |
| 1474 | | | | | | | | | | | | | | | | | | |
| Phage- | — | — | — | — | — | — | A | L | — | F | E | — | — | — | 0.111 | 2.668 | 0.331 | 1772 |
| 1475 | | | | | | | | | | | | | | | | | | |
| Phage- | — | — | — | — | — | — | Y | L | — | H | E | — | — | S | 0.063 | 2.654 | 0.303 | 1773 |
| 1476 | | | | | | | | | | | | | | | | | | |
| Phage- | — | — | — | — | — | — | D | L | — | N | L | — | — | — | 0.084 | 2.639 | 0.436 | 1774 |
| 1477 | | | | | | | | | | | | | | | | | | |
| Phage- | — | — | — | — | — | S | Y | L | — | H | E | — | — | — | 0.067 | 2.606 | 0.447 | 1775 |
| 1478 | | | | | | | | | | | | | | | | | | |
| Phage- | — | — | — | — | — | P | Y | L | — | S | E | — | — | A | 0.539 | 2.604 | 0.234 | 1776 |
| 1479 | | | | | | | | | | | | | | | | | | |
| Phage- | — | — | — | — | — | — | Y | L | — | H | V | — | — | N | 0.187 | 2.603 | 0.409 | 1777 |
| 1480 | | | | | | | | | | | | | | | | | | |
| Phage- | — | — | — | — | — | — | T | L | — | H | V | — | — | N | 0.317 | 2.601 | 0.551 | 1778 |
| 1481 | | | | | | | | | | | | | | | | | | |
| Phage- | — | — | — | — | — | — | H | L | — | H | E | — | Y | S | 0.066 | 2.584 | 0.374 | 1779 |
| 1482 | | | | | | | | | | | | | | | | | | |
| Phage- | — | — | — | — | — | K | H | L | — | T | E | — | — | A | 0.098 | 2.491 | 0.145 | 1780 |
| 1483 | | | | | | | | | | | | | | | | | | |
| Phage- | — | — | — | — | — | — | H | L | — | Y | E | — | — | N | 0.063 | 2.414 | 0.167 | 1781 |
| 1484 | | | | | | | | | | | | | | | | | | |
| Phage- | — | — | — | — | — | — | Y | L | — | H | E | — | — | D | 0.071 | 2.066 | 0.177 | 1782 |
| 1485 | | | | | | | | | | | | | | | | | | |
| Phage- | — | — | — | — | — | — | N | L | — | H | L | — | — | A | 0.082 | 1.397 | 0.164 | 1783 |
| 1486 | | | | | | | | | | | | | | | | | | |
| Phage- | — | — | — | — | — | K | H | L | — | N | K | — | — | N | 0.075 | 1.161 | 0.092 | 1784 |
| 1487 | | | | | | | | | | | | | | | | | | |
| Phage- | — | — | — | — | — | — | F | M | — | A | E | — | — | N | 0.073 | 2.603 | 0.341 | 1785 |
| 1488 | | | | | | | | | | | | | | | | | | |
| Phage- | — | — | — | — | — | — | N | M | — | H | H | — | — | D | 0.205 | 1.597 | 0.524 | 1786 |
| 1489 | | | | | | | | | | | | | | | | | | |
| Phage- | — | — | — | — | — | S | D | M | — | A | H | — | Y | N | 0.191 | 1.209 | 0.109 | 1787 |
| 1490 | | | | | | | | | | | | | | | | | | |
| Phage- | — | — | — | — | — | K | V | M | — | H | Y | — | — | A | 0.136 | 1.138 | 0.252 | 1788 |
| 1491 | | | | | | | | | | | | | | | | | | |
| Phage- | — | — | — | — | — | — | F | M | — | S | E | — | — | S | 0.092 | 1.008 | 0.137 | 1789 |
| 1492 | | | | | | | | | | | | | | | | | | |
| Phage- | — | — | — | — | — | — | H | M | — | I | N | — | — | A | 0.091 | 0.92 | 0.165 | 1790 |
| 1493 | | | | | | | | | | | | | | | | | | |
| Phage- | — | V | G | — | A | T | S | M | — | N | R | — | L | T | 0.256 | 0.77 | 0.746 | 1791 |
| 1494 | | | | | | | | | | | | | | | | | | |
| Phage- | — | — | — | — | — | — | Y | L | — | S | E | — | — | A | 0.078 | 2.705 | 0.513 | 1792 |
| 1495 | | | | | | | | | | | | | | | | | | |
| Phage- | — | — | — | — | — | — | H | L | — | S | V | — | — | N | 0.064 | 2.649 | 0.825 | 1793 |
| 1496 | | | | | | | | | | | | | | | | | | |
| Phage- | — | — | — | — | — | F | D | L | — | H | V | — | Y | N | 0.089 | 2.602 | 0.651 | 1794 |
| 1497 | | | | | | | | | | | | | | | | | | |
| Phage- | — | — | — | — | — | — | F | L | — | H | E | — | — | N | 0.079 | 2.586 | 0.351 | 1795 |
| 1498 | | | | | | | | | | | | | | | | | | |
| Phage- | — | — | — | — | — | — | S | L | — | H | V | — | — | N | 0.098 | 2.412 | 0.238 | 1796 |
| 1499 | | | | | | | | | | | | | | | | | | |
| Phage- | — | — | — | — | — | — | N | L | — | H | A | — | — | N | 0.071 | 1.571 | 0.112 | 1797 |
| 1500 | | | | | | | | | | | | | | | | | | |
| Phage- | — | — | — | — | — | V | F | M | — | N | E | — | — | N | 0.079 | 2.661 | 0.356 | 1798 |
| 1501 | | | | | | | | | | | | | | | | | | |
| Phage- | — | — | — | — | — | M | F | M | — | H | E | — | — | N | 0.102 | 2.235 | 1.045 | 1799 |
| 1502 | | | | | | | | | | | | | | | | | | |
| Phage- | — | Y | — | — | A | T | S | M | — | N | R | — | — | V | 0.248 | 0.398 | 0.415 | 1800 |
| 1503 | | | | | | | | | | | | | | | | | | |
| Phage- | — | — | — | — | — | — | H | M | — | H | E | — | — | A | 0.074 | 2.518 | 0.298 | 1801 |
| 1504 | | | | | | | | | | | | | | | | | | |
| Phage- | — | — | — | — | — | — | H | L | — | H | V | — | — | N | 0.07 | 2.667 | 0.636 | 1802 |
| 1505 | | | | | | | | | | | | | | | | | | |
| Phage- | — | — | — | — | — | — | F | M | — | D | L | — | — | A | 0.27 | 2.638 | 0.725 | 1803 |
| 1506 | | | | | | | | | | | | | | | | | | |
| Phage- | V | T | S | — | — | T | T | M | — | N | R | — | — | S | 0.396 | 1.22 | 0.546 | 1804 |
| 1507 | | | | | | | | | | | | | | | | | | |
| Phage- | — | T | L | — | L | S | V | D | L | — | H | — | W | Y | 0.114 | 0.632 | 0.093 | 1805 |
| 1508 | | | | | | | | | | | | | | | | | | |
| Phage- | D | S | F | — | T | W | S | A | — | Q | E | — | G | R | 0.072 | 0.13 | 0.101 | 1806 |
| 1509 | | | | | | | | | | | | | | | | | | |
| Phage- | — | — | — | — | — | — | D | F | — | A | F | — | — | — | 0.118 | 2.365 | 0.655 | 1807 |
| 1510 | | | | | | | | | | | | | | | | | | |
| Phage- | — | — | — | — | — | T | S | F | — | N | R | — | — | H | 0.102 | 0.087 | 0.092 | 1808 |
| 1511 | | | | | | | | | | | | | | | | | | |
| Phage- | — | V | — | — | M | S | V | G | — | A | V | — | L | N | 0.289 | 0.271 | 0.284 | 1809 |
| 1512 | | | | | | | | | | | | | | | | | | |
| Phage- | I | — | — | — | — | T | A | G | — | T | Y | — | W | — | 0.071 | 0.136 | 0.075 | 1810 |
| 1513 | | | | | | | | | | | | | | | | | | |
| Phage- | W | V | S | — | L | — | H | H | — | L | E | — | — | H | 0.781 | 0.723 | 0.858 | 1811 |
| 1514 | | | | | | | | | | | | | | | | | | |
| Phage- | — | — | — | — | — | — | D | H | — | — | — | — | Y | S | 0.172 | 0.671 | 0.092 | 1812 |
| 1515 | | | | | | | | | | | | | | | | | | |
| Phage- | I | — | — | — | — | K | I | H | — | D | L | — | Y | — | 0.072 | 0.295 | 0.082 | 1813 |
| 1516 | | | | | | | | | | | | | | | | | | |
| Phage- | — | — | — | — | — | — | S | H | — | S | E | — | — | S | 0.08 | 0.253 | 0.087 | 1814 |
| 1517 | | | | | | | | | | | | | | | | | | |
| Phage- | — | — | — | — | — | — | S | I | — | Y | L | — | — | N | 0.383 | 2.548 | 1.337 | 1815 |
| 1518 | | | | | | | | | | | | | | | | | | |
| Phage- | — | — | — | — | — | — | Y | L | — | A | E | — | — | N | 0.068 | 2.742 | 1.141 | 1816 |
| 1519 | | | | | | | | | | | | | | | | | | |
| Phage- | — | — | — | — | — | — | Y | L | — | D | Y | — | — | A | 0.073 | 2.709 | 0.382 | 1817 |
| 1520 | | | | | | | | | | | | | | | | | | |
| Phage- | — | — | — | — | — | — | S | L | — | F | L | — | — | N | 1.11 | 2.709 | 1.774 | 1818 |
| 1521 | | | | | | | | | | | | | | | | | | |
| Phage- | — | — | — | — | — | — | D | L | — | F | A | — | — | A | 0.06 | 2.658 | 0.794 | 1819 |
| 1522 | | | | | | | | | | | | | | | | | | |
| Phage- | — | — | — | — | — | — | H | L | — | N | E | — | — | N | 0.079 | 2.65 | 0.393 | 1820 |
| 1523 | | | | | | | | | | | | | | | | | | |
| Phage- | — | — | — | — | — | — | F | L | — | H | V | — | — | S | 2.091 | 2.643 | 2.48 | 1821 |
| 1524 | | | | | | | | | | | | | | | | | | |
| Phage- | — | — | — | — | — | — | D | L | — | A | M | — | — | A | 0.071 | 2.627 | 0.889 | 1822 |
| 1525 | | | | | | | | | | | | | | | | | | |
| Phage- | — | E | R | — | — | — | Y | L | — | H | M | — | Y | S | 0.164 | 2.589 | 0.84 | 1823 |
| 1526 | | | | | | | | | | | | | | | | | | |
| Phage- | — | — | — | — | — | I | A | L | — | D | F | — | — | A | 0.073 | 2.522 | 0.492 | 1824 |
| 1527 | | | | | | | | | | | | | | | | | | |
| Phage- | — | — | — | — | — | — | Y | L | — | N | V | — | — | A | 0.07 | 2.513 | 0.28 | 1825 |
| 1528 | | | | | | | | | | | | | | | | | | |
| Phage- | — | — | — | — | — | — | D | L | — | S | V | — | — | S | 0.09 | 2.505 | 0.116 | 1826 |
| 1529 | | | | | | | | | | | | | | | | | | |
| Phage- | — | — | — | — | — | — | Y | L | — | H | V | — | — | Y | 0.227 | 2.498 | 1.137 | 1827 |
| 1530 | | | | | | | | | | | | | | | | | | |
| Phage- | — | — | — | — | — | — | A | L | — | D | H | — | — | — | 0.081 | 2.492 | 0.394 | 1828 |
| 1531 | | | | | | | | | | | | | | | | | | |
| Phage- | — | — | — | — | — | — | H | L | — | F | E | — | — | N | 0.064 | 2.489 | 0.171 | 1829 |
| 1532 | | | | | | | | | | | | | | | | | | |
| Phage- | — | — | — | — | — | — | S | L | — | D | Y | — | — | A | 0.115 | 2.488 | 0.235 | 1830 |
| 1533 | | | | | | | | | | | | | | | | | | |
| Phage- | — | — | — | — | — | — | H | L | — | T | E | — | — | S | 0.074 | 2.482 | 0.243 | 1831 |
| 1534 | | | | | | | | | | | | | | | | | | |
| Phage- | — | — | — | — | — | L | Y | L | — | A | E | — | — | N | 0.094 | 2.476 | 0.193 | 1832 |
| 1535 | | | | | | | | | | | | | | | | | | |
| Phage- | — | — | — | — | — | I | Y | L | — | A | E | — | — | N | 0.078 | 2.453 | 0.307 | 1833 |
| 1536 | | | | | | | | | | | | | | | | | | |
| Phage- | — | — | — | — | — | — | H | L | — | F | E | — | — | — | 0.178 | 2.451 | 0.479 | 1834 |
| 1537 | | | | | | | | | | | | | | | | | | |
| Phage- | — | — | — | — | — | — | Y | L | — | H | E | — | — | — | 0.082 | 2.45 | 0.682 | 1835 |
| 1538 | | | | | | | | | | | | | | | | | | |
| Phage- | — | — | — | — | — | — | Y | L | — | H | V | — | — | D | 0.068 | 2.445 | 0.31 | 1836 |
| 1539 | | | | | | | | | | | | | | | | | | |
| Phage- | — | — | — | — | — | — | Y | L | — | T | E | — | — | S | 0.074 | 2.421 | 0.329 | 1837 |
| 1540 | | | | | | | | | | | | | | | | | | |
| Phage- | — | — | — | — | — | K | F | L | — | D | E | — | — | A | 0.311 | 2.406 | 0.376 | 1838 |
| 1541 | | | | | | | | | | | | | | | | | | |
| Phage- | — | — | — | — | — | — | D | L | — | T | E | — | — | S | 0.08 | 2.401 | 0.174 | 1839 |
| 1542 | | | | | | | | | | | | | | | | | | |
| Phage- | — | — | — | — | — | — | H | L | — | N | E | — | — | A | 0.068 | 2.394 | 0.17 | 1840 |
| 1543 | | | | | | | | | | | | | | | | | | |
| Phage- | — | — | — | — | — | — | Y | L | — | P | V | — | — | H | 0.179 | 2.392 | 0.602 | 1841 |
| 1544 | | | | | | | | | | | | | | | | | | |
| Phage- | — | — | — | — | — | K | S | L | — | A | E | — | — | N | 0.395 | 2.377 | 0.162 | 1842 |
| 1545 | | | | | | | | | | | | | | | | | | |
| Phage- | — | — | — | — | — | M | F | L | — | H | E | — | — | N | 0.17 | 2.375 | 0.591 | 1843 |
| 1546 | | | | | | | | | | | | | | | | | | |
| Phage- | — | — | — | — | — | — | D | L | — | D | E | — | — | N | 0.066 | 2.374 | 0.248 | 1844 |
| 1547 | | | | | | | | | | | | | | | | | | |
| Phage- | — | — | — | — | — | — | D | L | — | Y | Q | — | — | N | 0.069 | 2.353 | 0.609 | 1845 |
| 1548 | | | | | | | | | | | | | | | | | | |
| Phage- | — | — | — | — | — | — | S | L | — | N | Y | — | — | N | 0.076 | 2.35 | 0.209 | 1846 |
| 1549 | | | | | | | | | | | | | | | | | | |
| Phage- | — | — | — | — | — | I | H | L | — | N | E | — | — | N | 0.074 | 2.347 | 0.157 | 1847 |
| 1550 | | | | | | | | | | | | | | | | | | |
| Phage- | — | — | — | — | — | T | Y | L | — | H | V | — | — | S | 0.089 | 2.324 | 0.16 | 1848 |
| 1551 | | | | | | | | | | | | | | | | | | |
| Phage- | — | — | — | — | — | — | S | L | — | N | Y | — | — | A | 0.067 | 2.316 | 0.198 | 1849 |
| 1552 | | | | | | | | | | | | | | | | | | |
| Phage- | — | — | — | — | — | — | H | L | — | H | E | — | — | T | 0.103 | 2.297 | 0.32 | 1850 |
| 1553 | | | | | | | | | | | | | | | | | | |
| Phage- | — | — | — | — | — | M | F | L | — | D | E | — | — | N | 0.132 | 2.287 | 0.203 | 1851 |
| 1554 | | | | | | | | | | | | | | | | | | |
| Phage- | — | S | — | — | — | — | D | L | — | H | L | — | — | — | 0.077 | 2.265 | 0.319 | 1852 |
| 1555 | | | | | | | | | | | | | | | | | | |
| Phage- | — | — | — | — | — | T | Y | L | — | N | E | — | — | N | 0.12 | 2.244 | 0.606 | 1853 |
| 1556 | | | | | | | | | | | | | | | | | | |
| Phage- | — | — | — | — | — | — | D | L | — | Y | V | — | — | A | 0.095 | 2.221 | 0.138 | 1854 |
| 1557 | | | | | | | | | | | | | | | | | | |
| Phage- | — | — | — | — | — | — | H | L | — | I | E | — | — | N | 0.339 | 2.214 | 0.376 | 1855 |
| 1558 | | | | | | | | | | | | | | | | | | |
| Phage- | — | — | — | — | — | T | Y | L | — | N | V | — | — | A | 0.085 | 2.211 | 0.132 | 1856 |
| 1559 | | | | | | | | | | | | | | | | | | |
| Phage- | — | — | — | — | — | — | H | L | — | Y | E | — | — | S | 0.216 | 2.167 | 0.163 | 1857 |
| 1560 | | | | | | | | | | | | | | | | | | |
| Phage- | — | — | — | — | — | A | Y | L | — | D | E | — | — | A | 0.096 | 2.166 | 0.163 | 1858 |
| 1561 | | | | | | | | | | | | | | | | | | |
| Phage- | — | F | — | — | — | I | T | L | — | N | E | — | — | N | 0.075 | 2.153 | 0.143 | 1859 |
| 1562 | | | | | | | | | | | | | | | | | | |
| Phage- | — | H | — | — | — | — | D | L | — | H | V | — | — | N | 0.071 | 2.149 | 0.15 | 1860 |
| 1563 | | | | | | | | | | | | | | | | | | |
| Phage- | — | — | — | — | — | — | A | L | — | D | H | — | — | N | 0.323 | 2.145 | 0.293 | 1861 |
| 1564 | | | | | | | | | | | | | | | | | | |
| Phage- | — | — | — | — | — | — | A | L | — | N | V | — | — | A | 0.071 | 2.14 | 0.249 | 1862 |
| 1565 | | | | | | | | | | | | | | | | | | |
| Phage- | — | — | — | — | — | — | F | L | — | D | V | — | — | N | 0.1 | 2.139 | 0.309 | 1863 |
| 1566 | | | | | | | | | | | | | | | | | | |
| Phage- | — | — | — | — | — | — | H | L | — | D | E | — | — | Y | 0.098 | 2.127 | 0.146 | 1864 |
| 1567 | | | | | | | | | | | | | | | | | | |
| Phage- | I | — | — | — | — | — | A | L | — | D | A | — | L | A | 0.269 | 2.103 | 0.164 | 1865 |
| 1568 | | | | | | | | | | | | | | | | | | |
| Phage- | — | — | — | — | — | T | D | L | — | H | E | — | — | A | 0.078 | 2.071 | 0.154 | 1866 |
| 1569 | | | | | | | | | | | | | | | | | | |
| Phage- | — | — | — | — | — | — | Y | L | — | D | E | — | — | S | 0.069 | 2.064 | 0.164 | 1867 |
| 1570 | | | | | | | | | | | | | | | | | | |
| Phage- | — | — | — | — | — | — | F | L | — | D | Y | — | — | A | 0.146 | 2.034 | 0.207 | 1868 |
| 1571 | | | | | | | | | | | | | | | | | | |
| Phage- | — | — | — | — | — | — | D | L | — | N | W | — | — | N | 0.139 | 2.026 | 0.39 | 1869 |
| 1572 | | | | | | | | | | | | | | | | | | |
| Phage- | — | — | — | — | — | K | P | L | — | H | V | — | — | A | 0.069 | 2.022 | 0.152 | 1870 |
| 1573 | | | | | | | | | | | | | | | | | | |
| Phage- | — | — | — | — | — | — | F | L | — | N | E | — | — | Y | 0.143 | 1.996 | 0.287 | 1871 |
| 1574 | | | | | | | | | | | | | | | | | | |
| Phage- | — | — | — | — | — | — | T | L | — | D | Q | — | — | — | 0.088 | 1.992 | 0.314 | 1872 |
| 1575 | | | | | | | | | | | | | | | | | | |
| Phage- | — | S | — | — | — | I | D | L | — | S | E | — | — | A | 0.072 | 1.986 | 0.148 | 1873 |
| 1576 | | | | | | | | | | | | | | | | | | |
| Phage- | — | — | — | — | — | — | H | L | — | D | E | — | — | N | 0.084 | 1.978 | 0.268 | 1874 |
| 1577 | | | | | | | | | | | | | | | | | | |
| Phage- | — | — | — | — | — | — | N | L | — | H | E | — | — | A | 0.067 | 1.965 | 0.175 | 1875 |
| 1578 | | | | | | | | | | | | | | | | | | |
| Phage- | — | — | — | — | — | — | D | L | — | T | Q | — | — | — | 0.065 | 1.932 | 0.119 | 1876 |
| 1579 | | | | | | | | | | | | | | | | | | |
| Phage- | — | — | — | — | — | — | H | L | — | — | H | — | — | S | 0.096 | 1.914 | 0.173 | 1877 |
| 1580 | | | | | | | | | | | | | | | | | | |
| Phage- | — | — | F | — | — | — | F | L | — | H | E | — | — | N | 0.063 | 1.908 | 0.225 | 1878 |
| 1581 | | | | | | | | | | | | | | | | | | |
| Phage- | — | — | — | — | — | — | H | L | — | H | A | — | — | S | 0.073 | 1.826 | 0.167 | 1879 |
| 1582 | | | | | | | | | | | | | | | | | | |
| Phage- | — | — | — | — | — | L | F | L | — | D | Q | — | — | N | 0.116 | 1.819 | 0.264 | 1880 |
| 1583 | | | | | | | | | | | | | | | | | | |
| Phage- | — | A | — | — | — | W | Y | L | — | D | E | — | — | N | 0.2 | 1.81 | 0.267 | 1881 |
| 1584 | | | | | | | | | | | | | | | | | | |
| Phage- | L | — | — | — | — | — | H | L | — | A | L | — | — | N | 0.073 | 1.78 | 0.24 | 1882 |
| 1585 | | | | | | | | | | | | | | | | | | |
| Phage- | — | — | — | — | — | W | F | L | — | N | E | — | — | N | 0.158 | 1.774 | 0.279 | 1883 |
| 1586 | | | | | | | | | | | | | | | | | | |
| Phage- | — | — | — | — | — | M | N | L | — | H | E | — | — | A | 0.08 | 1.742 | 0.16 | 1884 |
| 1587 | | | | | | | | | | | | | | | | | | |
| Phage- | — | — | — | — | — | I | H | L | — | Y | E | — | — | N | 0.08 | 1.734 | 0.203 | 1885 |
| 1588 | | | | | | | | | | | | | | | | | | |
| Phage- | I | — | — | — | — | L | H | L | — | H | E | — | Y | H | 0.067 | 1.693 | 0.182 | 1886 |
| 1589 | | | | | | | | | | | | | | | | | | |
| Phage- | — | — | — | — | — | — | Y | L | — | L | E | — | — | N | 0.083 | 1.685 | 0.368 | 1887 |
| 1590 | | | | | | | | | | | | | | | | | | |
| Phage- | — | — | — | — | — | M | Y | L | — | D | E | — | — | — | 0.082 | 1.652 | 0.178 | 1888 |
| 1591 | | | | | | | | | | | | | | | | | | |
| Phage- | — | — | — | — | — | — | L | L | — | H | E | — | — | N | 0.071 | 1.601 | 0.109 | 1889 |
| 1592 | | | | | | | | | | | | | | | | | | |
| Phage- | — | — | — | — | — | P | H | L | — | H | E | — | — | — | 0.069 | 1.449 | 0.136 | 1890 |
| 1593 | | | | | | | | | | | | | | | | | | |
| Phage- | — | — | — | — | — | — | P | L | — | H | E | — | — | A | 0.063 | 1.439 | 0.138 | 1891 |
| 1594 | | | | | | | | | | | | | | | | | | |
| Phage- | — | — | — | — | — | M | F | L | — | H | E | — | — | S | 0.092 | 1.435 | 0.193 | 1892 |
| 1595 | | | | | | | | | | | | | | | | | | |
| Phage- | — | H | — | — | — | — | H | L | — | S | E | — | — | N | 0.165 | 1.43 | 0.478 | 1893 |
| 1596 | | | | | | | | | | | | | | | | | | |
| Phage- | — | — | — | — | — | — | I | L | — | H | E | — | — | S | 0.064 | 1.391 | 0.174 | 1894 |
| 1597 | | | | | | | | | | | | | | | | | | |
| Phage- | — | N | — | — | — | M | H | L | — | A | E | — | — | N | 0.087 | 1.39 | 0.14 | 1895 |
| 1598 | | | | | | | | | | | | | | | | | | |
| Phage- | — | — | — | — | — | — | S | L | — | A | Q | — | — | Y | 0.067 | 1.387 | 0.14 | 1896 |
| 1599 | | | | | | | | | | | | | | | | | | |
| Phage- | — | — | — | — | — | S | Y | L | — | P | V | — | — | N | 0.079 | 1.178 | 0.131 | 1897 |
| 1600 | | | | | | | | | | | | | | | | | | |
| Phage- | — | H | — | — | — | L | Y | L | — | D | E | — | — | Y | 0.153 | 1.159 | 0.376 | 1898 |
| 1601 | | | | | | | | | | | | | | | | | | |
| Phage- | — | — | — | — | — | S | F | L | — | Y | E | — | — | N | 0.063 | 1.13 | 0.104 | 1899 |
| 1602 | | | | | | | | | | | | | | | | | | |
| Phage- | — | S | — | — | — | P | Y | L | — | T | V | — | Y | N | 0.176 | 1.104 | 0.326 | 1900 |
| 1603 | | | | | | | | | | | | | | | | | | |
| Phage- | — | H | — | — | — | — | D | L | — | Y | E | — | — | A | 0.072 | 0.995 | 0.108 | 1901 |
| 1604 | | | | | | | | | | | | | | | | | | |
| Phage- | — | T | — | — | — | A | Y | L | — | H | E | — | — | N | 0.086 | 0.985 | 0.139 | 1902 |
| 1605 | | | | | | | | | | | | | | | | | | |
| Phage- | — | A | — | — | — | — | Y | L | — | A | E | — | — | — | 0.51 | 0.918 | 0.216 | 1903 |
| 1606 | | | | | | | | | | | | | | | | | | |
| Phage- | — | — | — | — | — | — | Y | L | — | — | — | — | — | A | 0.095 | 0.917 | 0.218 | 1904 |
| 1607 | | | | | | | | | | | | | | | | | | |
| Phage- | — | — | — | — | — | — | I | L | — | S | — | — | — | N | 0.082 | 0.847 | 0.113 | 1905 |
| 1608 | | | | | | | | | | | | | | | | | | |
| Phage- | — | A | — | — | — | L | D | L | — | D | K | — | — | Y | 0.079 | 0.77 | 0.141 | 1906 |
| 1609 | | | | | | | | | | | | | | | | | | |
| Phage- | — | N | — | — | — | — | A | L | — | H | E | — | — | L | 0.085 | 0.759 | 0.107 | 1907 |
| 1610 | | | | | | | | | | | | | | | | | | |
| Phage- | — | — | — | — | — | — | H | L | — | T | Y | — | — | H | 0.109 | 0.757 | 0.207 | 1908 |
| 1611 | | | | | | | | | | | | | | | | | | |
| Phage- | — | — | — | — | — | F | D | L | — | L | E | — | — | N | 0.087 | 0.65 | 0.12 | 1909 |
| 1612 | | | | | | | | | | | | | | | | | | |
| Phage- | — | H | — | — | — | L | H | L | — | N | E | — | — | A | 0.065 | 0.575 | 0.083 | 1910 |
| 1613 | | | | | | | | | | | | | | | | | | |
| Phage- | — | — | — | — | — | — | N | L | — | A | E | — | — | S | 0.061 | 0.538 | 0.08 | 1911 |
| 1614 | | | | | | | | | | | | | | | | | | |
| Phage- | — | — | Y | — | — | S | Y | L | — | H | E | — | — | A | 0.065 | 0.47 | 0.09 | 1912 |
| 1615 | | | | | | | | | | | | | | | | | | |
| Phage- | — | A | — | — | — | — | I | L | — | H | Q | — | — | N | 0.113 | 0.322 | 0.129 | 1913 |
| 1616 | | | | | | | | | | | | | | | | | | |
| Phage- | I | T | — | — | — | T | S | L | — | N | R | — | L | V | 0.077 | 0.242 | 0.089 | 1914 |
| 1617 | | | | | | | | | | | | | | | | | | |
| Phage- | L | A | G | — | Q | — | D | L | A | T | V | — | V | I | 0.068 | 0.229 | 0.116 | 1915 |
| 1618 | | | | | | | | | | | | | | | | | | |
| Phage- | I | M | — | — | — | T | S | L | — | N | R | — | V | T | 0.104 | 0.161 | 0.129 | 1916 |
| 1619 | | | | | | | | | | | | | | | | | | |
| Phage- | I | L | R | — | Q | M | N | L | Q | D | E | — | L | N | 0.083 | 0.083 | 0.077 | 1917 |
| 1620 | | | | | | | | | | | | | | | | | | |
| Phage- | N | H | — | — | — | T | T | L | — | N | R | — | V | A | 0.293 | 0.078 | 0.093 | 1918 |
| 1621 | | | | | | | | | | | | | | | | | | |
| Phage- | — | — | — | — | — | — | H | L | — | L | E | — | — | N | 0.064 | 0.066 | 0.074 | 1919 |
| 1622 | | | | | | | | | | | | | | | | | | |
| Phage- | — | — | — | — | — | — | F | M | — | N | F | — | — | — | 2.2 | 2.621 | 2.539 | 1920 |
| 1623 | | | | | | | | | | | | | | | | | | |
| Phage- | — | — | — | — | — | S | D | M | — | A | N | — | — | — | 0.11 | 2.621 | 0.316 | 1921 |
| 1624 | | | | | | | | | | | | | | | | | | |
| Phage- | I | — | — | — | — | M | H | M | — | D | F | — | Y | N | 0.078 | 2.544 | 0.232 | 1922 |
| 1625 | | | | | | | | | | | | | | | | | | |
| Phage- | — | — | — | — | — | F | D | M | — | A | — | — | — | — | 0.063 | 2.532 | 0.215 | 1923 |
| 1626 | | | | | | | | | | | | | | | | | | |
| Phage- | L | H | — | — | — | T | S | M | — | T | Y | — | Y | Y | 0.688 | 2.475 | 2.456 | 1924 |
| 1627 | | | | | | | | | | | | | | | | | | |
| Phage- | — | — | — | — | — | — | D | M | — | F | E | — | — | N | 0.069 | 2.422 | 0.778 | 1925 |
| 1628 | | | | | | | | | | | | | | | | | | |
| Phage- | — | — | — | — | — | — | Y | M | — | S | — | — | — | — | 0.082 | 2.28 | 0.377 | 1926 |
| 1629 | | | | | | | | | | | | | | | | | | |
| Phage- | — | — | — | — | — | K | D | M | — | A | E | — | — | N | 0.083 | 2.106 | 0.457 | 1927 |
| 1630 | | | | | | | | | | | | | | | | | | |
| Phage- | T | — | — | — | — | — | D | M | — | Y | L | — | — | N | 0.082 | 2.106 | 0.301 | 1928 |
| 1631 | | | | | | | | | | | | | | | | | | |
| Phage- | — | — | — | — | — | — | A | M | — | D | Y | — | — | Y | 0.123 | 2.106 | 0.339 | 1929 |
| 1632 | | | | | | | | | | | | | | | | | | |
| Phage- | — | — | — | — | — | — | N | M | — | N | E | — | — | — | 0.095 | 2.099 | 0.43 | 1930 |
| 1633 | | | | | | | | | | | | | | | | | | |
| Phage- | — | — | — | — | — | — | S | M | — | D | S | — | — | N | 0.332 | 2.019 | 0.142 | 1931 |
| 1634 | | | | | | | | | | | | | | | | | | |
| Phage- | — | H | — | — | — | T | Y | M | — | S | E | — | — | A | 0.07 | 1.839 | 0.161 | 1932 |
| 1635 | | | | | | | | | | | | | | | | | | |
| Phage- | — | — | — | — | — | L | D | M | — | — | L | — | — | A | 0.085 | 1.837 | 0.169 | 1933 |
| 1636 | | | | | | | | | | | | | | | | | | |
| Phage- | — | — | — | — | — | — | H | M | — | H | E | — | — | H | 0.067 | 1.815 | 0.171 | 1934 |
| 1637 | | | | | | | | | | | | | | | | | | |
| Phage- | — | S | — | — | — | W | D | M | — | N | E | — | — | A | 0.069 | 1.521 | 0.108 | 1935 |
| 1638 | | | | | | | | | | | | | | | | | | |
| Phage- | — | — | — | — | — | — | D | M | — | T | S | — | — | N | 0.1 | 1.414 | 0.179 | 1936 |
| 1639 | | | | | | | | | | | | | | | | | | |
| Phage- | — | — | — | — | — | M | S | M | — | A | F | — | — | D | 0.102 | 1.412 | 0.16 | 1937 |
| 1640 | | | | | | | | | | | | | | | | | | |
| Phage- | — | N | — | — | — | I | D | M | — | T | E | — | — | N | 0.245 | 1.403 | 0.175 | 1938 |
| 1641 | | | | | | | | | | | | | | | | | | |
| Phage- | — | N | — | — | — | I | H | M | — | N | Q | — | — | A | 0.085 | 1.373 | 0.133 | 1939 |
| 1642 | | | | | | | | | | | | | | | | | | |
| Phage- | — | F | — | — | — | K | D | M | — | A | E | — | — | A | 0.099 | 1.258 | 0.113 | 1940 |
| 1643 | | | | | | | | | | | | | | | | | | |
| Phage- | L | H | — | — | — | I | T | M | — | T | Y | — | Y | Y | 0.229 | 1.212 | 0.516 | 1941 |
| 1644 | | | | | | | | | | | | | | | | | | |
| Phage- | I | — | — | — | — | — | Y | M | — | H | E | — | — | — | 0.098 | 1.178 | 0.164 | 1942 |
| 1645 | | | | | | | | | | | | | | | | | | |
| Phage- | — | S | — | — | — | — | F | M | — | H | E | — | — | N | 0.081 | 1.111 | 0.246 | 1943 |
| 1646 | | | | | | | | | | | | | | | | | | |
| Phage- | Q | H | — | — | — | — | D | M | — | N | — | — | Y | A | 0.066 | 1.076 | 0.149 | 1944 |
| 1647 | | | | | | | | | | | | | | | | | | |
| Phage- | — | — | — | — | — | S | D | M | — | S | N | — | — | N | 0.07 | 1.075 | 0.121 | 1945 |
| 1648 | | | | | | | | | | | | | | | | | | |
| Phage- | L | Y | — | — | — | — | D | M | — | A | Y | — | Y | S | 0.068 | 1.019 | 0.157 | 1946 |
| 1649 | | | | | | | | | | | | | | | | | | |
| Phage- | — | — | — | — | — | M | Y | M | — | H | K | — | — | H | 0.099 | 0.987 | 0.168 | 1947 |
| 1650 | | | | | | | | | | | | | | | | | | |
| Phage- | — | — | — | — | — | S | N | M | — | N | E | — | — | — | 0.072 | 0.983 | 0.136 | 1948 |
| 1651 | | | | | | | | | | | | | | | | | | |
| Phage- | I | Y | — | — | — | T | A | M | — | N | R | — | S | A | 0.086 | 0.817 | 0.073 | 1949 |
| 1652 | | | | | | | | | | | | | | | | | | |
| Phage- | — | Y | — | — | — | K | Y | M | — | S | E | — | — | A | 0.072 | 0.741 | 0.099 | 1950 |
| 1653 | | | | | | | | | | | | | | | | | | |
| Phage- | V | — | — | — | — | A | H | M | — | N | E | — | Y | N | 0.208 | 0.664 | 0.085 | 1951 |
| 1654 | | | | | | | | | | | | | | | | | | |
| Phage- | — | F | — | — | — | S | T | M | — | N | R | — | — | D | 0.063 | 0.464 | 0.138 | 1952 |
| 1655 | | | | | | | | | | | | | | | | | | |
| Phage- | T | I | F | — | — | S | T | M | — | N | R | — | W | T | 0.138 | 0.262 | 0.085 | 1953 |
| 1656 | | | | | | | | | | | | | | | | | | |
| Phage- | — | Y | — | — | — | I | N | M | — | D | Y | — | Y | A | 0.122 | 0.109 | 0.088 | 1954 |
| 1657 | | | | | | | | | | | | | | | | | | |
| Phage- | I | N | L | — | — | T | P | M | — | N | R | — | W | L | 0.119 | 0.103 | 0.101 | 1955 |
| 1658 | | | | | | | | | | | | | | | | | | |
| Phage- | I | T | Q | — | — | S | T | M | — | N | R | — | S | V | 0.067 | 0.098 | 0.108 | 1956 |
| 1659 | | | | | | | | | | | | | | | | | | |
| Phage- | — | — | L | — | — | T | A | M | — | N | R | — | — | Y | 0.063 | 0.097 | 0.084 | 1957 |
| 1660 | | | | | | | | | | | | | | | | | | |
| Phage- | — | F | L | — | — | S | A | M | — | N | R | — | — | Y | 0.069 | 0.094 | 0.114 | 1958 |
| 1661 | | | | | | | | | | | | | | | | | | |
| Phage- | V | I | L | — | — | T | T | M | — | N | R | — | — | H | 0.1 | 0.072 | 0.084 | 1959 |
| 1662 | | | | | | | | | | | | | | | | | | |
| Phage- | — | N | — | — | — | S | T | M | — | N | R | — | L | T | 0.067 | 0.065 | 0.07 | 1960 |
| 1663 | | | | | | | | | | | | | | | | | | |
| Phage- | I | H | L | — | H | W | V | P | — | I | K | — | S | H | 2.516 | 2.55 | 2.508 | 1961 |
| 1664 | | | | | | | | | | | | | | | | | | |
| Phage- | — | A | — | — | — | A | D | Q | — | H | E | — | — | N | 0.076 | 1.845 | 0.456 | 1962 |
| 1665 | | | | | | | | | | | | | | | | | | |
| Phage- | I | — | — | — | — | I | I | Q | G | L | P | — | — | A | 0.058 | 0.368 | 0.065 | 1963 |
| 1666 | | | | | | | | | | | | | | | | | | |
| Phage- | — | A | — | — | — | W | A | Q | F | D | E | — | L | A | 0.073 | 0.071 | 0.078 | 1964 |
| 1667 | | | | | | | | | | | | | | | | | | |
| Phage- | I | V | — | — | — | T | T | Q | — | N | R | — | A | T | 0.113 | 0.067 | 0.083 | 1965 |
| 1668 | | | | | | | | | | | | | | | | | | |
| Phage- | — | — | — | — | — | — | D | — | — | — | — | — | Y | A | 0.157 | 1.484 | 0.403 | 1966 |
| 1669 | | | | | | | | | | | | | | | | | | |
| Phage- | — | — | — | — | — | — | D | — | — | A | — | — | — | N | 0.063 | 1.351 | 0.08 | 1967 |
| 1670 | | | | | | | | | | | | | | | | | | |
| Phage- | — | — | — | — | — | V | D | — | — | A | — | — | — | N | 0.071 | 1.156 | 0.315 | 1968 |
| 1671 | | | | | | | | | | | | | | | | | | |
| Phage- | — | — | — | — | — | M | S | — | — | A | F | — | — | D | 0.071 | 1 | 0.115 | 1969 |
| 1672 | | | | | | | | | | | | | | | | | | |
| Phage- | — | — | — | — | — | — | D | — | — | D | V | — | — | A | 0.073 | 0.924 | 0.096 | 1970 |
| 1673 | | | | | | | | | | | | | | | | | | |
| Phage- | — | — | — | — | — | — | D | — | — | — | — | — | — | N | 0.106 | 0.911 | 0.132 | 1971 |
| 1674 | | | | | | | | | | | | | | | | | | |
| Phage- | — | — | — | — | — | — | D | — | — | D | — | — | — | — | 0.114 | 0.899 | 0.183 | 1972 |
| 1675 | | | | | | | | | | | | | | | | | | |
| Phage- | I | Y | — | — | — | I | D | — | — | N | — | — | Y | N | 0.177 | 0.867 | 0.164 | 1973 |
| 1676 | | | | | | | | | | | | | | | | | | |
| Phage- | — | — | — | — | — | M | T | — | — | N | — | — | Y | — | 0.081 | 0.671 | 0.078 | 1974 |
| 1677 | | | | | | | | | | | | | | | | | | |
| Phage- | V | N | Q | — | T | — | Y | — | — | A | E | — | L | N | 0.465 | 0.566 | 0.135 | 1975 |
| 1678 | | | | | | | | | | | | | | | | | | |
| Phage- | — | — | — | — | — | — | A | — | — | H | — | — | — | — | 0.097 | 0.497 | 0.138 | 1976 |
| 1679 | | | | | | | | | | | | | | | | | | |
| Phage- | — | — | — | — | — | — | D | — | — | D | — | — | — | N | 0.069 | 0.45 | 0.077 | 1977 |
| 1680 | | | | | | | | | | | | | | | | | | |
| Phage- | — | — | — | — | — | — | D | — | — | D | V | — | Y | Y | 0.073 | 0.074 | 0.073 | 1978 |
| 1681 | | | | | | | | | | | | | | | | | | |
| Phage- | — | A | — | — | — | W | A | S | F | D | E | — | L | A | 0.104 | 0.089 | 0.109 | 1979 |
| 1682 | | | | | | | | | | | | | | | | | | |
| Phage- | — | V | D | — | — | L | I | S | F | D | E | — | L | A | 0.246 | 0.07 | 0.076 | 1980 |
| 1683 | | | | | | | | | | | | | | | | | | |
| Phage- | E | — | — | — | — | T | D | V | — | P | Y | — | — | S | 0.1 | 0.711 | 0.157 | 1981 |
| 1684 | | | | | | | | | | | | | | | | | | |
| Phage- | — | — | — | — | — | — | F | W | — | H | E | — | Y | A | 0.246 | 2.397 | 0.503 | 1982 |
| 1685 | | | | | | | | | | | | | | | | | | |
| Phage- | — | — | — | — | — | — | D | W | — | H | M | — | — | N | 0.1 | 2.115 | 0.243 | 1983 |
| 1686 | | | | | | | | | | | | | | | | | | |
| Phage- | — | — | — | — | — | S | D | Y | — | Y | V | — | — | A | 0.065 | 0.707 | 0.08 | 1984 |
| 1687 | | | | | | | | | | | | | | | | | | |

(—) indicates same amino acid as in anti-CD28 scFv Peptide-116 corresponding position.

### Example 14. Peptides Inhibit Anti-CD28 scFv from Binding CD28 Antigen by ELISA

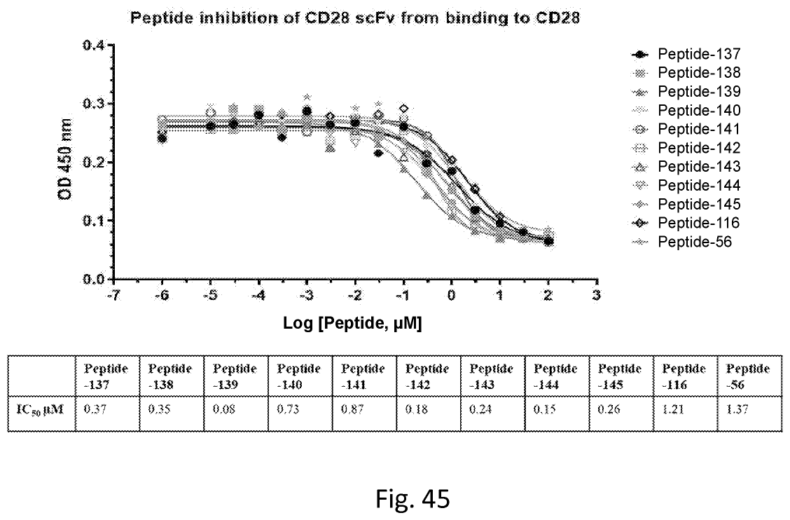

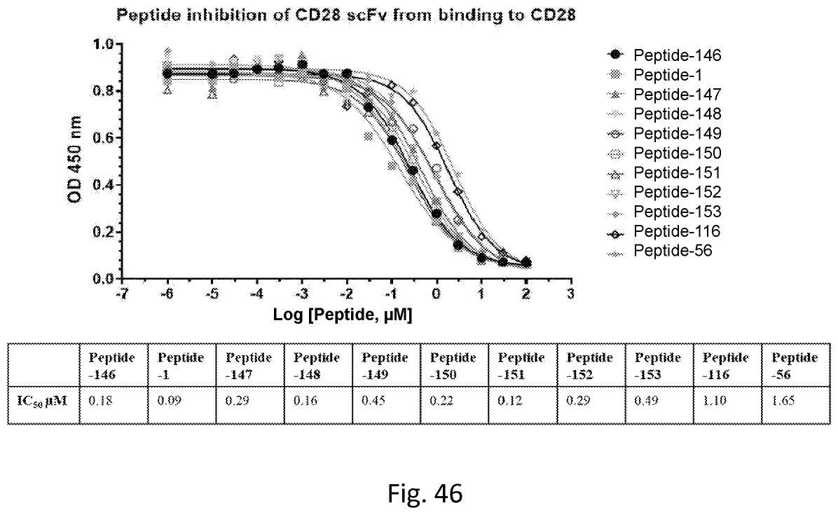

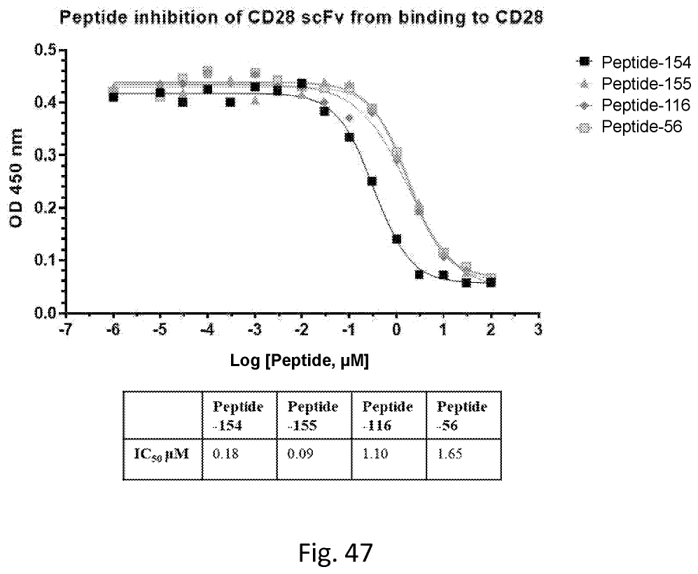

Peptides were evaluated for their ability to inhibit the anti-CD28 scFv (SEQ ID NO: 35) from binding to the CD28 antigen in a standard enzyme linked immunosorbent assay (ELISA) format. Briefly, biotinylated CD28 antigen was captured on neutravidin coated plates. Anti-CD28 scFv at 2 nM were pre-incubated with 0-100 μM titrated peptides. After a short pre-incubation period the mixture of titrated peptides with fixed anti-CD28 scFv (2 nM) were added to the CD28 antigen captured plates. After a short incubation on the plates, bound anti-CD28 scFv was detected with a standard horse radish peroxidase conjugated secondary antibody. The concentration of peptide required to reduce the max signal by 50% (IC₅₀) was calculated in Graphpad Prism software. FIGS. 45-47 illustrate peptides that inhibit the anti-CD28 scFv from binding the CD28 antigen measured by ELISA.

### Example 15. Anti-CD28 scFv Kinetic Binding to Peptides by Octet

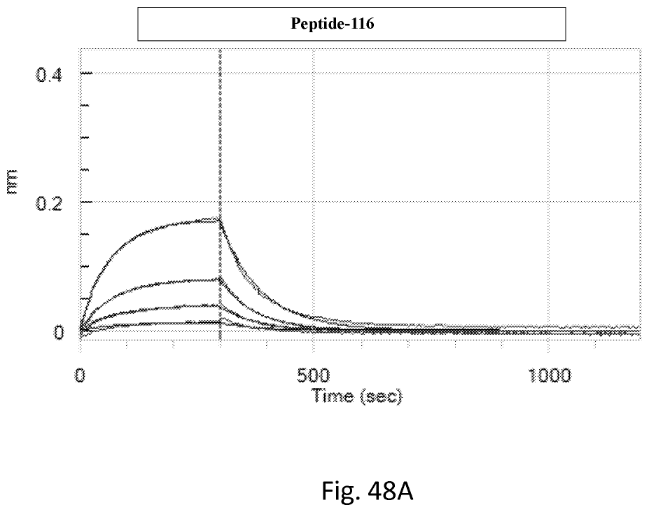

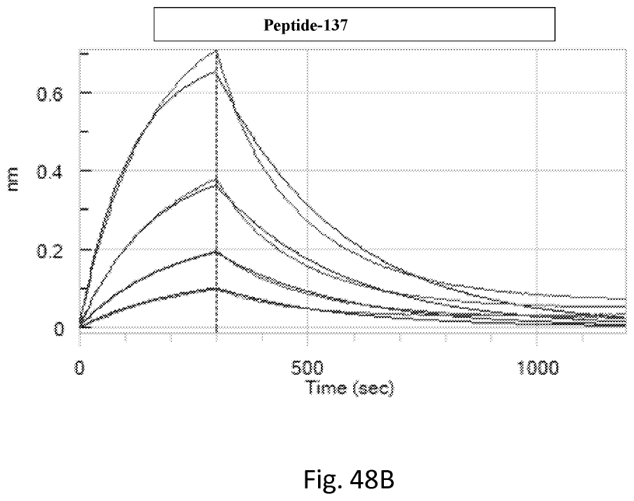

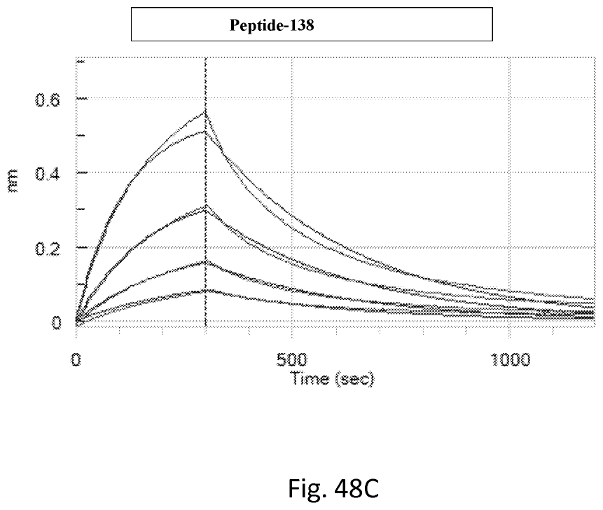

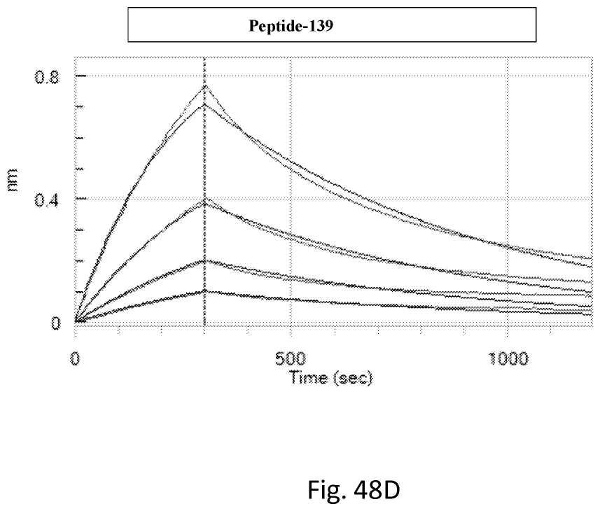

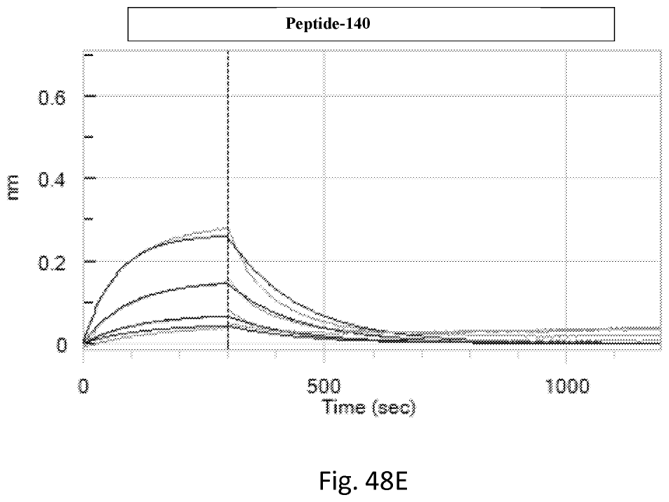

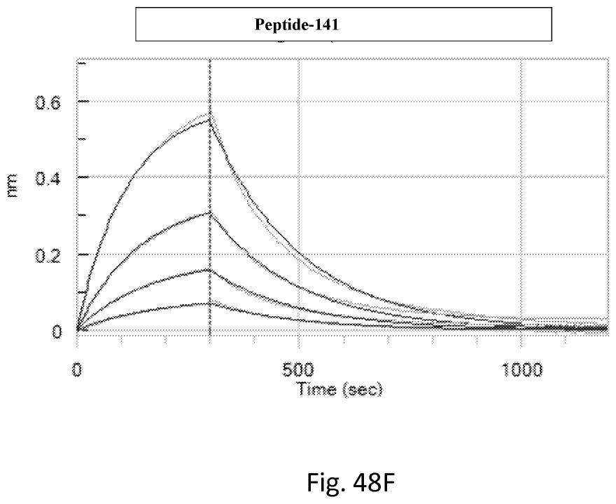

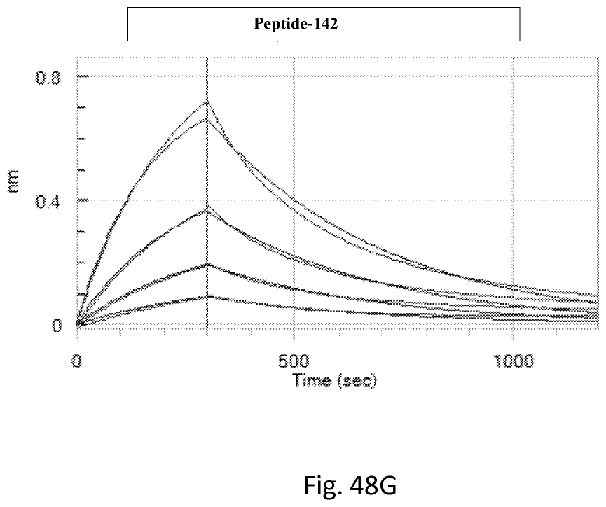

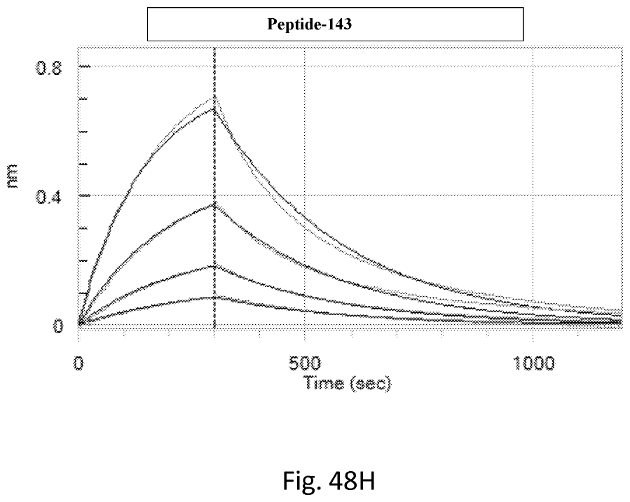

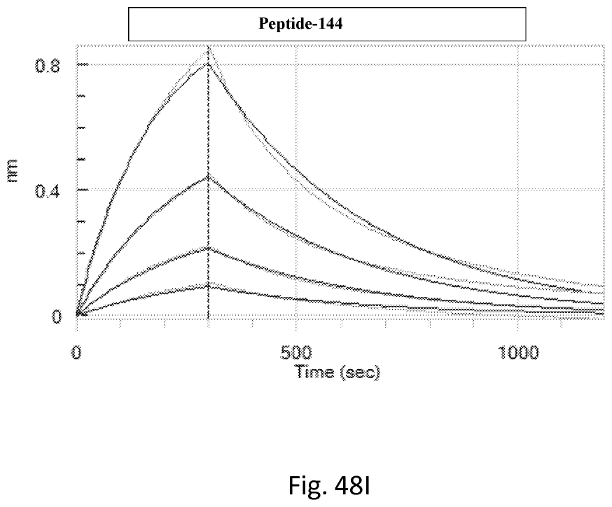

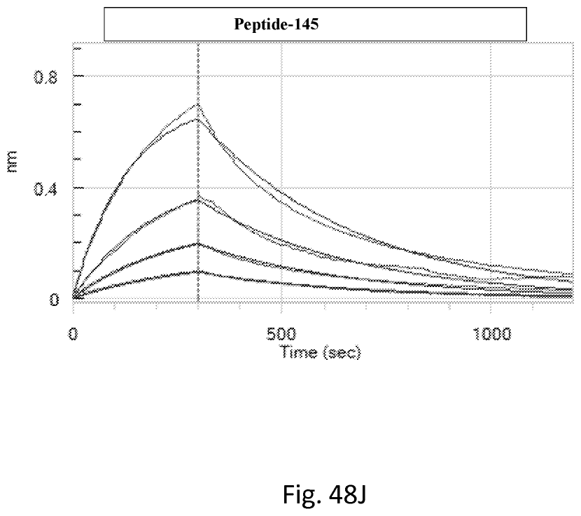

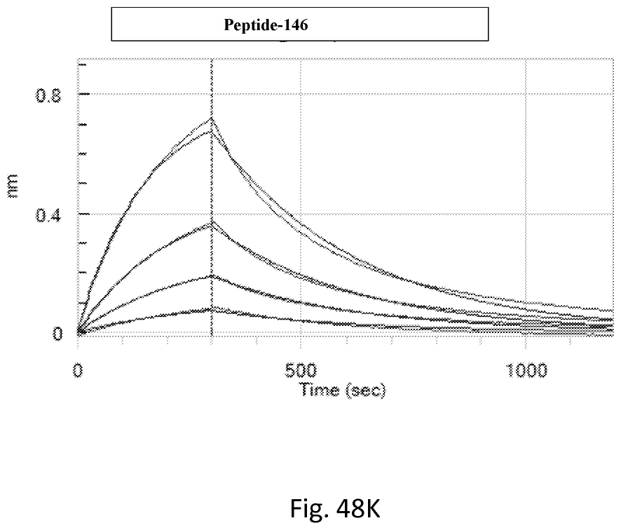

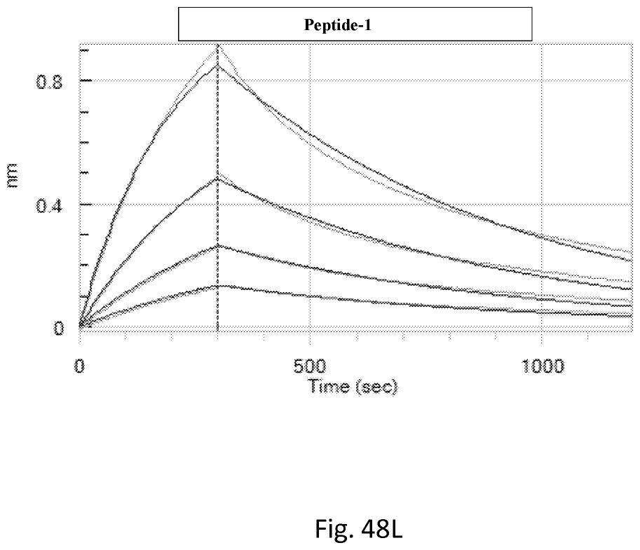

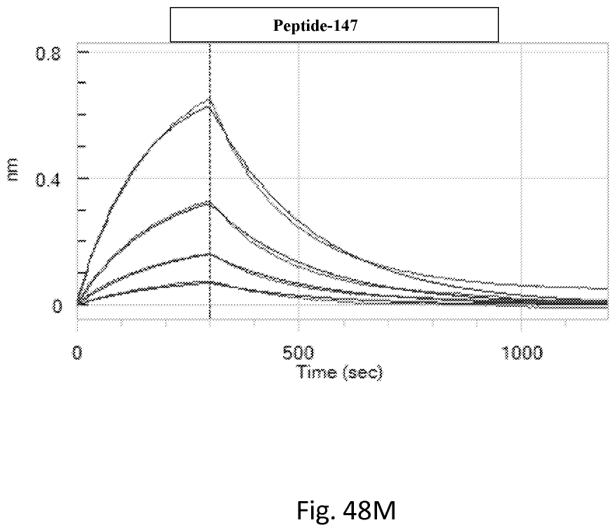

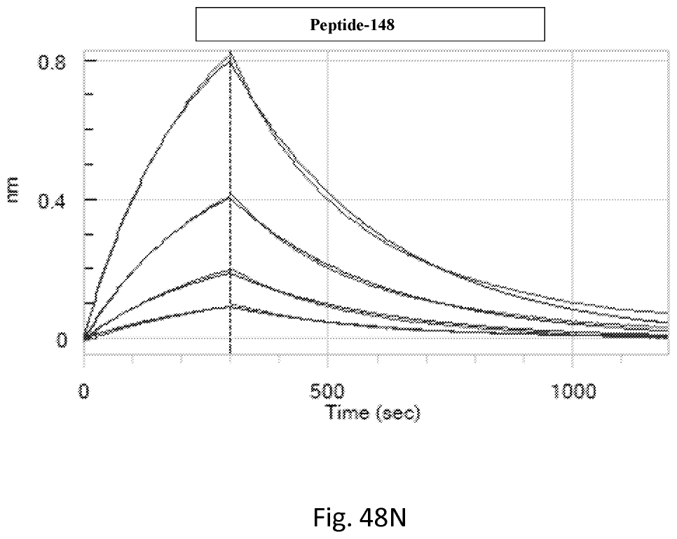

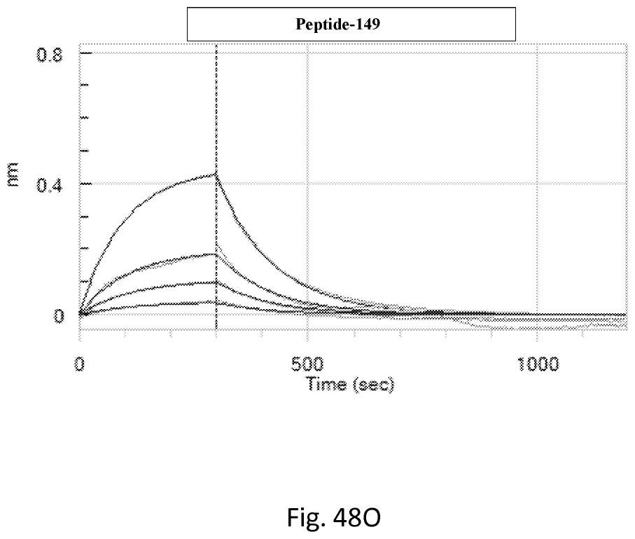

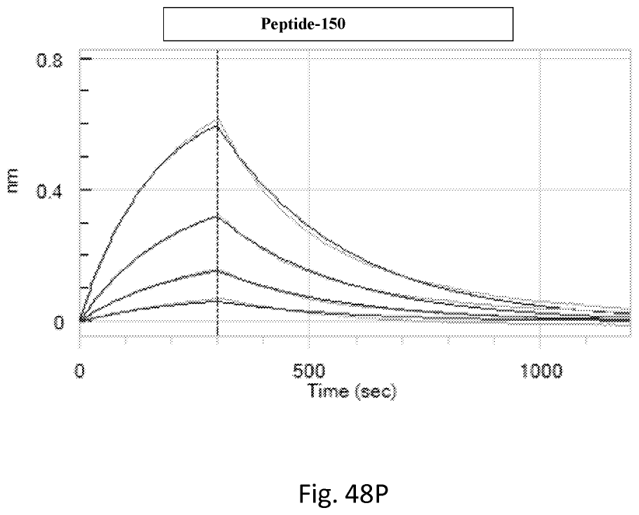

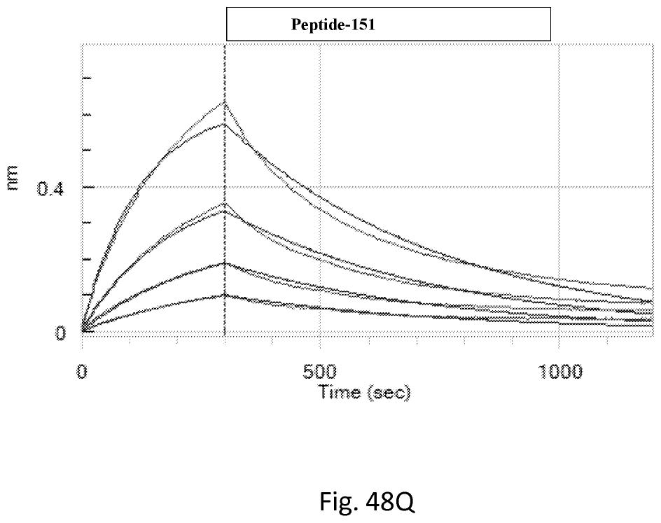

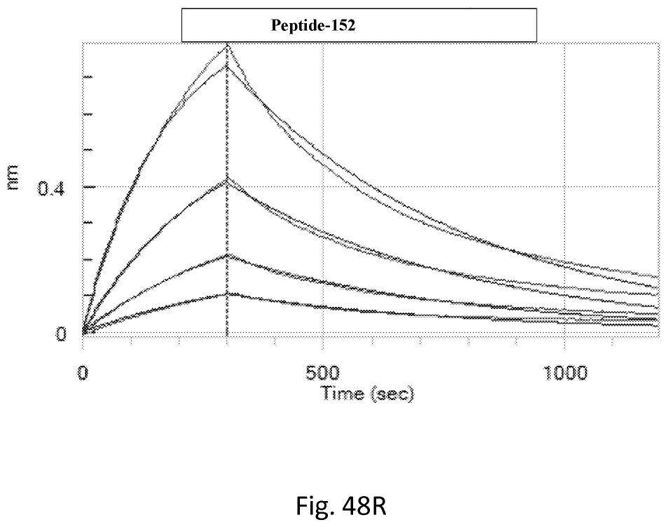

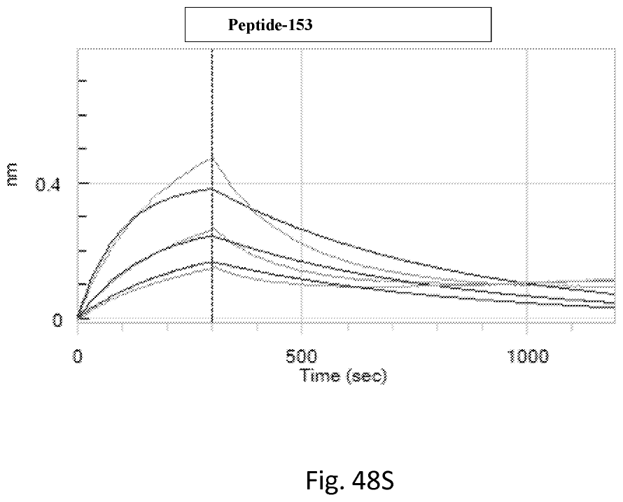

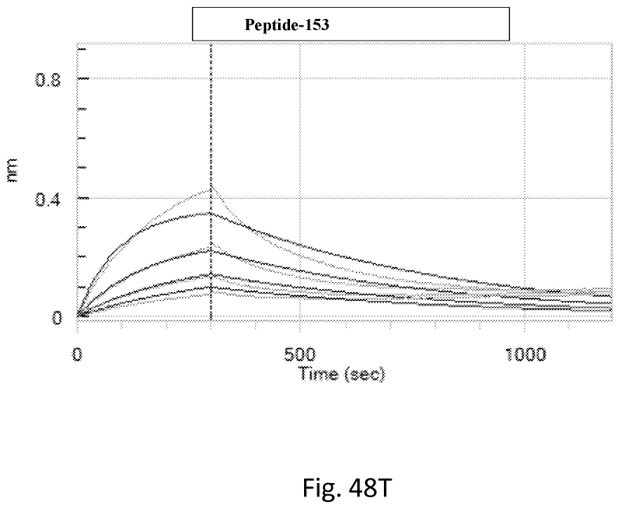

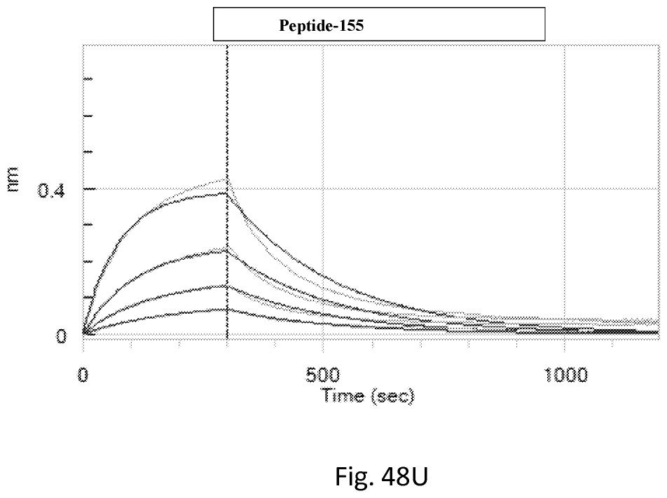

Kinetic binding of anti-CD28 scFv (SEQ ID NO: 35) to peptides were evaluated by bio-layer interferometry using an Octet RED96 instrument. Briefly, streptavidin biosensors were loaded with biotinylated peptides and baselined in buffer. Anti-CD28 scFv were titrated in solution at 100 nM, 50 nM, 25 nM, and 12.5 nM, then associated onto the peptide loaded sensors. After a short association period, sensors were transferred into buffer and the dissociation of bound anti-CD28 scFv was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. FIGS. 48A-48F illustrate kinetic binding of anti-CD28 scFv binding to peptides as measured by Octet. FIGS. 48G-48U illustrate kinetic binding of peptides to the anti-CD28 scFv as measured by Octet. Kinetic sensorgrams are summarized in Tables 30-31.

TABLE 30
Timing and Steps of Assay

| Step | Time |
|---|---|
| Baseline: Buffer | 60 sec |
| Load: | 300 sec |
| 200 nM Peptide | |
| Baseline: Buffer | 300 sec |
| Association in octet buffer | 300 sec |
| 100 nM CD28 scFv | |
| 50 nM CD28 scFv | |
| 25 nM CD28 scFv | |
| 12.5 nM CD28 scFv | |
| Dissociation: Buffer | 900 sec |

TABLE 31
Summary of Kinetic Data

| | Loading | | | |
|---|---|---|---|---|
| Sample ID | Sample ID | KD (M) | kon(1/Ms) | kdis(1/s) |
| Anti-CD28 scFv | Peptide-137 | 7.18E−08 | 5.14E+04 | 3.69E−03 |
| Anti-CD28 scFv | Peptide-138 | 4.99E−08 | 5.91E+04 | 2.95E−03 |
| Anti-CD28 scFv | Peptide-139 | 5.28E−08 | 2.90E+04 | 1.53E−03 |
| Anti-CD28 scFv | Peptide-140 | 8.88E−08 | 7.70E+04 | 6.84E−03 |
| Peptide-141 | Peptide-141 | 1.27E−07 | 3.90E+04 | 4.97E−03 |
| Anti-CD28 scFv | Peptide-142 | 6.87E−08 | 3.67E+04 | 2.52E−03 |
| Anti-CD28 scFv | Peptide-143 | 9.78E−08 | 3.59E+04 | 3.51E−03 |
| Anti-CD28 scFv | Peptide-144 | 9.23E−08 | 3.00E+04 | 2.77E−03 |
| Anti-CD28 scFv | Peptide-145 | 5.19E−08 | 5.11E+04 | 2.65E−03 |
| Anti-CD28 scFv | Peptide-146 | 7.99E−08 | 3.88E+04 | 3.10E−03 |
| Anti-CD28 scFv | Peptide-1 | 4.23E−08 | 3.64E+04 | 1.54E−03 |
| Anti-CD28 scFv | Peptide-147 | 1.52E−07 | 2.84E+04 | 4.33E−03 |
| Anti-CD28 scFv | Peptide-148 | 1.92E−07 | 1.67E+04 | 3.20E−03 |
| Anti-CD28 scFv | Peptide-149 | 3.74E−07 | 2.17E+04 | 8.12E−03 |
| Anti-CD28 scFv | Peptide-150 | 1.34E−07 | 2.71E+04 | 3.64E−03 |
| Anti-CD28 scFv | Peptide-151 | 3.52E−08 | 6.19E+04 | 2.18E−03 |
| Anti-CD28 scFv | Peptide-152 | 5.18E−08 | 3.89E+04 | 2.01E−03 |
| Anti-CD28 scFv | Peptide-153 | 2.11E−08 | 8.89E+04 | 1.87E−03 |
| Anti-CD28 scFv | Peptide-154 | 2.11E−08 | 8.82E+04 | 1.86E−03 |
| Anti-CD28 scFv | Peptide-155 | 4.80E−08 | 9.23E+04 | 4.43E−03 |
| Anti-CD28 scFv | Peptide-156 | 2.78E−07 | 4.15E+04 | 1.15E−02 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. An isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 1-26 or the peptide comprises 1, 2, or 3, amino acid substitutions, additions, or deletions relative to an amino acid sequence selected from any one of SEQ ID NOs: 1-26.

2. The isolated polypeptide or polypeptide complex of claim 1, wherein the peptide comprises an amino acid sequence selected from any one of SEQ ID NOs: 1-26.

3. The isolated polypeptide or polypeptide complex of claim 1, wherein the peptide consists of an amino acid sequence selected from any one of SEQ ID NOs: 1-26.

4. The isolated polypeptide or polypeptide complex of claim 1, wherein the peptide comprises the amino acid sequence of SEQ ID NO: 1.

5. The isolated polypeptide or polypeptide complex of claim 1, wherein the peptide consists of the amino acid sequence of SEQ ID NO: 1.

6. The isolated polypeptide or polypeptide complex of claim 1, wherein the peptide comprises an amino acid sequence selected from any one of SEQ ID NOs: 2-19 and 22-26 or the peptide comprises 1, 2, or 3 amino acid substitutions, additions, or deletions relative to an amino acid sequence selected from any one of SEQ ID NOs: 2-19 and 22-26.

7. The isolated polypeptide or polypeptide complex of claim 1, wherein the peptide comprises an amino acid sequence selected from any one of SEQ ID NOs: 2-19 and 22-26.

8. The isolated polypeptide or polypeptide complex of claim 1, wherein the peptide comprises an amino acid sequence selected from any one of SEQ ID NOs: 2-19.

9. The isolated polypeptide or polypeptide complex of claim 1, wherein the peptide consists of an amino acid sequence selected from any one of SEQ ID NOs: 2-19.

10. The isolated polypeptide or polypeptide complex of claim 1, wherein the peptide comprises an amino acid sequence selected from any one of SEQ ID NOs: 2-9 and 12-18.

11. The isolated polypeptide or polypeptide complex of claim 1, wherein the peptide consists of an amino acid sequence selected from any one of SEQ ID NOs: 2-9 and 12-18.

12. The isolated polypeptide or polypeptide complex of claim 1, wherein the peptide comprises an amino acid sequence selected from any one of SEQ ID NOs: 3, 6-7, 9-13, and 16-19.

13. The isolated polypeptide or polypeptide complex of claim 1, wherein the peptide comprises an amino acid sequence selected from any one of SEQ ID NOs: 3, 6, 11-12, and 16-17.

14. The isolated polypeptide or polypeptide complex of claim 1, wherein the peptide comprises an amino acid sequence selected from any one of SEQ ID NOs: 22-26.

15. The isolated polypeptide or polypeptide complex of claim 1, wherein the peptide consists of an amino acid sequence selected from any one of SEQ ID NOs: 22-26.

16. The isolated polypeptide or polypeptide complex of claim 1, wherein the peptide comprises an amino acid sequence selected from any one of SEQ ID NOs: 22-24 and 26.

17. The isolated polypeptide or polypeptide complex of claim 1, wherein the peptide comprises an amino acid sequence selected from any one of SEQ ID Nos: 22-23 and 26.

18. The isolated polypeptide or polypeptide complex of claim 1, wherein the peptide comprises the amino acid sequence of SEQ ID NO: 23.

19. The isolated polypeptide or polypeptide complex of claim 1, wherein the peptide consists of the amino acid sequence of SEQ ID NO: 23.

20. An isolated polypeptide or polypeptide complex comprising a peptide that impairs binding of a CD28 binding domain to CD28 expressed on an effector cell, wherein the peptide comprises an amino acid sequence according to: X₁-X₂-W-C-X₃-X₄-X₅-X₆-X₇-X₈-X₉-C-X₁₀-X₁₁, wherein
X₁ is selected from A, R, N, D, Q, E, G, H, I, L, K, F, P, S, T, W, Y, and V;
X₂ is selected from D, P, and Y;
X₃ is selected from P and Q;
X₄ is selected from R, L, and I;
X₅ is selected from D, H, Y, and I;
X₆ is selected from L, S, and R;
X₇ is selected from W and G;
X₈ is selected from V, A, D, H, L, and N;
X₉ is selected from H, L, E, and D;
X₁₀ is selected from F, Y, and L; and
X₁₁ is selected from A, N, H, and T.

21. The isolated polypeptide or polypeptide complex of claim 20, wherein:
X₁ is selected from A, R, N, D, Q, E, G, H, K, F, P, S, T, W, and Y.

22. The isolated polypeptide or polypeptide complex of claim 20, wherein:
X₁ is selected from R, Q, E, H, I, L, K, F, T, W, Y, and V;
X₂ is D;
X₃ is P;
X₄ is R;
X₅ is D;
X₆ is L;
X₇ is W;
X₈ is V;
X₉ is H;
X₁₀ is F; and
X₁₁ is A.

23. The isolated polypeptide or polypeptide complex of claim 20, wherein
X₁ is selected from R, Q, E, L, K, T, and W;
X₂ is D;
X₃ is P;
X₄ is R;
X₅ is D;
X₆ is L;
X₇ is W;
X₈ is V;
X₉ is H;
X₁₀ is F; and
X₁₁ is A.

24. The isolated polypeptide or polypeptide complex of claim 20, wherein:
X₁ is L or I;
X₂ is D or Y;
X₃ is P;
X₄ is R, L, or I;
X₅ is H, Y, or D;
X₆ is L or R;
X₇ is W;
X₈ is A, D, H, or N;
X₉ is L, E, or D;
X₁₀ is F or Y; and
X₁₁ is N or H.

25. The isolated polypeptide or polypeptide complex of claim 20, wherein:
X₁ is L or I;
X₂ is D or Y;
X₃ is P;
X₄ is R or I;
X₅ is H, Y, or D;
X₆ is L or R;
X₇ is W;
X₈ is A, D, or N;
X₉ is L, E, or D;
X₁₀ is F or Y; and
X₁₁ is N.

26. An isolated polypeptide or polypeptide complex comprising a CD28 binding domain that is linked to a peptide that impairs binding of the CD28 binding domain to CD28, wherein the peptide comprises an amino acid sequence selected from any one of SEQ ID NOs: 2-26 or the peptide comprises 1, 2, or 3 amino acid substitutions, additions, or deletions relative to an amino acid sequence selected from any one of SEQ ID NOs: 2-26.

27. The isolated polypeptide or polypeptide complex of claim 26, wherein the peptide comprises an amino acid sequence selected from any one of SEQ ID NOs: 2-26.

28. The isolated polypeptide or polypeptide complex of claim 26, wherein the peptide consists of an amino acid sequence selected from any one of SEQ ID NOs: 2-26.

29. An isolated polypeptide or polypeptide complex comprising a CD28 binding domain that is linked to a peptide that impairs binding of the CD28 binding domain to CD28, wherein the peptide comprises an amino acid sequence according to: Z₁-C-Z₂-Z₃-Z₄-Z₅-Z₆-Z₇-Z₈-Z₉-Z₁₀-Z₁₁-C-Z₁₂, wherein:
Z₁ is selected from Y, H, and A;
Z₂ is selected from D, E, A, Q, S, V, M, H, L, F, T, W, and N;
Z₃ is selected from G, E, S, and A;
Z₄ is selected from H, L, W, and A;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, Q, and A;
Z₉ is selected from M, I, L, R, V, K, F, Q, Y, W, and A;
Z₁₀ is selected from A, Q, S, W, E, L, G, D, T, M, N, V, H, R, Y, K, P, F, and I;
Z₁₁ is selected from K, Q, N, H, E, P, and A; and
Z₁₂ is selected from L, M, I, F, V, Y, Q, D, T, and A.

30. The isolated polypeptide or polypeptide complex of claim 29, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

31. The isolated polypeptide or polypeptide complex of claim 29, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

32. The isolated polypeptide or polypeptide complex of claim 29, wherein:
Z₁ is Y;
Z₂ is selected from D, E, A, and Q;
Z₃ is G;
Z₄ is H;
Z₅ is selected from L, S, A, T, I, V, and M;
Z₆ is selected from P, A, E, Q, S, L, W, G, V, and D;
Z₇ is selected from E, Q, I, M, V, A, and L;
Z₈ is selected from Y and H;
Z₉ is selected from M, I, and L;
Z₁₀ is selected from A, Q, S, W, E, L, G, D, T, M, and N;
Z₁₁ is K; and
Z₁₂ is selected from L and M.

33. The isolated polypeptide or polypeptide complex of claim 29, wherein:
Z₁ is Y;
Z₂ is selected from D, E, and A;
Z₃ is G;
Z₄ is H;
Z₅ is selected from L, S, A, and T;
Z₆ is selected from P, A, E, Q, S, L, and W;
Z₇ is selected from E, Q, I, and M;
Z₈ is Y;
Z₉ is selected from M, I, and L;
Z₁₀ is selected from A, Q, S, W, E, L, G, and D;
Z₁₁ is K; and
Z₁₂ is L.

34. The isolated polypeptide or polypeptide complex of claim 29, wherein:
Z₁ is Y;
Z₂ is selected from D and E;
Z₃ is G;
Z₄ is H;
Z₅ is selected from L, S, and A;
Z₆ is selected from P, A, E, Q, S, and L;
Z₇ is selected from E, Q, and I;
Z₈ is Y;
Z₉ is selected from M, I, and L;
Z₁₀ is selected from A, Q, S, W, E, L, G, and D;
Z₁₁ is K; and
Z₁₂ is L.

35. The isolated polypeptide or polypeptide complex of claim 29, wherein the peptide comprises the amino acid sequence of SEQ ID NO: 132.

36. The isolated polypeptide or polypeptide complex of claim 29, wherein the peptide consists of the amino acid sequence of SEQ ID NO: 132.

37. The isolated polypeptide or polypeptide complex of claim 29, wherein the peptide comprises an amino acid sequence according to any one of SEQ ID NOs: 162-181.

38. The isolated polypeptide or polypeptide complex of claim 29, wherein the peptide comprises the amino acid sequence according to SEQ ID NO: 172.

39. The isolated polypeptide or polypeptide complex of claim 29, wherein the peptide comprises an amino acid sequence according to any one of SEQ ID NOs: 152 to 161.

40. An isolated polypeptide or polypeptide complex comprising a CD28 binding domain that is linked to a peptide that impairs binding of the CD28 binding domain to CD28, wherein the peptide comprises an amino acid sequence according to: U₁-C-U₂-U₃-U₄-U₅-U₆-U₇-U₈-U₉-U₁₀-U₁₁-C-U₁₂, wherein:
U₁ is selected from T, S, L, M, W, Q, P, R, G, N, I, Y, D, E, V, H, and A;
U₂ is selected from A, S, T, K, E, R, G, N, W, and D;
U₃ is selected from A, E, P, D, G, W, Q, V, and F;
U₄ is selected from G, V, F, D, E, R, and A;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, I, and A;
U₈ is selected from P, S, T, A, E, K, Q, N, V, G, H, L, D, Y, I, M, and R;
U₉ is selected from W, L, and A;
U₁₀ is selected from P, S, R, L, E, and A;
U₁₁ is selected from L, A, T, M, V, W, Y, E, Q, I, F, S, P, K, R, D, H, N, and G; and
U₁₂ is selected from L, F, V, M, I, Y, E, W, and A.

41. The isolated polypeptide or polypeptide complex of claim 40, wherein:
U₁ is selected from T, S, L, M, W, Q, P, R, G, N, I, Y, D, E, V, and H;
U₂ is selected from A, S, T, K, E, R, G, N, W, and D;
U₃ is selected from A, E, P, D, G, W, Q, V, and F;
U₄ is selected from G, V, F, D, E, and R;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, and I;
U₈ is selected from P, S, T, A, E, K, Q, N, V, G, H, L, D, Y, I, M, and R;
U₉ is selected from W, L, and A;
U₁₀ is selected from P, S, R, L, and E;
U₁₁ is selected from L, A, T, M, V, W, Y, E, Q, I, F, S, P, K, R, D, H, N, and G; and
U₁₂ is selected from L, F, V, M, I, Y, E, and W.

42. The isolated polypeptide or polypeptide complex of claim 40, wherein:
U₁ is selected from T, S, L, M, and W;
U₂ is selected from A, S, and T;
U₃ is selected from A and E;
U₄ is selected from G, V, and F;
U₅ is selected from A, F, and V;
U₆ is selected from T, S, H, M, A, and N;
U₇ is selected from Y, W, and F;
U₈ is selected from P, S, T, A, E, K, Q, N, and V;
U₉ is selected from W and L;
U₁₀ is selected from P and S;
U₁₁ is selected from L, A, T, M, V, W, Y, E, Q, I, F, and S; and
U₁₂ is selected from L, F, V, and M.

43. The isolated polypeptide or polypeptide complex of claim 40, wherein:
U₁ is selected from T and S;
U₂ is selected from A and S;
U₃ is A;
U₄ is selected from G and V;
U₅ is selected from A and F;
U₆ is selected from T, S, and H;
U₇ is selected from Y and W;
U₈ is selected from P, S, T, and A;
U₉ is W;
U₁₀ is P;
U₁₁ is selected from L, A, T, M, V, W, and Y; and
U₁₂ is selected from L and F.

44. The isolated polypeptide or polypeptide complex of claim 40, wherein the peptide comprises the amino acid sequence of SEQ ID NO: 137.

45. The isolated polypeptide or polypeptide complex of claim 40, wherein the peptide consists of the amino acid sequence of SEQ ID NO: 137.

46. The isolated polypeptide or polypeptide complex of claim 40, wherein the peptide comprises an amino acid sequence according to any one of SEQ ID NOs: 144 to 151.

47. The isolated polypeptide or polypeptide complex of claim 40, wherein the peptide comprises an amino acid sequence according to any one of SEQ ID NOs: 182-201.

48. An isolated polypeptide or polypeptide complex comprising a CD28 binding domain that is linked to a peptide that impairs binding of the CD28 binding domain to CD28, wherein the peptide comprises an amino acid sequence according to any one of SEQ ID NOs: 120-201 and 204-1392.

49. An isolated polypeptide or polypeptide complex comprising a CD28 binding domain that is linked to a peptide that impair binding of the CD28 binding domain to CD28, wherein the peptide comprises an amino acid sequence according to X₁-X₂-X₃-C-X₄-X₅-X₆-X₇-X₈-X₉-X₁₀-C-X₁₁-X₁₂, wherein:
X₁ is selected from M, I, L, and V;
X₂ is selected from D, H, N, A, F, S, T, Y, and V;
X₃ is selected from W, L, and F;
X₄ is selected from P, A, and L;
X₅ is selected from R, T, I, M, S, K, L, V, W, F, A, P, and D;
X₆ is selected from E, D, Y, H, S, F, A, N, T, I, P, and V;
X₇ is selected from L, M, R, S, Q, and H;
X₈ is selected from W and Q;
X₉ is selected from H, N, D, A, S, Y, T, F, V, L, and I;
X₁₀ is selected from E, V, L, D, Y, R, Q, H, F, K, A, M, and N;
X₁₁ is selected from F, Y, L, W, and V; and
X₁₂ is selected from N, A, F, S, Y, H, D, T, and L.

50. The isolated polypeptide or polypeptide complex of claim 49, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y.

51. The isolated polypeptide or polypeptide complex of claim 49, wherein X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

52. The isolated polypeptide or polypeptide complex of claim 1, wherein the CD28 binding domain comprises an anti-CD28 antibody.

53. The isolated polypeptide or polypeptide complex of claim 52, wherein the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise the amino acid sequences of HC-CDR1: SEQ ID NO: 27, HC-CDR2: SEQ ID NO: 28, and HC-CDR3: SEQ ID NO: 29, and the anti-CD28 antibody comprises an anti-CD28 light chain variable domain that comprises complementarity determining regions (CDRs): LC-CDR1, LC-CDR2, and LC-CDR3, wherein the LC-CDR1, the LC-CDR2, and the LC-CDR3 comprise the amino acid sequences of LC-CDR1: SEQ ID NO: 30, LC-CDR2: (KAS), and LC-CDR3: SEQ ID: 32.

54. The isolated polypeptide or polypeptide complex of claim 53, wherein the anti-CD28 heavy chain variable domain comprises an amino acid sequence with at least 80%, 85%, 90%, 95%, 98%, 99%, or 100% sequence identity to SEQ ID NO: 33.

55. The isolated polypeptide or polypeptide complex of claim 53, wherein the anti-CD28 light chain variable domain comprises an amino acid sequence with at least 80%, 85%, 90%, 95%, 98%, 99%, or 100% sequence identity to SEQ ID NO: 34.

56. The isolated polypeptide or polypeptide complex of claim 52, wherein the anti-CD28 antibody comprises a single chain variable fragment (scFv), a single domain antibody, a Fab, or a Fab′.

57. The isolated polypeptide or polypeptide complex of claim 56, wherein the anti-CD28 antibody comprises the scFv.

58. The isolated polypeptide or polypeptide complex of claim 56, wherein the anti-CD28 antibody comprises the scFv, and the scFv comprises an amino acid sequence with at least 80%, 85%, 90%, 95%, 98%, 99%, or 100% sequence identity to SEQ ID NO: 35.

59. The isolated polypeptide or polypeptide complex of claim 56, wherein the anti-CD28 antibody comprises the scFv, and the scFv comprises an amino acid sequence with at least 80%, 85%, 90%, 95%, 98%, 99%, or 100% sequence identity to SEQ ID NO: 67.

60. The isolated polypeptide or polypeptide complex of claim 56, wherein the anti-CD28 antibody comprises the scFv, and the scFv comprises an amino acid sequence with at least 80%, 85%, 90%, 95%, 98%, 99%, or 100% sequence identity to SEQ ID NO: 68.

61. The isolated polypeptide or polypeptide complex of claim 56, wherein the anti-CD28 antibody comprises the scFv, and the scFv comprises an amino acid sequence with at least 80%, 85%, 90%, 95%, 98%, 99%, or 100% sequence identity to SEQ ID NO: 69.

62. The isolated polypeptide or polypeptide complex of claim 56, wherein the anti-CD28 antibody comprises the Fab, wherein the Fab comprises an immunoglobulin light chain with at least 80%, 85%, 90%, 95%, 98%, 99%, or 100% sequence identity to SEQ ID NO: 202, and wherein the Fab comprises an immunoglobulin heavy chain with 80%, 85%, 90%, 95%, 98%, 99%, or 100% sequence identity to SEQ ID NO: 203.

63. The isolated polypeptide or polypeptide complex of claim 1, wherein the isolated polypeptide or polypeptide complex is human or humanized.

64. The isolated polypeptide or polypeptide complex of claim 1, wherein the peptide is connected to the CD28 binding domain in a configuration according to A₁-L₁-P₁ wherein A₁ comprises the CD28 binding domain, P₁ comprises the peptide that impairs binding of the CD28 binding domain to CD28, and L₁ comprises a cleavable linker.

65. The isolated polypeptide or polypeptide complex of claim 64, wherein P₁ is connected N-terminal to the cleavable linker and A₁ is connected C-terminal to the cleavable linker.

66. The isolated polypeptide or polypeptide complex of claim 64, wherein P₁ is connected C-terminal to the cleavable linker and A₁ is connected N-terminal to the cleavable linker.

67. The isolated polypeptide or polypeptide complex of claim 64, wherein P₁ is bound to A₁ through ionic interactions, electrostatic interactions, hydrophobic interactions, Pi-stacking interactions, H-bonding interactions, or a combination thereof.

68. The isolated polypeptide or polypeptide complex of claim 64, wherein P₁ is bound to A₁ at or near an antigen binding site.

69. The isolated polypeptide of polypeptide complex of claim 64, wherein P₁ becomes unbound from A₁ when L₁ is cleaved by a tumor specific protease thereby exposing A₁ to CD28.

70. The isolated polypeptide or polypeptide complex of claim 64, wherein P₁ has less than 75% sequence identity to CD28.

71. The isolated polypeptide or polypeptide complex of claim 64, wherein P₁ comprises a de novo amino acid sequence that shares less than 10% sequence identity to CD28.

72. The isolated polypeptide or polypeptide complex of claim 64, wherein P₁ comprises a modified amino acid, or a modified non-natural amino acid, or a combination thereof.

73. The isolated polypeptide or polypeptide complex of claim 64, wherein P₁ does not comprise albumin or an albumin fragment.

74. The isolated polypeptide or polypeptide complex of claim 64, wherein P₁ does not comprise an albumin binding domain.

75. The isolated polypeptide or polypeptide complex of claim 64, wherein L₁ is bound to the N-terminus of A₁.

76. The isolated polypeptide or polypeptide complex of claim 64, wherein L₁ is bound to the C-terminus of A₁.

77. The isolated polypeptide or polypeptide complex of claim 64, wherein L₁ is a peptide sequence having at least 5 to no more than 50 amino acids.

78. The isolated polypeptide or polypeptide complex of claim 64, wherein L₁ is a peptide sequence having at least 10 to no more than 30 amino acids.

79. The isolated polypeptide or polypeptide complex of claim 64, wherein L₁ is a peptide sequence having at least 10 amino acids.

80. The isolated polypeptide or polypeptide complex of claim 64, wherein L₁ is a peptide sequence having at least 18 amino acids.

81. The isolated polypeptide or polypeptide complex of claim 64, wherein L₁ is a peptide sequence having at least 26 amino acids.

82. The isolated polypeptide or polypeptide complex of claim 64, wherein L₁ comprises a formula comprising (G₂S)ₙ, wherein n is an integer from 1 to 3 (SEQ ID NO: 1993).

83. The isolated polypeptide or polypeptide complex of claim 64, wherein L₁ comprises a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1.

84. The isolated polypeptide or polypeptide complex of claim 64, wherein L₁ comprises an amino acid sequence selected from any one of SEQ ID NOs: 36-65.

85. The isolated polypeptide or polypeptide complex of claim 64, wherein L₁ comprises the amino acid sequence of SEQ ID NO: 40.

86. The isolated polypeptide or polypeptide complex of claim 64, wherein L₁ comprises the amino acid sequence of SEQ ID NO: 65.

87. The isolated polypeptide or polypeptide complex of claim 64, wherein L₁ is a substrate for a tumor specific protease.

88. The isolated polypeptide or polypeptide complex of claim 64, wherein P₁ becomes unbound from A, when L₁ is cleaved by the tumor specific protease thereby exposing A₁ to CD28.

89. The isolated polypeptide or polypeptide complex of claim 87, wherein the tumor specific protease is selected from the group consisting of metalloprotease, serine protease, cysteine protease, threonine protease, and aspartic protease.

90. The isolated polypeptide or polypeptide complex of claim 64, wherein L₁ comprises a urokinase cleavable amino acid sequence, a matriptase cleavable amino acid sequence, or a matrix metalloprotease cleavable amino acid sequence.

91. The isolated polypeptide or polypeptide complex of claim 1 wherein the isolated polypeptide or polypeptide complex comprises an amino acid sequence with at least 80%, 85%, 90%, 95%, 99%, or 100% sequence identity to any one of SEQ ID NOs: 74-78, 80, 82, 84-85, 88, 90, and 1393-1444.

92. The isolated polypeptide or polypeptide complex of claim 1, wherein the isolated polypeptide or polypeptide complex comprises an amino acid sequence according to any one of SEQ ID NOs: 74-78, 80, 82, 84-85, 88, 90, and 1393-1444.

93. The isolated polypeptide or polypeptide complex of claim 48, wherein the isolated polypeptide or polypeptide complex comprises an amino acid sequence with at least 80%, 85%, 90%, 95%, 99%, or 100% sequence identity to any one of SEQ ID NOs: 1445-1450.

94. The isolated polypeptide or polypeptide complex of claim 48, wherein the isolated polypeptide or polypeptide complex comprises an amino acid sequence according to any one of SEQ ID NOs: 1445-1450.

95. The isolated polypeptide or polypeptide complex of claim 1, wherein the isolated polypeptide or polypeptide complex further comprises a half-life extending molecule, H₁.

96. The isolated polypeptide or polypeptide complex of claim 95, wherein H₁ comprises a polymer.

97. The isolated polypeptide or polypeptide complex of claim 96, wherein the polymer is polyethylene glycol (PEG).

98. The isolated polypeptide or polypeptide complex of claim 95, wherein H₁ comprises albumin.

99. The isolated polypeptide or polypeptide complex of claim 95, wherein H₁ comprises an Fc domain.

100. The isolated polypeptide or polypeptide complex of claim 98, wherein the albumin is serum albumin.

101. The isolated polypeptide or polypeptide complex of claim 98, wherein the albumin is human serum albumin.

102. The isolated polypeptide or polypeptide complex of claim 95, wherein H₁ comprises a polypeptide, a ligand, or a small molecule.

103. The isolated polypeptide or polypeptide complex of claim 102, wherein the polypeptide, the ligand or the small molecule binds serum protein or a fragment thereof, a circulating immunoglobulin or a fragment thereof, or CD35/CR1.

104. The isolated polypeptide or polypeptide complex of claim 103, wherein the serum protein comprises a thyroxine-binding protein, a transthyretin, a 1-acid glycoprotein, a transferrin, transferrin receptor or a transferrin-binding portion thereof, a fibrinogen, or an albumin.

105. The isolated polypeptide or polypeptide complex of claim 103, wherein the circulating immunoglobulin molecule comprises IgGl, IgG2, IgG3, IgG4, slgA, IgM or IgD.

106. The isolated polypeptide or polypeptide complex of claim 103, wherein the serum protein is albumin.

107. The isolated polypeptide or polypeptide complex of claim 102, wherein the polypeptide is an antibody.

108. The isolated polypeptide or polypeptide complex of claim 107, wherein the antibody comprises a single domain antibody, a single chain variable fragment, or a Fab.

109. The isolated polypeptide or polypeptide complex of claim 108, wherein the single domain antibody comprises a single domain antibody that binds to albumin.

110. The isolated polypeptide or polypeptide complex of claim 107, wherein the single domain antibody is a human or humanized antibody.

111. The isolated polypeptide or polypeptide complex of claim 108, wherein the single domain antibody is 645gH1gL1.

112. The isolated polypeptide or polypeptide complex of claim 108, wherein the single domain antibody is 645dsgH5gL4.

113. The isolated polypeptide or polypeptide complex of claim 108, wherein the single domain antibody is 23-13-A01-sc02.

114. The isolated polypeptide or polypeptide complex of claim 108, wherein the single domain antibody is A10m3 or a fragment thereof.

115. The isolated polypeptide or polypeptide complex of claim 108, wherein the single domain antibody is DOM7r-31.

116. The isolated polypeptide or polypeptide complex of claim 108, wherein the single domain antibody is DOM7h-11-15.

117. The isolated polypeptide or polypeptide complex of claim 108, wherein the single domain antibody is Alb-1, Alb-8, or Alb-23.

118. The isolated polypeptide or polypeptide complex of claim 108, wherein the single domain antibody is 10G or 10E.

119. The isolated polypeptide or polypeptide complex of claim 108, wherein the single domain antibody is SA21.

120. The isolated polypeptide or polypeptide complex of claim 108, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 92, HC-CDR2: SEQ ID NO: 93, and HC-CDR3: SEQ ID NO: 94.

121. The isolated polypeptide or polypeptide complex of claim 108, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 96, HC-CDR2: SEQ ID NO: 97, and HC-CDR3: SEQ ID NO: 98.

122. The isolated polypeptide or polypeptide complex of claim 108, wherein the single domain antibody comprises an amino acid sequence with at least 80%, 85%, 90%, 95%, 99%, or 100% sequence identity to SEQ ID NO: 95.

123. The isolated polypeptide or polypeptide complex of claim 108, wherein the single domain antibody comprises the amino acid sequence of SEQ ID NO: 95.

124. The isolated polypeptide or polypeptide complex of claim 108, wherein the single domain antibody comprises an amino acid sequence with at least 80%, 85%, 90%, 95%, 99%, or 100% sequence identity to SEQ ID NO: 118.

125. The isolated polypeptide or polypeptide complex of claim 108, wherein the single domain antibody comprises the amino acid sequence of SEQ ID NO: 118.

126. The isolated polypeptide or polypeptide complex of claim 108, wherein the single domain antibody comprises an amino acid sequence with at least 80%, 85%, 90%, 95%, 99%, or 100% sequence identity to SEQ ID NO: 1509.

127. The isolated polypeptide or polypeptide complex of claim 108, wherein the single domain antibody comprises the amino acid sequence of SEQ ID NO: 1509.

128. The isolated polypeptide or polypeptide complex of claim 1, wherein the polypeptide or polypeptide complex comprises a modified amino acid, a non-natural amino acid, a modified non-natural amino acid, or a combination thereof.

129. The isolated polypeptide or polypeptide complex of claim 128, wherein the modified amino acid or modified non-natural amino acid comprises a post-translational modification.

130. The isolated polypeptide or polypeptide complex of claim 95, wherein H₁ comprises a linking moiety (L₂) that connects H₁ to P₁.

131. The isolated polypeptide or polypeptide complex of claim 130, wherein L₂ is a peptide sequence having at least 5 to no more than 50 amino acids.

132. The isolated polypeptide or polypeptide complex of claim 130, wherein L₂ is a peptide sequence having at least 10 to no more than 30 amino acids.

133. The isolated polypeptide or polypeptide complex of claim 130, wherein L₂ is a peptide sequence having at least 10 amino acids.

134. The isolated polypeptide or polypeptide complex of claim 130, wherein L₂ is a peptide sequence having at least 18 amino acids.

135. The isolated polypeptide or polypeptide complex of claim 130, wherein L₂ has a formula selected from the group consisting of (G₂S)ₙ, (GS)ₙ, (GSGGS)ₙ (SEQ ID NO: 1994), (GGGS)ₙ (SEQ ID NO: 1995), (GGGGS)ₙ (SEQ ID NO: 1996), and (GSSGGS)ₙ (SEQ ID NO: 1997), wherein n is an integer of at least 1.

136. The isolated polypeptide or polypeptide complex of claim 130, wherein L₂ comprises an amino acid sequence according to GGGGSGGGS (SEQ ID NO: 1998).

137. The isolated polypeptide or polypeptide complex of claim 1, wherein the isolated polypeptide or polypeptide complex comprises an amino acid sequence with at least 80%, 85%, 90%, 95%, 99%, or 100% sequence identity to any one of SEQ ID NOs: 100-104, 106, 108, 110-111, 114, 116, and 1451-1502.

138. The isolated polypeptide or polypeptide complex of claim 1, wherein the isolated polypeptide or polypeptide complex comprises an amino acid sequence according to any one of SEQ ID NOs: 100-104, 106, 108, 110-111, 114, 116, and 1451-1502.

139. The isolated polypeptide or polypeptide complex of claim 48, wherein the isolated polypeptide or polypeptide complex comprises an amino acid sequence with at least 80%, 85%, 90%, 95%, 99%, or 100% sequence identity to any one of SEQ ID NOs: 1503-1508.

140. The isolated polypeptide or polypeptide complex of claim 48, wherein the isolated polypeptide or polypeptide complex comprises an amino acid sequence according to any one of SEQ ID NOs: 1503-1508.

141. A pharmaceutical composition comprising:
(i) the isolated polypeptide or polypeptide complex of claim 1; and
(ii) a pharmaceutically acceptable excipient.

142. An isolated recombinant nucleic acid molecule encoding an isolated polypeptide or polypeptide complex of claim 1.

143. A vector comprising the isolated recombinant nucleic acid molecule of claim 142.

144. A host cell comprising the isolated recombinant nucleic acid molecule of claim 142.

145. A method of treating cancer in a subject in need thereof comprising administering to the subject the isolated polypeptide or polypeptide complex of claim 1.
